SEQUENCE LISTING (100 sequences)

SEQ ID NO: 1 - DNA, 20 bp, Homo sapiens
ggccgagatgtctcgctccg

SEQ ID NO: 2 - DNA, 130 bp, Artificial Sequence
  Synthetic
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 3 - DNA, 800 bp, Homo sapiens
gttctagggtggaaactaagagaatgatgtacctagagggcgctggaagctctaaagccctagcagttactgcttttactattagtggtcgtttttttctcccccccgccccccgacaaatcaacagaacaaagaaaattacctaaacagcaaggacatagggaggaacttcttggcacagaactttccaaacactttttcctgaagggatacaagaagcaagaaaggtactctttcactaggaccttctctgagctgtcctcaggatgcttttgggactatttttcttacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagct

SEQ ID NO: 4 - DNA, 380 bp, Artificial Sequence
  Synthetic
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 5 - DNA, 276 bp, Gallus gallus
tcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 6 - DNA, 1009 bp, Artificial Sequence
  Synthetic
ggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacag

SEQ ID NO: 7 - DNA, 1203 bp, Artificial Sequence
  Synthetic
ggctccggtgcccgtgtgcggaccgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 8 - DNA, 1128 bp, Homo sapiens
atggaaactctttctaatgcaagtggtacttttgccatacgccttttaaagatactgtgtcaagataacccttcgcacaacgtgttctgttctcctgtgagcatctcctctgccctggccatggttctcctaggggcaaagggaaacaccgcaacccagatggcccaggcactgtctttaaacacagaggaagacattcatcgggctttccagtcgcttctcactgaagtgaacaaggctggcacacagtacctgctgagaacggccaacaggctctttggagagaaaacttgtcagttcctctcaacgtttaaggaatcctgtcttcaattctaccatgctgagctgaaggagctttcctttatcagagctgcagaagagtccaggaaacacatcaacacctgggtctcaaaaaagaccgaaggtaaaattgaagagttgttgccgggtagctcaattgatgcagaaaccaggctggttcttgtcaatgccatctacttcaaaggaaagtggaatgaaccgtttgacgaaacatacacaagggaaatgccctttaaaataaaccaggaggagcaaaggccagtgcagatgatgtatcaggaggccacgtttaagctcgcccacgtgggcgaggtgcgcgcgcagctgctggagctgccctacgccaggaaggagctgagcctgctggtgctgctgcctgacgacggcgtggagctcagcacggtggaaaaaagtctcacttttgagaaactcacagcctggaccaagccagactgtatgaagagtactgaggttgaagttctccttccaaaatttaaactacaagaggattatgacatggaatctgtgcttcggcatttgggaattgttgatgccttccaacagggcaaggctgacttgtcggcaatgtcagcggagagagacctgtgtctgtccaagttcgtgcacaagagttttgtggaggtgaatgaagaaggcaccgaggcagcggcagcgtcgagctgctttgtagttgcagagtgctgcatggaatctggccccaggttctgtgctgaccaccctttccttttcttcatcaggcacaacagagccaacagcattctgttctgtggcaggttctcatcgcca

SEQ ID NO: 9 - DNA, 873 bp, Homo sapiens
atgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaaacttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaa

SEQ ID NO: 10 - DNA, 57 bp, Artificial Sequence
  Synthetic
gctactaacttcagcctgctgaagcaggctggagacgtggaggagaaccctggacct

SEQ ID NO: 11 - DNA, 60 bp, Artificial Sequence
  Synthetic
atgtctcgctccgttgccttagctgtgctcgcgctactctctctttctggattagaggct

SEQ ID NO: 12 - DNA, 27 bp, Homo sapiens
gtcatggcgccccgaaccctcttcctg

SEQ ID NO: 13 - DNA, 45 bp, Artificial Sequence
  Synthetic
ggtggaggcggttcaggcggaggtggctctggcggtggcggatcg

SEQ ID NO: 14 - DNA, 297 bp, Homo sapiens
atccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatg

SEQ ID NO: 15 - DNA, 60 bp, Artificial Sequence
  Synthetic
ggtggtggtggttctggtggtggtggttctggcggcggcggctccggtggtggtggatcc

SEQ ID NO: 16 - DNA, 1011 bp, Homo sapiens
ggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttg

SEQ ID NO: 17 - DNA, 122 bp, Artificial Sequence
  Synthetic
aacttgtttattgcagcttataatggttacaaataaagcaatagcatcacaaatttcacaaataaagcatttttttcactgcattctagttgtggtttgtccaaactcatcaatgtatctta

SEQ ID NO: 18 - DNA, 225 bp, Bos taurus
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 19 - DNA, 800 bp, Homo sapiens
ccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttcgcatgtcctagcacctctgggtctatgtggggccacaccgtggggaggaaacagcacgcgacgtttgtagaatgcttggctgtgatacaaagcggtttcgaataattaacttatttgttcccatcacatgtcacttttaaaaaattataagaactacccgttattgacatctttctgtgtgccaaggactttatgtgctttgcgtcatttaattttgaaaacagttatcttccgccatagataactactatggttatcttct

SEQ ID NO: 20 - DNA, 141 bp, Artificial Sequence
  Synthetic
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 21 - DNA, 2944 bp, Artificial Sequence
  Synthetic
atggaaactctttctaatgcaagtggtacttttgccatacgccttttaaagatactgtgtcaagataacccttcgcacaacgtgttctgttctcctgtgagcatctcctctgccctggccatggttctcctaggggcaaagggaaacaccgcaacccagatggcccaggcactgtctttaaacacagaggaagacattcatcgggctttccagtcgcttctcactgaagtgaacaaggctggcacacagtacctgctgagaacggccaacaggctctttggagagaaaacttgtcagttcctctcaacgtttaaggaatcctgtcttcaattctaccatgctgagctgaaggagctttcctttatcagagctgcagaagagtccaggaaacacatcaacacctgggtctcaaaaaagaccgaaggtaaaattgaagagttgttgccgggtagctcaattgatgcagaaaccaggctggttcttgtcaatgccatctacttcaaaggaaagtggaatgaaccgtttgacgaaacatacacaagggaaatgccctttaaaataaaccaggaggagcaaaggccagtgcagatgatgtatcaggaggccacgtttaagctcgcccacgtgggcgaggtgcgcgcgcagctgctggagctgccctacgccaggaaggagctgagcctgctggtgctgctgcctgacgacggcgtggagctcagcacggtggaaaaaagtctcacttttgagaaactcacagcctggaccaagccagactgtatgaagagtactgaggttgaagttctccttccaaaatttaaactacaagaggattatgacatggaatctgtgcttcggcatttgggaattgttgatgccttccaacagggcaaggctgacttgtcggcaatgtcagcggagagagacctgtgtctgtccaagttcgtgcacaagagttttgtggaggtgaatgaagaaggcaccgaggcagcggcagcgtcgagctgctttgtagttgcagagtgctgcatggaatctggccccaggttctgtgctgaccaccctttccttttcttcatcaggcacaacagagccaacagcattctgttctgtggcaggttctcatcgccaggaagcggagctactaacttcagcctgctgaagcaggctggagacgtggaggagaaccctggacctatgtctcgctccgttgccttagctgtgctcgcgctactctctctttctggattagaggctgtcatggcgccccgaaccctcttcctgggtggaggcggttcaggcggaggtggctctggcggtggcggatcgatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggtggtggttctggtggtggtggttctggcggcggcggctccggtggtggtggatccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaatgatagccgctgatcagcctcgactgtgccttctagttgccagccatctgttgtttgcccctcccccgtgccttccttgaccctggaaggtgccactcccactgtcctttcctaataaaatgaggaaattgcatcgcattgtcgagtaggtgtcattctattctggggggtggggtggggcaggacagcaagggggaggattgggaagacaatagcaggcatgctggggatgcggtgggctctatgg

SEQ ID NO: 22 - DNA, 4656 bp, Artificial Sequence
  Synthetic
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacaggggggatccgtttatctgcagaattcgcccttgacgtcgccaccatggaaactctttctaatgcaagtggtacttttgccatacgccttttaaagatactgtgtcaagataacccttcgcacaacgtgttctgttctcctgtgagcatctcctctgccctggccatggttctcctaggggcaaagggaaacaccgcaacccagatggcccaggcactgtctttaaacacagaggaagacattcatcgggctttccagtcgcttctcactgaagtgaacaaggctggcacacagtacctgctgagaacggccaacaggctctttggagagaaaacttgtcagttcctctcaacgtttaaggaatcctgtcttcaattctaccatgctgagctgaaggagctttcctttatcagagctgcagaagagtccaggaaacacatcaacacctgggtctcaaaaaagaccgaaggtaaaattgaagagttgttgccgggtagctcaattgatgcagaaaccaggctggttcttgtcaatgccatctacttcaaaggaaagtggaatgaaccgtttgacgaaacatacacaagggaaatgccctttaaaataaaccaggaggagcaaaggccagtgcagatgatgtatcaggaggccacgtttaagctcgcccacgtgggcgaggtgcgcgcgcagctgctggagctgccctacgccaggaaggagctgagcctgctggtgctgctgcctgacgacggcgtggagctcagcacggtggaaaaaagtctcacttttgagaaactcacagcctggaccaagccagactgtatgaagagtactgaggttgaagttctccttccaaaatttaaactacaagaggattatgacatggaatctgtgcttcggcatttgggaattgttgatgccttccaacagggcaaggctgacttgtcggcaatgtcagcggagagagacctgtgtctgtccaagttcgtgcacaagagttttgtggaggtgaatgaagaaggcaccgaggcagcggcagcgtcgagctgctttgtagttgcagagtgctgcatggaatctggccccaggttctgtgctgaccaccctttccttttcttcatcaggcacaacagagccaacagcattctgttctgtggcaggttctcatcgccaggaagcggagctactaacttcagcctgctgaagcaggctggagacgtggaggagaaccctggacctatgtctcgctccgttgccttagctgtgctcgcgctactctctctttctggattagaggctgtcatggcgccccgaaccctcttcctgggtggaggcggttcaggcggaggtggctctggcggtggcggatcgatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggtggtggttctggtggtggtggttctggcggcggcggctccggtggtggtggatccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaatgatagccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 23 - DNA, 8963 bp, Artificial Sequence
  Synthetic
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgttctagggtggaaactaagagaatgatgtacctagagggcgctggaagctctaaagccctagcagttactgcttttactattagtggtcgtttttttctcccccccgccccccgacaaatcaacagaacaaagaaaattacctaaacagcaaggacatagggaggaacttcttggcacagaactttccaaacactttttcctgaagggatacaagaagcaagaaaggtactctttcactaggaccttctctgagctgtcctcaggatgcttttgggactatttttcttacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctaagcttgtggacgatatcgaattcgcacgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacaggggggatccgtttatctgcagaattcgcccttgacgtcgccaccatggaaactctttctaatgcaagtggtacttttgccatacgccttttaaagatactgtgtcaagataacccttcgcacaacgtgttctgttctcctgtgagcatctcctctgccctggccatggttctcctaggggcaaagggaaacaccgcaacccagatggcccaggcactgtctttaaacacagaggaagacattcatcgggctttccagtcgcttctcactgaagtgaacaaggctggcacacagtacctgctgagaacggccaacaggctctttggagagaaaacttgtcagttcctctcaacgtttaaggaatcctgtcttcaattctaccatgctgagctgaaggagctttcctttatcagagctgcagaagagtccaggaaacacatcaacacctgggtctcaaaaaagaccgaaggtaaaattgaagagttgttgccgggtagctcaattgatgcagaaaccaggctggttcttgtcaatgccatctacttcaaaggaaagtggaatgaaccgtttgacgaaacatacacaagggaaatgccctttaaaataaaccaggaggagcaaaggccagtgcagatgatgtatcaggaggccacgtttaagctcgcccacgtgggcgaggtgcgcgcgcagctgctggagctgccctacgccaggaaggagctgagcctgctggtgctgctgcctgacgacggcgtggagctcagcacggtggaaaaaagtctcacttttgagaaactcacagcctggaccaagccagactgtatgaagagtactgaggttgaagttctccttccaaaatttaaactacaagaggattatgacatggaatctgtgcttcggcatttgggaattgttgatgccttccaacagggcaaggctgacttgtcggcaatgtcagcggagagagacctgtgtctgtccaagttcgtgcacaagagttttgtggaggtgaatgaagaaggcaccgaggcagcggcagcgtcgagctgctttgtagttgcagagtgctgcatggaatctggccccaggttctgtgctgaccaccctttccttttcttcatcaggcacaacagagccaacagcattctgttctgtggcaggttctcatcgccaggaagcggagctactaacttcagcctgctgaagcaggctggagacgtggaggagaaccctggacctatgtctcgctccgttgccttagctgtgctcgcgctactctctctttctggattagaggctgtcatggcgccccgaaccctcttcctgggtggaggcggttcaggcggaggtggctctggcggtggcggatcgatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggtggtggttctggtggtggtggttctggcggcggcggctccggtggtggtggatccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaatgatagccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtcgacccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttcgcatgtcctagcacctctgggtctatgtggggccacaccgtggggaggaaacagcacgcgacgtttgtagaatgcttggctgtgatacaaagcggtttcgaataattaacttatttgttcccatcacatgtcacttttaaaaaattataagaactacccgttattgacatctttctgtgtgccaaggactttatgtgctttgcgtcatttaattttgaaaacagttatcttccgccatagataactactatggttatcttctggtaaccacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 24 - DNA, 1500 bp, Artificial Sequence
  Synthetic
atgtctcgctccgttgccttagctgtgctcgcgctactctctctttctggattagaggctgtcatggcgccccgaaccctcttcctgggtggaggcggttcaggcggaggtggctctggcggtggcggatcgatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggtggtggttctggtggtggtggttctggcggcggcggctccggtggtggtggatccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttg

SEQ ID NO: 25 - DNA, 42 bp, Artificial Sequence
  Synthetic
aggattgggaagacaatagcaggcatgctggggatgcggtgg

SEQ ID NO: 26 - DNA, 59 bp, Artificial Sequence
  Synthetic
gctctggagaatctcacgcagaaggcaggcgtttttcttaaaaaaaaatgcacgaatta

SEQ ID NO: 27 - DNA, 57 bp, Artificial Sequence
  Synthetic
cccttaacgtgagttttcgttccactgagcgtcagaccccgtagaaaagatcaaagg

SEQ ID NO: 28 - DNA, 54 bp, Artificial Sequence
  Synthetic
gtccaacccggtaagacacgacttatcgccactggcagcagccactggtaacag

SEQ ID NO: 29 - DNA, 53 bp, Artificial Sequence
  Synthetic
cacttgccagcgccctagcgcccgctcctttcgctttcttcccttcctttctc

SEQ ID NO: 30 - DNA, 33 bp, Artificial Sequence
  Synthetic
gggcgcgtcagcgggtgttggcgggtgtcgggg

SEQ ID NO: 31 - DNA, 20 bp, Artificial Sequence
  Synthetic
cagacagcaaactcacccag

SEQ ID NO: 32 - DNA, 20 bp, Artificial Sequence
  Synthetic
aaactttgtcccgaccctcc

SEQ ID NO: 33 - DNA, 54 bp, Artificial Sequence
  Synthetic
atggactggacctggatcctgttcctggtggccgccgccaccagggtgcacagc

SEQ ID NO: 34 - DNA, 399 bp, Homo sapiens
ggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttct

SEQ ID NO: 35 - DNA, 78 bp, Artificial Sequence
  Synthetic
agcggcggcggcagcggcggcggcggcagcggcggcggcggcagcggcggcggcggcagcggcggcggcagcctgcag

SEQ ID NO: 36 - DNA, 711 bp, Homo sapiens
atcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccaccta

SEQ ID NO: 37 - DNA, 2684 bp, Artificial Sequence
  Synthetic
atggaaactctttctaatgcaagtggtacttttgccatacgccttttaaagatactgtgtcaagataacccttcgcacaacgtgttctgttctcctgtgagcatctcctctgccctggccatggttctcctaggggcaaagggaaacaccgcaacccagatggcccaggcactgtctttaaacacagaggaagacattcatcgggctttccagtcgcttctcactgaagtgaacaaggctggcacacagtacctgctgagaacggccaacaggctctttggagagaaaacttgtcagttcctctcaacgtttaaggaatcctgtcttcaattctaccatgctgagctgaaggagctttcctttatcagagctgcagaagagtccaggaaacacatcaacacctgggtctcaaaaaagaccgaaggtaaaattgaagagttgttgccgggtagctcaattgatgcagaaaccaggctggttcttgtcaatgccatctacttcaaaggaaagtggaatgaaccgtttgacgaaacatacacaagggaaatgccctttaaaataaaccaggaggagcaaaggccagtgcagatgatgtatcaggaggccacgtttaagctcgcccacgtgggcgaggtgcgcgcgcagctgctggagctgccctacgccaggaaggagctgagcctgctggtgctgctgcctgacgacggcgtggagctcagcacggtggaaaaaagtctcacttttgagaaactcacagcctggaccaagccagactgtatgaagagtactgaggttgaagttctccttccaaaatttaaactacaagaggattatgacatggaatctgtgcttcggcatttgggaattgttgatgccttccaacagggcaaggctgacttgtcggcaatgtcagcggagagagacctgtgtctgtccaagttcgtgcacaagagttttgtggaggtgaatgaagaaggcaccgaggcagcggcagcgtcgagctgctttgtagttgcagagtgctgcatggaatctggccccaggttctgtgctgaccaccctttccttttcttcatcaggcacaacagagccaacagcattctgttctgtggcaggttctcatcgccaggaagcggagctactaacttcagcctgctgaagcaggctggagacgtggaggagaaccctggacctatggactggacctggatcctgttcctggtggccgccgccaccagggtgcacagcggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctagcggcggcggcagcggcggcggcggcagcggcggcggcggcagcggcggcggcggcagcggcggcggcagcctgcagatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccacctatgataaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 38 - DNA, 4395 bp, Artificial Sequence
  Synthetic
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacaggggggatccgtttatctgcagaattcgcccttgacgtcgccaccatggaaactctttctaatgcaagtggtacttttgccatacgccttttaaagatactgtgtcaagataacccttcgcacaacgtgttctgttctcctgtgagcatctcctctgccctggccatggttctcctaggggcaaagggaaacaccgcaacccagatggcccaggcactgtctttaaacacagaggaagacattcatcgggctttccagtcgcttctcactgaagtgaacaaggctggcacacagtacctgctgagaacggccaacaggctctttggagagaaaacttgtcagttcctctcaacgtttaaggaatcctgtcttcaattctaccatgctgagctgaaggagctttcctttatcagagctgcagaagagtccaggaaacacatcaacacctgggtctcaaaaaagaccgaaggtaaaattgaagagttgttgccgggtagctcaattgatgcagaaaccaggctggttcttgtcaatgccatctacttcaaaggaaagtggaatgaaccgtttgacgaaacatacacaagggaaatgccctttaaaataaaccaggaggagcaaaggccagtgcagatgatgtatcaggaggccacgtttaagctcgcccacgtgggcgaggtgcgcgcgcagctgctggagctgccctacgccaggaaggagctgagcctgctggtgctgctgcctgacgacggcgtggagctcagcacggtggaaaaaagtctcacttttgagaaactcacagcctggaccaagccagactgtatgaagagtactgaggttgaagttctccttccaaaatttaaactacaagaggattatgacatggaatctgtgcttcggcatttgggaattgttgatgccttccaacagggcaaggctgacttgtcggcaatgtcagcggagagagacctgtgtctgtccaagttcgtgcacaagagttttgtggaggtgaatgaagaaggcaccgaggcagcggcagcgtcgagctgctttgtagttgcagagtgctgcatggaatctggccccaggttctgtgctgaccaccctttccttttcttcatcaggcacaacagagccaacagcattctgttctgtggcaggttctcatcgccaggaagcggagctactaacttcagcctgctgaagcaggctggagacgtggaggagaaccctggacctatggactggacctggatcctgttcctggtggccgccgccaccagggtgcacagcggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctagcggcggcggcagcggcggcggcggcagcggcggcggcggcagcggcggcggcggcagcggcggcggcagcctgcagatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccacctatgataaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 39 - DNA, 8702 bp, Artificial Sequence
  Synthetic
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgttctagggtggaaactaagagaatgatgtacctagagggcgctggaagctctaaagccctagcagttactgcttttactattagtggtcgtttttttctcccccccgccccccgacaaatcaacagaacaaagaaaattacctaaacagcaaggacatagggaggaacttcttggcacagaactttccaaacactttttcctgaagggatacaagaagcaagaaaggtactctttcactaggaccttctctgagctgtcctcaggatgcttttgggactatttttcttacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctaagcttgtggacgatatcgaattcgcacgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacaggggggatccgtttatctgcagaattcgcccttgacgtcgccaccatggaaactctttctaatgcaagtggtacttttgccatacgccttttaaagatactgtgtcaagataacccttcgcacaacgtgttctgttctcctgtgagcatctcctctgccctggccatggttctcctaggggcaaagggaaacaccgcaacccagatggcccaggcactgtctttaaacacagaggaagacattcatcgggctttccagtcgcttctcactgaagtgaacaaggctggcacacagtacctgctgagaacggccaacaggctctttggagagaaaacttgtcagttcctctcaacgtttaaggaatcctgtcttcaattctaccatgctgagctgaaggagctttcctttatcagagctgcagaagagtccaggaaacacatcaacacctgggtctcaaaaaagaccgaaggtaaaattgaagagttgttgccgggtagctcaattgatgcagaaaccaggctggttcttgtcaatgccatctacttcaaaggaaagtggaatgaaccgtttgacgaaacatacacaagggaaatgccctttaaaataaaccaggaggagcaaaggccagtgcagatgatgtatcaggaggccacgtttaagctcgcccacgtgggcgaggtgcgcgcgcagctgctggagctgccctacgccaggaaggagctgagcctgctggtgctgctgcctgacgacggcgtggagctcagcacggtggaaaaaagtctcacttttgagaaactcacagcctggaccaagccagactgtatgaagagtactgaggttgaagttctccttccaaaatttaaactacaagaggattatgacatggaatctgtgcttcggcatttgggaattgttgatgccttccaacagggcaaggctgacttgtcggcaatgtcagcggagagagacctgtgtctgtccaagttcgtgcacaagagttttgtggaggtgaatgaagaaggcaccgaggcagcggcagcgtcgagctgctttgtagttgcagagtgctgcatggaatctggccccaggttctgtgctgaccaccctttccttttcttcatcaggcacaacagagccaacagcattctgttctgtggcaggttctcatcgccaggaagcggagctactaacttcagcctgctgaagcaggctggagacgtggaggagaaccctggacctatggactggacctggatcctgttcctggtggccgccgccaccagggtgcacagcggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctagcggcggcggcagcggcggcggcggcagcggcggcggcggcagcggcggcggcggcagcggcggcggcagcctgcagatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccacctatgataaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtcgacccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttcgcatgtcctagcacctctgggtctatgtggggccacaccgtggggaggaaacagcacgcgacgtttgtagaatgcttggctgtgatacaaagcggtttcgaataattaacttatttgttcccatcacatgtcacttttaaaaaattataagaactacccgttattgacatctttctgtgtgccaaggactttatgtgctttgcgtcatttaattttgaaaacagttatcttccgccatagataactactatggttatcttctggtaaccacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 40 - DNA, 1242 bp, Artificial Sequence
  Synthetic
atggactggacctggatcctgttcctggtggccgccgccaccagggtgcacagcggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctagcggcggcggcagcggcggcggcggcagcggcggcggcggcagcggcggcggcggcagcggcggcggcagcctgcagatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccaccta

SEQ ID NO: 41 - DNA, 20 bp, Homo sapiens
ggtccatctggtcatagaag

SEQ ID NO: 42 - DNA, 800 bp, Homo sapiens
catatttatggggtatatgtgaatatttattacatgcatagaaggtataatgatcatgtcaggatatttgaggtatccacatttgggattgtttaaagattaaatgaaatagtgttaaaagtatttaatatgcccttcaacaaatgatgaggaaatcttagaatctgctcagactccttcagtttacatattaggaaactgaggcacagaaaggagcagagacttgctcaagtccacccaaagcagtagagcattgtggttaaatgcaggacttcagtcagactgtctgggttcaaatcctggttccacttggacatgggtttccttacataaatcacttcacctctctgagcctcagttttctcatatgcaaagtgaggataataataataccttccttacatggttactgatatgagtattaaatgtgccagctcatgtgcctggcgtataggaggtgctttataaaccttagctgttaccactcatggcattgccaaatgtgggacgggtctcctgactctctggtgtgagattgatggaatccacactttccagttcccttttctacctcctgggtatcttctcatatggttgtaagttccttggaggaagggaatgtggcttgctctctccaccacgctgagcatataagaggtgctgaatgagcgcttttattcactcctctcatccccagccctcaccagctgggagttgttgtaggtgtcaattttctgcctctttccaacaccctgtgaggtgactgagcattgtcttccctcccaggcagctcacagtgt

SEQ ID NO: 43 - DNA, 800 bp, Homo sapiens
tgaccagatggacctggctggagaagaagagattgagctctactcaggtgggccctcctccctctggtctcttccggtatcccccacccctcagcttgctgtagagacggcaatcaggggaaattctggtccctgccctcccgtcagcaccacggacagctcccacgtctgtgggacgctctctgcagatggggatgatctcccagccctgccccgcctctccctcgttccccaccagccctctttccagaaatttccttcttcatccaagggacttttcctcccagaacccgacacagacaccatcaactgcgaccagttcagcaggctgttgtgtgacatggaaggtgatgaagagaccagggaggcttatgccaatatcggtgaggaagcacctgagcccagaaaaggacaatcaagggcaagagttctttgctgccacttgtcaatatcacccattcatcatgagccacgtcagtcccctcccacagaaatcattgcaagggggatgcggagcaatggctggaggaacggagactccagggaagagaggggagatggaggccagtgggggaaataggccccttcactaatgaccaccaagaaaacaaaatctcatgtttacatcctccacctccatttctatacgcatttctgcttcttgctcttctgtccatcctttctacaaagcccataccatacacccctttcccttttcctcccagctccttagccaagctactctagtatttgtaataactagcatttactggatactcatagtatgctcattgctgtcc

SEQ ID NO: 44 - DNA, 8963 bp, Artificial Sequence
  Synthetic
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtcatatttatggggtatatgtgaatatttattacatgcatagaaggtataatgatcatgtcaggatatttgaggtatccacatttgggattgtttaaagattaaatgaaatagtgttaaaagtatttaatatgcccttcaacaaatgatgaggaaatcttagaatctgctcagactccttcagtttacatattaggaaactgaggcacagaaaggagcagagacttgctcaagtccacccaaagcagtagagcattgtggttaaatgcaggacttcagtcagactgtctgggttcaaatcctggttccacttggacatgggtttccttacataaatcacttcacctctctgagcctcagttttctcatatgcaaagtgaggataataataataccttccttacatggttactgatatgagtattaaatgtgccagctcatgtgcctggcgtataggaggtgctttataaaccttagctgttaccactcatggcattgccaaatgtgggacgggtctcctgactctctggtgtgagattgatggaatccacactttccagttcccttttctacctcctgggtatcttctcatatggttgtaagttccttggaggaagggaatgtggcttgctctctccaccacgctgagcatataagaggtgctgaatgagcgcttttattcactcctctcatccccagccctcaccagctgggagttgttgtaggtgtcaattttctgcctctttccaacaccctgtgaggtgactgagcattgtcttccctcccaggcagctcacagtgtaagcttgtggacgatatcgaattcgcacgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacaggggggatccgtttatctgcagaattcgcccttgacgtcgccaccatggaaactctttctaatgcaagtggtacttttgccatacgccttttaaagatactgtgtcaagataacccttcgcacaacgtgttctgttctcctgtgagcatctcctctgccctggccatggttctcctaggggcaaagggaaacaccgcaacccagatggcccaggcactgtctttaaacacagaggaagacattcatcgggctttccagtcgcttctcactgaagtgaacaaggctggcacacagtacctgctgagaacggccaacaggctctttggagagaaaacttgtcagttcctctcaacgtttaaggaatcctgtcttcaattctaccatgctgagctgaaggagctttcctttatcagagctgcagaagagtccaggaaacacatcaacacctgggtctcaaaaaagaccgaaggtaaaattgaagagttgttgccgggtagctcaattgatgcagaaaccaggctggttcttgtcaatgccatctacttcaaaggaaagtggaatgaaccgtttgacgaaacatacacaagggaaatgccctttaaaataaaccaggaggagcaaaggccagtgcagatgatgtatcaggaggccacgtttaagctcgcccacgtgggcgaggtgcgcgcgcagctgctggagctgccctacgccaggaaggagctgagcctgctggtgctgctgcctgacgacggcgtggagctcagcacggtggaaaaaagtctcacttttgagaaactcacagcctggaccaagccagactgtatgaagagtactgaggttgaagttctccttccaaaatttaaactacaagaggattatgacatggaatctgtgcttcggcatttgggaattgttgatgccttccaacagggcaaggctgacttgtcggcaatgtcagcggagagagacctgtgtctgtccaagttcgtgcacaagagttttgtggaggtgaatgaagaaggcaccgaggcagcggcagcgtcgagctgctttgtagttgcagagtgctgcatggaatctggccccaggttctgtgctgaccaccctttccttttcttcatcaggcacaacagagccaacagcattctgttctgtggcaggttctcatcgccaggaagcggagctactaacttcagcctgctgaagcaggctggagacgtggaggagaaccctggacctatgtctcgctccgttgccttagctgtgctcgcgctactctctctttctggattagaggctgtcatggcgccccgaaccctcttcctgggtggaggcggttcaggcggaggtggctctggcggtggcggatcgatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggtggtggttctggtggtggtggttctggcggcggcggctccggtggtggtggatccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaatgatagccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtcgactgaccagatggacctggctggagaagaagagattgagctctactcaggtgggccctcctccctctggtctcttccggtatcccccacccctcagcttgctgtagagacggcaatcaggggaaattctggtccctgccctcccgtcagcaccacggacagctcccacgtctgtgggacgctctctgcagatggggatgatctcccagccctgccccgcctctccctcgttccccaccagccctctttccagaaatttccttcttcatccaagggacttttcctcccagaacccgacacagacaccatcaactgcgaccagttcagcaggctgttgtgtgacatggaaggtgatgaagagaccagggaggcttatgccaatatcggtgaggaagcacctgagcccagaaaaggacaatcaagggcaagagttctttgctgccacttgtcaatatcacccattcatcatgagccacgtcagtcccctcccacagaaatcattgcaagggggatgcggagcaatggctggaggaacggagactccagggaagagaggggagatggaggccagtgggggaaataggccccttcactaatgaccaccaagaaaacaaaatctcatgtttacatcctccacctccatttctatacgcatttctgcttcttgctcttctgtccatcctttctacaaagcccataccatacacccctttcccttttcctcccagctccttagccaagctactctagtatttgtaataactagcatttactggatactcatagtatgctcattgctgtccggtaaccacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 45 - DNA, 8963 bp, Artificial Sequence; identical to SEQ ID NO: 44
  Synthetic

SEQ ID NO: 46 - DNA, 3050 bp, Artificial Sequence
  Synthetic
atgacttttaacagttttgaaggatctaaaacttgtgtacctgcagacatcaataaggaagaagaatttgtagaagagtttaatagattaaaaacttttgctaattttccaagtggtagtcctgtttcagcatcaacactggcacgagcagggtttctttatactggtgaaggagataccgtgcggtgctttagttgtcatgcagctgtagatagatggcaatatggagactcagcagttggaagacacaggaaagtatccccaaattgcagatttatcaacggcttttatcttgaaaatagtgccacgcagtctacaaattctggtatccagaatggtcagtacaaagttgaaaactatctgggaagcagagatcattttgccttagacaggccatctgagacacatgcagactatcttttgagaactgggcaggttgtagatatatcagacaccatatacccgaggaaccctgccatgtatagtgaagaagctagattaaagtcctttcagaactggccagactatgctcacctaaccccaagagagttagcaagtgctggactctactacacaggtattggtgaccaagtgcagtgcttttgttgtggtggaaaactgaaaaattgggaaccttgtgatcgtgcctggtcagaacacaggcgacactttcctaattgcttctttgttttgggccggaatcttaatattcgaagtgaatctgatgctgtgagttctgataggaatttcccaaattcaacaaatcttccaagaaatccatccatggcagattatgaagcacggatctttacttttgggacatggatatactcagttaacaaggagcagcttgcaagagctggattttatgctttaggtgaaggtgataaagtaaagtgctttcactgtggaggagggctaactgattggaagcccagtgaagacccttgggaacaacatgctaaatggtatccagggtgcaaatatctgttagaacagaagggacaagaatatataaacaatattcatttaactcattcacttgaggagtgtctggtaagaactactgagaaaacaccatcactaactagaagaattgatgataccatcttccaaaatcctatggtacaagaagctatacgaatggggttcagtttcaaggacattaagaaaataatggaggaaaaaattcagatatctgggagcaactataaatcacttgaggttctggttgcagatctagtgaatgctcagaaagacagtatgcaagatgagtcaagtcagacttcattacagaaagagattagtactgaagagcagctaaggcgcctgcaagaggagaagctttgcaaaatctgtatggatagaaatattgctatcgtttttgttccttgtggacatctagtcacttgtaaacaatgtgctgaagcagttgacaagtgtcccatgtgctacacagtcattactttcaagcaaaaaatttttatgtctggaagcggagctactaacttcagcctgctgaagcaggctggagacgtggaggagaaccctggacctatggactggacctggatcctgttcctggtggccgccgccaccagggtgcacagcggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctagcggcggcggcagcggcggcggcggcagcggcggcggcggcagcggcggcggcggcagcggcggcggcagcctgcagatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccacctataatgatagccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 47 - DNA, 4761 bp, Artificial Sequence
  Synthetic
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacaggggggatccgtttatctgcagaattcgcccttgacgtcgccaccatgacttttaacagttttgaaggatctaaaacttgtgtacctgcagacatcaataaggaagaagaatttgtagaagagtttaatagattaaaaacttttgctaattttccaagtggtagtcctgtttcagcatcaacactggcacgagcagggtttctttatactggtgaaggagataccgtgcggtgctttagttgtcatgcagctgtagatagatggcaatatggagactcagcagttggaagacacaggaaagtatccccaaattgcagatttatcaacggcttttatcttgaaaatagtgccacgcagtctacaaattctggtatccagaatggtcagtacaaagttgaaaactatctgggaagcagagatcattttgccttagacaggccatctgagacacatgcagactatcttttgagaactgggcaggttgtagatatatcagacaccatatacccgaggaaccctgccatgtatagtgaagaagctagattaaagtcctttcagaactggccagactatgctcacctaaccccaagagagttagcaagtgctggactctactacacaggtattggtgaccaagtgcagtgcttttgttgtggtggaaaactgaaaaattgggaaccttgtgatcgtgcctggtcagaacacaggcgacactttcctaattgcttctttgttttgggccggaatcttaatattcgaagtgaatctgatgctgtgagttctgataggaatttcccaaattcaacaaatcttccaagaaatccatccatggcagattatgaagcacggatctttacttttgggacatggatatactcagttaacaaggagcagcttgcaagagctggattttatgctttaggtgaaggtgataaagtaaagtgctttcactgtggaggagggctaactgattggaagcccagtgaagacccttgggaacaacatgctaaatggtatccagggtgcaaatatctgttagaacagaagggacaagaatatataaacaatattcatttaactcattcacttgaggagtgtctggtaagaactactgagaaaacaccatcactaactagaagaattgatgataccatcttccaaaatcctatggtacaagaagctatacgaatggggttcagtttcaaggacattaagaaaataatggaggaaaaaattcagatatctgggagcaactataaatcacttgaggttctggttgcagatctagtgaatgctcagaaagacagtatgcaagatgagtcaagtcagacttcattacagaaagagattagtactgaagagcagctaaggcgcctgcaagaggagaagctttgcaaaatctgtatggatagaaatattgctatcgtttttgttccttgtggacatctagtcacttgtaaacaatgtgctgaagcagttgacaagtgtcccatgtgctacacagtcattactttcaagcaaaaaatttttatgtctggaagcggagctactaacttcagcctgctgaagcaggctggagacgtggaggagaaccctggacctatggactggacctggatcctgttcctggtggccgccgccaccagggtgcacagcggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctagcggcggcggcagcggcggcggcggcagcggcggcggcggcagcggcggcggcggcagcggcggcggcagcctgcagatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccacctataatgatagccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 48 - DNA, 9068 bp, Artificial Sequence
  Synthetic
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgttctagggtggaaactaagagaatgatgtacctagagggcgctggaagctctaaagccctagcagttactgcttttactattagtggtcgtttttttctcccccccgccccccgacaaatcaacagaacaaagaaaattacctaaacagcaaggacatagggaggaacttcttggcacagaactttccaaacactttttcctgaagggatacaagaagcaagaaaggtactctttcactaggaccttctctgagctgtcctcaggatgcttttgggactatttttcttacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctaagcttgtggacgatatcgaattcgcacgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacaggggggatccgtttatctgcagaattcgcccttgacgtcgccaccatgacttttaacagttttgaaggatctaaaacttgtgtacctgcagacatcaataaggaagaagaatttgtagaagagtttaatagattaaaaacttttgctaattttccaagtggtagtcctgtttcagcatcaacactggcacgagcagggtttctttatactggtgaaggagataccgtgcggtgctttagttgtcatgcagctgtagatagatggcaatatggagactcagcagttggaagacacaggaaagtatccccaaattgcagatttatcaacggcttttatcttgaaaatagtgccacgcagtctacaaattctggtatccagaatggtcagtacaaagttgaaaactatctgggaagcagagatcattttgccttagacaggccatctgagacacatgcagactatcttttgagaactgggcaggttgtagatatatcagacaccatatacccgaggaaccctgccatgtatagtgaagaagctagattaaagtcctttcagaactggccagactatgctcacctaaccccaagagagttagcaagtgctggactctactacacaggtattggtgaccaagtgcagtgcttttgttgtggtggaaaactgaaaaattgggaaccttgtgatcgtgcctggtcagaacacaggcgacactttcctaattgcttctttgttttgggccggaatcttaatattcgaagtgaatctgatgctgtgagttctgataggaatttcccaaattcaacaaatcttccaagaaatccatccatggcagattatgaagcacggatctttacttttgggacatggatatactcagttaacaaggagcagcttgcaagagctggattttatgctttaggtgaaggtgataaagtaaagtgctttcactgtggaggagggctaactgattggaagcccagtgaagacccttgggaacaacatgctaaatggtatccagggtgcaaatatctgttagaacagaagggacaagaatatataaacaatattcatttaactcattcacttgaggagtgtctggtaagaactactgagaaaacaccatcactaactagaagaattgatgataccatcttccaaaatcctatggtacaagaagctatacgaatggggttcagtttcaaggacattaagaaaataatggaggaaaaaattcagatatctgggagcaactataaatcacttgaggttctggttgcagatctagtgaatgctcagaaagacagtatgcaagatgagtcaagtcagacttcattacagaaagagattagtactgaagagcagctaaggcgcctgcaagaggagaagctttgcaaaatctgtatggatagaaatattgctatcgtttttgttccttgtggacatctagtcacttgtaaacaatgtgctgaagcagttgacaagtgtcccatgtgctacacagtcattactttcaagcaaaaaatttttatgtctggaagcggagctactaacttcagcctgctgaagcaggctggagacgtggaggagaaccctggacctatggactggacctggatcctgttcctggtggccgccgccaccagggtgcacagcggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctagcggcggcggcagcggcggcggcggcagcggcggcggcggcagcggcggcggcggcagcggcggcggcagcctgcagatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccacctataatgatagccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtcgacccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttcgcatgtcctagcacctctgggtctatgtggggccacaccgtggggaggaaacagcacgcgacgtttgtagaatgcttggctgtgatacaaagcggtttcgaataattaacttatttgttcccatcacatgtcacttttaaaaaattataagaactacccgttattgacatctttctgtgtgccaaggactttatgtgctttgcgtcatttaattttgaaaacagttatcttccgccatagataactactatggttatcttctggtaaccacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 49 - DNA, 20 bp, Homo sapiens
tcgccgctgccgcggggaca

SEQ ID NO: 50 - DNA, 20 bp, Homo sapiens
gacatggtcctctgcgttca

SEQ ID NO: 51 - DNA, 20 bp, Homo sapiens
gtccgctccacagccagcaa

SEQ ID NO: 52 - DNA, 20 bp, Homo sapiens
gttccagggacggggcccac

SEQ ID NO: 53 - DNA, 20 bp, Homo sapiens
tcgggcctcgctggccgtaa

SEQ ID NO: 54 - DNA, 20 bp, Homo sapiens
cgtactaagaacgtgccttc

SEQ ID NO: 55 - DNA, 20 bp, Homo sapiens
gggttccattacggccagcg

SEQ ID NO: 56 - DNA, 20 bp, Homo sapiens
caggtgttgtcgggcctcgc

SEQ ID NO: 57 - DNA, 20 bp, Homo sapiens
tactcaatgcgtacattggt

SEQ ID NO: 58 - DNA, 20 bp, Homo sapiens
aaggctgaccacatccggaa

SEQ ID NO: 59 - DNA, 20 bp, Homo sapiens
tacattggtggggccacgag

SEQ ID NO: 60 - DNA, 20 bp, Homo sapiens
ctgtcagtgaaaaccactcg

SEQ ID NO: 61 - DNA, 20 bp, Homo sapiens
ggattgctcaacaaccatgc

SEQ ID NO: 62 - DNA, 20 bp, Homo sapiens
gattgctcaacaaccatgct

SEQ ID NO: 63 - DNA, 20 bp, Homo sapiens
gtgactgacatcaactccaa

SEQ ID NO: 64 - DNA, 20 bp, Homo sapiens
cacttgggcattaacacttt

SEQ ID NO: 65 - DNA, 20 bp, Homo sapiens
ttggaaggcctgcatcatga

SEQ ID NO: 66 - DNA, 20 bp, Homo sapiens
actccaagggattggaattg

SEQ ID NO: 67 - DNA, 20 bp, Homo sapiens
ctagggactgcacagtcaat

SEQ ID NO: 68 - DNA, 10645 bp, Artificial Sequence
  Synthetic
agtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacaggggggatccgtttatctgcagaattcgcccttgacgtcgccaccatggaaactctttctaatgcaagtggtacttttgccatacgccttttaaagatactgtgtcaagataacccttcgcacaacgtgttctgttctcctgtgagcatctcctctgccctggccatggttctcctaggggcaaagggaaacaccgcaacccagatggcccaggcactgtctttaaacacagaggaagacattcatcgggctttccagtcgcttctcactgaagtgaacaaggctggcacacagtacctgctgagaacggccaacaggctctttggagagaaaacttgtcagttcctctcaacgtttaaggaatcctgtcttcaattctaccatgctgagctgaaggagctttcctttatcagagctgcagaagagtccaggaaacacatcaacacctgggtctcaaaaaagaccgaaggtaaaattgaagagttgttgccgggtagctcaattgatgcagaaaccaggctggttcttgtcaatgccatctacttcaaaggaaagtggaatgaaccgtttgacgaaacatacacaagggaaatgccctttaaaataaaccaggaggagcaaaggccagtgcagatgatgtatcaggaggccacgtttaagctcgcccacgtgggcgaggtgcgcgcgcagctgctggagctgccctacgccaggaaggagctgagcctgctggtgctgctgcctgacgacggcgtggagctcagcacggtggaaaaaagtctcacttttgagaaactcacagcctggaccaagccagactgtatgaagagtactgaggttgaagttctccttccaaaatttaaactacaagaggattatgacatggaatctgtgcttcggcatttgggaattgttgatgccttccaacagggcaaggctgacttgtcggcaatgtcagcggagagagacctgtgtctgtccaagttcgtgcacaagagttttgtggaggtgaatgaagaaggcaccgaggcagcggcagcgtcgagctgctttgtagttgcagagtgctgcatggaatctggccccaggttctgtgctgaccaccctttccttttcttcatcaggcacaacagagccaacagcattctgttctgtggcaggttctcatcgccaggaagcggagctactaacttcagcctgctgaagcaggctggagacgtggaggagaaccctggacctatggactggacctggatcctgttcctggtggccgccgccaccagggtgcacagcggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctagcggcggcggcagcggcggcggcggcagcggcggcggcggcagcggcggcggcggcagcggcggcggcagcctgcagatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccacctatgataaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtcgacccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttcgcatgtcctagcacctctgggtctatgtggggccacaccgtggggaggaaacagcacgcgacgtttgtagaatgcttggctgtgatacaaagcggtttcgaataattaacttatttgttcccatcacatgtcacttttaaaaaattataagaactacccgttattgacatctttctgtgtgccaaggactttatgtgctttgcgtcatttaattttgaaaacagttatcttccgccatagataactactatggttatcttctggtaaccacgtgcggaccgggctccggtgcccgtgtgcggaccgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacttgacgtcgccaccatgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaaacttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaaccgctgatcagcctcgaaacttgtttattgcagcttataatggttacaaataaagcaatagcatcacaaatttcacaaataaagcatttttttcactgcattctagttgtggtttgtccaaactcatcaatgtatcttaggcgcctgatgcggtattttctccttacgcatctgtgcggtatttcacaccgcatacagtactgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtgcggccgcacgcgtgttctagggtggaaactaagagaatgatgtacctagagggcgctggaagctctaaagccctagcagttactgcttttactattagtggtcgtttttttctcccccccgccccccgacaaatcaacagaacaaagaaaattacctaaacagcaaggacatagggaggaacttcttggcacagaactttccaaacactttttcctgaagggatacaagaagcaagaaaggtactctttcactaggaccttctctgagctgtcctcaggatgcttttgggactatttttcttacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctaagcttgtggacgatatcgaattcgcacgacattgattattgactagttattaat

SEQ ID NO: 69 - DNA, 63 bp, Homo sapiens
atggcgcttccggtgacagcactgctcctccccttggcgctgttgctccacgcagcaaggccg

SEQ ID NO: 70 - DNA, 729 bp, Artificial Sequence
  Synthetic
cagatccagctgcagcagagcggccccgaggtggtgaagcccggcgccagcgtgaagatcagctgcaaggccagcggctacaccttcaccgactactacatcacctgggtgaagcagaagcccggccagggcctggagtggatcggctggatctaccccggcagcggcaacaccaagtacaacgagaagttcaagggcaaggccaccctgaccgtggacaccagcagcagcaccgccttcatgcagctgagcagcctgaccagcgaggacaccgccgtgtacttctgcgccaactacggcaactactggttcgcctactggggccagggcacccaggtgaccgtgagcgccggcggcggcggcagcggcggcggcggcagcggcggcggcggcagcgacatcgtgctgacccagagccccgccagcctggccgtgagcctgggccagagagccaccatcagctgcaaggccagccagagcgtggacttcgacggcgacagctacatgaactggtaccagcagaagcccggccagccccccaaggtgctgatctacgccgccagcaacctggagagcggcatccccgccagattcagcggcagcggcagcggcaccgacttcaccctgaacatccaccccgtggaggaggaggacgccgccacctactactgccagcagagcaacgaggacccctggaccttcggcggcggcaccaagctggagatcaag

SEQ ID NO: 71 - DNA, 264 bp, Artificial Sequence
  Synthetic
agcgccgccgccttcgtgcccgtgttcctgcccgccaagcccaccaccacccccgcccccagaccccccacccccgcccccaccatcgccagccagcccctgagcctgagacccgaggcctgcagacccgccgccggcggcgccgtgcacaccagaggcctggacttcgcctgcgacatctacatctgggcccccctggccggcacctgcggcgtgctgctgctgagcctggtgatcaccctgtactgcaaccacagaaacaga

SEQ ID NO: 72 - DNA, 120 bp, Artificial Sequence
  Synthetic
agcaagagaagcagactgctgcacagcgactacatgaacatgacccccagaagacccggccccaccagaaagcactaccagccctacgccccccccagagacttcgccgcctacagaagc

SEQ ID NO: 73 - DNA, 336 bp, Artificial Sequence
  Synthetic
agagtgaagttcagcagaagcgccgacgcccccgcctaccagcagggccagaaccagctgtacaacgagctgaacctgggcagaagagaggagtacgacgtgctggacaagagaagaggcagagaccccgagatgggcggcaagcccagaagaaagaacccccaggagggcctgtacaacgagctgcagaaggacaagatggccgaggcctacagcgagatcggcatgaagggcgagagaagaagaggcaagggccacgacggcctgtaccagggcctgagcaccgccaccaaggacacctacgacgccctgcacatgcaggccctgccccccaga

SEQ ID NO: 74 - DNA, 1512 bp, Artificial Sequence
  Synthetic
atggcgcttccggtgacagcactgctcctccccttggcgctgttgctccacgcagcaaggccgcagatccagctgcagcagagcggccccgaggtggtgaagcccggcgccagcgtgaagatcagctgcaaggccagcggctacaccttcaccgactactacatcacctgggtgaagcagaagcccggccagggcctggagtggatcggctggatctaccccggcagcggcaacaccaagtacaacgagaagttcaagggcaaggccaccctgaccgtggacaccagcagcagcaccgccttcatgcagctgagcagcctgaccagcgaggacaccgccgtgtacttctgcgccaactacggcaactactggttcgcctactggggccagggcacccaggtgaccgtgagcgccggcggcggcggcagcggcggcggcggcagcggcggcggcggcagcgacatcgtgctgacccagagccccgccagcctggccgtgagcctgggccagagagccaccatcagctgcaaggccagccagagcgtggacttcgacggcgacagctacatgaactggtaccagcagaagcccggccagccccccaaggtgctgatctacgccgccagcaacctggagagcggcatccccgccagattcagcggcagcggcagcggcaccgacttcaccctgaacatccaccccgtggaggaggaggacgccgccacctactactgccagcagagcaacgaggacccctggaccttcggcggcggcaccaagctggagatcaagagcgccgccgccttcgtgcccgtgttcctgcccgccaagcccaccaccacccccgcccccagaccccccacccccgcccccaccatcgccagccagcccctgagcctgagacccgaggcctgcagacccgccgccggcggcgccgtgcacaccagaggcctggacttcgcctgcgacatctacatctgggcccccctggccggcacctgcggcgtgctgctgctgagcctggtgatcaccctgtactgcaaccacagaaacagaagcaagagaagcagactgctgcacagcgactacatgaacatgacccccagaagacccggccccaccagaaagcactaccagccctacgccccccccagagacttcgccgcctacagaagcagagtgaagttcagcagaagcgccgacgcccccgcctaccagcagggccagaaccagctgtacaacgagctgaacctgggcagaagagaggagtacgacgtgctggacaagagaagaggcagagaccccgagatgggcggcaagcccagaagaaagaacccccaggagggcctgtacaacgagctgcagaaggacaagatggccgaggcctacagcgagatcggcatgaagggcgagagaagaagaggcaagggccacgacggcctgtaccagggcctgagcaccgccaccaaggacacctacgacgccctgcacatgcaggccctgccccccaga

SEQ ID NO: 75 - DNA, 3329 bp, Artificial Sequence
  Synthetic
atggcgcttccggtgacagcactgctcctccccttggcgctgttgctccacgcagcaaggccgcagatccagctgcagcagagcggccccgaggtggtgaagcccggcgccagcgtgaagatcagctgcaaggccagcggctacaccttcaccgactactacatcacctgggtgaagcagaagcccggccagggcctggagtggatcggctggatctaccccggcagcggcaacaccaagtacaacgagaagttcaagggcaaggccaccctgaccgtggacaccagcagcagcaccgccttcatgcagctgagcagcctgaccagcgaggacaccgccgtgtacttctgcgccaactacggcaactactggttcgcctactggggccagggcacccaggtgaccgtgagcgccggcggcggcggcagcggcggcggcggcagcggcggcggcggcagcgacatcgtgctgacccagagccccgccagcctggccgtgagcctgggccagagagccaccatcagctgcaaggccagccagagcgtggacttcgacggcgacagctacatgaactggtaccagcagaagcccggccagccccccaaggtgctgatctacgccgccagcaacctggagagcggcatccccgccagattcagcggcagcggcagcggcaccgacttcaccctgaacatccaccccgtggaggaggaggacgccgccacctactactgccagcagagcaacgaggacccctggaccttcggcggcggcaccaagctggagatcaagagcgccgccgccttcgtgcccgtgttcctgcccgccaagcccaccaccacccccgcccccagaccccccacccccgcccccaccatcgccagccagcccctgagcctgagacccgaggcctgcagacccgccgccggcggcgccgtgcacaccagaggcctggacttcgcctgcgacatctacatctgggcccccctggccggcacctgcggcgtgctgctgctgagcctggtgatcaccctgtactgcaaccacagaaacagaagcaagagaagcagactgctgcacagcgactacatgaacatgacccccagaagacccggccccaccagaaagcactaccagccctacgccccccccagagacttcgccgcctacagaagcagagtgaagttcagcagaagcgccgacgcccccgcctaccagcagggccagaaccagctgtacaacgagctgaacctgggcagaagagaggagtacgacgtgctggacaagagaagaggcagagaccccgagatgggcggcaagcccagaagaaagaacccccaggagggcctgtacaacgagctgcagaaggacaagatggccgaggcctacagcgagatcggcatgaagggcgagagaagaagaggcaagggccacgacggcctgtaccagggcctgagcaccgccaccaaggacacctacgacgccctgcacatgcaggccctgccccccagaggaagcggagctactaacttcagcctgctgaagcaggctggagacgtggaggagaaccctggacctatgtctcgctccgttgccttagctgtgctcgcgctactctctctttctggattagaggctgtcatggcgccccgaaccctcttcctgggtggaggcggttcaggcggaggtggctctggcggtggcggatcgatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggtggtggttctggtggtggtggttctggcggcggcggctccggtggtggtggatccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaatgatagccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 76 - DNA, 5040 bp, Artificial Sequence
  Synthetic
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacaggggggatccgtttatctgcagaattcgcccttgacgtcgccaccatggcgcttccggtgacagcactgctcctccccttggcgctgttgctccacgcagcaaggccgcagatccagctgcagcagagcggccccgaggtggtgaagcccggcgccagcgtgaagatcagctgcaaggccagcggctacaccttcaccgactactacatcacctgggtgaagcagaagcccggccagggcctggagtggatcggctggatctaccccggcagcggcaacaccaagtacaacgagaagttcaagggcaaggccaccctgaccgtggacaccagcagcagcaccgccttcatgcagctgagcagcctgaccagcgaggacaccgccgtgtacttctgcgccaactacggcaactactggttcgcctactggggccagggcacccaggtgaccgtgagcgccggcggcggcggcagcggcggcggcggcagcggcggcggcggcagcgacatcgtgctgacccagagccccgccagcctggccgtgagcctgggccagagagccaccatcagctgcaaggccagccagagcgtggacttcgacggcgacagctacatgaactggtaccagcagaagcccggccagccccccaaggtgctgatctacgccgccagcaacctggagagcggcatccccgccagattcagcggcagcggcagcggcaccgacttcaccctgaacatccaccccgtggaggaggaggacgccgccacctactactgccagcagagcaacgaggacccctggaccttcggcggcggcaccaagctggagatcaagagcgccgccgccttcgtgcccgtgttcctgcccgccaagcccaccaccacccccgcccccagaccccccacccccgcccccaccatcgccagccagcccctgagcctgagacccgaggcctgcagacccgccgccggcggcgccgtgcacaccagaggcctggacttcgcctgcgacatctacatctgggcccccctggccggcacctgcggcgtgctgctgctgagcctggtgatcaccctgtactgcaaccacagaaacagaagcaagagaagcagactgctgcacagcgactacatgaacatgacccccagaagacccggccccaccagaaagcactaccagccctacgccccccccagagacttcgccgcctacagaagcagagtgaagttcagcagaagcgccgacgcccccgcctaccagcagggccagaaccagctgtacaacgagctgaacctgggcagaagagaggagtacgacgtgctggacaagagaagaggcagagaccccgagatgggcggcaagcccagaagaaagaacccccaggagggcctgtacaacgagctgcagaaggacaagatggccgaggcctacagcgagatcggcatgaagggcgagagaagaagaggcaagggccacgacggcctgtaccagggcctgagcaccgccaccaaggacacctacgacgccctgcacatgcaggccctgccccccagaggaagcggagctactaacttcagcctgctgaagcaggctggagacgtggaggagaaccctggacctatgtctcgctccgttgccttagctgtgctcgcgctactctctctttctggattagaggctgtcatggcgccccgaaccctcttcctgggtggaggcggttcaggcggaggtggctctggcggtggcggatcgatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggtggtggttctggtggtggtggttctggcggcggcggctccggtggtggtggatccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaatgatagccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 77 - DNA, 11265 bp, Artificial Sequence
  Synthetic
agaatctgctcagactccttcagtttacatattaggaaactgaggcacagaaaggagcagagacttgctcaagtccacccaaagcagtagagcattgtggttaaatgcaggacttcagtcagactgtctgggttcaaatcctggttccacttggacatgggtttccttacataaatcacttcacctctctgagcctcagttttctcatatgcaaagtgaggataataataataccttccttacatggttactgatatgagtattaaatgtgccagctcatgtgcctggcgtataggaggtgctttataaaccttagctgttaccactcatggcattgccaaatgtgggacgggtctcctgactctctggtgtgagattgatggaatccacactttccagttcccttttctacctcctgggtatcttctcatatggttgtaagttccttggaggaagggaatgtggcttgctctctccaccacgctgagcatataagaggtgctgaatgagcgcttttattcactcctctcatccccagccctcaccagctgggagttgttgtaggtgtcaattttctgcctctttccaacaccctgtgaggtgactgagcattgtcttccctcccaggcagctcacagtgtaagcttgtggacgatatcgaattcgcacgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacaggggggatccgtttatctgcagaattcgcccttgacgtcgccaccatggcgcttccggtgacagcactgctcctccccttggcgctgttgctccacgcagcaaggccgcagatccagctgcagcagagcggccccgaggtggtgaagcccggcgccagcgtgaagatcagctgcaaggccagcggctacaccttcaccgactactacatcacctgggtgaagcagaagcccggccagggcctggagtggatcggctggatctaccccggcagcggcaacaccaagtacaacgagaagttcaagggcaaggccaccctgaccgtggacaccagcagcagcaccgccttcatgcagctgagcagcctgaccagcgaggacaccgccgtgtacttctgcgccaactacggcaactactggttcgcctactggggccagggcacccaggtgaccgtgagcgccggcggcggcggcagcggcggcggcggcagcggcggcggcggcagcgacatcgtgctgacccagagccccgccagcctggccgtgagcctgggccagagagccaccatcagctgcaaggccagccagagcgtggacttcgacggcgacagctacatgaactggtaccagcagaagcccggccagccccccaaggtgctgatctacgccgccagcaacctggagagcggcatccccgccagattcagcggcagcggcagcggcaccgacttcaccctgaacatccaccccgtggaggaggaggacgccgccacctactactgccagcagagcaacgaggacccctggaccttcggcggcggcaccaagctggagatcaagagcgccgccgccttcgtgcccgtgttcctgcccgccaagcccaccaccacccccgcccccagaccccccacccccgcccccaccatcgccagccagcccctgagcctgagacccgaggcctgcagacccgccgccggcggcgccgtgcacaccagaggcctggacttcgcctgcgacatctacatctgggcccccctggccggcacctgcggcgtgctgctgctgagcctggtgatcaccctgtactgcaaccacagaaacagaagcaagagaagcagactgctgcacagcgactacatgaacatgacccccagaagacccggccccaccagaaagcactaccagccctacgccccccccagagacttcgccgcctacagaagcagagtgaagttcagcagaagcgccgacgcccccgcctaccagcagggccagaaccagctgtacaacgagctgaacctgggcagaagagaggagtacgacgtgctggacaagagaagaggcagagaccccgagatgggcggcaagcccagaagaaagaacccccaggagggcctgtacaacgagctgcagaaggacaagatggccgaggcctacagcgagatcggcatgaagggcgagagaagaagaggcaagggccacgacggcctgtaccagggcctgagcaccgccaccaaggacacctacgacgccctgcacatgcaggccctgccccccagaggaagcggagctactaacttcagcctgctgaagcaggctggagacgtggaggagaaccctggacctatgtctcgctccgttgccttagctgtgctcgcgctactctctctttctggattagaggctgtcatggcgccccgaaccctcttcctgggtggaggcggttcaggcggaggtggctctggcggtggcggatcgatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggtggtggttctggtggtggtggttctggcggcggcggctccggtggtggtggatccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaatgatagccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtcgactgaccagatggacctggctggagaagaagagattgagctctactcaggtgggccctcctccctctggtctcttccggtatcccccacccctcagcttgctgtagagacggcaatcaggggaaattctggtccctgccctcccgtcagcaccacggacagctcccacgtctgtgggacgctctctgcagatggggatgatctcccagccctgccccgcctctccctcgttccccaccagccctctttccagaaatttccttcttcatccaagggacttttcctcccagaacccgacacagacaccatcaactgcgaccagttcagcaggctgttgtgtgacatggaaggtgatgaagagaccagggaggcttatgccaatatcggtgaggaagcacctgagcccagaaaaggacaatcaagggcaagagttctttgctgccacttgtcaatatcacccattcatcatgagccacgtcagtcccctcccacagaaatcattgcaagggggatgcggagcaatggctggaggaacggagactccagggaagagaggggagatggaggccagtgggggaaataggccccttcactaatgaccaccaagaaaacaaaatctcatgtttacatcctccacctccatttctatacgcatttctgcttcttgctcttctgtccatcctttctacaaagcccataccatacacccctttcccttttcctcccagctccttagccaagctactctagtatttgtaataactagcatttactggatactcatagtatgctcattgctgtccggtaaccacgtgcggaccgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacttgacgtcgccaccatgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaaacttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaaccgctgatcagcctcgaaacttgtttattgcagcttataatggttacaaataaagcaatagcatcacaaatttcacaaataaagcatttttttcactgcattctagttgtggtttgtccaaactcatcaatgtatcttaggcgcctgatgcggtattttctccttacgcatctgtgcggtatttcacaccgcatacagtactgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtgcggccgcacgcgtcatatttatggggtatatgtgaatatttattacatgcatagaaggtataatgatcatgtcaggatatttgaggtatccacatttgggattgtttaaagattaaatgaaatagtgttaaaagtatttaatatgcccttcaacaaatgatgaggaaatctt

SEQ ID NO: 78 - protein, 483 aa, Artificial Sequence
  Synthetic
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSAGGGGSGGGGSGGGGSDIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIKSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 79 - DNA, 702 bp, Artificial Sequence
  Synthetic
caggtgcagctgcagcagtggggcgccggcctgctgaagcccagcgagaccctgagcctgacctgcgccgtgtacggcggcagcttcagcgcctactactggagctggatcagacagccccccggcaagggcctggagtggatcggcgacatcaaccacggcggcggcaccaactacaaccccagcctgaagagcagagtgaccatcagcgtggacaccagcaagaaccagttcagcctgaagctgaacagcgtgaccgccgccgacaccgccgtgtactactgcgccagcctgaccgcctactggggccagggcagcctggtgaccgtgagcagcggcggcggcggcagcggcggcggcggcagcggcggcggcggcagcgacatccagatgacccagagccccaccagcctgagcgccagcgtgggcgacagagtgaccatcacctgcagagccagccagggcatcagcagctggctgacctggtaccagcagaagcccgagaaggcccccaagagcctgatctacgccgccagcagcctgcagagcggcgtgcccagcagattcagcggcagcggcagcggcaccgacttcaccctgaccatcagcagcctgcagcccgaggacttcgccacctactactgccagcagtacgacagctaccccatcaccttcggccagggcaccagactggagatcaag

SEQ ID NO: 80 - DNA, 126 bp, Artificial Sequence
  Synthetic
aagagaggcagaaagaagctgctgtacatcttcaagcagcccttcatgagacccgtgcagaccacccaggaggaggacggctgcagctgcagattccccgaggaggaggagggcggctgcgagctg

SEQ ID NO: 81 - DNA, 1491 bp, Artificial Sequence
  Synthetic
atggcgcttccggtgacagcactgctcctccccttggcgctgttgctccacgcagcaaggccgcaggtgcagctgcagcagtggggcgccggcctgctgaagcccagcgagaccctgagcctgacctgcgccgtgtacggcggcagcttcagcgcctactactggagctggatcagacagccccccggcaagggcctggagtggatcggcgacatcaaccacggcggcggcaccaactacaaccccagcctgaagagcagagtgaccatcagcgtggacaccagcaagaaccagttcagcctgaagctgaacagcgtgaccgccgccgacaccgccgtgtactactgcgccagcctgaccgcctactggggccagggcagcctggtgaccgtgagcagcggcggcggcggcagcggcggcggcggcagcggcggcggcggcagcgacatccagatgacccagagccccaccagcctgagcgccagcgtgggcgacagagtgaccatcacctgcagagccagccagggcatcagcagctggctgacctggtaccagcagaagcccgagaaggcccccaagagcctgatctacgccgccagcagcctgcagagcggcgtgcccagcagattcagcggcagcggcagcggcaccgacttcaccctgaccatcagcagcctgcagcccgaggacttcgccacctactactgccagcagtacgacagctaccccatcaccttcggccagggcaccagactggagatcaagagcgccgccgccttcgtgcccgtgttcctgcccgccaagcccaccaccacccccgcccccagaccccccacccccgcccccaccatcgccagccagcccctgagcctgagacccgaggcctgcagacccgccgccggcggcgccgtgcacaccagaggcctggacttcgcctgcgacatctacatctgggcccccctggccggcacctgcggcgtgctgctgctgagcctggtgatcaccctgtactgcaaccacagaaacagaaagagaggcagaaagaagctgctgtacatcttcaagcagcccttcatgagacccgtgcagaccacccaggaggaggacggctgcagctgcagattccccgaggaggaggagggcggctgcgagctgagagtgaagttcagcagaagcgccgacgcccccgcctaccagcagggccagaaccagctgtacaacgagctgaacctgggcagaagagaggagtacgacgtgctggacaagagaagaggcagagaccccgagatgggcggcaagcccagaagaaagaacccccaggagggcctgtacaacgagctgcagaaggacaagatggccgaggcctacagcgagatcggcatgaagggcgagagaagaagaggcaagggccacgacggcctgtaccagggcctgagcaccgccaccaaggacacctacgacgccctgcacatgcaggccctgccccccaga

SEQ ID NO: 82 - DNA, 3299 bp, Artificial Sequence
  Synthetic
atggcgcttccggtgacagcactgctcctccccttggcgctgttgctccacgcagcaaggccgcaggtgcagctgcagcagtggggcgccggcctgctgaagcccagcgagaccctgagcctgacctgcgccgtgtacggcggcagcttcagcgcctactactggagctggatcagacagccccccggcaagggcctggagtggatcggcgacatcaaccacggcggcggcaccaactacaaccccagcctgaagagcagagtgaccatcagcgtggacaccagcaagaaccagttcagcctgaagctgaacagcgtgaccgccgccgacaccgccgtgtactactgcgccagcctgaccgcctactggggccagggcagcctggtgaccgtgagcagcggcggcggcggcagcggcggcggcggcagcggcggcggcggcagcgacatccagatgacccagagccccaccagcctgagcgccagcgtgggcgacagagtgaccatcacctgcagagccagccagggcatcagcagctggctgacctggtaccagcagaagcccgagaaggcccccaagagcctgatctacgccgccagcagcctgcagagcggcgtgcccagcagattcagcggcagcggcagcggcaccgacttcaccctgaccatcagcagcctgcagcccgaggacttcgccacctactactgccagcagtacgacagctaccccatcaccttcggccagggcaccagactggagatcaagagcgccgccgccttcgtgcccgtgttcctgcccgccaagcccaccaccacccccgcccccagaccccccacccccgcccccaccatcgccagccagcccctgagcctgagacccgaggcctgcagacccgccgccggcggcgccgtgcacaccagaggcctggacttcgcctgcgacatctacatctgggcccccctggccggcacctgcggcgtgctgctgctgagcctggtgatcaccctgtactgcaaccacagaaacagaaagagaggcagaaagaagctgctgtacatcttcaagcagcccttcatgagacccgtgcagaccacccaggaggaggacggctgcagctgcagattccccgaggaggaggagggcggctgcgagctgagagtgaagttcagcagaagcgccgacgcccccgcctaccagcagggccagaaccagctgtacaacgagctgaacctgggcagaagagaggagtacgacgtgctggacaagagaagaggcagagaccccgagatgggcggcaagcccagaagaaagaacccccaggagggcctgtacaacgagctgcagaaggacaagatggccgaggcctacagcgagatcggcatgaagggcgagagaagaagaggcaagggccacgacggcctgtaccagggcctgagcaccgccaccaaggacacctacgacgccctgcacatgcaggccctgccccccagaggaagcggattcagcctgctgaagcaggctggagacgtggaggagaaccctggacctatgtctcgctccgttgccttagctgtgctcgcgctactctctctttctggattagaggctgtcatggcgccccgaaccctcttcctgggtggaggcggttcaggcggaggtggctctggcggtggcggatcgatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggtggtggttctggtggtggtggttctggcggcggcggctccggtggtggtggatccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaatgatagccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 83 - DNA, 5010 bp, Artificial Sequence
  Synthetic
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacaggggggatccgtttatctgcagaattcgcccttgacgtcgccaccatggcgcttccggtgacagcactgctcctccccttggcgctgttgctccacgcagcaaggccgcaggtgcagctgcagcagtggggcgccggcctgctgaagcccagcgagaccctgagcctgacctgcgccgtgtacggcggcagcttcagcgcctactactggagctggatcagacagccccccggcaagggcctggagtggatcggcgacatcaaccacggcggcggcaccaactacaaccccagcctgaagagcagagtgaccatcagcgtggacaccagcaagaaccagttcagcctgaagctgaacagcgtgaccgccgccgacaccgccgtgtactactgcgccagcctgaccgcctactggggccagggcagcctggtgaccgtgagcagcggcggcggcggcagcggcggcggcggcagcggcggcggcggcagcgacatccagatgacccagagccccaccagcctgagcgccagcgtgggcgacagagtgaccatcacctgcagagccagccagggcatcagcagctggctgacctggtaccagcagaagcccgagaaggcccccaagagcctgatctacgccgccagcagcctgcagagcggcgtgcccagcagattcagcggcagcggcagcggcaccgacttcaccctgaccatcagcagcctgcagcccgaggacttcgccacctactactgccagcagtacgacagctaccccatcaccttcggccagggcaccagactggagatcaagagcgccgccgccttcgtgcccgtgttcctgcccgccaagcccaccaccacccccgcccccagaccccccacccccgcccccaccatcgccagccagcccctgagcctgagacccgaggcctgcagacccgccgccggcggcgccgtgcacaccagaggcctggacttcgcctgcgacatctacatctgggcccccctggccggcacctgcggcgtgctgctgctgagcctggtgatcaccctgtactgcaaccacagaaacagaaagagaggcagaaagaagctgctgtacatcttcaagcagcccttcatgagacccgtgcagaccacccaggaggaggacggctgcagctgcagattccccgaggaggaggagggcggctgcgagctgagagtgaagttcagcagaagcgccgacgcccccgcctaccagcagggccagaaccagctgtacaacgagctgaacctgggcagaagagaggagtacgacgtgctggacaagagaagaggcagagaccccgagatgggcggcaagcccagaagaaagaacccccaggagggcctgtacaacgagctgcagaaggacaagatggccgaggcctacagcgagatcggcatgaagggcgagagaagaagaggcaagggccacgacggcctgtaccagggcctgagcaccgccaccaaggacacctacgacgccctgcacatgcaggccctgccccccagaggaagcggattcagcctgctgaagcaggctggagacgtggaggagaaccctggacctatgtctcgctccgttgccttagctgtgctcgcgctactctctctttctggattagaggctgtcatggcgccccgaaccctcttcctgggtggaggcggttcaggcggaggtggctctggcggtggcggatcgatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggtggtggttctggtggtggtggttctggcggcggcggctccggtggtggtggatccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaatgatagccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 84 - DNA, 11235 bp, Artificial Sequence
  Synthetic
atgtcaggatatttgaggtatccacatttgggattgtttaaagattaaatgaaatagtgttaaaagtatttaatatgcccttcaacaaatgatgaggaaatcttagaatctgctcagactccttcagtttacatattaggaaactgaggcacagaaaggagcagagacttgctcaagtccacccaaagcagtagagcattgtggttaaatgcaggacttcagtcagactgtctgggttcaaatcctggttccacttggacatgggtttccttacataaatcacttcacctctctgagcctcagttttctcatatgcaaagtgaggataataataataccttccttacatggttactgatatgagtattaaatgtgccagctcatgtgcctggcgtataggaggtgctttataaaccttagctgttaccactcatggcattgccaaatgtgggacgggtctcctgactctctggtgtgagattgatggaatccacactttccagttcccttttctacctcctgggtatcttctcatatggttgtaagttccttggaggaagggaatgtggcttgctctctccaccacgctgagcatataagaggtgctgaatgagcgcttttattcactcctctcatccccagccctcaccagctgggagttgttgtaggtgtcaattttctgcctctttccaacaccctgtgaggtgactgagcattgtcttccctcccaggcagctcacagtgtaagcttgtggacgatatcgaattcgcacgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacaggggggatccgtttatctgcagaattcgcccttgacgtcgccaccatggcgcttccggtgacagcactgctcctccccttggcgctgttgctccacgcagcaaggccgcaggtgcagctgcagcagtggggcgccggcctgctgaagcccagcgagaccctgagcctgacctgcgccgtgtacggcggcagcttcagcgcctactactggagctggatcagacagccccccggcaagggcctggagtggatcggcgacatcaaccacggcggcggcaccaactacaaccccagcctgaagagcagagtgaccatcagcgtggacaccagcaagaaccagttcagcctgaagctgaacagcgtgaccgccgccgacaccgccgtgtactactgcgccagcctgaccgcctactggggccagggcagcctggtgaccgtgagcagcggcggcggcggcagcggcggcggcggcagcggcggcggcggcagcgacatccagatgacccagagccccaccagcctgagcgccagcgtgggcgacagagtgaccatcacctgcagagccagccagggcatcagcagctggctgacctggtaccagcagaagcccgagaaggcccccaagagcctgatctacgccgccagcagcctgcagagcggcgtgcccagcagattcagcggcagcggcagcggcaccgacttcaccctgaccatcagcagcctgcagcccgaggacttcgccacctactactgccagcagtacgacagctaccccatcaccttcggccagggcaccagactggagatcaagagcgccgccgccttcgtgcccgtgttcctgcccgccaagcccaccaccacccccgcccccagaccccccacccccgcccccaccatcgccagccagcccctgagcctgagacccgaggcctgcagacccgccgccggcggcgccgtgcacaccagaggcctggacttcgcctgcgacatctacatctgggcccccctggccggcacctgcggcgtgctgctgctgagcctggtgatcaccctgtactgcaaccacagaaacagaaagagaggcagaaagaagctgctgtacatcttcaagcagcccttcatgagacccgtgcagaccacccaggaggaggacggctgcagctgcagattccccgaggaggaggagggcggctgcgagctgagagtgaagttcagcagaagcgccgacgcccccgcctaccagcagggccagaaccagctgtacaacgagctgaacctgggcagaagagaggagtacgacgtgctggacaagagaagaggcagagaccccgagatgggcggcaagcccagaagaaagaacccccaggagggcctgtacaacgagctgcagaaggacaagatggccgaggcctacagcgagatcggcatgaagggcgagagaagaagaggcaagggccacgacggcctgtaccagggcctgagcaccgccaccaaggacacctacgacgccctgcacatgcaggccctgccccccagaggaagcggattcagcctgctgaagcaggctggagacgtggaggagaaccctggacctatgtctcgctccgttgccttagctgtgctcgcgctactctctctttctggattagaggctgtcatggcgccccgaaccctcttcctgggtggaggcggttcaggcggaggtggctctggcggtggcggatcgatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggtggtggttctggtggtggtggttctggcggcggcggctccggtggtggtggatccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaatgatagccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtcgactgaccagatggacctggctggagaagaagagattgagctctactcaggtgggccctcctccctctggtctcttccggtatcccccacccctcagcttgctgtagagacggcaatcaggggaaattctggtccctgccctcccgtcagcaccacggacagctcccacgtctgtgggacgctctctgcagatggggatgatctcccagccctgccccgcctctccctcgttccccaccagccctctttccagaaatttccttcttcatccaagggacttttcctcccagaacccgacacagacaccatcaactgcgaccagttcagcaggctgttgtgtgacatggaaggtgatgaagagaccagggaggcttatgccaatatcggtgaggaagcacctgagcccagaaaaggacaatcaagggcaagagttctttgctgccacttgtcaatatcacccattcatcatgagccacgtcagtcccctcccacagaaatcattgcaagggggatgcggagcaatggctggaggaacggagactccagggaagagaggggagatggaggccagtgggggaaataggccccttcactaatgaccaccaagaaaacaaaatctcatgtttacatcctccacctccatttctatacgcatttctgcttcttgctcttctgtccatcctttctacaaagcccataccatacacccctttcccttttcctcccagctccttagccaagctactctagtatttgtaataactagcatttactggatactcatagtatgctcattgctgtccggtaaccacgtgcggaccgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacttgacgtcgccaccatgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaaacttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaaccgctgatcagcctcgaaacttgtttattgcagcttataatggttacaaataaagcaatagcatcacaaatttcacaaataaagcatttttttcactgcattctagttgtggtttgtccaaactcatcaatgtatcttaggcgcctgatgcggtattttctccttacgcatctgtgcggtatttcacaccgcatacagtactgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtgcggccgcacgcgtcatatttatggggtatatgtgaatatttattacatgcatagaaggtataatgatc

SEQ ID NO: 85 - protein, 476 aa, Artificial Sequence
  Synthetic
QVQLQQWGAGLLKPSETLSLTCAVYGGSFSAYYWSWIRQPPGKGLEWIGDINHGGGTNYNPSLKSRVTISVDTSKNQFSLKLNSVTAADTAVYYCASLTAYWGQGSLVTVSSGGGGSGGGGSGGGGSDIQMTQSPTSLSASVGDRVTITCRASQGISSWLTWYQQKPEKAPKSLIYAASSLQSGVPSRFSGSGSGTDFTLTISSLQPEDFATYYCQQYDSYPITFGQGTRLEIKSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 86 - DNA, 700 bp, Artificial Sequence
  Synthetic
gacatccagatgacccagagccccaccagcctgagcgccagcgtgggcgacagagtgaccatcacctgcagagccagccagggcatcagcagctggctgacctggtaccagcagaagcccgagaaggcccccaagagcctgatctacgccgccagcagcctgcagagcggcgtgcccagcagattcagcggcagcggcagcggcaccgacttcaccctgaccatcagcagcctgcagcccgaggacttcgccacctactactgccagcagtacgacagctaccccatcaccttcggccagggcaccagactggagatcaagggcggcggcggcagcggcggcggcggcagcggcggcggcggcagccaggtgcagctgcagcagtggggcgccggcctgctgaagcccagcgagaccctgagcctgacctgcgccgtgtacggcggcagcttcagcgcctactactggagctggatcagacagccccccggcaagggcctggagtggatcggcgacatcaaccacggcggcggcaccaactacaaccccagcctgaagagcagagtgaccatcagcgtggacaccagcaagaaccagttcagcctgaagctgaacagcgtgaccgccgccgacaccgccgtgtactactgcgccagcctgaccgcctactggggccagggcagcctggtgaccgtgagcg

SEQ ID NO: 87 - DNA, 1489 bp, Artificial Sequence
  Synthetic
atggcgcttccggtgacagcactgctcctccccttggcgctgttgctccacgcagcaaggccggacatccagatgacccagagccccaccagcctgagcgccagcgtgggcgacagagtgaccatcacctgcagagccagccagggcatcagcagctggctgacctggtaccagcagaagcccgagaaggcccccaagagcctgatctacgccgccagcagcctgcagagcggcgtgcccagcagattcagcggcagcggcagcggcaccgacttcaccctgaccatcagcagcctgcagcccgaggacttcgccacctactactgccagcagtacgacagctaccccatcaccttcggccagggcaccagactggagatcaagggcggcggcggcagcggcggcggcggcagcggcggcggcggcagccaggtgcagctgcagcagtggggcgccggcctgctgaagcccagcgagaccctgagcctgacctgcgccgtgtacggcggcagcttcagcgcctactactggagctggatcagacagccccccggcaagggcctggagtggatcggcgacatcaaccacggcggcggcaccaactacaaccccagcctgaagagcagagtgaccatcagcgtggacaccagcaagaaccagttcagcctgaagctgaacagcgtgaccgccgccgacaccgccgtgtactactgcgccagcctgaccgcctactggggccagggcagcctggtgaccgtgagcgagcgccgccgccttcgtgcccgtgttcctgcccgccaagcccaccaccacccccgcccccagaccccccacccccgcccccaccatcgccagccagcccctgagcctgagacccgaggcctgcagacccgccgccggcggcgccgtgcacaccagaggcctggacttcgcctgcgacatctacatctgggcccccctggccggcacctgcggcgtgctgctgctgagcctggtgatcaccctgtactgcaaccacagaaacagaaagagaggcagaaagaagctgctgtacatcttcaagcagcccttcatgagacccgtgcagaccacccaggaggaggacggctgcagctgcagattccccgaggaggaggagggcggctgcgagctgagagtgaagttcagcagaagcgccgacgcccccgcctaccagcagggccagaaccagctgtacaacgagctgaacctgggcagaagagaggagtacgacgtgctggacaagagaagaggcagagaccccgagatgggcggcaagcccagaagaaagaacccccaggagggcctgtacaacgagctgcagaaggacaagatggccgaggcctacagcgagatcggcatgaagggcgagagaagaagaggcaagggccacgacggcctgtaccagggcctgagcaccgccaccaaggacacctacgacgccctgcacatgcaggccctgccccccaga

SEQ ID NO: 88 - DNA, 3302 bp, Artificial Sequence
  Synthetic
atggcgcttccggtgacagcactgctcctccccttggcgctgttgctccacgcagcaaggccggacatccagatgacccagagccccaccagcctgagcgccagcgtgggcgacagagtgaccatcacctgcagagccagccagggcatcagcagctggctgacctggtaccagcagaagcccgagaaggcccccaagagcctgatctacgccgccagcagcctgcagagcggcgtgcccagcagattcagcggcagcggcagcggcaccgacttcaccctgaccatcagcagcctgcagcccgaggacttcgccacctactactgccagcagtacgacagctaccccatcaccttcggccagggcaccagactggagatcaagggcggcggcggcagcggcggcggcggcagcggcggcggcggcagccaggtgcagctgcagcagtggggcgccggcctgctgaagcccagcgagaccctgagcctgacctgcgccgtgtacggcggcagcttcagcgcctactactggagctggatcagacagccccccggcaagggcctggagtggatcggcgacatcaaccacggcggcggcaccaactacaaccccagcctgaagagcagagtgaccatcagcgtggacaccagcaagaaccagttcagcctgaagctgaacagcgtgaccgccgccgacaccgccgtgtactactgcgccagcctgaccgcctactggggccagggcagcctggtgaccgtgagcgccgccgccttcgtgcccgtgttcctgcccgccaagcccaccaccacccccgcccccagaccccccacccccgcccccaccatcgccagccagcccctgagcctgagacccgaggcctgcagacccgccgccggcggcgccgtgcacaccagaggcctggacttcgcctgcgacatctacatctgggcccccctggccggcacctgcggcgtgctgctgctgagcctggtgatcaccctgtactgcaaccacagaaacagaaagagaggcagaaagaagctgctgtacatcttcaagcagcccttcatgagacccgtgcagaccacccaggaggaggacggctgcagctgcagattccccgaggaggaggagggcggctgcgagctgagagtgaagttcagcagaagcgccgacgcccccgcctaccagcagggccagaaccagctgtacaacgagctgaacctgggcagaagagaggagtacgacgtgctggacaagagaagaggcagagaccccgagatgggcggcaagcccagaagaaagaacccccaggagggcctgtacaacgagctgcagaaggacaagatggccgaggcctacagcgagatcggcatgaagggcgagagaagaagaggcaagggccacgacggcctgtaccagggcctgagcaccgccaccaaggacacctacgacgccctgcacatgcaggccctgccccccagaggaagcggagctactaacttcagcctgctgaagcaggctggagacgtggaggagaaccctggacctatgtctcgctccgttgccttagctgtgctcgcgctactctctctttctggattagaggctgtcatggcgccccgaaccctcttcctgggtggaggcggttcaggcggaggtggctctggcggtggcggatcgatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggtggtggttctggtggtggtggttctggcggcggcggctccggtggtggtggatccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaatgatagccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 89 - DNA, 5013 bp, Artificial Sequence
  Synthetic
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacaggggggatccgtttatctgcagaattcgcccttgacgtcgccaccatggcgcttccggtgacagcactgctcctccccttggcgctgttgctccacgcagcaaggccggacatccagatgacccagagccccaccagcctgagcgccagcgtgggcgacagagtgaccatcacctgcagagccagccagggcatcagcagctggctgacctggtaccagcagaagcccgagaaggcccccaagagcctgatctacgccgccagcagcctgcagagcggcgtgcccagcagattcagcggcagcggcagcggcaccgacttcaccctgaccatcagcagcctgcagcccgaggacttcgccacctactactgccagcagtacgacagctaccccatcaccttcggccagggcaccagactggagatcaagggcggcggcggcagcggcggcggcggcagcggcggcggcggcagccaggtgcagctgcagcagtggggcgccggcctgctgaagcccagcgagaccctgagcctgacctgcgccgtgtacggcggcagcttcagcgcctactactggagctggatcagacagccccccggcaagggcctggagtggatcggcgacatcaaccacggcggcggcaccaactacaaccccagcctgaagagcagagtgaccatcagcgtggacaccagcaagaaccagttcagcctgaagctgaacagcgtgaccgccgccgacaccgccgtgtactactgcgccagcctgaccgcctactggggccagggcagcctggtgaccgtgagcgccgccgccttcgtgcccgtgttcctgcccgccaagcccaccaccacccccgcccccagaccccccacccccgcccccaccatcgccagccagcccctgagcctgagacccgaggcctgcagacccgccgccggcggcgccgtgcacaccagaggcctggacttcgcctgcgacatctacatctgggcccccctggccggcacctgcggcgtgctgctgctgagcctggtgatcaccctgtactgcaaccacagaaacagaaagagaggcagaaagaagctgctgtacatcttcaagcagcccttcatgagacccgtgcagaccacccaggaggaggacggctgcagctgcagattccccgaggaggaggagggcggctgcgagctgagagtgaagttcagcagaagcgccgacgcccccgcctaccagcagggccagaaccagctgtacaacgagctgaacctgggcagaagagaggagtacgacgtgctggacaagagaagaggcagagaccccgagatgggcggcaagcccagaagaaagaacccccaggagggcctgtacaacgagctgcagaaggacaagatggccgaggcctacagcgagatcggcatgaagggcgagagaagaagaggcaagggccacgacggcctgtaccagggcctgagcaccgccaccaaggacacctacgacgccctgcacatgcaggccctgccccccagaggaagcggagctactaacttcagcctgctgaagcaggctggagacgtggaggagaaccctggacctatgtctcgctccgttgccttagctgtgctcgcgctactctctctttctggattagaggctgtcatggcgccccgaaccctcttcctgggtggaggcggttcaggcggaggtggctctggcggtggcggatcgatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggtggtggttctggtggtggtggttctggcggcggcggctccggtggtggtggatccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaatgatagccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 90 - DNA, 11238 bp, Artificial Sequence
  Synthetic
tgcatagaaggtataatgatcatgtcaggatatttgaggtatccacatttgggattgtttaaagattaaatgaaatagtgttaaaagtatttaatatgcccttcaacaaatgatgaggaaatcttagaatctgctcagactccttcagtttacatattaggaaactgaggcacagaaaggagcagagacttgctcaagtccacccaaagcagtagagcattgtggttaaatgcaggacttcagtcagactgtctgggttcaaatcctggttccacttggacatgggtttccttacataaatcacttcacctctctgagcctcagttttctcatatgcaaagtgaggataataataataccttccttacatggttactgatatgagtattaaatgtgccagctcatgtgcctggcgtataggaggtgctttataaaccttagctgttaccactcatggcattgccaaatgtgggacgggtctcctgactctctggtgtgagattgatggaatccacactttccagttcccttttctacctcctgggtatcttctcatatggttgtaagttccttggaggaagggaatgtggcttgctctctccaccacgctgagcatataagaggtgctgaatgagcgcttttattcactcctctcatccccagccctcaccagctgggagttgttgtaggtgtcaattttctgcctctttccaacaccctgtgaggtgactgagcattgtcttccctcccaggcagctcacagtgtaagcttgtggacgatatcgaattcgcacgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacaggggggatccgtttatctgcagaattcgcccttgacgtcgccaccatggcgcttccggtgacagcactgctcctccccttggcgctgttgctccacgcagcaaggccggacatccagatgacccagagccccaccagcctgagcgccagcgtgggcgacagagtgaccatcacctgcagagccagccagggcatcagcagctggctgacctggtaccagcagaagcccgagaaggcccccaagagcctgatctacgccgccagcagcctgcagagcggcgtgcccagcagattcagcggcagcggcagcggcaccgacttcaccctgaccatcagcagcctgcagcccgaggacttcgccacctactactgccagcagtacgacagctaccccatcaccttcggccagggcaccagactggagatcaagggcggcggcggcagcggcggcggcggcagcggcggcggcggcagccaggtgcagctgcagcagtggggcgccggcctgctgaagcccagcgagaccctgagcctgacctgcgccgtgtacggcggcagcttcagcgcctactactggagctggatcagacagccccccggcaagggcctggagtggatcggcgacatcaaccacggcggcggcaccaactacaaccccagcctgaagagcagagtgaccatcagcgtggacaccagcaagaaccagttcagcctgaagctgaacagcgtgaccgccgccgacaccgccgtgtactactgcgccagcctgaccgcctactggggccagggcagcctggtgaccgtgagcgccgccgccttcgtgcccgtgttcctgcccgccaagcccaccaccacccccgcccccagaccccccacccccgcccccaccatcgccagccagcccctgagcctgagacccgaggcctgcagacccgccgccggcggcgccgtgcacaccagaggcctggacttcgcctgcgacatctacatctgggcccccctggccggcacctgcggcgtgctgctgctgagcctggtgatcaccctgtactgcaaccacagaaacagaaagagaggcagaaagaagctgctgtacatcttcaagcagcccttcatgagacccgtgcagaccacccaggaggaggacggctgcagctgcagattccccgaggaggaggagggcggctgcgagctgagagtgaagttcagcagaagcgccgacgcccccgcctaccagcagggccagaaccagctgtacaacgagctgaacctgggcagaagagaggagtacgacgtgctggacaagagaagaggcagagaccccgagatgggcggcaagcccagaagaaagaacccccaggagggcctgtacaacgagctgcagaaggacaagatggccgaggcctacagcgagatcggcatgaagggcgagagaagaagaggcaagggccacgacggcctgtaccagggcctgagcaccgccaccaaggacacctacgacgccctgcacatgcaggccctgccccccagaggaagcggagctactaacttcagcctgctgaagcaggctggagacgtggaggagaaccctggacctatgtctcgctccgttgccttagctgtgctcgcgctactctctctttctggattagaggctgtcatggcgccccgaaccctcttcctgggtggaggcggttcaggcggaggtggctctggcggtggcggatcgatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggtggtggttctggtggtggtggttctggcggcggcggctccggtggtggtggatccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaatgatagccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtcgactgaccagatggacctggctggagaagaagagattgagctctactcaggtgggccctcctccctctggtctcttccggtatcccccacccctcagcttgctgtagagacggcaatcaggggaaattctggtccctgccctcccgtcagcaccacggacagctcccacgtctgtgggacgctctctgcagatggggatgatctcccagccctgccccgcctctccctcgttccccaccagccctctttccagaaatttccttcttcatccaagggacttttcctcccagaacccgacacagacaccatcaactgcgaccagttcagcaggctgttgtgtgacatggaaggtgatgaagagaccagggaggcttatgccaatatcggtgaggaagcacctgagcccagaaaaggacaatcaagggcaagagttctttgctgccacttgtcaatatcacccattcatcatgagccacgtcagtcccctcccacagaaatcattgcaagggggatgcggagcaatggctggaggaacggagactccagggaagagaggggagatggaggccagtgggggaaataggccccttcactaatgaccaccaagaaaacaaaatctcatgtttacatcctccacctccatttctatacgcatttctgcttcttgctcttctgtccatcctttctacaaagcccataccatacacccctttcccttttcctcccagctccttagccaagctactctagtatttgtaataactagcatttactggatactcatagtatgctcattgctgtccggtaaccacgtgcggaccgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacttgacgtcgccaccatgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaaacttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaaccgctgatcagcctcgaaacttgtttattgcagcttataatggttacaaataaagcaatagcatcacaaatttcacaaataaagcatttttttcactgcattctagttgtggtttgtccaaactcatcaatgtatcttaggcgcctgatgcggtattttctccttacgcatctgtgcggtatttcacaccgcatacagtactgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtgcggccgcacgcgtcatatttatggggtatatgtgaatatttattaca

SEQ ID NO: 91 - protein, 474 aa, Artificial Sequence
  Synthetic
DIQMTQSPTSLSASVGDRVTITCRASQGISSWLTWYQQKPEKAPKSLIYAASSLQSGVPSRFSGSGSGTDFTLTISSLQPEDFATYYCQQYDSYPITFGQGTRLEIKGGGGSGGGGSGGGGSQVQLQQWGAGLLKPSETLSLTCAVYGGSFSAYYWSWIRQPPGKGLEWIGDINHGGGTNYNPSLKSRVTISVDTSKNQFSLKLNSVTAADTAVYYCASLTAYWGQGSLVTVSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 92 - DNA, 1178 bp, Artificial Sequence
  Synthetic
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 93 - DNA, 882 bp, Artificial Sequence
  Synthetic
atgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaaacttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaaggaagcgga

SEQ ID NO: 94 - DNA, 1500 bp, Artificial Sequence
  Synthetic
atgacttttaacagttttgaaggatctaaaacttgtgtacctgcagacatcaataaggaagaagaatttgtagaagagtttaatagattaaaaacttttgctaattttccaagtggtagtcctgtttcagcatcaacactggcacgagcagggtttctttatactggtgaaggagataccgtgcggtgctttagttgtcatgcagctgtagatagatggcaatatggagactcagcagttggaagacacaggaaagtatccccaaattgcagatttatcaacggcttttatcttgaaaatagtgccacgcagtctacaaattctggtatccagaatggtcagtacaaagttgaaaactatctgggaagcagagatcattttgccttagacaggccatctgagacacatgcagactatcttttgagaactgggcaggttgtagatatatcagacaccatatacccgaggaaccctgccatgtatagtgaagaagctagattaaagtcctttcagaactggccagactatgctcacctaaccccaagagagttagcaagtgctggactctactacacaggtattggtgaccaagtgcagtgcttttgttgtggtggaaaactgaaaaattgggaaccttgtgatcgtgcctggtcagaacacaggcgacactttcctaattgcttctttgttttgggccggaatcttaatattcgaagtgaatctgatgctgtgagttctgataggaatttcccaaattcaacaaatcttccaagaaatccatccatggcagattatgaagcacggatctttacttttgggacatggatatactcagttaacaaggagcagcttgcaagagctggattttatgctttaggtgaaggtgataaagtaaagtgctttcactgtggaggagggctaactgattggaagcccagtgaagacccttgggaacaacatgctaaatggtatccagggtgcaaatatctgttagaacagaagggacaagaatatataaacaatattcatttaactcattcacttgaggagtgtctggtaagaactactgagaaaacaccatcactaactagaagaattgatgataccatcttccaaaatcctatggtacaagaagctatacgaatggggttcagtttcaaggacattaagaaaataatggaggaaaaaattcagatatctgggagcaactataaatcacttgaggttctggttgcagatctagtgaatgctcagaaagacagtatgcaagatgagtcaagtcagacttcattacagaaagagattagtactgaagagcagctaaggcgcctgcaagaggagaagctttgcaaaatctgtatggatagaaatattgctatcgtttttgttccttgtggacatctagtcacttgtaaacaatgtgctgaagcagttgacaagtgtcccatgtgctacacagtcattactttcaagcaaaaaatttttatgtctggaagcgga

SEQ ID NO: 95 - DNA, 2694 bp, Artificial Sequence
  Synthetic
atggaaactctttctaatgcaagtggtacttttgccatacgccttttaaagatactgtgtcaagataacccttcgcacaacgtgttctgttctcctgtgagcatctcctctgccctggccatggttctcctaggggcaaagggaaacaccgcaacccagatggcccaggcactgtctttaaacacagaggaagacattcatcgggctttccagtcgcttctcactgaagtgaacaaggctggcacacagtacctgctgagaacggccaacaggctctttggagagaaaacttgtcagttcctctcaacgtttaaggaatcctgtcttcaattctaccatgctgagctgaaggagctttcctttatcagagctgcagaagagtccaggaaacacatcaacacctgggtctcaaaaaagaccgaaggtaaaattgaagagttgttgccgggtagctcaattgatgcagaaaccaggctggttcttgtcaatgccatctacttcaaaggaaagtggaatgaaccgtttgacgaaacatacacaagggaaatgccctttaaaataaaccaggaggagcaaaggccagtgcagatgatgtatcaggaggccacgtttaagctcgcccacgtgggcgaggtgcgcgcgcagctgctggagctgccctacgccaggaaggagctgagcctgctggtgctgctgcctgacgacggcgtggagctcagcacggtggaaaaaagtctcacttttgagaaactcacagcctggaccaagccagactgtatgaagagtactgaggttgaagttctccttccaaaatttaaactacaagaggattatgacatggaatctgtgcttcggcatttgggaattgttgatgccttccaacagggcaaggctgacttgtcggcaatgtcagcggagagagacctgtgtctgtccaagttcgtgcacaagagttttgtggaggtgaatgaagaaggcaccgaggcagcggcagcgtcgagctgctttgtagttgcagagtgctgcatggaatctggccccaggttctgtgctgaccaccctttccttttcttcatcaggcacaacagagccaacagcattctgttctgtggcaggttctcatcgccaggaagcggagctactaacttcagcctgctgaagcaggctggagacgtggaggagaaccctggacctatgtctcgctccgttgccttagctgtgctcgcgctactctctctttctggattagaggctgtcatggcgccccgaaccctcttcctgggtggaggcggttcaggcggaggtggctctggcggtggcggatcgatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggtggtggttctggtggtggtggttctggcggcggcggctccggtggtggtggatccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttg

SEQ ID NO: 96 - DNA, 2436 bp, Artificial Sequence
  Synthetic
atggaaactctttctaatgcaagtggtacttttgccatacgccttttaaagatactgtgtcaagataacccttcgcacaacgtgttctgttctcctgtgagcatctcctctgccctggccatggttctcctaggggcaaagggaaacaccgcaacccagatggcccaggcactgtctttaaacacagaggaagacattcatcgggctttccagtcgcttctcactgaagtgaacaaggctggcacacagtacctgctgagaacggccaacaggctctttggagagaaaacttgtcagttcctctcaacgtttaaggaatcctgtcttcaattctaccatgctgagctgaaggagctttcctttatcagagctgcagaagagtccaggaaacacatcaacacctgggtctcaaaaaagaccgaaggtaaaattgaagagttgttgccgggtagctcaattgatgcagaaaccaggctggttcttgtcaatgccatctacttcaaaggaaagtggaatgaaccgtttgacgaaacatacacaagggaaatgccctttaaaataaaccaggaggagcaaaggccagtgcagatgatgtatcaggaggccacgtttaagctcgcccacgtgggcgaggtgcgcgcgcagctgctggagctgccctacgccaggaaggagctgagcctgctggtgctgctgcctgacgacggcgtggagctcagcacggtggaaaaaagtctcacttttgagaaactcacagcctggaccaagccagactgtatgaagagtactgaggttgaagttctccttccaaaatttaaactacaagaggattatgacatggaatctgtgcttcggcatttgggaattgttgatgccttccaacagggcaaggctgacttgtcggcaatgtcagcggagagagacctgtgtctgtccaagttcgtgcacaagagttttgtggaggtgaatgaagaaggcaccgaggcagcggcagcgtcgagctgctttgtagttgcagagtgctgcatggaatctggccccaggttctgtgctgaccaccctttccttttcttcatcaggcacaacagagccaacagcattctgttctgtggcaggttctcatcgccaggaagcggagctactaacttcagcctgctgaagcaggctggagacgtggaggagaaccctggacctatggactggacctggatcctgttcctggtggccgccgccaccagggtgcacagcggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctagcggcggcggcagcggcggcggcggcagcggcggcggcggcagcggcggcggcggcagcggcggcggcagcctgcagatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccaccta

SEQ ID NO: 97 - DNA, 2800 bp, Artificial Sequence
  Synthetic
atgacttttaacagttttgaaggatctaaaacttgtgtacctgcagacatcaataaggaagaagaatttgtagaagagtttaatagattaaaaacttttgctaattttccaagtggtagtcctgtttcagcatcaacactggcacgagcagggtttctttatactggtgaaggagataccgtgcggtgctttagttgtcatgcagctgtagatagatggcaatatggagactcagcagttggaagacacaggaaagtatccccaaattgcagatttatcaacggcttttatcttgaaaatagtgccacgcagtctacaaattctggtatccagaatggtcagtacaaagttgaaaactatctgggaagcagagatcattttgccttagacaggccatctgagacacatgcagactatcttttgagaactgggcaggttgtagatatatcagacaccatatacccgaggaaccctgccatgtatagtgaagaagctagattaaagtcctttcagaactggccagactatgctcacctaaccccaagagagttagcaagtgctggactctactacacaggtattggtgaccaagtgcagtgcttttgttgtggtggaaaactgaaaaattgggaaccttgtgatcgtgcctggtcagaacacaggcgacactttcctaattgcttctttgttttgggccggaatcttaatattcgaagtgaatctgatgctgtgagttctgataggaatttcccaaattcaacaaatcttccaagaaatccatccatggcagattatgaagcacggatctttacttttgggacatggatatactcagttaacaaggagcagcttgcaagagctggattttatgctttaggtgaaggtgataaagtaaagtgctttcactgtggaggagggctaactgattggaagcccagtgaagacccttgggaacaacatgctaaatggtatccagggtgcaaatatctgttagaacagaagggacaagaatatataaacaatattcatttaactcattcacttgaggagtgtctggtaagaactactgagaaaacaccatcactaactagaagaattgatgataccatcttccaaaatcctatggtacaagaagctatacgaatggggttcagtttcaaggacattaagaaaataatggaggaaaaaattcagatatctgggagcaactataaatcacttgaggttctggttgcagatctagtgaatgctcagaaagacagtatgcaagatgagtcaagtcagacttcattacagaaagagattagtactgaagagcagctaaggcgcctgcaagaggagaagctttgcaaaatctgtatggatagaaatattgctatcgtttttgttccttgtggacatctagtcacttgtaaacaatgtgctgaagcagttgacaagtgtcccatgtgctacacagtcattactttcaagcaaaaaatttttatgtctggaagcggagctactaacttcagcctgctgaagcaggctggagacgtggaggagaaccctggacctatggactggacctggatcctgttcctggtggccgccgccaccagggtgcacagcggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctagcggcggcggcagcggcggcggcggcagcggcggcggcggcagcggcggcggcggcagcggcggcggcagcctgcagatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccacctat

SEQ ID NO: 98 - DNA, 3078 bp, Artificial Sequence
  Synthetic
atggcgcttccggtgacagcactgctcctccccttggcgctgttgctccacgcagcaaggccgcagatccagctgcagcagagcggccccgaggtggtgaagcccggcgccagcgtgaagatcagctgcaaggccagcggctacaccttcaccgactactacatcacctgggtgaagcagaagcccggccagggcctggagtggatcggctggatctaccccggcagcggcaacaccaagtacaacgagaagttcaagggcaaggccaccctgaccgtggacaccagcagcagcaccgccttcatgcagctgagcagcctgaccagcgaggacaccgccgtgtacttctgcgccaactacggcaactactggttcgcctactggggccagggcacccaggtgaccgtgagcgccggcggcggcggcagcggcggcggcggcagcggcggcggcggcagcgacatcgtgctgacccagagccccgccagcctggccgtgagcctgggccagagagccaccatcagctgcaaggccagccagagcgtggacttcgacggcgacagctacatgaactggtaccagcagaagcccggccagccccccaaggtgctgatctacgccgccagcaacctggagagcggcatccccgccagattcagcggcagcggcagcggcaccgacttcaccctgaacatccaccccgtggaggaggaggacgccgccacctactactgccagcagagcaacgaggacccctggaccttcggcggcggcaccaagctggagatcaagagcgccgccgccttcgtgcccgtgttcctgcccgccaagcccaccaccacccccgcccccagaccccccacccccgcccccaccatcgccagccagcccctgagcctgagacccgaggcctgcagacccgccgccggcggcgccgtgcacaccagaggcctggacttcgcctgcgacatctacatctgggcccccctggccggcacctgcggcgtgctgctgctgagcctggtgatcaccctgtactgcaaccacagaaacagaagcaagagaagcagactgctgcacagcgactacatgaacatgacccccagaagacccggccccaccagaaagcactaccagccctacgccccccccagagacttcgccgcctacagaagcagagtgaagttcagcagaagcgccgacgcccccgcctaccagcagggccagaaccagctgtacaacgagctgaacctgggcagaagagaggagtacgacgtgctggacaagagaagaggcagagaccccgagatgggcggcaagcccagaagaaagaacccccaggagggcctgtacaacgagctgcagaaggacaagatggccgaggcctacagcgagatcggcatgaagggcgagagaagaagaggcaagggccacgacggcctgtaccagggcctgagcaccgccaccaaggacacctacgacgccctgcacatgcaggccctgccccccagaggaagcggagctactaacttcagcctgctgaagcaggctggagacgtggaggagaaccctggacctatgtctcgctccgttgccttagctgtgctcgcgctactctctctttctggattagaggctgtcatggcgccccgaaccctcttcctgggtggaggcggttcaggcggaggtggctctggcggtggcggatcgatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggtggtggttctggtggtggtggttctggcggcggcggctccggtggtggtggatccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttg

SEQ ID NO: 99 - DNA, 3048 bp, Artificial Sequence
  Synthetic
atggcgcttccggtgacagcactgctcctccccttggcgctgttgctccacgcagcaaggccgcaggtgcagctgcagcagtggggcgccggcctgctgaagcccagcgagaccctgagcctgacctgcgccgtgtacggcggcagcttcagcgcctactactggagctggatcagacagccccccggcaagggcctggagtggatcggcgacatcaaccacggcggcggcaccaactacaaccccagcctgaagagcagagtgaccatcagcgtggacaccagcaagaaccagttcagcctgaagctgaacagcgtgaccgccgccgacaccgccgtgtactactgcgccagcctgaccgcctactggggccagggcagcctggtgaccgtgagcagcggcggcggcggcagcggcggcggcggcagcggcggcggcggcagcgacatccagatgacccagagccccaccagcctgagcgccagcgtgggcgacagagtgaccatcacctgcagagccagccagggcatcagcagctggctgacctggtaccagcagaagcccgagaaggcccccaagagcctgatctacgccgccagcagcctgcagagcggcgtgcccagcagattcagcggcagcggcagcggcaccgacttcaccctgaccatcagcagcctgcagcccgaggacttcgccacctactactgccagcagtacgacagctaccccatcaccttcggccagggcaccagactggagatcaagagcgccgccgccttcgtgcccgtgttcctgcccgccaagcccaccaccacccccgcccccagaccccccacccccgcccccaccatcgccagccagcccctgagcctgagacccgaggcctgcagacccgccgccggcggcgccgtgcacaccagaggcctggacttcgcctgcgacatctacatctgggcccccctggccggcacctgcggcgtgctgctgctgagcctggtgatcaccctgtactgcaaccacagaaacagaaagagaggcagaaagaagctgctgtacatcttcaagcagcccttcatgagacccgtgcagaccacccaggaggaggacggctgcagctgcagattccccgaggaggaggagggcggctgcgagctgagagtgaagttcagcagaagcgccgacgcccccgcctaccagcagggccagaaccagctgtacaacgagctgaacctgggcagaagagaggagtacgacgtgctggacaagagaagaggcagagaccccgagatgggcggcaagcccagaagaaagaacccccaggagggcctgtacaacgagctgcagaaggacaagatggccgaggcctacagcgagatcggcatgaagggcgagagaagaagaggcaagggccacgacggcctgtaccagggcctgagcaccgccaccaaggacacctacgacgccctgcacatgcaggccctgccccccagaggaagcggattcagcctgctgaagcaggctggagacgtggaggagaaccctggacctatgtctcgctccgttgccttagctgtgctcgcgctactctctctttctggattagaggctgtcatggcgccccgaaccctcttcctgggtggaggcggttcaggcggaggtggctctggcggtggcggatcgatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggtggtggttctggtggtggtggttctggcggcggcggctccggtggtggtggatccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttg

SEQ ID NO: 100 - DNA, 3051 bp, Artificial Sequence
  Synthetic
atggcgcttccggtgacagcactgctcctccccttggcgctgttgctccacgcagcaaggccggacatccagatgacccagagccccaccagcctgagcgccagcgtgggcgacagagtgaccatcacctgcagagccagccagggcatcagcagctggctgacctggtaccagcagaagcccgagaaggcccccaagagcctgatctacgccgccagcagcctgcagagcggcgtgcccagcagattcagcggcagcggcagcggcaccgacttcaccctgaccatcagcagcctgcagcccgaggacttcgccacctactactgccagcagtacgacagctaccccatcaccttcggccagggcaccagactggagatcaagggcggcggcggcagcggcggcggcggcagcggcggcggcggcagccaggtgcagctgcagcagtggggcgccggcctgctgaagcccagcgagaccctgagcctgacctgcgccgtgtacggcggcagcttcagcgcctactactggagctggatcagacagccccccggcaagggcctggagtggatcggcgacatcaaccacggcggcggcaccaactacaaccccagcctgaagagcagagtgaccatcagcgtggacaccagcaagaaccagttcagcctgaagctgaacagcgtgaccgccgccgacaccgccgtgtactactgcgccagcctgaccgcctactggggccagggcagcctggtgaccgtgagcgccgccgccttcgtgcccgtgttcctgcccgccaagcccaccaccacccccgcccccagaccccccacccccgcccccaccatcgccagccagcccctgagcctgagacccgaggcctgcagacccgccgccggcggcgccgtgcacaccagaggcctggacttcgcctgcgacatctacatctgggcccccctggccggcacctgcggcgtgctgctgctgagcctggtgatcaccctgtactgcaaccacagaaacagaaagagaggcagaaagaagctgctgtacatcttcaagcagcccttcatgagacccgtgcagaccacccaggaggaggacggctgcagctgcagattccccgaggaggaggagggcggctgcgagctgagagtgaagttcagcagaagcgccgacgcccccgcctaccagcagggccagaaccagctgtacaacgagctgaacctgggcagaagagaggagtacgacgtgctggacaagagaagaggcagagaccccgagatgggcggcaagcccagaagaaagaacccccaggagggcctgtacaacgagctgcagaaggacaagatggccgaggcctacagcgagatcggcatgaagggcgagagaagaagaggcaagggccacgacggcctgtaccagggcctgagcaccgccaccaaggacacctacgacgccctgcacatgcaggccctgccccccagaggaagcggagctactaacttcagcctgctgaagcaggctggagacgtggaggagaaccctggacctatgtctcgctccgttgccttagctgtgctcgcgctactctctctttctggattagaggctgtcatggcgccccgaaccctcttcctgggtggaggcggttcaggcggaggtggctctggcggtggcggatcgatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggtggtggttctggtggtggtggttctggcggcggcggctccggtggtggtggatccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttg

(19) United States
(12) Patent Application Publication
(10) Pub. No.: US 2023/0016422 A1
(43) Pub. Date: Jan. 19, 2023

(54) ENGINEERED CELLS WITH IMPROVED PROTECTION FROM NATURAL KILLER CELL KILLING

(71) Applicant: CRISPR THERAPEUTICS AG, Zug (CH)

(72) Inventors: Valentin SLUCH, Cambridge, MA (US); Danielle SWAIN, Cambridge, MA (US); Alireza REZANIA, Cambridge, MA (US)

(73) Assignee: CRISPR THERAPEUTICS AG, Zug (CH)

(21) Appl. No.: 17/830,273

(22) Filed: Jun. 1, 2022

Related U.S. Application Data
(60) Provisional application No. 63/214,134, filed on Jun. 23, 2021.
(60) Provisional application No. 63/283,878, filed on Nov. 29, 2021.

(51) Int. Cl. C12N 15/90; C12N 9/22; C12N 5/074

(52) U.S. Cl. CPC C12N 15/907; C12N 9/22; C12N 5/0696; C12N 2310/20

(57) ABSTRACT

Provided herein are cells engineered to have improved protection against natural killer cell killing. The cells are engineered to comprise an insertion of a polynucleotide encoding SERPINB9. Also provided herein are methods of making the engineered cells and therapeutic uses of the engineered cells. The engineered cells can also comprise at least one genetic modification within or near at least one gene that encodes one or more MHC-I or MHC-II human leukocyte antigens or component or transcriptional regulator of the MHC-I or MHC-II complex, at least one genetic modification that increases the expression of at least one polynucleotide that encodes a tolerogenic factor, and optionally at least one genetic modification that increases or decreases the expression of at least one gene that encodes a survival factor. The engineered cells can be stem cells and the engineered stem cells can be differentiated into various lineages having protection against NK cell killing.

### CROSS-REFERENCE TO RELATED APPLICATION

This application claims the benefit of U.S. Provisional Application No. U.S. 63/214,134, filed Jun. 23, 2021 and U.S. Provisional Application No. U.S. 63/283,878 filed Nov. 29, 2021 the disclosure of each is hereby incorporated by reference in its entirety.

### INCORPORATION BY REFERENCE OF SEQUENCE LISTING

This application contains a Sequence Listing that has been submitted in ASCII format via EFS-Web and is hereby incorporated by reference in its entirety. The ASCII copy, created on May 31, 2022, is named 100867-729998_CT168-US2-Sequence_Listing_ST25.txt, and is about 261,000 bytes in size.

### FIELD OF THE INVENTION

The invention relates to the use of gene-editing technology to engineer cells, such as engineered stem cells, having improved protection from natural killer cell killing and the differentiation of said engineered stem cells.

### BACKGROUND

There is a need for adoptive cell therapy and other cell transplantation therapies that do not rely on the use of cells obtained from patients or donors and do not induce allogeneic rejection. Therapeutically useful cells, such as natural killer (NK) cells, hepatocytes, and pancreatic beta cells, can be differentiated in vitro from stem cells (e.g., iPSCs) that may also be gene edited. For example, gene editing strategies may include modifying a gene that encodes one or more MHC-I or MHC-II human leukocyte antigens or a component or a transcriptional regulator of a MHC-I or MHC-II complex in order to facilitate immune evasion, e.g., a B2M KO. However, such edits may make the edited cell susceptible to NK cell killing as the edit(s) could create an edited cell that lacks a “self” marker. Despite advances in recent years, there still is a need for improved efficacy, persistence, functionality, immune evasion, and survivability of therapeutic cells. Similarly, there is a need for a uniform pool of therapeutic cells that can be manufactured in a consistent manner for use in any patients in need thereof.

### SUMMARY OF THE INVENTION

In some aspects, the present disclosure provides engineered cells that have been edited using, for example, CRISPR/Cas9 gene editing technology.

In some aspects the current disclosure encompasses an in vitro method for generating an engineered cell, the method comprising delivering to a cell: (a) an RNA-guided nuclease and a gRNA targeting a target site in a B2M gene locus; and (b) a vector comprising a nucleic acid, the nucleic acid comprising: (i) nucleotide sequence encoding a SERPINB9 protein; (ii) a nucleotide sequence having sequence homology with a genomic region located left of the target site in the B2M gene locus; and (iii) a nucleotide sequence having sequence homology with a genomic region located right of the target site in the B2M gene locus, wherein (i) is flanked by (ii) and (iii); wherein the B2M gene locus is cleaved at the target site and the nucleotide sequences encoding the SERPINB9 protein are inserted into the B2M gene locus, thereby disrupting the B2M gene. In some aspects the gRNA comprises a spacer sequence corresponding to a sequence consisting of SEQ ID NO: 1. In some aspects, the RNA-guided nuclease and the gRNA targeting a target site in a B2M gene locus are delivered as a ribonucleoprotein (RNP) complex.

In some aspects the nucleotide sequence of (b)(i) further comprises a nucleotide sequence encoding a IL15/IL15Rα fusion protein. In some aspects the nucleotide sequence of (b)(i) comprises the nucleotide sequence encoding the SERPINB9 protein linked to a nucleotide sequence encoding a P2A peptide sequence linked to the nucleotide sequence encoding the IL15/IL15Rα fusion protein to form a SERPINB9-P2A-IL15/IL15Rα construct. In some aspects the SERPINB9-P2A-IL15/IL15Rα construct consists essentially of SEQ ID NO: 37. In some aspects the SERPINB9-P2A-IL15/IL15Rα construct is operably linked to an exogenous promoter, non-limiting examples of exogenous promoters include a CAG, CMV, EF1α, PGK, or UBC promoter. In some exemplary aspects the exogenous promoter is CAG and CAG-SERPINB9-P2A-IL15/IL15Rα consists essentially of SEQ ID NO: 38.

In some aspects the current disclosure encompasses an in vitro method, wherein the nucleotide sequence of (b)(i) further comprises a nucleotide sequence encoding an HLA-E trimer. In some aspects the nucleotide sequence of (b)(i) comprises the nucleotide sequence encoding the SERPINB9 linked to a nucleotide sequence encoding a P2A peptide sequence linked to the nucleotide sequence encoding the HLA-E trimer to form a SERPINB9-P2A-HLA-E construct. In some aspects the SERPINB9-P2A-HLA-E construct consists essentially of SEQ ID NO: 21. In some aspects the SERPINB9-P2A-HLA-E construct is operably linked to an exogenous promoter for example a CAG, CMV, EF1α, PGK, or UBC promoter. In some exemplary aspects the exogenous promoter is CAG and CAG-SERPINB9-P2A-HLA-E consists essentially of SEQ ID NO: 22

In some aspects the current disclosure encompasses an in vitro method, wherein the nucleotide sequence of (b)(ii) consists essentially of SEQ ID NO: 3, and the nucleotide sequence of (b)(iii) consists essentially of SEQ ID NO: 19. In some aspects the first vector consists essentially of SEQ ID NO: 39. In some aspects the first vector consists essentially of SEQ ID NO: 23.

In some aspects the in vitro method of the current disclosure may further comprises delivering to the cell an RNA-guided nuclease and a gRNA targeting a target site in a CISH gene locus. In some aspects the RNA-guided nuclease and gRNA are delivered as a ribonucleoprotein (RNP) complex. In some aspects the gRNA targeting a target site in a CISH gene locus comprises a spacer sequence corresponding to a sequence consisting of any one of SEQ ID NOS: 49-60.

In some aspects the in vitro method of the current disclosure may further comprises delivering to the cell an RNA-guided nuclease and a gRNA targeting a target site in a FAS gene locus. In some aspects the RNA-guided nuclease and gRNA are delivered as a ribonucleoprotein (RNP) complex. In some aspects, the gRNA targeting a target site in a FAS gene locus comprises a spacer sequence corresponding to a sequence consisting of any one of SEQ ID NOS: 61-67.

In some aspects of the in vitro method of the current disclosure, the cell is a pluripotent stem cell or an adult stem cell. In some aspects the cell is an induced pluripotent stem cell, or an embryonic stem cell. In some aspects the cell is a terminally differentiated somatic cell or a lineage restricted progenitor cell. In some aspects the lineage restricted progenitor cell is hematopoietic progenitor cells, mesodermal cells, definitive hemogenic endothelium, definitive hematopoietic stem or progenitor cells, CD34+ cells, multipotent progenitors (MPP), common lymphoid progenitor cells, T cell progenitors, NK cell progenitors, pancreatic endoderm progenitors, pancreatic endocrine progenitors, mesenchymal progenitor cells, muscle progenitor cells, blast cells, or neural progenitor cells, and the fully differentiated somatic cell is selected from a hematopoietic cell, a pancreatic beta cell, an epithelial cell, an endodermal cell, a macrophages, a hepatocyte, an adipocyte, a kidney cell, a blood cell, a cardiomyocyte, or an immune system cell. In some aspects the cell is a mammalian cell.

Other aspects and iterations of the disclosure are described in more detail below.

### BRIEF DESCRIPTION OF THE DRAWINGS

FIG. 1 presents a map of the B2M-CAGGS-SERPINB9-P2A-HLA-E donor plasmid.

FIG. 2 shows FACS plots generated during the single cell sorting of the B2M-SERPINB9-P2A-HLA-E bulk population previously enriched by MACS.

FIG. 3 presents PCR analysis of SERPINB9-P2A-HLA-E KI at the B2M locus. The gel shows PCR amplification of B2M region of the genome with the 3′ primer stationed outside the knock-in (KI) site (not present in the plasmid donor) and the 5′ primer stationed inside the KI-only region. Presence of a 1.1 kilo base (kb) band indicates successful integration of the KI construct into the B2M locus, the absence of a band indicates a WT genotype.

FIG. 4 shows PCR 1 analysis of random plasmid insertions during knock-in of SERPINB9-P2A-HLA-E in the B2M locus. PCR was performed with 5′ and 3′ primers that bind outside of the homology arms within the KI plasmid. Presence of a 340 base pair (bp) band indicates that there is random integration of the plasmid backbone within the genome, clones without bands do not have random plasmid insertion.

FIG. 5 shows PCR 2 analysis of random plasmid insertions during knock-in of SERPINB9-P2A-HLA-E in the B2M locus. PCR was performed with 5′ and 3′ primers that bind outside of the homology arms within the KI plasmid. Presence of a 476 bp band indicates that there is random integration of the plasmid backbone within the genome, clones without bands do not have random plasmid insertion.

FIG. 6 shows zygosity at the B2M locus following knock-in of SERPINB9-P2A-HLA-E. Gel shows PCR products after amplification using primers spanning the gRNA cut site. Presence of a 573 bp band indicates a wildtype (WT) genotype which will be found in clones that are unedited or are heterozygous for the KI construct, a clone with a homozygous KI would not produce a band in this PCR because the KI size would be too large for the elongation time of this reaction.

FIG. 7 presents a time course of NK cell differentiation.

FIG. 8 shows the development of CD45⁺/CD56⁺ iNK over the differentiation time course, derived from WT or SERPINB9 KI/HLA-E KI/B2M KO clonal iPSCs.

FIG. 9 shows a protocol of hepatocyte differentiation.

FIG. 10A presents a plot of the percentage of target (iNK) cells killed by peripheral blood NK (PB-NK) cells from PBNK donor 4. Various iNK cells were incubated with PB-NK cells at various E:T ratios for 24 hours.

FIG. 10B shows a plot of the percentage of target iNK cells killed by PB-NK cells from PBNK donor 6. Various iNK cells were incubated with PB-NK cells at various E:T ratios for 24 hours.

FIG. 10C shows a plot of the percentage of target iNK cells killed by PB-NK cells from PBNK-CLL donor 1. Various iNK cells were incubated with PB-NK cells at various E:T ratios for 24 hours.

FIG. 10D shows a plot of the percentage of target iNK cells killed by PB-NK cells from PBNK donor 4. Various iNK cells were incubated with PB-NK cells at various E:T ratios for 24 hours.

FIG. 10E shows a plot of the percentage of target iNK cells killed by PB-NK cells from PBNK donor 6. Various iNK cells were incubated with PB-NK cells at various E:T ratios for 24 hours.

FIG. 11 presents of plot of target cells (PHH and hepatoblasts differentiated from iPSC WT, edited iPSC PD-L1 KI/HLA-E KI/B2M KO/CIITA KO, and edited iPSC SERPINB9 KI/HLA-E KI/B2M KO) killed by PB-NK cells. Hepatoblasts differentiated from wild type iPSCs (“WT”) or genome edited iPSCs (“PDL1+/HLA-E+/B2M KO/CIITAKO” and “SERPINB9+/HLA-E+/B2M KO”) were incubated with PB-NK cells at various E:T ratios for 24 hours.

FIG. 12 shows a plot of target cells (hepatoblasts differentiated from genome edited iPSC PDL1 KI/HLA-E KI/B2M KO/CIITA KO or genome edited iPSC SERPINB9 KI/HLA-E KI/B2M KO) incubated with or without NKG2A antibodies killed by PB-NK cells.

FIG. 13A shows a plot of the percentage of lentivirus transduced K562 cells killed by NK92 cells after 24 hrs.

FIG. 13B shows a plot of the percentage of lentivirus transduced K562 cells killed by NK92 cells after 72 hrs.

FIG. 14A presents a plot the percentage of genome edited and lentivirus transduced B2M KO Jurkat cells killed by PB-NK cells vs WT Jurkat cells.

FIG. 14B presents a plot of the percentage of genome edited and lentivirus transduced B2M KO Jurkat cells with or without SERPINB KI killed by PB-NK cells vs WT Jurkat cells.

FIG. 15 shows a plot of the percent killing of K562 cancer cell by lentivirus transduced NK92 cells.

FIG. 16 presents a map the B2M-CAGGS-SERPINB9-P2A-IL15/IL15Rα donor plasmid.

FIG. 17 presents a map the B2M-CAGGS-SERPINB9-P2A-IL15/IL15Rα (Flashlight) donor plasmid.

FIG. 18 shows percentage of cells in a bulk population that had HLA-ABC⁺ expression or IL15 surface expression. Cells were analyzed by flow cytometry.

FIGS. 19A and 19B provide graphs demonstrating expression of differentiation markers in iPSC WT derived iNK cells with edited iPSC derived iNK cells (B2M KO/SERPINB9 KI/IL15/IL15Rα KI). Cells were analyzed by flow cytometry for CD56⁺/NKp44⁺, CD56⁺/NKp46⁺, CD56⁺/CD16⁺, CD56⁺/NKG2D⁺, CD56⁺/CD57⁺, and CD56⁺/CD33⁺ (FIG. 19A) and CD56⁺/NKG2A⁺, CD56⁺/FAS⁺, CD56⁺/FAS-L⁺, CD56⁺/KIR⁺, and PD1⁺/TIGIT⁻ (FIG. 19B).

FIG. 20 presents a map the CIITA-CAGGS-SERPINB9-P2A-HLA-E donor plasmid.

FIG. 21 presents a map the B2M-CAGGS-XIAP-P2A-HIL15/IL15Rα fusion protein donor plasmid.

FIG. 22 presents the plasmid map of CIITA-CAGGS-CD30 CAR 4-P2A-HLA-E trimer donor plasmid.

FIG. 23 presents the plasmid map of CIITA-CAGGS-CD30 CAR 5-P2A-HLA-E trimer donor plasmid.

FIG. 24 presents the plasmid map of CIITA-CAGGS-CD30 CAR 6-P2A-HLA-E trimer donor plasmid.

FIG. 25 presents a map of the B2M-CAGGS-SERPINB9-P2A-HLA-E (Flashlight) donor plasmid.

FIG. 26A-26D present percent of killing by day 29 iNK cells differentiated from cells with base edits (B2M KO, SERPINB9 KI, IL15/IL15Rα KI), prototype (B2M KO, SERPINB9 KI, IL15/IL15Rα KI, CISH KO, and FAS KO), and prototype+CD30 CAR (4, 5, or 6) KI and HLA-E KI of K562 cancer cells (FIG. 26A), KMH2 cancer cells (FIG. 26B), L428 cancer cells (FIG. 26C), or L540 cancer cells (FIG. 26D).

FIG. 27 present a schematic for an in vivo protocol to test the cytotoxicity of iNK cells comprising B2M KO, SERPINB9 KI, IL15/IL15Rα KI, CISH KO. FAS KO, CD30 CAR KI, HLA-E KI, and CIITA KO.

### DETAILED DESCRIPTION OF THE INVENTION

The present disclosure provides engineered cells having increased protection against natural killer cell killing. The increased protection can be provided by inserting a polynucleotide encoding a SERPINB9 protein, such that the engineered cells express SERPINB9, and optionally inserting a polynucleotide encoding a HLA-E protein. For example, an engineered gene that has a modified gene that encodes one or more MHC-I or MHC-II human leukocyte antigens or a component or a transcriptional regulator of a MHC-I or MHC-II complex in order to facilitate immune evasion, can be further engineered to have increased protection against natural killer cell killing by inserting a polynucleotide encoding a SERPINB9 protein.

The present disclosure provides engineered cells comprising an insertion of a polynucleotide encoding a SERPINB9 protein, such that the engineered cells express SERPINB9. In some embodiments, the engineered cells comprise (a) an insertion of a polynucleotide encoding a SERPINB9 and (b) a disruption of at least one gene encoding a MHC-I or MHC-II human leukocyte antigen, a component of a MHC-I or MHC-II complex, or a transcriptional regulator of a MHC-I or MHC-II complex, wherein the engineered cell expresses SERPINB9 and has disrupted expression of one or more of the MHC-I or MHC-II human leukocyte antigens, the component of the MHC-I or MHC-II complex, or the transcriptional regulator of the MHC-I or MHC-II complex. In some embodiments, the engineered cells are stem cells (e.g., iPSCs). In some embodiments, the engineered cells are lineage-restricted progenitor cells or fully differentiated somatic cells (e.g., hematopoietic cells such as NK cells, or hepatoblasts/hepatocytes) derived from the engineered stem cells.

In certain embodiments, the engineered cells described herein evade immune response and/or survive following engraftment into a subject at higher success rates than an unmodified cell. In some embodiments, the engineered cells are hypoimmunogenic. In some embodiments, the engineered cells have (i) improved persistence, (ii) improved immune evasiveness, (iii) improved cytotoxic activity, (iv) improved ADCC activity, and/or (v) improved anti-tumor activity as compared to an unmodified or wild-type cell, e.g., a wild-type iPSC or a wild-type NK cell. In other embodiments, the engineered cells have (i) improved persistence, (ii) improved immune evasiveness, (iii) improved functionality, (iv) improved post-transplantation survivability, and/or (v) improved engraftment as compared to an unmodified or wild-type cell, e.g., a wild-type iPSC or a wild-type hepatocyte.

In some embodiments, the engineered cells lack a functional major histocompatibility complex (MHC). In some embodiments, the engineered cells described herein are gene-edited to disrupt one or more of the genes of an MHC-I or MHC-II complex. In some embodiments, the engineered cells have a disrupted B2M gene and have a reduced expression of B2M (e.g., express less than 30%, less than 25%, less than 20%, less than 10%, less than 5% of the level of an unmodified cell) or do not express a detectable level of B2M. In some embodiments, the engineered cells have a disrupted CIITA gene and have a reduced expression of CIITA (e.g., express less than 30%, less than 25%, less than 20%, less than 10%, less than 5% of the level of an unmodified cell) or do not express a detectable level of CIITA.

In some embodiments, the genome of the engineered cells has a disrupted B2M gene, an inserted polynucleotide encoding SERPINB9, and one or more inserted polynucleotide(s) encoding one or all of: IL15, IL15Rα, IL15/IL15Rα fusion protein, HLA-E trimer (e.g., the HLA-E trimer comprising a B2M signal peptide fused to an HLA-G presentation peptide fused to the B2M membrane protein fused to the HLA-E protein without a signal peptide), one or more CARs, and/or XIAP. The inserted polynucleotide(s) can be inserted in the disrupted B2M gene locus (e.g., in exon 1 of the B2M gene locus).

In some embodiments, the genome of the engineered cells has a disrupted CIITA gene, an inserted polynucleotide encoding SERPINB9, and one or more inserted polynucleotide(s) encoding one or all of: IL15, IL15Rα, IL15/IL15Rα fusion protein, HLA-E trimer, one or more CARs, and/or XIAP. The inserted polynucleotide(s) can be inserted in the disrupted CIITA gene locus (e.g., in exon 2 of the CIITA gene locus).

In some embodiments, the genome of the engineered cells has a disrupted B2M gene and a polynucleotide encoding SERPINB9 inserted into the disrupted B2M gene. The polynucleotide encoding SERPINB9 can be linked to a polynucleotide encoding IL15/IL15Rα fusion protein or HLA-E trimer, such that the engineered cells expresses SERPINB9, SERPINB9 and IL15/IL15Rα, or SERPINB9 and HLA-E. In some embodiments, the genome of the engineered cell can further have a disrupted CIITA gene and a polynucleotide encoding a CAR and/or a polynucleotide encoding an IL15/IL15Rα fusion protein or HLA-E trimer inserted into the disrupted CIITA gene, such that the cell expresses the CAR and/or IL15/IL15Rα fusion protein or HLA-E trimer. In certain embodiments, the engineered cell can further have a disrupted FAS gene and/or a disrupted CISH gene.

In some embodiments, the genome of the engineered cells has a disrupted CIITA gene and a polynucleotide encoding SERPINB9 inserted into the disrupted CIITA gene. The polynucleotide encoding SERPINB9 can be linked to a polynucleotide encoding IL15/IL15Rα fusion protein or HLA-E trimer, such that the engineered cells expresses SERPINB9, SERPINB9 and IL15/IL15Rα, or SERPINB9 and HLA-E. In some embodiments, the genome of the engineered cell can further have a disrupted B2M gene and a polynucleotide encoding XIAP and/or a polynucleotide encoding an IL15/IL15Rα fusion protein or HLA-E trimer inserted into the disrupted CIITA gene, such that the cell expresses XIAP and/or IL15/IL15Rα fusion protein or HLA-E trimer. In some embodiments, the genome of the engineered cell can further have a disrupted B2M gene and a polynucleotide encoding XIAP and/or a polynucleotide encoding a CD16 and/or a CD64, such that the cell expresses XIAP and/or CD16 and/or CD64.

In some embodiments, the engineered cells described herein are stem cells. In some embodiments, the engineered cells described herein are iPSCs. In some embodiments, the engineered cells described herein are mesodermal cells. In some embodiments, the engineered cells described herein are hemogenic endothelium (HE) cells (e.g., definitive hemogenic endothelium cells). In some embodiments, the engineered cells described herein are hematopoietic stem or progenitor cells (HSPCs) (e.g., definitive hematopoietic stem or progenitor cells). In some embodiments, the engineered cells described herein are common lymphoid progenitor (CLP) cells. In some embodiments, the engineered cells described herein are NK progenitor cells. In some embodiments, the engineered cells described herein are immature NK cells. In some embodiments, the engineered cells described herein are NK cells. In some embodiments, the engineered cells described herein are fully differentiated hematopoietic cells (e.g., NK cells). In some embodiments, the engineered cells described herein are definitive endoderm, hepatoblasts, or hepatocytes.

In some embodiments, stem cells (e.g., iPSCs) are gene-edited as described herein and then differentiated into one, two, three, four, five, six or more of the following cell types: mesodermal cells, HE cells, HSPCs, CLP cells, NK progenitor cells, immature NK cells, NK cells, definitive endoderm, hepatoblasts, or hepatocytes. In some embodiments, the differentiated cells maintain all edits made in the cells from which they were derived (e.g., NK cells maintain all edits of gene-edited stem cells (e.g., iPSC cells) from which they were derived, or hepatoblasts/hepatocytes maintain all edits of gene-edited stem cells (e.g., iPSC cells) from which they were derived). In some embodiments, the engineered cells described herein are CD34+ cells. In some embodiments, the engineered cells described herein are multipotent progenitors (MPP). In some embodiments, the engineered cells described herein are common lymphoid progenitor cells. In some embodiments, the engineered cells described herein are T cell progenitors.

### Definitions

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% compared to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

As used herein, the term “induced pluripotent stem cells” or, iPSCs, means that the stem cells are produced from differentiated adult, neonatal or fetal cells that have been induced or changed, i.e., reprogrammed into cells capable of differentiating into tissues of all three germ or dermal layers: mesoderm, endoderm, and ectoderm. The iPSCs produced do not refer to cells as they are found in nature.

The term “hematopoietic stem and progenitor cells,” “hematopoietic stem cells,” “hematopoietic progenitor cells,” or “hematopoietic precursor cells” refers to cells which are committed to a hematopoietic lineage but are capable of further hematopoietic differentiation and include, multipotent hematopoietic stem cells (hematoblasts), myeloid progenitors, megakaryocyte progenitors, erythrocyte progenitors, and lymphoid progenitors. Hematopoietic stem and progenitor cells (HSCs) are multipotent stem cells that give rise to all the blood cell types including myeloid (monocytes and macrophages, neutrophils, basophils, eosinophils, erythrocytes, megakaryocytes/platelets, dendritic cells), and lymphoid lineages (T cells, B cells, NK cells). The term “definitive hematopoietic stem cell” as used herein, refers to CD34+ hematopoietic cells capable of giving rise to both mature myeloid and lymphoid cell types including T cells (also referred interchangeably herein as “T-cell”), NK cells and B-cells. Hematopoietic cells also include various subsets of primitive hematopoietic cells that give rise to primitive erythrocytes, megakarocytes and macrophages.

As used herein, the term “NK cell” or “Natural Killer cell” refer to a subset of peripheral blood lymphocytes defined by the expression of CD56 or CD16 and the absence of the T-cell receptor (CD3). As used herein, the terms “adaptive NK cell” and “memory NK cell” are interchangeable and refer to a subset of NK cells that are phenotypically CD3⁻ and CD56⁺, expressing at least one of NKG2C and CD57, and optionally, CD16, but lack expression of one or more of the following: PLZF, SYK, FceRy, and EAT-2. In some embodiments, isolated subpopulations of CD56⁺ NK cells comprise expression of CD16, NKG2C, CD57, NKG2D, NCR ligands, NKp30, NKp40, NKp46, activating and inhibitory KIRs, NKG2A and/or DNAM-1.

As used herein, the term “hepatoblast” refers to bi-potential progenitor cells that can differentiate into hepatocytes or biliary epithelial cells. Hepatoblasts express alpha-fetoprotein (AFP) and albumin (ALB). The term “hepatocyte” refers to the major parenchymal cells in the liver, as they play key roles in metabolism, detoxification, and protein synthesis. Hepatocytes express ALB, but not AFP. In some embodiments, isolated subpopulations of hepatoblasts comprise expression of AFP, ALB, and/or HNF-4a. In some embodiments, isolated subpopulations of hepatocytes comprise expression of ALB, G6PC, CPS1, ABCC2, UGT2B4, CYP1A2, and/or CYP3A4.

As used herein, the terms “disruption,” “genetic modification” or “gene-edit” generally refer to a genetic modification wherein a site or region of genomic DNA is altered, e.g., by a deletion or insertion, by any molecular biology method, e.g., methods described herein, e.g., by delivering to a site of genomic DNA an endonuclease and at least one gRNA. Exemplary genetic modifications include insertions, deletions, duplications, inversions, and translocations, and combinations thereof. In some embodiments, a genetic modification is a deletion. In some embodiments, a genetic modification is an insertion. In other embodiments, a genetic modification is an insertion-deletion mutation (or indel), such that the reading frame of the target gene is shifted leading to an altered gene product or no gene product. As used herein, the term “engineered cell” refers to a cell with any disruption, genetic modification or gene-edit.

As used herein, the term “deletion” which may be used interchangeably with the terms “genetic deletion”, “knock-out”, or “KO”, generally refers to a genetic modification wherein a site or region of genomic DNA is removed by any molecular biology method, e.g., methods described herein, e.g., by delivering to a site of genomic DNA an endonuclease and at least one gRNA. Any number of nucleotides can be deleted. In some embodiments, a deletion involves the removal of at least one, at least two, at least three, at least four, at least five, at least ten, at least fifteen, at least twenty, or at least 25 nucleotides. In some embodiments, a deletion involves the removal of 10-50, 25-75, 50-100, 50-200, or more than 100 nucleotides. In some embodiments, a deletion involves the removal of an entire target gene, e.g., a B2M gene, a CIITA gene, a FAS gene, or a CISH gene. In some embodiments, a deletion involves the removal of part of a target gene, e.g., all or part of a promoter and/or coding sequence of a B2M gene, a CIITA gene, a FAS gene, or a CISH gene. In some embodiments, a deletion involves the removal of a transcriptional regulator, e.g., a promoter region, of a target gene. In some embodiments, a deletion involves the removal of all or part of a coding region such that the product normally expressed by the coding region is no longer expressed, is expressed as a truncated form, or expressed at a reduced level. In some embodiments, a deletion involves the removal of a splice site resulting in a product KO. In some embodiments, a deletion leads to a decrease in expression of a gene relative to an unmodified cell. In some embodiments, the decrease in expression can be a reduced level of expression (e.g., express less than 30%, less than 25%, less than 20%, less than 10%, less than 5% of the level of an unmodified cell). In some embodiments, the decrease in expression can be eliminated expression (e.g., no expression or do not express a detectable level of RNA and/or protein). Expression can be measured using any standard RNA-based, protein-based, and/or antibody-based detection method (e.g., RT-PCR, ELISA, flow cytometry, immunocytochemistry, and the like). Detectable levels are defined as being higher that the limit of detection (LOD), which is the lowest concentration that can be measured (detected) with statistical significance by means of a given detection method.

As used herein, the term “endonuclease” generally refers to an enzyme that cleaves phosphodiester bonds within a polynucleotide. In some embodiments, an endonuclease specifically cleaves phosphodiester bonds within a DNA polynucleotide. In some embodiments, an endonuclease is a zinc finger nuclease (ZFN), transcription activator like effector nuclease (TALEN), homing endonuclease (HE), meganuclease, MegaTAL, or a CRISPR-associated endonuclease. In some embodiments, an endonuclease is an RNA-guided endonuclease. In certain aspects, the RNA-guided endonuclease is a CRISPR nuclease, e.g., a Type II CRISPR Cas9 endonuclease or a Type V CRISPR Cpf1 endonuclease. In some embodiments, an endonuclease is a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease, or a homolog thereof, a recombination of the naturally occurring molecule thereof, a codon-optimized version thereof, or a modified version thereof, or combinations thereof. In some embodiments, an endonuclease may introduce one or more single-stranded breaks (SSBs) and/or one or more double-stranded breaks (DSBs).

As used herein, the term “guide RNA” or “gRNA” generally refers to short ribonucleic acid that can interact with, e.g., bind to, to an endonuclease and bind, or hybridize to a target genomic site or region. In some embodiments, a gRNA is a single-molecule guide RNA (sgRNA). In some embodiments, a gRNA may comprise a spacer extension region. In some embodiments, a gRNA may comprise a tracrRNA extension region. In some embodiments, a gRNA is single-stranded. In some embodiments, a gRNA comprises naturally occurring nucleotides. In some embodiments, a gRNA is a chemically modified gRNA. In some embodiments, a chemically modified gRNA is a gRNA that comprises at least one nucleotide with a chemical modification, e.g., a 2′-O-methyl sugar modification. In some embodiments, a chemically modified gRNA comprises a modified nucleic acid backbone. In some embodiments, a chemically modified gRNA comprises a 2′-O-methyl-phosphorothioate residue. In some embodiments, a gRNA may be pre-complexed with a DNA endonuclease.

As used herein, the term “insertion” which may be used interchangeably with the terms “genetic insertion” or “knock-in”, generally refers to a genetic modification wherein a polynucleotide is introduced or added into a site or region of genomic DNA by any molecular biological method, e.g., methods described herein, e.g., by delivering to a site of genomic DNA an endonuclease and at least one gRNA. In some embodiments, an insertion may occur within or near a site of genomic DNA that has been the site of a prior genetic modification, e.g., a deletion or insertion-deletion mutation. In some embodiments, an insertion occurs at a site of genomic DNA that partially overlaps, completely overlaps, or is contained within a site of a prior genetic modification, e.g., a deletion or insertion-deletion mutation. In some embodiments, an insertion involves the introduction of a polynucleotide that encodes a protein of interest. In some embodiments, an insertion involves the introduction of one or more polynucleotides that encode SERPINB9, IL15/ILRα fusion protein, HLA-E trimer, a CAR, and/or XIAP. In some embodiments, an insertion involves the introduction of an exogenous promoter, e.g., a constitutive promoter, e.g., a CAG promoter. In some embodiments, an insertion involves the introduction of a polynucleotide that encodes a noncoding gene. In general, a polynucleotide to be inserted is flanked by sequences (e.g., homology arms) having substantial sequence homology with genomic DNA at or near the site of insertion.

As used herein, the terms “Major histocompatibility complex class I” or “MHC-I” generally refer to a class of biomolecules that are found on the cell surface of all nucleated cells in vertebrates, including mammals, e.g., humans; and function to display peptides of non-self or foreign antigens, e.g., proteins, from within the cell (i.e. cytosolic) to cytotoxic T-cells, e.g., CD8⁺ T-cells, in order to stimulate an immune response. In some embodiments, a MHC-I biomolecule is a MHC-I gene or a MHC-I protein. Complexation of MHC-I proteins with beta-2 microglobulin (B2M) protein is required for the cell surface expression of all MHC-I proteins. In some embodiments, decreasing the expression of a MHC-I human leukocyte antigen (HLA) relative to an unmodified cell involves a decrease (or reduction) in the expression of a MHC-I gene. In some embodiments, decreasing the expression of a MHC-I human leukocyte antigen (HLA) relative to an unmodified cell involves a decrease (or reduction) in the cell surface expression of a MHC-I protein. In some embodiments, a MHC-I biomolecule is HLA-A (NCBI Gene ID No: 3105), HLA-B (NCBI Gene ID No: 3106), HLA-C(NCBI Gene ID No: 3107), or B2M (NCBI Gene ID No: 567).

As used herein, the term “Major histocompatibility complex class II” or “MHC-II” generally refer to a class of biomolecules that are typically found on the cell surface of antigen-presenting cells in vertebrates, including mammals, e.g., humans; and function to display peptides of non-self or foreign antigens, e.g., proteins, from outside of the cell (extracellular) to cytotoxic T cells, e.g., CD8⁺ T cells, in order to stimulate an immune response. In some embodiments, an antigen-presenting cell is a dendritic cell, macrophage, or a B cell. In some embodiments, a MHC-II biomolecule is a MHC-II gene or a MHC-II protein. In some embodiments, decreasing the expression of a MHC-II human leukocyte antigen (HLA) relative to an unmodified cell involves a decrease (or reduction) in the expression of a MHC-II gene. In some embodiments, decreasing the expression of a MHC-II human leukocyte antigen (HLA) relative to an unmodified cell involves a decrease (or reduction) in the cell surface expression of a MHC-II protein. In some embodiments, a MHC-II biomolecule is HLA-DPA (NCBI Gene ID No: 3113), HLA-DPB (NCBI Gene ID No: 3115), HLA-DMA (NCBI Gene ID No: 3108), HLA-DMB (NCBI Gene ID No: 3109), HLA-DOA (NCBI Gene ID No: 3111), HLA-DOB (NCBI Gene ID No: 3112), HLA-DQA (NCBI Gene ID No: 3117), HLA-DQB (NCBI Gene ID No: 3119), HLA-DRA (NCBI Gene ID No: 3122), or HLA-DRB (NCBI Gene ID No: 3123).

As used herein, the term “polynucleotide”, which may be used interchangeably with the term “nucleic acid” generally refers to a biomolecule that comprises two or more nucleotides. In some embodiments, a polynucleotide comprises at least two, at least five at least ten, at least twenty, at least 30, at least 40, at least 50, at least 100, at least 200, at least 250, at least 500, or any number of nucleotides. A polynucleotide may be a DNA or RNA molecule or a hybrid DNA/RNA molecule. A polynucleotide may be single-stranded or double-stranded. In some embodiments, a polynucleotide is a site or region of genomic DNA. In some embodiments, a polynucleotide is an endogenous gene that is comprised within the genome of an unmodified cell or gene-edited iPSC. In some embodiments, a polynucleotide is an exogenous polynucleotide that is not integrated into genomic DNA. In some embodiments, a polynucleotide is an exogenous polynucleotide that is integrated into genomic DNA. In some embodiments, a polynucleotide is a plasmid or an adeno-associated viral vector. In some embodiments, a polynucleotide is a circular or linear molecule.

As used herein, the term “safe harbor locus” generally refers to any location, site, or region of genomic DNA that may be able to accommodate a genetic insertion into said location, site, or region without adverse effects on a cell. In some embodiments, a safe harbor locus is an intragenic or extragenic region. In some embodiments, a safe harbor locus is a region of genomic DNA that is typically transcriptionally silent. In some embodiments, a safe harbor locus is described in Sadelain, M. et al., “Safe harbours for the integration of new DNA in the human genome,” Nature Reviews Cancer, 2012, Vol 12, pages 51-58.

As used herein, the term “safety switch” generally refers to a biomolecule that leads a cell to undergo apoptosis. In some embodiments, a safety switch is a protein or gene. In some embodiments, a safety switch is a suicide gene. In some embodiments, a safety switch, e.g., herpes simplex virus thymidine kinase (HSV-tk), leads a cell to undergo apoptosis by metabolizing a prodrug, e.g., ganciclovir. In some embodiments, the overexpressed presence of a safety switch on its own leads a cell to undergo apoptosis.

As used herein, the term “subject” refers to a mammal. In some embodiments, a subject is non-human primate or rodent. In some embodiments, a subject is a human. In some embodiments, a subject has, is suspected of having, or is at risk for, a disease or disorder. In some embodiments, a subject has one or more symptoms of a disease or disorder.

As used herein, the term “survival factor” generally refers to a protein (e.g., expressed by a polynucleotide as described herein) that, when increased or decreased in a cell, enables the cell, e.g., an engineered cell, to survive after transplantation or engraftment into a host subject at higher survival rates relative to an unmodified cell. In some embodiments, a survival factor is a human survival factor. In some embodiments, a survival factor is a member of a critical pathway involved in cell survival. In some embodiments, a critical pathway involved in cell survival has implications on hypoxia, reactive oxygen species, nutrient deprivation, and/or oxidative stress. In some embodiments, the survival factor is involved with apoptosis. In some embodiments, the genetic modification, e.g., deletion or insertion, of at least one survival factor enables an engineered cell to survive for a longer time period, e.g., at least 1.05, at least 1.1, at least 1.25, at least 1.5, at least 2, at least 3, at least 4, at least 5, at least 10, at least 20, or at least 50 times longer time period, than an unmodified cell following engraftment. In some embodiments, a survival factor is ZNF143 (NCBI Gene ID No: 7702), TXNIP (NCBI Gene ID No: 10628), FOXO1 (NCBI Gene ID No: 2308), INK (NCBI Gene ID No: 5599), SOCS3 (NCBI Gene ID No: 9021), tissue factor (NCBI Gene ID No: 2152), or MANF (NCBI Gene ID No: 7873). In some embodiments, a survival factor is inserted into a cell, e.g., an engineered cell. In some embodiments, a survival factor is deleted from a cell, e.g., an engineered cell. In some embodiments, an insertion of a polynucleotide that encodes MANF enables a cell, e.g., an engineered cell, to survive after transplantation or engraftment into a host subject at higher survival rates relative to an unmodified cell. In some embodiments, a deletion or insertion-deletion mutation within or near a ZNF143, TXNIP, FOXO1, SOCS3, tissue factor, or JNK gene enables a cell, e.g., an engineered cell, to survive after transplantation or engraftment into a host subject at higher survival rates relative to an unmodified cell.

As used herein, the term “transcriptional regulator of MHC-I or MHC-II” generally refers to a biomolecule that modulates, e.g., increases or decreases, the expression of a MHC-I and/or MHC-II human leukocyte antigen. In some embodiments, a biomolecule is a polynucleotide, e.g., a gene, or a protein. In some embodiments, a transcriptional regulator of MHC-I or MHC-II will increase or decrease the cell surface expression of at least one MHC-I or MHC-II protein. In some embodiments, a transcriptional regulator of MHC-I or MHC-II will increase or decrease the expression of at least one MHC-I or MHC-II gene. In some embodiments, the transcriptional regulator is CIITA (NCBI Gene ID No: 4261) or NLRC5 (NCBI Gene ID No: 84166). In some embodiments, deletion or reduction of expression of CIITA or NLRC5 decreases expression of at least one MHC-I or MHC-II gene.

As used herein, the term “engineered cell” generally refers to a genetically modified cell that is less susceptible to allogeneic rejection during a cellular transplant and/or demonstrates increased survival after transplantation, relative to an unmodified cell. In some embodiments, a genetically modified cell as described herein is an engineered cell. In some embodiments, the engineered cell has increased immune evasion and/or cell survival compared to an unmodified cell. In some embodiments, the engineered cell has increased cell survival compared to an unmodified cell. In some embodiments, the engineered cell has (i) improved persistence, (ii) improved immune evasiveness, (iii) improved cytotoxic activity, (iv) improved ADCC activity, and/or (v) improved anti-tumor activity compared to an unmodified cell. In other embodiments, the engineered cell has (i) improved persistence, (ii) improved immune evasiveness, (iii) improved functionality, (iv) improved post-transplantation survivability, and/or (v) improved engraftment as compared to an unmodified cell. In some embodiments, an engineered cell may be a stem cell. In some embodiments, an engineered cell may be an embryonic stem cell (ESC), an adult stem cell (ASC), an induced pluripotent stem cell (iPSC), or a hematopoietic stem or progenitor cell (HSPC). In some embodiments, an engineered cell may be a differentiated cell. In some embodiments, an engineered cell may be a somatic cell (e.g., immune system cells). In some embodiments, an engineered cell is administered to a subject. In some embodiments, an engineered cell is administered to a subject who has, is suspected of having, or is at risk for a disease. In some embodiments, the engineered cell is capable of being differentiated into lineage-restricted progenitor cells or fully differentiated somatic cells. In some embodiments, the lineage-restricted progenitor cells are pancreatic endoderm progenitors, pancreatic endocrine progenitors, mesenchymal progenitor cells, hepatoblasts, muscle progenitor cells, blast cells, or neural progenitor cells. In some embodiments, the fully differentiated somatic cells are endocrine secretory cells such as pancreatic beta cells, hepatocytes, endodermal cells, and in some embodiments the fully differentiated cells are epithelial cells, macrophages, adipocytes, kidney cells, blood cells, or immune system cells.

As used herein, the term “unmodified cell” refers to a cell that has not been subjected to a genetic modification involving a polynucleotide or gene that encodes any of the genes described herein. In some embodiments, an unmodified cell may be a stem cell. In some embodiments, an unmodified cell may be an embryonic stem cell (ESC), an adult stem cell (ASC), an induced pluripotent stem cell (iPSC), or a hematopoietic stem or progenitor cell (HSPC). In some embodiments, an unmodified cell may be a differentiated cell. In some embodiments, an unmodified cell may be selected from somatic cells (e.g., hepatocytes or immune system cells, e.g., a T cell, e.g., a CD8⁺ T cell). If a gene-edited iPSC or NK cell is compared “relative to an unmodified cell”, the iPSC or NK cell and the unmodified cell are the same cell type or share a common parent cell line, e.g., a gene-edited NK cell is compared relative to an unmodified NK cell.

As used herein, the term “within or near a gene” refers to a site or region of genomic DNA that is an intronic or exonic component of a said gene or is located proximal to a said gene. In some embodiments, a site of genomic DNA is within a gene if it comprises at least a portion of an intron or exon of said gene. In some embodiments, a site of genomic DNA located near a gene may be at the 5′ or 3′ end of said gene (e.g., the 5′ or 3′ end of the coding region of said gene). In some embodiments, a site of genomic DNA located near a gene may be a promoter region or repressor region that modulates the expression of said gene. In some embodiments, a site of genomic DNA located near a gene may be on the same chromosome as said gene. In some embodiments, a site or region of genomic DNA is near a gene if it is within 50 kb, 40 kb, 30 kb, 20 kb, 10 kb, 5 kb, 1 kb, or closer to the 5′ or 3′ end of said gene (e.g., the 5′ or 3′ end of the coding region of said gene).

As used herein, the term “tolerogenic factor” generally refers to a protein (e.g., expressed by a polynucleotide as described herein) that, when increased or decreased in a cell, enables the cell, e.g., an engineered cell, to inhibit or evade immune rejection after transplantation or engraftment into a host subject at higher rates relative to an unmodified cell. In some embodiments, a tolerogenic factor is a human tolerogenic factor. In some embodiments, the genetic modification of at least one tolerogenic factor (e.g., the insertion or deletion of at least one tolerogenic factor) enables a cell, e.g., an engineered cell. to inhibit or evade immune rejection with rates at least 1.05, at least 1.1, at least 1.25, at least 1.5, at least 2, at least 3, at least 4, at least 5, at least 10, at least 20, or at least 50 times higher than an unmodified cell following engraftment. In some embodiments, a tolerogenic factor is HLA-E (NCBI Gene ID No: 3133), HLA-G (NCBI Gene ID No: 3135), CTLA-4 (NCBI Gene ID No: 1493), CD47 (NCBI Gene ID No: 961), TNFAIP3 (NCBI Gene ID No: 7128), CD39 (NCBI Gene ID No: 953), or PD-L1 (NCBI Gene ID No: 29126). In some embodiments, a tolerogenic factor is inserted into a cell, e.g., an engineered cell. In some embodiments, a tolerogenic factor is deleted from a cell, e.g., an engineered cell. In some embodiments, an insertion of a polynucleotide that encodes HLA-E, HLA-G, CTLA-4, CD47, TNFAIP3, CD39, and/or PD-L1 enables a cell, e.g., an engineered cell, to inhibit or evade immune rejection after transplantation or engraftment into a host subject.

As used herein, the term “comprising” or “comprises” is inclusive or open-ended and does not exclude additional, unrecited elements, ingredients, or method steps; the phrase “consisting of” or “consists of” is closed and excludes any element, step, or ingredient not specified; and the phrase “consisting essentially of” or “consists essentially” means that specific further components can be present, namely those not materially affecting the essential characteristics of the compound, composition, or method. When used in the context of a sequence, the phrase “consisting essentially of” or “consists essentially” means that the sequence can comprise substitutions and/or additional sequences that do not change the essential function or properties of the sequence.

### Gene Editing

Described herein are strategies to enable genetically modified cells to evade immune response and/or increase their survival, or viability following engraftment into a subject. In some embodiments, these strategies enable gene-edited cells to evade immune response and/or survive at higher success frequencies than an unmodified cell.

In certain embodiments, any cells described herein are gene-edited using any of the gene-editing methods described herein (e.g., using CRISPR/Cas gene editing to insert or delete one or more nucleotides). In some embodiments, a disrupted gene is a gene that does not encode functional protein. In some embodiments, a cell that comprises a disrupted gene does not express (e.g., at the cell surface) a detectable level (e.g. by antibody, e.g., by flow cytometry) of the protein encoded by the gene. A cell that does not express a detectable level of the protein may be referred to as a knockout (or null) cell.

In some embodiments, the cells described herein are gene-edited to disrupt one or more of the genes encoding an MHC-I or MHC-II human leukocyte antigen, a component of a MHC-I or MHC-II complex, or a transcriptional regulator of a MHC-I or MHC-II complex. In some embodiments, the cells described herein are gene-edited to disrupt one or more of the genes encoding an MHC-I or MHC-II human leukocyte antigen. In some embodiments, the cells described herein are gene-edited to disrupt one or more of the genes encoding one or more components of a MHC-I or MHC-II complex. In some embodiments, the cells described herein are gene-edited to disrupt one or more of the genes encoding one or more transcriptional regulator of a MHC-I or MHC-II complex. In some embodiments, the gene that encodes the one or more MHC-I or MHC-II human leukocyte antigens or the component or the transcriptional regulator of the MHC-I or MHC-II complex is a MHC-I gene chosen from HLA-A, HLA-B, or HLA-C, a MHC-II gene chosen from HLA-DP, HLA-DM, HLA-DOA, HLA-DOB, HLA-DQ, or HLA-DR, or a gene chosen from B2M, NLRC5, CIITA, RFX5, RFXAP, or RFXANK.

In some embodiments, the cells described herein are gene-edited to disrupt one or more genes including but not limited to: B2M, CIITA, CISH, FAS, REGNASE-1, ADAM17, PD-1, NKG2A, CD70, and/or ALK4, type I activin receptor (e.g., conditionally). In some embodiments, the cells described herein are gene-edited to disrupt B2M and/or CIITA. In some embodiments, the cells described herein are gene-edited to disrupt B2M. In some embodiments, the cells described herein are gene-edited to disrupt CIITA. In some embodiments, the cells described herein are gene-edited to disrupt CISH. In some embodiments, the cells described herein are gene-edited to disrupt FAS. In some embodiments, the cells described herein are gene-edited to disrupt REGNASE-1. In some embodiments, the cells described herein are gene-edited to disrupt ADAM17. In some embodiments, the cells described herein are gene-edited to disrupt FAS. In some embodiments, the cells described herein are gene-edited to disrupt TIGIT In some embodiments, the cells described herein are gene-edited to disrupt PD-1. In some embodiments, the cells described herein are gene-edited to disrupt NKG2A. In some embodiments, the cells described herein are gene-edited to disrupt CD70. In some embodiments, the cells described herein are gene-edited to disrupt ALK4, type I activin receptor (e.g., conditionally).

In some embodiments, the cells described herein are gene-edited to insert a polynucleotide encoding, without limitation, one or more of the following: SERPINB9, IL15, IL15Rα, IL15/IL15Rα fusion protein, HLA-E trimer, a CAR, XIAP, CD16, CD64. In some embodiments, the cells described herein are gene-edited to insert a polynucleotide encoding SERPINB9. In some embodiments, the cells described herein are gene-edited to insert a polynucleotide encoding IL15. In some embodiments, the cells described herein are gene-edited to insert a polynucleotide encoding IL15Rα. In some embodiments, the cells described herein are gene-edited to insert a polynucleotide encoding a fusion protein of IL15 and IL15Rα. In some embodiments, the cells described herein are gene-edited to insert a polynucleotide encoding HLA-E (e.g., wherein the HLA-E is a trimer comprising a B2M signal peptide fused to an HLA-G presentation peptide fused to the B2M membrane protein fused to the HLA-E protein without a signal peptide). In some embodiments, the cells described herein are gene-edited to insert a polynucleotide encoding a CAR. In some embodiments, the cells described herein are gene-edited to insert a polynucleotide encoding XIAP. In some embodiments, the cells described herein are gene-edited to insert a polynucleotide encoding CD16. In some embodiments, the cells described herein are gene-edited to insert a polynucleotide encoding CD64. In some embodiments, the cells described herein are gene-edited to insert a polynucleotide encoding SERPINB9-P2A-IL15/IL15Rα. In some embodiments, the cells described herein are gene-edited to insert a polynucleotide encoding SERPINB9-P2A-HAL-E. In some embodiments, the cells described herein are gene-edited to insert a polynucleotide encoding XIAP-P2A-IL15/IL15Rα. In some embodiments, the cells described herein are gene-edited to insert a polynucleotide encoding a CAR-P2A-HLA-E. In some embodiments, the CAR is a CD30 CAR.

In some embodiments, the cells described herein are gene-edited to insert a polynucleotide encoding SERPINB9 alone or in combination with a polynucleotide encoding IL15/IL15Rα fusion protein and/or HLA-E trimer, wherein the cell has a disrupted expression of B2M (e.g., the cell is gene-edited to disrupt B2M leading to, e.g., elimination of B2M expression). In some embodiments, the polynucleotide encoding SERPINB9 alone or in combination with a polynucleotide encoding IL15/IL15Rα fusion protein and/or HLA-E trimer is inserted in the B2M gene locus (e.g., in exon 1 of the B2M gene locus). In some embodiments, the cells described herein are gene-edited to insert any of the polynucleotides described herein wherein the cell has a disrupted expression of CIITA (e.g., the cell is gene-edited to disrupt CIITA leading to, e.g., elimination of CIITA expression). In some embodiments, the cells described herein are gene-edited to insert any of the polynucleotides described herein in the disrupted CIITA gene locus (e.g., in exon 2 of the CIITA gene locus).

In some embodiments, the cells described herein are gene-edited to insert a polynucleotide encoding one or more chimeric antigen receptors (CARs). In some embodiments, and without limitation, the CAR is a BCMA (i.e., B cell maturation antigen) CAR, CD30 CAR, CD19 CAR, CD33 CAR, NKG2D (i.e., natural killer group 2D receptor) CAR (or a CAR or receptor comprising an NKG2D ectodomain), CD70 CAR, NKp30 (i.e., natural killer protein 30) CAR, CD73 CAR, GPR87 (i.e., G protein-coupled receptor 87) CAR, or SLC7A11 (i.e., solute carrier family 7 member 11, which is also called xCT) CAR. In some embodiments, the CAR is a BCMA CAR. In some embodiments, the s a CD30 CAR. In some embodiments, the CAR is a GPC3 CAR. In some embodiments, the CAR is a CD33 CAR. In some embodiments, the CAR is a CD19 CAR. In some embodiments, the CAR is a CD33 CAR. In some embodiments, the CAR is a NKG2D CAR (or a CAR or receptor comprising an NKG2D ectodomain). In some embodiments, the CAR is a CD70 CAR. In some embodiments, the CAR is a NKp30 CAR. In some embodiments, the CAR is a CD73 CAR. In some embodiments, the CAR is a GPR87 CAR. In some embodiments, the CAR is a L1V1A CAR. In some embodiments, the CAR is a A33 CAR. In some embodiments, the CAR is a EGFR CAR. In some embodiments, the CAR is a CD20 CAR. In some embodiments, the CAR is a SLC7A11 CAR. In some embodiments, the cells described herein are gene-edited to insert a polynucleotide encoding a CAR, wherein the cell has a disrupted expression of CIITA (e.g., the cell is gene-edited to disrupt CIITA leading to, e.g., elimination of CIITA expression). In some embodiments, the CAR is inserted in the disrupted CIITA gene. In some embodiments, the CAR is inserted in exon 2 of the CIITA gene locus. In some embodiments, the cells described herein are gene-edited to insert a polynucleotide encoding a CAR, wherein the cell has a disrupted expression of B2M (e.g., the cell is gene-edited to disrupt B2M leading to, e.g., elimination of B2M expression). In some embodiments, the CAR is inserted in the disrupted B2M gene locus (e.g., in exon 1 of the B2M gene locus).

In some embodiments, the present disclosure provides a method of generating genome-engineered stem cells (e.g., iPSCs), wherein the stem cells comprise at least one targeted genomic modification at one or more selected sites in genome, the method comprising genetically engineering a cell type as described herein by introducing into said cells one or more constructs to allow targeted modification at a selected site; introducing into said cells one or more double strand breaks at the selected sites using one or more endonuclease capable of selected site recognition; and culturing the edited cells to allow endogenous DNA repair to generate targeted insertions or deletions at the selected sites; thereby obtaining genome-modified stem cells. The stem cells (e.g., iPSCs) generated by this method will comprise at least one functional targeted genomic modification, and wherein the genome-modified cells, are then capable of being differentiated into progenitor cells or fully-differentiated cells (e.g., natural killer (NK) cells or hepatocytes). In some embodiments, the differentiated cells (e.g., NK cells or hepatocytes) maintain all of the gene-edits of the cells from which they were derived.

In some embodiments, a ribonucleoprotein particle (RNP) containing an RNA-guided nuclease (e.g., a Cas nuclease, such as a Cas9 nuclease) and a gRNA targeting the gene to be disrupted are delivered to any cell described herein (e.g., iPSC). A RNP is an RNA-guided nuclease (e.g., Cas9) pre-complexed/complexed with a gRNA. In other embodiments, the RNA-guided nuclease and gRNA are delivered separately to cells. In some embodiments, at least 50% of the engineered cells of a population of cells does not express a detectable level of the protein encoded by the disrupted gene. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered cells of a population do not express a detectable level of the disrupted gene product.

In some embodiments, at least 50% of the engineered cells of a population of cells expresses a detectable level of the protein encoded by the inserted polynucleotide. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered cells of a population express a detectable level of the protein encoded by the inserted polynucleotide.

### MHC I and MHC II Edits

Major histocompatibility complex I and II (MHC I and MHC II respectively) are cell surface proteins which perform an essential role in the adaptive immune system. The genes that encode the major histocompatibility complex (MHC) are located on human Chr. 6p21. The resultant proteins coded by the MHC genes are a series of surface proteins that are essential in donor compatibility during cellular transplantation. MHC genes are divided into MHC class I (MHC-I) and MHC class II (MHC-II). MHC-I genes (HLA-A, HLA-B, and HLA-C) are expressed in almost all tissue cell types, presenting “non-self” antigen-processed peptides to CD8⁺ T cells, thereby promoting their activation to cytolytic CD8⁺ T cells. Transplanted or engrafted cells expressing “non-self” MHC-I molecules will cause a robust cellular immune response directed at these cells and ultimately resulting in their demise by activated cytolytic CD8⁺ T cells. MHC-I proteins are intimately associated with beta-2-microglobulin (B2M) in the endoplasmic reticulum, which is essential for forming functional MHC-I molecules on the cell surface. In addition, there are three non-classical MHC-II molecules (HLA-E, HLA-F, and HLA-G), which have immune regulatory functions. MHC-II biomolecule include HLA-DP, HLA-DM, HLA-DOA, HLA-DOB, HLA-DQ, and HLA-DR. Due to their primary function in the immune response, MHC-I and MHC-II biomolecules contribute to immune rejection following cellular engraftment of non-host cells, e.g., cellular engraftment for purposes of regenerative medicine.

In some embodiments, a cell comprises a genomic modification of one or more MHC-I or MHC-II genes. In some embodiments, a cell comprises a genomic modification of one or more polynucleotide sequences that regulates the expression of MHC-I and/or MHC-II. In some embodiments, a genetic modification of the disclosure is performed using any gene editing method including but not limited to those methods described herein.

In some embodiments, any of the cells described herein have MHC I and/or MHCII genetic modifications. In some embodiments, MHC I is disrupted. In some embodiments, MHC II is disrupted. In some embodiments, both MHC I and MHC II are disrupted. In some embodiments, a MHC I encoding gene is inserted. In some embodiments, a MHC II encoding gene is inserted. In some embodiments, any genetically modified cell described herein comprises the introduction of at least one genetic modification within or near at least one gene that decreases the expression of one or more MHC-I and MHC-II human leukocyte antigens relative to an unmodified cell; at least one genetic modification that increases the expression of at least one polynucleotide that encodes a tolerogenic factor relative to an unmodified cell. In some embodiments, genetically modified cells comprise the introduction of at least one genetic modification within or near at least one gene that decreases the expression of one or more MHC-I and MHC-II human leukocyte antigens relative to an unmodified cell; at least one genetic modification that increases the expression of at least one polynucleotide that encodes a tolerogenic factor relative to an unmodified cell. In other embodiments, genetically modified cells comprise at least one deletion or insertion-deletion mutation within or near at least one gene that alters the expression of one or more MHC-I and MHC-II human leukocyte antigens relative to an unmodified cell; and at least one insertion of a polynucleotide that encodes at least one tolerogenic factor at a site that partially overlaps, completely overlaps, or is contained within, the site of a deletion of a gene that alters the expression of one or more MHC-I and MHC-II HLAs.

In some embodiments, decreasing the expression of one or more MHC-I and MHC-II human leukocyte antigens relative to an unmodified cell is accomplished by targeting, e.g., for genetic deletion and/or insertion of at least one base pair, in a MHC-I and/or MHC-II gene directly. In some embodiments, decreasing the expression of one or more MHC-I and MHC-II human leukocyte antigens relative to an unmodified cell is accomplished by targeting, e.g., for genetic deletion, a CIITA gene or a B2M gene. In some embodiments, decreasing the expression of one or more MHC-I and MHC-II human leukocyte antigens relative to an unmodified cell is accomplished by targeting, e.g., for genetic deletion, at least one transcriptional regulator of MHC-I or MHC-II. In some embodiments, a transcriptional regulator of MHC-I or MHC-II is a NLRC5, or CIITA gene. In some embodiments, a transcriptional regulator of MHC-I or MHC-II is a RFX5, RFXAP, RFXANK, NFY-A, NFY-B, NFY-C, IRF-1, and/or TAP1 gene.

In some embodiments, the genome of a cell has been modified to delete the entirety or a portion of a HLA-A, HLA-B, and/or HLA-C gene. In some embodiments, the genome of a cell has been modified to delete the entirety or a portion of a promoter region of a HLA-A, HLA-B, and/or HLA-C gene. In some embodiments, the genome of a cell has been modified to delete the entirety or a portion of a gene that encodes a transcriptional regulator of MHC-I or MHC-II. In some embodiments, the genome of a cell has been modified to delete the entirety or a portion of a promoter region of a gene that encodes a transcriptional regulator of MHC-I or MHC-II.

MHC-I cell surface molecules are composed of MHC-encoded heavy chains (HLA-A, HLA-B, or HLA-C) and the invariant subunit beta-2-microglobulin (B2M). Thus, a reduction in the concentration of B2M within a cell allows for an effective method of reducing the cell surface expression of MHC-I cell surface molecules. In some embodiments, polynucleotides encoding at least one tolerogenic factor can be inserted or reinserted into genetically modified cells to create immune-privileged iPSC or differentiated cells. In some embodiments, the iPSC or differentiated cells disclosed herein have been further modified to express one or more tolerogenic factors. Exemplary tolerogenic factors include, without limitation, one or more of HLA-C, HLA-E, HLA-F, HLA-G, PD-L1, CTLA-4-Ig, CD47, CI-inhibitor, and IL-35. In some embodiments, the genetic modification, e.g., insertion, of at least one polynucleotide encoding at least one tolerogenic factor enables a gene-edited iPSC or differentiated cell to inhibit or evade immune rejection with rates at least 1.05, at least 1.1, at least 1.25, at least 1.5, at least 2, at least 3, at least 4, at least 5, at least 10, at least 20, or at least 50 times higher than an unmodified cell following engraftment. In some embodiments, an insertion of a polynucleotide that encodes HLA-E, HLA-G, CTLA-4, CD47, and/or PD-L1 enables an iPSC or a differentiated cell to inhibit or evade immune rejection after transplantation or engraftment into a host subject.

The polynucleotide encoding SERPINB9 and/or other proteins of interest generally comprises left and right homology arms that flank the sequence encoding SERPINB9 and/or the proteins of interest. The homology arms have substantial sequence homology to genomic DNA at or near the targeted insertion site. For example, the left homology arm can be a nucleotide sequence homologous with a region located to the left or upstream of the target site or cut site and the right homology arm can be a nucleotide sequence homologous with a region located to the right or downstream of the target site or cut site. The proximal end of each homology arm can be homologous to genomic DNA sequence abutting the cut site. Alternatively, the proximal end of each homology arm can be homologous to genomic DNA sequence located up to about 10, 20, 30, 40, 50, 60, or 70 nucleobases away from the cut site. As such, the polynucleotide encoding the tolerogenic factor can be inserted into the targeted gene locus within about 10, 20, 30, 40, 50, 60, or 70 base pairs of the cut site, and additional genomic DNA bordering the cut site (and having no homology to a homolog arm) can be deleted. The homology arms can range in length from about 50 nucleotides to several of thousands of nucleotides. In some embodiments, the homology arms can range in length from about 500 nucleotides to about 1000 nucleotides. In some embodiments, the homology arms are 600 bp, 700 bp, 800 bp, or 900 bp. In some embodiments, the homology arms are 800 base pairs. In some embodiments, the substantial sequence homology between the homology arms and the genomic DNA is at least about 80%, at least about 85%, at least about 90%, at least about 95%, or at least about 99%. In some embodiments, the homology arms have 100% sequence identity with genomic DNA flanking the target site.

In some embodiments, the at least one polynucleotide encoding SERPINB9 and/or other proteins of interest is operably linked to an exogenous promoter. In some embodiments, the exogenous promoter can be a constitutive, inducible, temporal-, tissue-, or cell type-specific promoter. In some embodiments, the exogenous promoter is a CAGGS, CMV, EF1a, PGK, CAG, or UBC promoter.

In some embodiments, the at least one polynucleotide encoding SERPINB9 and/or other proteins of interest is inserted into a safe harbor locus, e.g., the AAVS 1 locus. In some embodiments, a safe harbor locus for inserting any gene described herein is selected from, but not limited to AAVS1 (PPP1 R12C), ALB, Angpt13, ApoC3, ASGR2, CCR5, FIX (F9), G6PC, Gys2, HGD, Lp(a), Pcsk9, Serpinal, TF, and TTR.

In some embodiments, the at least one polynucleotide encoding SERPINB9 and/or other proteins of interest is inserted into a site or region of genomic DNA that partially overlaps, completely overlaps, or is contained within (i.e., is within or near) a MHC-I gene, MHC-II gene, or a transcriptional regulator of MHC-I or MHC-II.

In some embodiments, the cells further comprise increased or decreased expression, e.g., by a genetic modification, of one or more additional genes that are not necessarily implicated in either immune evasion or cell survival post-engraftment. In some embodiments, the cells further comprise increased expression of one or more safety switch proteins relative to an unmodified cell. In some embodiments, the cells comprise increased expression of one or more additional genes that encode a safety switch protein. In some embodiments, a safety switch is also a suicide gene. In some embodiments, a safety switch is a p53-based molecule, herpes simplex virus-1 thymidine kinase (HSV-tk) or inducible caspase-9. In some embodiments, a polynucleotide that encodes at least one safety switch is inserted into a genome, e.g., into a safe harbor locus. In some other embodiments, the one or more additional genes that are genetically modified encode one or more of safety switch proteins; targeting modalities; receptors; signaling molecules; transcription factors; pharmaceutically active proteins or peptides; drug target candidates; and proteins promoting engraftment, trafficking, homing, viability, self-renewal, persistence, and/or survival thereof integrated with the construct.

### B2M Gene Edits

In some embodiments, the genome of any cell described herein is modified to disrupt beta-2-microglobulin (B2M or p2M) gene (NCBI Gene ID: 567). B2M is a non-polymorphic gene that encodes a common protein subunit required for surface expression of all polymorphic MHC class I heavy chains. HLA-I proteins are intimately associated with B2M in the endoplasmic reticulum, which is essential for forming functional, cell-surface expressed HLA-I molecules. Disrupting its expression by gene editing will prevent host versus therapeutic cell responses leading to increased therapeutic cell persistence. In some embodiments, expression of the endogenous B2M gene is eliminated to prevent a host-versus-graft response. In some embodiments, the disrupted B2M can prevent allo-immune response due to MHC-I.

In some embodiments, any of the gene-editing methods described herein are used to disrupt the B2M gene. In some embodiments, any engineered cell described herein comprises a disrupted B2M gene. In some embodiments, an iPSC described herein comprises a disrupted B2M gene. In some embodiments, a differentiated cell described herein comprises a disrupted B2M gene.

In some embodiments, a ribonucleoprotein particle (RNP) containing an RNA-guided nuclease (e.g., a Cas nuclease, such as a Cas9 nuclease) and a gRNA targeting the B2M gene (or any other gene of interest) are delivered to any cell described herein (e.g., iPSC). A ribonucleoprotein particle (RNP) comprises an RNA-guided nuclease (e.g., Cas9) pre-complexed/complexed with a gRNA. In other embodiments, the RNA-guided nuclease and gRNA are delivered separately to cells. In some embodiments, the gRNA targets a site in the B2M gene. Non-limiting examples of modified and unmodified B2M gRNA sequences that may be used as provided herein to create a genomic disruption in the B2M gene include sequences corresponding to a sequence of SEQ ID NO: 1. In some embodiments, a gRNA is used to target the B2M site for gene-editing. Other gRNA sequences may be designed using the B2M gene sequence located on Chromosome 15 (GRCh38 coordinates: Chromosome 15: 44,711,477-44,718,877; Ensembl: ENSG00000166710). Additional B2M gRNAs are disclosed in U.S. Pat. No. 10,724,052. In some embodiments, any B2M RNP described herein is used in combination with a donor plasmid containing B2M homology arms for insertion of any polynucleotide described herein.

In some embodiments, the gRNA comprises a polynucleotide sequence corresponding to a sequence consisting of SEQ ID NO: 1. In some embodiments, a gRNA/CRISPR nuclease complex targets and cleaves a target site in the B2M locus. Repair of a double-stranded break by NHEJ can result in a deletion of at least on nucleotide and/or an insertion of at least one nucleotide, thereby disrupting or eliminating expression of B2M. In some embodiments, the B2M locus is targeted by at least two CRISPR systems each comprising a different gRNA, such that cleavage at two sites in the B2M locus leads to a deletion of the sequence between the two cuts, thereby eliminating expression of B2M.

In some embodiments, the homology arms are used with a B2M guide. In some embodiments, the homology arms are designed to be used with any B2M guide that would eliminate the start site of the B2M gene. In some embodiments, the B2M homology arms comprise or consist essentially of a polynucleotides of the sequence of SEQ ID NOs: 3 and 19, or polynucleotides having at least 85%, 90%, 95%, or 99% sequence identity with that of SEQ ID NOs: 3 or 19. In some embodiments, the left B2M homology arm can comprise or consist essentially of SEQ ID NO: 3, or a polynucleotide sequence having at least 85%, 90%, 95%, or 99% sequence identity with that of SEQ ID NO: 3. In some embodiments, the right B2M homology arm can comprise or consist essentially of SEQ ID NO: 19, or a polynucleotide sequence having at least 85%, 90%, 95%, or 99% sequence identity with that of SEQ ID NO: 19.

In some embodiments, gRNAs targeting the B2M genomic region create Indels in the B2M gene disrupting expression of the mRNA or protein.

In some embodiments, at least 50% of the engineered cells of a population of cells does not express a detectable level of B2M surface protein. For example, at least 55%, at least 60%, at least 70%, at least 75%, at least 80%, at least 85%, at least 90%, or at least 95% of the engineered cells of a population may not express a detectable level of B2M surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered cells of a population does not express a detectable level of B2M surface protein.

In some embodiments, less than 50% of the engineered cells of a population of cells express a detectable level of B2M surface protein. In some embodiments, less than 30% of the engineered cells of a population of cells express a detectable level of B2M surface protein. For example, less than 50%, less than 30%, less than 25%, less than 20%, less than 15%, less than 10%, or less than 5% of the engineered cells of a population of cells express a detectable level of B2M surface protein. In some embodiments, 40%-30%, 40%-20%, 40%-10%, 40%-5%, 30%-20%, 30%-10%, 30%-5%, 20%-10%, 20%-5%, or 10%-5% of the engineered cells of a population of cells express a detectable level of B2M surface protein.

### CIITA Gene Edits

In some embodiments, the genome of any cell described herein is modified to disrupt Class II major histocompatibility complex transactivator (CIITA) gene (NCBI Gene ID: 4261). CIITA is a member of the LR or nucleotide binding domain (NBD) leucine-rich repeat (LRR) family of proteins and regulates the transcription of MHC-II by associating with the MHC enhanceosome. The expression of CIITA is induced in B cells and dendritic cells as a function of developmental stage and is inducible by IFN-7 in most cell types. In some embodiments, the disrupted CIITA gene locus can prevent allo-immune response due to MHC-II.

In some embodiments, any of the gene-editing methods described herein are used to disrupt the CIITA gene. In some embodiments, any engineered cell described herein comprises a disrupted CIITA gene. In some embodiments, an iPSC described herein comprises a disrupted CIITA gene. In some embodiments, a differentiated cell described herein comprises a disrupted CIITA gene.

In some embodiments, a ribonucleoprotein particle (RNP) containing an RNA-guided nuclease (e.g., a Cas nuclease, such as a Cas9 nuclease) and a gRNA targeting the CIITA gene (or any other gene of interest) are delivered to any cell described herein (e.g., iPSC). A ribonucleoprotein particle (RNP) comprises an RNA-guided nuclease (e.g., Cas9) pre-complexed/complexed with a gRNA. In other embodiments, the RNA-guided nuclease and gRNA are delivered separately to cells. In some embodiments, modified or unmodified CIITA gRNA sequences may be used to create a genomic disruption in the CIITA gene. In some embodiments, the gRNA targets a site within the CIITA gene. In some embodiments, the CIITA gRNA targets a sequence comprising SEQ ID NO: 41. In some embodiments, the gRNA comprises a spacer sequence corresponding to a sequence consisting of SEQ ID NO:41. Additional CIITA gRNAs are disclosed in U.S. Provisional Application No. 63/250,048. In some embodiments, any CIITA RNP can be used in combination with a donor plasmid containing CIITA homology arms for insertion of any polynucleotide described herein.

In some embodiments, gRNAs targeting the CIITA genomic region create indels in the CIITA gene disrupting expression of the mRNA or protein. In some embodiments, a gRNA/CRISPR nuclease complex targets and cleaves a target site in the CIITA locus. Repair of a double-stranded break by NHEJ can result in a deletion of at least on nucleotide and/or an insertion of at least one nucleotide, thereby disrupting or eliminating expression of CIITA. In some embodiments, the CIITA gene locus is targeted by at least two CRISPR systems each comprising a different gRNA, such that cleavage at two sites in the CIITA locus leads to a deletion of the sequence between the two cuts, thereby eliminating expression of CIITA.

In some embodiments, the homology arms are used with a CIITA guides (e.g., gRNA comprising a spacer corresponding to a sequence consisting of SEQ ID NO: 41). In some embodiments, the homology arms are designed to be used with any CIITA guide that would eliminate the start site of the CIITA gene. In some embodiments, the CIITA homology arms comprise or consist essentially of polynucleotides of SEQ ID NOs: 42 and 43, or polynucleotide sequences having at least 85%, 90%, 95%, or 99% sequence identity with that of SEQ ID NOs: 42 or 43. In some embodiments, the left CIITA homology arm can comprise or consist essentially of SEQ ID NO: 42, or a polynucleotide sequence having at least 85%, 90%, 95%, or 99% sequence identity with that of SEQ ID NO: 42. In some embodiments, the right CIITA homology arm can comprise or consist essentially of SEQ ID NO: 43, or a polynucleotide sequence having at least 85%, 90%, 95%, or 99% sequence identity with that of SEQ ID NO: 43.

In some embodiments, at least 50% of the engineered cells of a population of cells does not express a detectable level of CIITA protein. For example, at least 55%, at least 60%, at least 70%, at least 75%, at least 80%, at least 85%, at least 90%, or at least 95% of the engineered cells of a population may not express a detectable level of CIITA surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered cells of a population does not express a detectable level of CIITA protein.

In some embodiments, less than 50% of the engineered cells of a population of cells express a detectable level of CIITA protein. In some embodiments, less than 30% of the engineered cells of a population of cells express a detectable level of CIITA protein. For example, less than 50%, less than 30%, less than 25%, less than 20%, less than 15%, less than 10%, or less than 5% of the engineered cells of a population of cells express a detectable level of CIITA protein. In some embodiments, 40%-30%, 40%-20%, 40%-10%, 40%-5%, 30%-20%, 30%-10%, 30%-5%, 20%-10%, 20%-5%, or 10%-5% of the engineered cells of a population of cells express a detectable level of CIITA protein.

In some embodiments, any polynucleotide described herein is inserted into the CIITA locus such that 86 base pairs (bp) of the CIITA exon 2 are removed after homology directed repair.

### SERPINB9 Gene Edits

In some embodiments, the genome of any cell described herein comprises an insertion of a polynucleotide encoding SERPINB9. SERPINB9, which is encoded by SERPINB9 gene (NCBI Gene ID: 5272), is a member of a large family of apoptosis inhibitors that mainly function by targeting intermediate proteases (e.g., covalently bind a protease in 1:1 complex, thereby inhibiting the protease). As such, expression of SERPINB9 may increase survival of the engineered cell. For example, iNK cells engineered to express SERPINB9 can survive NK cell attack by inhibiting activity of the released granzymes. In some embodiments, expression of SERPINB9 is increased in cells. In some embodiments, an iPSC comprises an insertion of a polynucleotide encoding SERPINB9 (or a SERPINB9 knock-in). In some embodiments, an NK cell comprises an insertion of a polynucleotide encoding SERPINB9 (or a SERPINB9 knock-in). In some embodiments, a hepatoblast/hepatocyte comprises a insertion of a polynucleotide encoding SERPINB9 (or a SERPINB9 knock-in).

An example of a SERPINB9 cDNA sequence that may be used as provided herein to create a SERPINB9 knock-in SEQ ID NO: 8. In some embodiments, the SERPINB9 polynucleotide has at least 90%, 91%, 92%, 93%, 94%, 95%, 96%, 97%, 98%, or 99% identity to SEQ ID NO: 8. In some embodiments, the polynucleotide encoding SERPINB9 is linked to a GSG tag sequence and comprises SEQ ID NO: 93. In some embodiments, the SERPINB9-GSG polynucleotide has at least 90%, 91%, 92%, 93%, 94%, 95%, 96%, 97%, 98%, or 99% identity to SEQ ID NO: 93.

In some embodiments, at least 50% of the engineered cells of a population of cells express a detectable level of SERPINB9 protein. For example, at least 55%, at least 60%, at least 70%, at least 75%, at least 80%, at least 85%, at least 90%, or at least 95% of the engineered cells of a population express a detectable level of SERPINB9 protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered cells of a population express a detectable level of SERPINB9 protein.

In some embodiments, less than 50% of the engineered cells of a population of cells do not express a detectable level of SERPINB9. In some embodiments, less than 30% of the engineered cells of a population of cells do not express a detectable level of SERPINB9. For example, less than 50%, less than 30%, less than 25%, less than 20%, less than 15%, less than 10%, or less than 5% of the engineered cells of a population of cells do not express a detectable level of SERPINB9. In some embodiments, 40% 30%, 40%-20%, 40%-10%, 40%-5%, 30%-20%, 30%-10%, 30%-5%, 20%-10%, 20%-5%, or 10%-5% of the engineered cells of a population of cells do not express a detectable level of SERPINB9.

In some embodiments, any of the SERPINB9 polynucleotides described herein are inserted into any safe-harbor locus described herein. In some embodiments, any of the SERPINB9 polynucleotides described herein are inserted into any B2M locus described herein. In some embodiments, any of the SERPINB9 polynucleotides described herein are inserted into any CIITA locus described herein

### HLA-E Gene Edits

In some embodiments, the genome of any cell described herein comprises an insertion of a polynucleotide encoding human leukocyte antigen E (HLA-E; also called major histocompatibility complex, class I, E). HLA-E is encoded by HLA-E gene (gene (NCBI Gene ID: 3133). HLA-E is a heterodimer class I molecule. HLA-E primarily functions as a ligand for the NK cell inhibitory receptor KLRD1-KLRC1. HLA-E enables NK cells to monitor other MHC class I molecule expression and to tolerate self-expression. In some embodiments, the insertion of the HLA-E can protect the iNK from PB-NK “missing self” response. In some embodiments, expression of HLA-E is increased in cells. In some embodiments, an iPSC comprises an inserted polynucleotide encoding in HLA-E E (or HLA-E knock-in). In some embodiments, an NK cell comprises an inserted polynucleotide encoding in HLA-E (or HLA-E knock-in). In some embodiments, a hepatoblast/hepatocyte comprises an inserted polynucleotide encoding in HLA-E (or HLA-E knock-in).

An example of an HLA-E cDNA sequence that may be used as provided herein to create a genomic knock-in of the HLA-E CDS comprises SEQ ID NO: 16. In some embodiments, the HLA-E polynucleotide is an HLA-E trimer composed of a B2M signal peptide fused to an HLA-G presentation peptide fused to the B2M membrane protein fused to the HLA-E protein without its signal peptide. In some embodiments, the HLA-E trimer comprises or consists essentially of SEQ ID NO: 24 (e.g., SEQ ID NOS: 11-16). In some embodiments, the HLA-E trimer polynucleotide has at least 90%, 91%, 92%, 93%, 94%, 95%, 96%, 97%, 98%, or 99% identity to SEQ ID NO: 24. In some embodiments, the trimer design is that described in Gornalusse et al. (2017) Nat. Biotechnol. 35(8): 765-772, which is incorporated herein in its entirety.

In some embodiments, at least 50% of the engineered cells of a population of cells express a detectable level of HLA-E surface protein. For example, at least 55%, at least 60%, at least 70%, at least 75%, at least 80%, at least 85%, at least 90%, or at least 95% of the engineered cells of a population express a detectable level of HLA-E surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered cells of a population express a detectable level of HLA-E surface protein.

In some embodiments, less than 50% of the engineered cells of a population of cells do not express a detectable level of HLA-E surface protein. In some embodiments, less than 30% of the engineered cells of a population of cells do not express a detectable level of HLA-E surface protein. For example, less than 50%, less than 30%, less than 25%, less than 20%, less than 15%, less than 10%, or less than 5% of the engineered cells of a population of cells do not express a detectable level of HLA-E surface protein. In some embodiments, 40%-30%, 40%-20%, 40%-10%, 40%-5%, 30%-20%, 30%-10%, 30%-5%, 20%-10%, 20%-5%, or 10%-5% of the engineered cells of a population of cells do not express a detectable level of HLA-E surface protein.

In some embodiments, any of the HLA-E polynucleotides described herein are inserted into any safe-harbor locus described herein. In some embodiments, any of the HLA-E polynucleotides described herein are inserted into any B2M locus described herein. In some embodiments, any of the HLA-E polynucleotides described herein are inserted into any CIITA locus described herein.

### IL15 and IL15Rα Gene Edits

In some embodiments, the genome of any cell described herein comprises an insertion of polynucleotide encoding interleukin-15 (IL15), IL15Rα, and/or a fusion protein of IL15 and IL15Rα (IL15/IL15Rα). IL15 is a cytokine that functions in regulating NK cell proliferation and activation, and is encoded by IL15 gene (MCBI Gene ID: 3600). IL15Rα (also called IR15α) is the receptor that binds IL15, and is encoded by IL15Rα gene (MCBI Gene ID: 16169). In some embodiments, the insertion of the polynucleotide encoding IL15, IL15Rα, and/or fusion protein of IL15 and IL15Rα can lead to increased iNK persistence of the engineered cell.

In some embodiments, a cell has insertion of a polynucleotide encoding IL15 and the polynucleotide encoding IL15 comprises nSEQ ID NO: 34. In some embodiments, a cell has insertion of a polynucleotide encoding IL15Rα and the polynucleotide encoding IL15Rα comprises SEQ ID NO: 36. In some embodiments, a cell has insertion of a polynucleotide encoding both IL15 and IL15Rα and the polynucleotide comprises SEQ ID NO: 40. In some embodiments, the IL15/IL15Rα polynucleotide has at least 90%, 91%, 92%, 93%, 94%, 95%, 96%, 97%, 98%, or 99% identity to SEQ ID NO: 40. In some embodiments, IL15 and IL15Rα are co-expressed. In some embodiments, the IL15/IL15Rα fusion protein sequence is used as described in Hurton et al. (2016) Proc Natl Acad Sci USA.; 113(48):E7788-E7797. doi: 10.1073/pnas.1610544113, which is incorporated herein in its entirety. In some embodiments, an iPSC comprises a knock-in of the IL15/IL15Rα polynucleotide. In some embodiments, an NK cell comprises a knock-in of the IL15/IL15Rα polynucleotide. In some embodiments, a hepatoblast/hepatocyte comprises a knock-in of the IL15/IL15Rα polynucleotide.

In some embodiments, at least 50% of the engineered cells of a population of cells express a detectable level of IL15/IL15Rα fusion protein. For example, at least 55%, at least 60%, at least 70%, at least 75%, at least 80%, at least 85%, at least 90%, or at least 95% of the engineered cells of a population express a detectable level of IL15/IL15Rα fusion protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered cells of a population expresses a detectable level of IL15/IL15Rα fusion protein.

In some embodiments, less than 50% of the engineered cells of a population of cells do not express a detectable level of IL15, IL15Rα, and/or IL15/IL15Rα. In some embodiments, less than 30% of the engineered cells of a population of cells do not express a detectable level of IL15, IL15Rα, and/or IL15/IL15Rα. For example, less than 50%, less than 30%, less than 25%, less than 20%, less than 15%, less than 10%, or less than 5% of the engineered cells of a population of cells do not express a detectable level of IL15, IL15Rα, and/or IL15/IL15Rα. In some embodiments, 40%-30%, 40%-20%, 40%-10%, 40%-5%, 30%-20%, 30%-10%, 30%-5%, 20%-10%, 20%-5%, or 10%-5% of the engineered cells of a population of cells do not express a detectable level of IL15, IL15Rα, and/or IL15/IL15Rα.

In some embodiments, any of the IL15/IL15Rα polynucleotides described herein are inserted into any safe-harbor locus described herein. In some embodiments, any of the IL15/IL15Rα polynucleotides described herein are inserted into any B2M locus described herein. In some embodiments, any of the IL15/IL15Rα polynucleotides described herein are inserted into any CIITA locus described herein.

### XIAP Gene Edits

In some embodiments, the genome of any cell described herein comprises an insertion of a polynucleotide encoding X-linked inhibitor of apoptosis protein (XIAP), which is encoded by XIAP gene (NCBI Gene ID: 331). XIAP protein helps protect cells from undergoing apoptosis, e.g., by inhibiting the activity of certain caspase enzymes, in particular caspases 3, 7, and 9. XIAP protein also modulates inflammatory signaling and immunity.

In some embodiments, a cell has insertion of a polynucleotide encoding XIAP and comprising SEQ ID NO: 45. In some embodiments, the XIAP polynucleotide has at least 90%, 91%, 92%, 93%, 94%, 95%, 96%, 97%, 98%, or 99% identity to SEQ ID NO: 45. In some embodiments, the polynucleotide encoding XIAP is linked to a GSG tag sequence and comprises SEQ ID NO: 94. In some embodiments, the AAP-GSG polynucleotide has at least 90%, 91%, 92%, 93%, 94%, 95%, 96%, 97%, 98%, or 99% identity to SEQ ID NO: 94. In some embodiments, an iPSC comprises a knock-in of the MAP polynucleotide. In some embodiments, an NK cell comprises a knock-in of the XIAP polynucleotide. In some embodiments, a hepatoblast/hepatocyte comprises a knock-in of the MAP polynucleotide.

In some embodiments, at least 50% of the engineered cells of a population of cells express a detectable level of XIAP. For example, at least 55%, at least 60%, at least 70%, at least 75%, at least 80%, at least 85%, at least 90%, or at least 95% of the engineered cells of a population express a detectable level of XIAP. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered cells of a population expresses a detectable level of XIAP.

In some embodiments, less than 50% of the engineered cells of a population of cells do not express a detectable level of XIAP. In some embodiments, less than 30% of the engineered cells of a population of cells do not express a detectable level of XIAP. For example, less than 50%, less than 30%, less than 25%, less than 20%, less than 15%, less than 10%, or less than 5% of the engineered cells of a population of cells do not express a detectable level of XIAP. In some embodiments, 40%-30%, 40%-20%, 40%-10%, 40%-5%, 30%-20%, 30%-10%, 30%-5%, 20%-10%, 20%-5%, or 10%-5% of the engineered cells of a population of cells do not express a detectable level of XIAP.

In some embodiments, any of the XIAP polynucleotides described herein are inserted into any safe-harbor locus described herein. In some embodiments, any of the XIAP polynucleotides described herein are inserted into any B2M locus described herein. In some embodiments, any of the MAP polynucleotides described herein are inserted into any CIITA locus described herein.

### CISH Gene Edits

In some embodiments, the genome of any cell described herein is modified to disrupt a cytokine inducible SH2 containing protein (CISH, also called CIS) gene (NCBI Gene ID: 1154). CISH is a cytokine-inducible negative regulator of cytokine signaling. CISH participates within a multi-molecular E3 ubiquitin ligase complex to ubiquitinate target proteins. CISH has an inhibitory effect on T cell activation mediated by PLC-71 regulation, and it functions as a potent checkpoint in CD8⁺ T cell tumor immunotherapy. In some embodiments, the disrupted CISH can increase iNK sensitivity to cytokines, improve iNK persistence, and/or increase tumor killing. In some embodiments, an iPSC comprises a disrupted CISH gene. In some embodiments, an NK cell comprises a disrupted CISH gene. In some embodiments, a differentiated cell comprises a disrupted CISH gene

In some embodiments, a ribonucleoprotein particle (RNP) containing an RNA-guided nuclease (e.g., a Cas nuclease, such as a Cas9 nuclease) and a gRNA targeting the CISH gene (or any other gene of interest) are delivered to any cell described herein (e.g., iPSC). A ribonucleoprotein particle (RNP) comprises an RNA-guided nuclease (e.g., Cas9) pre-complexed/complexed with a gRNA. In other embodiments, the RNA-guided nuclease and gRNA are delivered separately to cells. In some embodiments, modified or unmodified CISH gRNA sequences may be used to create a genomic disruption in the CISH gene. In some embodiments, the gRNA targets a site within the CISH gene. In some embodiments, the CISH gRNA targets a sequence comprising any one of SEQ ID NOS: 49, 50, 51, 52, 53, 54, 55, 56, 57, 58, 59, or 60 (see Table A). In some embodiments, the gRNA comprises a spacer sequence corresponding to a sequence consisting of and one of SEQ ID NOS: 49, 50, 51, 52, 53, 54, 55, 56, 57, 58, 59, or 60.

In some embodiments, gRNAs targeting the CISH genomic region create indels in the CISH gene disrupting expression of the mRNA or protein. In some embodiments, a gRNA/CRISPR nuclease complex targets and cleaves a target site in the CISH locus. Repair of a double-stranded break by NHEJ can result in a deletion of at least on nucleotide and/or an insertion of at least one nucleotide, thereby disrupting or eliminating expression of CISH. In some embodiments, the CISH locus is targeted by at least two CRISPR systems each comprising a different gRNA, such that cleavage at two sites in the CISH locus leads to a deletion of the sequence between the two cuts, thereby eliminating expression CISH.

TABLE A
CISH Target Sequences

| | Target Sequence | SEQ | |
|---|---|---|---|
| Name | (5′-3′) | ID NO: | PAM |
| CISH Ex1 T2 | TCGCCGCTGCCGCGGGGACA | 49 | TGG |
| CISH Ex1 T18 | GACATGGTCCTCTGCGTTCA | 50 | GGG |
| CISH Ex2 T1 | GTCCGCTCCACAGCCAGCAA | 51 | AGG |
| CISH Ex2 T2 | GTTCCAGGGACGGGGCCCAC | 52 | AGG |
| CISH Ex3 T1 | TCGGGCCTCGCTGGCCGTAA | 53 | TGG |
| CISH Ex3 T2 | CGTACTAAGAACGTGCCTTC | 54 | TGG |
| CISH Ex3 T3 | GGGTTCCATTACGGCCAGCG | 55 | AGG |
| CISH Ex3 T5 | CAGGTGTTGTCGGGCCTCGC | 56 | TGG |
| CISH Ex3 T6 | TACTCAATGCGTACATTGGT | 57 | GGG |
| CISH Ex3 T9 | AAGGCTGACCACATCCGGAA | 58 | AGG |
| CISH Ex3 T11 | TACATTGGTGGGGCCACGAG | 59 | TGG |
| CISH Ex3 T14 | CTGTCAGTGAAAACCACTCG | 60 | TGG |

In some embodiments, at least 50% of the engineered cells of a population of cells does not express a detectable level of CISH protein. For example, at least 5500, at least 6000, at least 70%, at least 75%, at least 80%, at least 85%, at least 90%, or at least 95% of the engineered cells of a population may not express a detectable level of CISH surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered cells of a population does not express a detectable level of CISH protein.

In some embodiments, less than 50% of the engineered cells of a population of cells express a detectable level of CISH protein. In some embodiments, less than 30% of the engineered cells of a population of cells express a detectable level of CISH protein. For example, less than 50%, less than 30%, less than 25%, less than 20%, less than 15%, less than 10%, or less than 5% of the engineered cells of a population of cells express a detectable level of CISH surface protein. In some embodiments, 40%-30%, 40%-20%, 40%-10%, 40%-5%, 30%-20%, 30%-10%, 30%-5%, 20%-10%, 20%-5%, or 10%-5% of the engineered cells of a population of cells express a detectable level of CISH protein.

### FAS Gene Edits

In some embodiments, the genome of any cell described herein is modified to disrupt a Fas cell surface death receptor (FAS) gene (NCBI Gene ID: 355). FAS is a member of the TNF-receptor superfamily and contributes to the regulation of programmed cell death. In some embodiments, the disrupted FAS can reduce activation-induced cell death (AICD), resist apoptosis, and/or increase tumor killing. In some embodiments, an iPSC comprises a disrupted FAS gene. In some embodiments, an NK cell comprises a disrupted FAS gene. In some embodiments, a differentiated cell comprises a disrupted FAS gene

In some embodiments, gRNAs targeting the FAS genomic region create indels in the FAS gene disrupting expression of the mRNA or protein. In some embodiments, a ribonucleoprotein particle (RNP) containing an RNA-guided nuclease (e.g., a Cas nuclease, such as a Cas9 nuclease) and a gRNA targeting the FAS gene (or any other gene of interest) are delivered to any cell described herein (e.g., iPSC). A ribonucleoprotein particle (RNP) comprises an RNA-guided nuclease (e.g., Cas9) pre-complexed/complexed with a gRNA. In other embodiments, the RNA-guided nuclease and gRNA are delivered separately to cells. In some embodiments, modified or unmodified FAS gRNA sequences may be used to create a genomic disruption in the FAS gene. In some embodiments, the gRNA targets a site within the FAS gene. In some embodiments, the FAS gRNA targets a sequence comprising any one of SEQ ID NOS: 61, 62, 63, 64, 65, or 67 (see Table B). In some embodiments, the gRNA comprises a spacer sequence corresponding to a sequence consisting of and one of SEQ ID NOS: 61, 62, 63, 64, 65, or 67.

TABLE B
FAS Target Sequences

| | Target Sequence | SEQ | |
|---|---|---|---|
| Name | (5′-3′) | ID NO: | PAM |
| FAS Ex1 T7 | GGATTGCTCAACAACCATGC | 61 | TGG |
| FAS Ex1 T9 | GATTGCTCAACAACCATGCT | 62 | GGG |
| FAS Ex2 T1 | GTGACTGACATCAACTCCAA | 63 | GGG |
| FAS Ex2 T2 | CACTTGGGCATTAACACTTT | 64 | TGG |
| FAS Ex2 T3 | TTGGAAGGCCTGCATCATGA | 65 | TGG |
| FAS Ex2 T7 | ACTCCAAGGGATTGGAATTG | 66 | AGG |
| FAS Ex3 T1 | CTAGGGACTGCACAGTCAAT | 67 | GGG |

In some embodiments, at least 50% of the engineered cells of a population of cells does not express a detectable level of FAS protein. For example, at least 55%, at least 60%, at least 70%, at least 75%, at least 80%, at least 85%, at least 90%, or at least 95% of the engineered cells of a population may not express a detectable level of FAS surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered cells of a population does not express a detectable level of FAS protein.

In some embodiments, less than 50% of the engineered cells of a population of cells express a detectable level of FAS protein. In some embodiments, less than 30% of the engineered cells of a population of cells express a detectable level of FAS protein. For example, less than 50%, less than 30%, less than 25%, less than 20%, less than 15%, less than 10%, or less than 5% of the engineered cells of a population of cells express a detectable level of FAS protein. In some embodiments, 40%-30%, 40%-20%, 40%-10%, 40%-5%, 30%-20%, 30%-10%, 30%-5%, 20%-10%, 20%-5%, or 10%-5% of the engineered cells of a population of cells express a detectable level of FAS protein.

### Edits to Knock-In Chimeric Antigen Receptors

A chimeric antigen receptor (CAR) refers to an artificial immune cell receptor that is engineered to recognize and bind to an antigen expressed by tumor cells. CARs can be inserted into any cells described herein. CARs are a chimera of a signaling domain of the T-cell receptor (TCR) complex and an antigen-recognizing domain (e.g., a single chain fragment (scFv) of an antibody or other antibody fragment) (Enblad et al., Human Gene Therapy. 2015; 26(8):498-505). CARs have the ability to redirect cell specificity and reactivity toward a selected target in a non-MHC-restricted manner. The non-MHC-restricted antigen recognition gives cells expressing CARs the ability to recognize an antigen independent of antigen processing, thus bypassing a major mechanism of tumor escape. CARs are often referenced to by the antigen they bind. For example, a “CD30 CAR”, “CD19 CAR”, a “CD70 CAR”, a “CD33 CAR” and a “BCMA CAR” are CARs comprising antigen binding domains that specifically bind to CD30, CD19, CD70, CD33 or BCMA, respectively. Accordingly, such terms are interchangeable with anti-CD30 CAR, anti-CD19 CAR, anti-CD70 CAR, anti-CD33 CAR and anti-BCMA CAR. It will be understood by those of ordinary skill in the art that a CAR that specifically binds an antigen can be referred to with either terminology.

In some embodiments, any iPSC described herein expresses a CAR. In some embodiments, any NK cell described herein expresses a CAR. In some embodiments, any HSPC described herein expresses a CAR.

There are four generations of CARs, each of which contains different components. First generation CARs join an antibody-derived scFv to the CD3zeta (ζ or z) intracellular signaling domain of the T-cell receptor through hinge and transmembrane domains. Second generation CARs incorporate an additional domain, e.g., CD28, 4-1BB (41BB), or ICOS, to supply a costimulatory signal. Third-generation CARs contain two costimulatory domains fused with the TCR CD3ζ chain. Third-generation costimulatory domains may include, e.g., a combination of CD3ζ, CD27, CD28, 4-1BB, ICOS, or OX40. Fourth-generation CARs include immune stimulatory cytokines to improve cell persistence and expansion. Cytokines for fourth-generation CARS include individually or in combination any of IL-7, IL-12, IL-15, IL-18, or IL-23. CARs, in some embodiments, contain an ectodomain, commonly derived from a single chain variable fragment (scFv), a hinge, a transmembrane domain, and an endodomain with one (first generation), two (second generation), or three (third generation) signaling domains derived from CD3Z and/or co-stimulatory molecules (Maude et al., Blood. 2015; 125(26):4017-4023; Kakarla and Gottschalk, Cancer J. 2014; 20(2):151-155).

CARs typically differ in their functional properties. The CD3ζ signaling domain of the T-cell receptor, when engaged, will activate and induce proliferation of T-cells but can lead to anergy (a lack of reaction by the body's defense mechanisms, resulting in direct induction of peripheral lymphocyte tolerance). Lymphocytes are considered anergic when they fail to respond to a specific antigen. The addition of a costimulatory domain in second-generation CARs improved replicative capacity and persistence of modified T-cells. Similar antitumor effects are observed in vitro with CD28 or 4-1BB CARs, but preclinical in vivo studies suggest that 4-1BB CARs may produce superior proliferation and/or persistence. Clinical trials suggest that both of these second-generation CARs are capable of inducing substantial T-cell proliferation in vivo, but CARs containing the 4-1BB costimulatory domain appear to persist longer. Third generation CARs combine multiple signaling domains (costimulatory) to augment potency.

In some embodiments, a chimeric antigen receptor is a first generation CAR. In other embodiments, a chimeric antigen receptor is a second generation CAR. In yet other embodiments, a chimeric antigen receptor is a third generation CAR. In some embodiments, a chimeric antigen receptor is a fourth-generation CAR.

A CAR, in some embodiments, comprises an extracellular (ecto) domain comprising an antigen binding domain (e.g., an antibody, such as an scFv), a transmembrane domain, and a cytoplasmic (endo) domain.

### Ectodomain of CARs

The ectodomain is the region of the CAR that is exposed to the extracellular fluid and, in some embodiments, includes an antigen binding domain, and optionally a signal peptide, a spacer domain, and/or a hinge domain. In some embodiments, the antigen binding domain is a single-chain variable fragment (scFv) that includes the VL and VH of immunoglobulins connected with a short linker peptide. The linker, in some embodiments, includes hydrophilic residues with stretches of glycine and serine for flexibility as well as stretches of glutamate and lysine for added solubility. A single-chain variable fragment (scFv) is not actually a fragment of an antibody, but instead is a fusion protein of the variable regions of the heavy (VH) and light chains (VL) of immunoglobulins, connected with a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original immunoglobulin, despite removal of the constant regions and the introduction of the linker. In some embodiments, the scFv of the present disclosure is humanized. In other embodiments, the scFv is fully human. In yet other embodiments, the scFv is a chimera (e.g., of mouse and human sequence).

In some embodiments, the scFv is an anti-CD30 scFv (binds specifically to CD30, also called TNF receptor superfamily member 8 or TNFRSF8). In some embodiments, anti-CD30 scFv may comprise variable domains from mouse monoclonal AC10 (e.g., Brentuximab). In other embodiments, anti-CD30 scFv may comprise variable domains from human 5F11 antibody (U.S. Pat. No. 7,387,776). In some embodiments the scFV of a CD30 CAR may comprise the nucleotide sequence of SEQ ID NO: 70, SEQ ID NO: 79, or SEQ ID NO: 86. In some embodiments, the anti-CD30 CAR coding sequence is SEQ ID NO: 74, SEQ ID NO: 81, or SEQ ID NO: 87. In some embodiment the anti-CD30 CAR coding sequence has at least 90%, 91%, 92%, 93%, 94%, 95%, 96%, 97%, 98%, or 99% identity to SEQ ID NO: 74, SEQ ID NO: 81, or SEQ ID NO: 87. Non-limiting examples of a CD30 CAR that may be used as provided herein may include the amino acid sequence of SEQ ID NO: 78, SEQ ID NO: 85, or SEQ ID NO: 91.

In some embodiments, the scFv is an anti-BCMA scFv (binds specifically to BCMA).

In some embodiments, the scFv is an anti-GPC3 scFv (binds specifically to GPC3).

In some embodiments, the scFv is an anti-CD19 scFv (binds specifically to CD19).

In some embodiments, the scFv is an anti-CD70 scFv (binds specifically to CD70).

In some embodiments, the scFv is an anti-CD33 scFv (binds specifically to CD33).

In some embodiments, the scFv is an anti-NKp30 scFv (binds specifically to NKp30).

In some embodiments, the scFv is an anti-CD73 scFv (binds specifically to CD73).

In some embodiments, the scFv is an anti-GPR87 scFv (binds specifically to GPR87).

In some embodiments, the scFv is an anti-LIV1A scFv (binds specifically to LIV1A).

In some embodiments, the scFv is an anti-A33 scFv (binds specifically to A33).

In some embodiments, the scFv is an anti-EGFR scFv (binds specifically to EGFR).

In some embodiments, the scFv is an anti-CD20 scFv (binds specifically to CD20).

In some embodiments, the scFv is an anti-SLC7A11 scFv (binds specifically to SLC7A11).

Other scFv proteins may be used.

The signal peptide can enhance the antigen specificity of CAR binding. Signal peptides can be derived from antibodies, such as, but not limited to, CD8, as well as epitope tags such as, but not limited to, GST or FLAG. Other signal peptides may be used.

In some embodiments, the ectodomain is the region of the CAR that is exposed to the extracellular fluid and, in some embodiments, includes a NKG2D receptor, and optionally a signal peptide, a spacer domain, and/or a hinge domain.

In some embodiments, a spacer domain or hinge domain is located between an extracellular domain (comprising the antigen binding domain) and a transmembrane domain of a CAR, or between a cytoplasmic domain and a transmembrane domain of the CAR. A spacer domain is any oligopeptide or polypeptide that functions to link the transmembrane domain to the extracellular domain and/or the cytoplasmic domain in the polypeptide chain. A hinge domain is any oligopeptide or polypeptide that functions to provide flexibility to the CAR, or domains thereof, or to prevent steric hindrance of the CAR, or domains thereof. In some embodiments, a spacer domain or a hinge domain may comprise up to 300 amino acids (e.g., 10 to 100 amino acids, or 5 to 20 amino acids). In some embodiments, one or more spacer domain(s) may be included in other regions of a CAR. In some embodiments, the hinge domain is a CD8 hinge domain. Other hinge domains may be used.

### Transmembrane Domain of CARs

The transmembrane domain is a hydrophobic alpha helix that spans the membrane. The transmembrane domain provides stability of the CAR. In some embodiments, the transmembrane domain of a CAR as provided herein is a CD8 transmembrane domain. In other embodiments, the transmembrane domain is a CD28 transmembrane domain. In yet other embodiments, the transmembrane domain is a chimera of a CD8 and CD28 transmembrane domain. Other transmembrane domains may be used as provided herein.

In some embodiments, the transmembrane domain is selected from transmembrane domains of: NKG2D, FcYRIIIa, NKp44, NKp30, NKp46, actKIR, NKG2C, CD8a, and IL15Rb. In some embodiments, the transmembrane domain is an NKG2D transmembrane domain.

### Endodomain of CARs

The endodomain is the functional end of the receptor. Following antigen recognition, receptors cluster and a signal is transmitted to the cell. The most commonly used endodomain component is CD3-zeta, which contains three (3) immunoreceptor tyrosine-based activation motif (ITAM)s. This transmits an activation signal to the T cell and/or NK cells after the antigen is bound. In many cases, CD3-zeta may not provide a fully competent activation signal and, thus, a co-stimulatory signaling is used. For example, CD28 and/or 4-1BB may be used with CD3-zeta (CD3ζ) to transmit a proliferative/survival signal. Thus, in some embodiments, the co-stimulatory molecule of a CAR as provided herein is a CD28 co-stimulatory molecule. In other embodiments, the co-stimulatory molecule is a 4-1BB co-stimulatory molecule. In some embodiments, a CAR includes CD3ζ and CD28. In other embodiments, a CAR includes CD3-zeta and 4-1BB. In still other embodiments, a CAR includes CD3ζ, CD28, and 4-1BB.

In some embodiments, any of the CARs described herein have one, two or more intracellular signaling domains from, e.g., CD137/41 BB, DNAM-1, NKrdO, 2B4, NTBA, CRACC, CD2, CD27, one or more integrins (e.g., ITGB1, ITGB2, or ITGB3), IL-15R, IL-18R, IL-12R, IL-21 R, or IRE1a (e.g., any combination of signaling domains from two or more of these molecules).

Natural Killer cells express a number of transmembrane adapters providing them with signal enhancement. In some embodiments, the intracellular signaling domain of any CAR described herein comprises a transmembrane adapter. In some embodiments, the transmembrane adapter is a transmembrane adaptor from one or more of. FceR1 y, CD3ζ, DAP 12, and DAP 10.

In some embodiments, any CARs described herein have one of more co-stimulatory domains. In some embodiments, a 2B4 co-stimulatory domain is used. In some embodiments, a CD3ζ intracellular signaling domain is used. In some embodiments, a DAP10 or DAP12 co-stimulatory domains are used with a CD3ζ intracellular signaling domain. In some embodiments, a DAP10 co-stimulatory signaling domain is used with an NKG2D transmembrane domain. In some embodiments, the transmembrane domain is from NKG2D, and the endodomain is from DAP10 and CD3ζ (e.g., as described in Chang Y H et al. Cancer Res. 2013. 73(6):1777-86). In some embodiments, the CAR comprises an NKG2D transmembrane domain fused to 4-1BB and DAP10 signaling and/or co-stimulatory domains (e.g., as described in Guo C. et al. Mol Immunol. 2019. 114:108-113). In some embodiments, the CAR comprises a co-stimulatory domain from 2B4. In some embodiments, the CAR comprises a CD8 transmembrane domain and 4-1BB-CD3ζ signaling domains (e.g., as in a construct as described by Imai C, et al. Blood. 2005, 106(1). 376-383).

In some embodiments, the CAR has a CD8 transmembrane domain, a 4-1BB intracellular domain, and a CD3ζ signaling domain. In some embodiments, the CAR has a CD28 transmembrane domain, a CD28 intracellular domain, and a CD3ζ signaling domain. In some embodiments, the CAR has a DAP12 transmembrane and intracellular domains. In some embodiments, the CAR has a 2134 transmembrane and intracellular domains and a CD3ζ signaling domain. In some embodiments, the CAR has a CD8 transmembrane domain, a 2B4 intracellular domain, and a CD3ζ signaling domain. In some embodiments, the CAR has a CD28 transmembrane and intracellular domains, a 4-1BB intracellular domain, and a CD3ζ signaling domain. In some embodiments, the CAR has a CD16 transmembrane domain, a 2134 intracellular domain, and a CD3ζ signaling domain. In some embodiments, the CAR has a NKp44 transmembrane domain, a DAP10 intracellular domain, and a CD3ζ signaling domain. In some embodiments, the CAR has a NKp46 transmembrane domain, a 2134 intracellular domain, and a CD3ζ signaling domain. In some embodiments, the CAR has a NKG2D transmembrane domain, a 4-1BB intracellular domain, and a CD3ζ signaling domain. In some embodiments, the CAR has a NKG2D transmembrane domain, a 4-1BB intracellular domain, and a CD3ζ signaling domain. In some embodiments, the CAR has an NKG2D transmembrane domain, a DAP12 intracellular domain, a 2B4 intracellular domain, and a CD3ζ signaling domain. In some embodiments, the CAR has an NKG2D transmembrane domain, a DAP10 intracellular domain, a 2B4 intracellular domain, and a CD3ζ signaling domain. In some embodiments, the CAR has an NKG2D transmembrane domain, a 4-1BB intracellular domain, a 2B4 intracellular domain, and a CD3ζ signaling domain. In some embodiments, the CAR has an NKG2D transmembrane domain and a CD3ζ signaling domain.

In some embodiments, at least 50% of the engineered cells of a population of cells express a detectable level of the CAR. For example, at least 55%, at least 60%, at least 70%, at least 75%, at least 80%, at least 85%, at least 90%, or at least 95% of the engineered cells of a population express a detectable level of the CAR. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered cells of a population expresses a detectable level of the CAR.

### Multi-Gene Editing

In some embodiments, the engineered cells of the present disclosure include more than one gene edit, for example, in more than one gene. In some embodiments, two, three, four, five, six or more genes are edited. In some embodiments, the gene-edit is an insertion (KI). In some embodiments, the gene-edit is a disruption (KO). In some embodiments, the combination of two or more gene edits described herein is a combination of insertions (KI) and disruptions (KO). In some embodiments, the gene-edits are any combination of one, two, three, four, five, six or more of the gene-edits selected from: SERPINB9 KI, B2M KO, IL15/IL15Rα KI, HLA-E KI, CIITA KO, XIAP KI, CAR (e.g., CD30, BCMA, GPC3, CD16, CD70, CD19, GPR87, CD33, NKG2D, etc.) KI, CD16 KI, CD64 KI, CISH KO, FAS KO, ADAM17 KO, REGNASE-1 KO, TIGIT KO, PD-1 KO, NKG2A KO, CD70 KO, ALK4, type I activin receptor KO (e.g., a conditional KO), SOCS3 KO, tissue factor KO, and CD39 KI. In some embodiments, the editing of two or more genes is simultaneous, such as in the same method step. For example, an engineered cell may comprise a disrupted CIITA gene, a disrupted B2M gene, or a combination thereof. In some embodiments, any iPSC cell described herein has a disrupted CIITA gene and a disrupted B2M gene. In some embodiments, any engineered NK cell described herein comprises a disrupted CIITA gene and a disrupted B2M gene.

In some embodiments, any of the polynucleotides described herein are linked to a promoter. In some embodiments, any of the polynucleotides described herein are linked to an exogenous promoter. In some embodiments, the promoter is selected from but not limited to CAGGS, CMV, EF1a, PGK, CAG, UBC, or other constitutive, inducible, temporal-, tissue-, or cell type-specific promoter.

In some embodiments, the genome-engineered cells comprise introduced or increased expression of in at least one of SERPINB9, HLA-E, IL15/IL15Rα, and XIAP. In some embodiments, any genome-engineered cell is HLA class I and/or class II deficient. In some embodiments, the genome-engineered cells comprise integrated or non-integrated exogenous polynucleotide encoding one or more of SERPINB9, HLA-E, IL15/IL15Rα, and XIAP proteins. In some embodiments, said introduced expression is an increased expression from either non-expressed or lowly expressed genes comprised in said cells. In some embodiments, the non-integrated exogenous polynucleotides are introduced using Sendai virus, AAV, episomal, or plasmid. In some embodiments, the cells are B2M null, with introduced expression of SERPINB9 and/or HLA-E. In some embodiments, the cells are HLA-A, HLA-B, and HLA-C null, with introduced expression of HLA-E. In some embodiments, the cells are B2M null, with introduced expression of one or more of SERPINB9, HLA-E, IL15/IL15Rα, and XIAP. Methods of generating any of the genetically modified cells described herein are contemplated to be performed using but not limited to, any of the gene editing methods described herein.

In some embodiments, a polynucleotide encoding SERPINB9 is inserted at a site within or near a B2M gene locus in any cell described herein. In some embodiments, a polynucleotide encoding SERPINB9 is inserted at a site within or near a B2M gene locus concurrent with or following a deletion of all or part of a B2M gene or promoter. In some embodiments, the polynucleotide encoding SERPINB9 is operably linked to an exogenous promoter. In some embodiments, the polynucleotide encoding SERPINB9 is operably linked to the CAGGS promoter. In some embodiments, any cell described herein is gene edited to express a polynucleotide encoding SERPINB9 operably linked to the CAGGS promoter.

In some embodiments, a polynucleotide encoding SERPINB9 is inserted at a site within or near a CIITA gene locus in any cell described herein. In some embodiments, a polynucleotide encoding SERPINB9 is inserted at a site within or near a CIITA gene locus concurrent with or following a deletion of all or part of a CIITA gene or promoter. In some embodiments, the polynucleotide encoding SERPINB9 is operably linked to an exogenous promoter. In some embodiments, the polynucleotide encoding SERPINB9 is operably linked to the CAGGS promoter. In some embodiments, any cell described herein is gene edited to express a polynucleotide encoding SERPINB9 operably linked to the CAGGS promoter.

In some embodiments, a polynucleotide encoding HLA-E is inserted at a site within or near a B2M gene locus in any cell described herein. In some embodiments, a polynucleotide encoding HLA-E is inserted at a site within or near a B2M gene locus concurrent with or following a deletion of all or part of a B2M gene or promoter. In some embodiments, the polynucleotide encoding HLA-E is operably linked to an exogenous promoter. In some embodiments, the polynucleotide encoding HLA-E is operably linked to the CAGGS promoter. In some embodiments, any cell described herein is gene edited to express a polynucleotide encoding HLA-E operably linked to the CAGGS promoter.

In some embodiments, a polynucleotide encoding HLA-E is inserted at a site within or near a CIITA gene locus in any cell described herein. In some embodiments, a polynucleotide encoding HLA-E is inserted at a site within or near a CIITA gene locus concurrent with or following a deletion of all or part of a CIITA gene or promoter. In some embodiments, the polynucleotide encoding HLA-E is operably linked to an exogenous promoter. In some embodiments, the polynucleotide encoding HLA-E is operably linked to the CAGGS promoter. In some embodiments, any cell described herein is gene edited to express a polynucleotide encoding HLA-E operably linked to the CAGGS promoter.

In some embodiments, a polynucleotide encoding IL15/IL15Rα is inserted at a site within or near a B2M gene locus in any cell described herein. In some embodiments, a polynucleotide encoding IL15/IL15Rα is inserted at a site within or near a B2M gene locus concurrent with or following a deletion of all or part of a B2M gene or promoter. In some embodiments, the polynucleotide encoding IL15/IL15Rα is operably linked to an exogenous promoter. In some embodiments, the polynucleotide encoding IL15/IL15Rα is operably linked to the CAGGS promoter. In some embodiments, any cell described herein is gene edited to express a polynucleotide encoding IL15/IL15Rα operably linked to the CAGGS promoter.

In some embodiments, a polynucleotide encoding IL15/IL15Rα is inserted at a site within or near a CIITA gene locus in any cell described herein. In some embodiments, a polynucleotide encoding IL15/IL15Rα is inserted at a site within or near a CIITA gene locus concurrent with or following a deletion of all or part of a CIITA gene or promoter. In some embodiments, the polynucleotide encoding IL15/IL15Rα is operably linked to an exogenous promoter. In some embodiments, the polynucleotide encoding IL15/IL15Rα is operably linked to the CAGGS promoter. In some embodiments, any cell described herein is gene edited to express a polynucleotide encoding IL15/IL15Rα operably linked to the CAGGS promoter.

In some embodiments, the edited cells described herein express at least one chimeric antigen receptor (CAR). In some embodiments, the CAR is inserted at a specific gene locus. In some embodiments, the CAR is inserted at a specific locus to simultaneously disrupt expression of a target gene.

In some embodiments, a polynucleotide encoding any CAR described herein is inserted within or near a CIITA gene locus. In some embodiments, a polynucleotide encoding any CAR described herein is inserted within or near a CIITA gene locus concurrent with, or following a deletion of CIITA. In some embodiments, a polynucleotide encoding a CD30-CAR is inserted within the CIITA locus. In some embodiments, a polynucleotide encoding CD30-CAR is inserted at a site within or near a CIITA gene locus concurrent with, or following a deletion of a CIITA gene or promoter. In some embodiments, the CD30 CAR is inserted into the CIITA locus wherein 86 base pairs (bp) of CIITA exon 2 are removed after homology directed repair. In some embodiments, the CD30 CAR is inserted into CIITA locus using a donor plasmid. In some embodiments, a CD30 CAR donor plasmid is electroporated into any cell described herein along with the ribonucleoprotein (RNP) complex made of up of any CIITA targeting gRNA and Cas9 protein. In some embodiments, the CD30-CAR inserted into the CIITA locus is driven by any promoter described herein. In some embodiments, the CD30-CAR inserted into the CIITA locus is driven by the CAG promoter. In some embodiments, any cell described herein is gene-edited to express a CD30-CAR within the CIITA locus. In some embodiments, an iPSC is gene-edited to express a CD30-CAR within the CIITA locus.

In some embodiments, a polynucleotide encoding XIAP is inserted at a site within or near a B2M gene locus in any cell described herein. In some embodiments, a polynucleotide encoding XIAP is inserted at a site within or near a B2M gene locus concurrent with or following a deletion of all or part of a B2M gene or promoter. In some embodiments, the polynucleotide encoding XIAP is operably linked to an exogenous promoter. In some embodiments, the polynucleotide encoding XIAP is operably linked to the CAGGS promoter. In some embodiments, any cell described herein is gene edited to express a polynucleotide encoding XIAP operably linked to the CAGGS promoter.

In some embodiments, the (B2M)-SERPINB9-P2A-IL15/IR15α donor plasmid (SEQ ID NO: 23) is electroporated into any cell described herein along with the RNP complex made of up of B2M targeting gRNA (corresponding to a sequence of SEQ ID NO: 1) and Cas9 protein to yield a B2M null, SERPINB9 expressing, and IL15/IR15α expressing cell. In some embodiments, the B2M null, SERPINB9 expressing, and IL15/IR15α expressing cell can be electroporated with (CIITA)-CAR-P2A-HLA-E donor plasmid herein along with the RNP complex made of up of CIITA targeting gRNA (corresponding to a sequence of SEQ ID NO: 41) and Cas9 protein to yield a B2M null, SERPINB9 expressing, IL15/IR15α expressing, CIITA null, CAR expressing, and HLA-E expressing cell.

In some embodiments, the (B2M)-SERPINB9-P2A-HLA-E donor plasmid (SEQ ID NO: 39) is electroporated into any cell described herein along with the RNP complex made of up of B2M targeting gRNA (corresponding to a sequence of SEQ ID NO: 1) and Cas9 protein to yield a B2M null, SERPINB9 expressing, and HLA-E expressing cell.

In some embodiments, the (CIITA)-SERPINB9-P2A-HLA-E donor plasmid (SEQ ID NO: 44) is electroporated into any cell described herein along with the RNP complex made of up of CIITA targeting gRNA (corresponding to a sequence of SEQ ID NO: 41) and Cas9 protein to yield a CIITA null, SERPINB9 expressing, and HLA-E expressing cell. In some embodiments the CIITA null, SERPINB9 expressing, and HLA-E expressing cell can be electroporated with the (B2M)-XIAP-P2A-IL15/IR15α donor plasmid (SEQ ID NO:48) along with the RNP complex made of up of B2M targeting gRNA (corresponding to a sequence of SEQ ID NO: 1) and Cas9 protein to yield a CIITA null, SERPINB9 expressing, HLA-E expressing, B2M null, XIAP expressing, and IL15/IR15α expressing cell.

### Homology-Directed Repair (HDR)

The donor nucleic acid (e.g., encoding a protein of interest) can be inserted by homology directed repair (HDR) into the target gene locus. Both strands of the DNA at the target locus are cut by a CRISPR Cas9 enzyme. HDR then occurs to repair the double-strand break (DSB) and insert the donor DNA. For this to occur correctly, the donor sequence is designed with flanking residues which are complementary to the sequence surrounding the DSB site in the target gene (hereinafter “homology arms”). These homology arms serve as the template for DSB repair and allow HDR to be an essentially error-free mechanism. The frequency of homology directed repair (HDR) is a function of the distance between the mutation and the cut site so choosing overlapping or nearby target sites is important. Templates can include extra sequences flanked by the homologous regions or can contain a sequence that differs from the genomic sequence, thus allowing sequence editing.

### Genome Editing Methods

Genome editing generally refers to the process of modifying the nucleotide sequence of a genome, preferably in a precise or pre-determined manner. In some embodiments, genome editing methods as described herein, e.g., the CRISPR-endonuclease system, are used to genetically modify a cell as described herein, e.g., to create a gene-edited iPSC cell. In some embodiments, genome editing methods as described herein, e.g., the CRISPR-endonuclease system, are used to genetically modify a cell as described herein, e.g., to introduce at least one genetic modification within or near at least one gene that increases the expression of one or more MIC-I and/or MHC-II human leukocyte antigens or other components of the MHC-I or MHC-II complex relative to an unmodified cell; to introduce at least one genetic modification that increases the expression of at least one polynucleotide that encodes a tolerogenic factor relative to an unmodified cell; and/or introduce at least one genetic modification that increases or decreases the expression of at least one gene that encodes a targeting factor that improves immunogenicity.

Examples of methods of genome editing described herein include methods of using site-directed nucleases to cut deoxyribonucleic acid (DNA) at precise target locations in the genome, thereby creating single-strand or double-strand DNA breaks at particular locations within the genome. Such breaks can be and regularly are repaired by natural, endogenous cellular processes, such as homology-directed repair (HDR) and non-homologous end joining (NHEJ), as described in Cox et al., “Therapeutic genome editing: prospects and challenges,”, Nature Medicine, 2015, 21(2), 121-31. These two main DNA repair processes consist of a family of alternative pathways. NHEJ directly joins the DNA ends resulting from a double-strand break, sometimes with the loss or addition of nucleotide sequence, which may disrupt or enhance gene expression. HDR utilizes a homologous sequence, or donor sequence, as a template for inserting a defined DNA sequence at the break point. The homologous sequence can be in the endogenous genome, such as a sister chromatid. Alternatively, the donor sequence can be an exogenous polynucleotide, such as a plasmid, a single-strand oligonucleotide, a double-stranded oligonucleotide, a duplex oligonucleotide or a virus, that has regions (e.g., left and right homology arms) of high homology with the nuclease-cleaved locus, but which can also contain additional sequence or sequence changes including deletions that can be incorporated into the cleaved target locus. A third repair mechanism can be microhomology-mediated end joining (MMEJ), also referred to as “Alternative NHEJ,” in which the genetic outcome is similar to NHEJ in that small deletions and insertions can occur at the cleavage site. MMEJ can make use of homologous sequences of a few base pairs flanking the DNA break site to drive a more favored DNA end joining repair outcome, and recent reports have further elucidated the molecular mechanism of this process; see, e.g., Cho and Greenberg, Nature, 2015, 518, 174-76; Kent et al., Nature Structural and Molecular Biology, 2015, 22(3):230-7; Mateos-Gomez et al., Nature, 2015, 518, 254-57; Ceccaldi et al., Nature, 2015, 528, 258-62. In some instances, it may be possible to predict likely repair outcomes based on analysis of potential microhomologies at the site of the DNA break.

Each of these genome editing mechanisms can be used to create desired genetic modifications. A step in the genome editing process can be to create one or two DNA breaks, the latter as double-strand breaks or as two single-stranded breaks, in the target locus as near the site of intended mutation. This can be achieved via the use of endonucleases, as described and illustrated herein.

In general, the genome editing methods described herein can be in vitro or ex vivo methods. In some embodiments, the genome editing methods disclosed herein are not methods for treatment of the human or animal body by therapy and/or are not processes for modifying the germ line genetic identity of human beings.

### CRISPR Endonuclease System

The CRISPR-endonuclease system is a naturally occurring defense mechanism in prokaryotes that has been repurposed as a RNA-guided DNA-targeting platform used for gene editing. CRISPR systems include Types I, II, III, IV, V, and VI systems. In some aspects, the CRISPR system is a Type II CRISPR/Cas9 system. In other aspects, the CRISPR system is a Type V CRISPR/Cprf system. CRISPR systems rely on a DNA endonuclease, e.g., Cas9, and two noncoding RNAs—crisprRNA (crRNA) and trans-activating RNA (tracrRNA)—to target the cleavage of DNA.

The crRNA drives sequence recognition and specificity of the CRISPR-endonuclease complex through Watson-Crick base pairing, typically with a ˜20 nucleotide (nt) sequence in the target DNA. Changing the sequence of the 5′ 20 nt in the crRNA allows targeting of the CRISPR-endonuclease complex to specific loci. The CRISPR-endonuclease complex only binds DNA sequences that contain a sequence match to the first 20 nt of the single-guide RNA (sgRNA) if the target sequence is followed by a specific short DNA motif (with the sequence NGG) referred to as a protospacer adjacent motif (PAM).

TracrRNA hybridizes with the 3′ end of crRNA to form an RNA-duplex structure that is bound by the endonuclease to form the catalytically active CRISPR-endonuclease complex, which can then cleave the target DNA.

Once the CRISPR-endonuclease complex is bound to DNA at a target site, two independent nuclease domains within the endonuclease each cleave one of the DNA strands three bases upstream of the PAM site, leaving a double-strand break (DSB) where both strands of the DNA terminate in a base pair (a blunt end).

In some embodiments, the endonuclease is a Cas9 (CRISPR associated protein 9). In some embodiments, the Cas9 endonuclease is from Streptococcus pyogenes, although other Cas9 homologs may be used, e.g., S. aureus Cas9, N. meningitidis Cas9, S. thermophilus CRISPR 1 Cas9, S. thermophilus CRISPR 3 Cas9, or T. denticola Cas9. In some embodiments, the CRISPR endonuclease is Cpf1, e.g., L. bacterium ND2006 Cpf1 or Acidaminococcus sp. BV3L6 Cpf1. In some embodiments, the endonuclease is Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease. In some embodiments, wild-type variants may be used. In some embodiments, modified versions (e.g., a homolog thereof, a recombination of the naturally occurring molecule thereof, codon-optimized thereof, or modified versions thereof) of the preceding endonucleases may be used.

The CRISPR nuclease can be linked to at least one nuclear localization signal (NLS). The at least one NLS can be located at or within 50 amino acids of the amino-terminus of the CRISPR nuclease and/or at least one NLS can be located at or within 50 amino acids of the carboxy-terminus of the CRISPR nuclease.

Exemplary CRISPR/Cas polypeptides include the Cas9 polypeptides as published in Fonfara et al., “Phylogeny of Cas9 determines functional exchangeability of dual-RNA and Cas9 among orthologous type II CRISPR-Cas systems,” Nucleic Acids Research, 2014, 42: 2577-2590. The CRISPR/Cas gene naming system has undergone extensive rewriting since the Cas genes were discovered. Fonfara et al. also provides PAM sequences for the Cas9 polypeptides from various species.

### Zinc Finger Nucleases

Zinc finger nucleases (ZFNs) are modular proteins comprised of an engineered zinc finger DNA binding domain linked to the catalytic domain of the type II endonuclease FokI. Because FokI functions only as a dimer, a pair of ZFNs must be engineered to bind to cognate target “half-site” sequences on opposite DNA strands and with precise spacing between them to enable the catalytically active FokI dimer to form. Upon dimerization of the FokI domain, which itself has no sequence specificity per se, a DNA double-strand break is generated between the ZFN half-sites as the initiating step in genome editing.

The DNA binding domain of each ZFN is typically comprised of 3-6 zinc fingers of the abundant Cys2-His2 architecture, with each finger primarily recognizing a triplet of nucleotides on one strand of the target DNA sequence, although cross-strand interaction with a fourth nucleotide also can be important. Alteration of the amino acids of a finger in positions that make key contacts with the DNA alters the sequence specificity of a given finger. Thus, a four-finger zinc finger protein will selectively recognize a 12 bp target sequence, where the target sequence is a composite of the triplet preferences contributed by each finger, although triplet preference can be influenced to varying degrees by neighboring fingers. An important aspect of ZFNs is that they can be readily re-targeted to almost any genomic address simply by modifying individual fingers. In most applications of ZFNs, proteins of 4-6 fingers are used, recognizing 12-18 bp respectively. Hence, a pair of ZFNs will typically recognize a combined target sequence of 24-36 bp, not including the typical 5-7 bp spacer between half-sites. The binding sites can be separated further with larger spacers, including 15-17 bp. A target sequence of this length is likely to be unique in the human genome, assuming repetitive sequences or gene homologs are excluded during the design process. Nevertheless, the ZFN protein-DNA interactions are not absolute in their specificity so off-target binding and cleavage events do occur, either as a heterodimer between the two ZFNs, or as a homodimer of one or the other of the ZFNs. The latter possibility has been effectively eliminated by engineering the dimerization interface of the FokI domain to create “plus” and “minus” variants, also known as obligate heterodimer variants, which can only dimerize with each other, and not with themselves. Forcing the obligate heterodimer prevents formation of the homodimer. This has greatly enhanced specificity of ZFNs, as well as any other nuclease that adopts these FokI variants.

A variety of ZFN-based systems have been described in the art, modifications thereof are regularly reported, and numerous references describe rules and parameters that are used to guide the design of ZFNs; see, e.g., Segal et al., Proc Natl Acad Sci, 1999 96(6):2758-63; Dreier B et al., J Mol Biol., 2000, 303(4):489-502; Liu Q et al., J Biol Chem., 2002, 277(6):3850-6; Dreier et al., J Biol Chem., 2005, 280(42):35588-97; and Dreier et al., J Biol Chem. 2001, 276(31):29466-78.

### Transcription Activator-Like Effector Nucleases (TALENs)

TALENs represent another format of modular nucleases whereby, as with ZFNs, an engineered DNA binding domain is linked to the FokI nuclease domain, and a pair of TALENs operate in tandem to achieve targeted DNA cleavage. The major difference from ZFNs is the nature of the DNA binding domain and the associated target DNA sequence recognition properties. The TALEN DNA binding domain derives from TALE proteins, which were originally described in the plant bacterial pathogen Xanthomonas sp. TALEs are comprised of tandem arrays of 33-35 amino acid repeats, with each repeat recognizing a single base pair in the target DNA sequence that is typically up to 20 bp in length, giving a total target sequence length of up to 40 bp. Nucleotide specificity of each repeat is determined by the repeat variable diresidue (RVD), which includes just two amino acids at positions 12 and 13. The bases guanine, adenine, cytosine and thymine are predominantly recognized by the four RVDs: Asn-Asn, Asn-Ile, His-Asp and Asn-Gly, respectively. This constitutes a much simpler recognition code than for zinc fingers, and thus represents an advantage over the latter for nuclease design. Nevertheless, as with ZFNs, the protein-DNA interactions of TALENs are not absolute in their specificity, and TALENs have also benefitted from the use of obligate heterodimer variants of the FokI domain to reduce off-target activity.

Additional variants of the FokI domain have been created that are deactivated in their catalytic function. If one half of either a TALEN or a ZFN pair contains an inactive FokI domain, then only single-strand DNA cleavage (nicking) will occur at the target site, rather than a DSB. The outcome is comparable to the use of CRISPR/Cas9 or CRISPR/Cpf1 “nickase” mutants in which one of the Cas9 cleavage domains has been deactivated. DNA nicks can be used to drive genome editing by HDR, but at lower efficiency than with a DSB. The main benefit is that off-target nicks are quickly and accurately repaired, unlike the DSB, which is prone to NHEJ-mediated mis-repair.

A variety of TALEN-based systems have been described in the art, and modifications thereof are regularly reported; see, e.g., Boch, Science, 2009 326(5959):1509-12; Mak et al., Science, 2012, 335(6069):716-9; and Moscou et al., Science, 2009, 326(5959):1501. The use of TALENs based on the “Golden Gate” platform, or cloning scheme, has been described by multiple groups; see, e.g., Cermak et al., Nucleic Acids Res., 2011, 39(12):e82; Li et al., Nucleic Acids Res., 2011, 39(14):6315-25; Weber et al., PLoS One., 2011, 6(2):e16765; Wang et al., J Genet Genomics, 2014, 41(6):339-47.; and Cermak T et al., Methods Mol Biol., 2015 1239:133-59.

### Homing Endonucleases

Homing endonucleases (HEs) are sequence-specific endonucleases that have long recognition sequences (14-44 base pairs) and cleave DNA with high specificity—often at sites unique in the genome. There are at least six known families of HEs as classified by their structure, including GIY-YIG, His-Cis box, H-N-H, PD-(D/E)×K, and Vsr-like that are derived from a broad range of hosts, including eukarya, protists, bacteria, archaea, cyanobacteria and phage. As with ZFNs and TALENs, HEs can be used to create a DSB at a target locus as the initial step in genome editing. In addition, some natural and engineered HEs cut only a single strand of DNA, thereby functioning as site-specific nickases. The large target sequence of HEs and the specificity that they offer have made them attractive candidates to create site-specific DSBs.

A variety of HE-based systems have been described in the art, and modifications thereof are regularly reported; see, e.g., the reviews by Steentoft et al., Glycobiology, 2014, 24(8):663-80; Belfort and Bonocora, Methods Mol Biol., 2014, 1123:1-26; and Hafez and Hausner, Genome, 2012, 55(8):553-69.

### MegaTAL/Tev-mTALEN/MegaTev

As further examples of hybrid nucleases, the MegaTAL platform and Tev-mTALEN platform use a fusion of TALE DNA binding domains and catalytically active HEs, taking advantage of both the tunable DNA binding and specificity of the TALE, as well as the cleavage sequence specificity of the HE; see, e.g., Boissel et al., Nucleic Acids Res., 2014, 42: 2591-2601; Kleinstiver et al., G3, 2014, 4:1155-65; and Boissel and Scharenberg, Methods Mol. Biol., 2015, 1239: 171-96.

In a further variation, the MegaTev architecture is the fusion of a meganuclease (Mega) with the nuclease domain derived from the GIY-YIG homing endonuclease I-TevI (Tev). The two active sites are positioned ˜30 bp apart on a DNA substrate and generate two DSBs with non-compatible cohesive ends; see, e.g., Wolfs et al., Nucleic Acids Res., 2014, 42, 8816-29. It is anticipated that other combinations of existing nuclease-based approaches will evolve and be useful in achieving the targeted genome modifications described herein.

dCas9-FokI or dCpf1-FokI and Other Nucleases

Combining the structural and functional properties of the nuclease platforms described above offers a further approach to genome editing that can potentially overcome some of the inherent deficiencies. As an example, the CRISPR genome editing system typically uses a single Cas9 endonuclease to create a DSB. The specificity of targeting is driven by a 20 or 24 nucleotide sequence in the guide RNA that undergoes Watson-Crick base-pairing with the target DNA (plus an additional 2 bases in the adjacent NAG or NGG PAM sequence in the case of Cas9 from S. pyogenes). Such a sequence is long enough to be unique in the human genome, however, the specificity of the RNA/DNA interaction is not absolute, with significant promiscuity sometimes tolerated, particularly in the 5′ half of the target sequence, effectively reducing the number of bases that drive specificity. One solution to this has been to completely deactivate the Cas9 or Cpf1 catalytic function—retaining only the RNA-guided DNA binding function—and instead fusing a FokI domain to the deactivated Cas9; see, e.g., Tsai et al., Nature Biotech, 2014, 32: 569-76; and Guilinger et al., Nature Biotech., 2014, 32: 577-82. Because FokI must dimerize to become catalytically active, two guide RNAs are required to tether two FokI fusions in close proximity to form the dimer and cleave DNA. This essentially doubles the number of bases in the combined target sites, thereby increasing the stringency of targeting by CRISPR-based systems.

As further example, fusion of the TALE DNA binding domain to a catalytically active HE, such as I-TevI, takes advantage of both the tunable DNA binding and specificity of the TALE, as well as the cleavage sequence specificity of I-TevI, with the expectation that off-target cleavage can be further reduced.

### Base Editing

In some embodiments, a gene is edited in a cell using base editing. Base Editing is a technique enabling the conversion of one nucleotide into another without double-stranded breaks in the DNA. Base editing allows for conversion of a C to T, G to A, or vice versa. An example editor for cytosine includes rAPOBEC1 which is fused to a catalytically inactive form of Cas9. The Cas9 helps to bind a site of interest and the rAPOBEC1 cytidine deaminase induces the point mutation. Conversion of adenine requires a mutant transfer RNA adenosine deaminase (TadA), a Cas9 nickase, and a sgRNA, as described herein. The construct is able to introduce the site-specific mutation without introducing a strand break. In some embodiments, Base Editing is used to introduce one or more mutations in a cell described herein.

### RNA-Guided Endonucleases

The RNA-guided endonuclease systems as used herein can comprise an amino acid sequence having at least 10%, at least 15%, at least 20%, at least 30%, at least 40%, at least 50%, at least 60%, at least 70%, at least 75%, at least 80%, at least 85%, at least 90%, at least 95%, at least 99%, or 100% amino acid sequence identity to a wild-type exemplary endonuclease, e.g., Cas9 from S. pyogenes, US2014/0068797 Sequence ID No. 8 or Sapranauskas et al., Nucleic Acids Res, 39(21): 9275-9282 (2011). The endonuclease can comprise at least 70, 75, 80, 85, 90, 95, 97, 99, or 100% identity to a wild-type endonuclease (e.g., Cas9 from S. pyogenes, supra) over 10 contiguous amino acids. The endonuclease can comprise at most: 70, 75, 80, 85, 90, 95, 97, 99, or 100% identity to a wild-type endonuclease (e.g., Cas9 from S. pyogenes, supra) over 10 contiguous amino acids. The endonuclease can comprise at least: 70, 75, 80, 85, 90, 95, 97, 99, or 100% identity to a wild-type endonuclease (e.g., Cas9 from S. pyogenes, supra) over 10 contiguous amino acids in a HNH nuclease domain of the endonuclease. The endonuclease can comprise at most: 70, 75, 80, 85, 90, 95, 97, 99, or 100% identity to a wild-type endonuclease (e.g., Cas9 from S. pyogenes, supra) over 10 contiguous amino acids in a HNH nuclease domain of the endonuclease. The endonuclease can comprise at least: 70, 75, 80, 85, 90, 95, 97, 99, or 100% identity to a wild-type endonuclease (e.g., Cas9 from S. pyogenes, supra) over 10 contiguous amino acids in a RuvC nuclease domain of the endonuclease. The endonuclease can comprise at most: 70, 75, 80, 85, 90, 95, 97, 99, or 100% identity to a wild-type endonuclease (e.g., Cas9 from S. pyogenes, supra) over 10 contiguous amino acids in a RuvC nuclease domain of the endonuclease.

The endonuclease can comprise a modified form of a wild-type exemplary endonuclease. The modified form of the wild-type exemplary endonuclease can comprise a mutation that reduces the nucleic acid-cleaving activity of the endonuclease. The modified form of the wild-type exemplary endonuclease can have less than 90%, less than 80%, less than 70%, less than 60%, less than 50%, less than 40%, less than 30%, less than 20%, less than 10%, less than 5%, or less than 1% of the nucleic acid-cleaving activity of the wild-type exemplary endonuclease (e.g., Cas9 from S. pyogenes, supra). The modified form of the endonuclease can have no substantial nucleic acid-cleaving activity. When an endonuclease is a modified form that has no substantial nucleic acid-cleaving activity, it is referred to herein as “enzymatically inactive.”

Mutations contemplated can include substitutions, additions, and deletions, or any combination thereof. The mutation converts the mutated amino acid to alanine. The mutation converts the mutated amino acid to another amino acid (e.g., glycine, serine, threonine, cysteine, valine, leucine, isoleucine, methionine, proline, phenylalanine, tyrosine, tryptophan, aspartic acid, glutamic acid, asparagine, glutamine, histidine, lysine, or arginine). The mutation converts the mutated amino acid to a non-natural amino acid (e.g., selenomethionine). The mutation converts the mutated amino acid to amino acid mimics (e.g., phosphomimics). The mutation can be a conservative mutation. For example, the mutation converts the mutated amino acid to amino acids that resemble the size, shape, charge, polarity, conformation, and/or rotamers of the mutated amino acids (e.g., cysteine/serine mutation, lysine/asparagine mutation, histidine/phenylalanine mutation). The mutation can cause a shift in reading frame and/or the creation of a premature stop codon. Mutations can cause changes to regulatory regions of genes or loci that affect expression of one or more genes.

### Guide RNAs

The present disclosure provides a guide RNAs (gRNAs) that can direct the activities of an associated endonuclease to a specific target site within a polynucleotide. A guide RNA can comprise at least a spacer sequence that hybridizes to a target nucleic acid sequence of interest, and a CRISPR repeat sequence. In CRISPR Type II systems, the gRNA also comprises a second RNA called the tracrRNA sequence. In the CRISPR Type II guide RNA (gRNA), the CRISPR repeat sequence and tracrRNA sequence hybridize to each other to form a duplex. In CRISPR Type V systems, the gRNA comprises a crRNA that forms a duplex. In some embodiments, a gRNA can bind an endonuclease, such that the gRNA and endonuclease form a complex. The gRNA can provide target specificity to the complex by virtue of its association with the endonuclease. The genome-targeting nucleic acid thus can direct the activity of the endonuclease.

Exemplary guide RNAs include a spacer sequences that comprises 15-200 nucleotides wherein the gRNA targets a genome location based on the GRCh38 human genome assembly. As is understood by the person of ordinary skill in the art, each gRNA can be designed to include a spacer sequence complementary to its genomic target site or region, ie., the “target sequence”. The “target sequence” is in a target gene that is adjacent to a PAM sequence and is the sequence to be modified by Cas9. The “target sequence” is on the so-called PAM-strand in a “target nucleic acid,” which is a double-stranded molecule containing the PAM-strand and a complementary non-PAM strand. One of skill in the art recognizes that the gRNA spacer sequence hybridizes to the complementary sequence located in the non-PAM strand of the target nucleic acid of interest. Thus, the gRNA spacer sequence is the RNA equivalent of the target sequence. See Jinek et al., Science, 2012, 337, 816-821 and Deltcheva et al., Nature, 2011, 471, 602-607.

The gRNA can be a double-molecule guide RNA. The gRNA can be a single-molecule guide RNA.

A double-molecule guide RNA can comprise two strands of RNA. The first strand comprises in the 5′ to 3′ direction, an optional spacer extension sequence, a spacer sequence and a minimum CRISPR repeat sequence. The second strand can comprise a minimum tracrRNA sequence (complementary to the minimum CRISPR repeat sequence), a 3′ tracrRNA sequence and an optional tracrRNA extension sequence.

A single-molecule guide RNA (sgRNA) can comprise, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension can comprise elements that contribute additional functionality (e.g., stability) to the guide RNA. The single-molecule guide linker can link the minimum CRISPR repeat and the minimum tracrRNA sequence to form a hairpin structure. The optional tracrRNA extension can comprise one or more hairpins.

In some embodiments, a sgRNA comprises a 20 nucleotide spacer sequence at the 5′ end of the sgRNA sequence. In some embodiments, a sgRNA comprises a less than a 20 nucleotide spacer sequence at the 5′ end of the sgRNA sequence. In some embodiments, a sgRNA comprises a more than 20 nucleotide spacer sequence at the 5′ end of the sgRNA sequence. In some embodiments, a sgRNA comprises a variable length spacer sequence with 17-30 nucleotides at the 5′ end of the sgRNA sequence. In some embodiments, a sgRNA comprises a spacer extension sequence with a length of more than 1, 5, 10, 15, 20, 25, 30, 35, 40, 45, 50, 60, 70, 80, 90, 100, 120, 140, 160, 180, or 200 nucleotides. In some embodiments, a sgRNA comprises a spacer extension sequence with a length of less than 3, 5, 10, 15, 20, 25, 30, 35, 40, 45, 50, 60, 70, 80, 90, or 100 nucleotides.

In some embodiments, a sgRNA comprises a spacer extension sequence that comprises another moiety (e.g., a stability control sequence, an endoribonuclease binding sequence, or a ribozyme). The moiety can decrease or increase the stability of a nucleic acid targeting nucleic acid. The moiety can be a transcriptional terminator segment (i.e., a transcription termination sequence). The moiety can function in a eukaryotic cell. The moiety can function in a prokaryotic cell. The moiety can function in both eukaryotic and prokaryotic cells. Non-limiting examples of suitable moieties include: a 5′ cap (e.g., a 7-methylguanylate cap (m7 G)), a riboswitch sequence (e.g., to allow for regulated stability and/or regulated accessibility by proteins and protein complexes), a sequence that forms a dsRNA duplex (i.e., a hairpin), a sequence that targets the RNA to a subcellular location (e.g., nucleus, mitochondria, chloroplasts, and the like), a modification or sequence that provides for tracking (e.g., direct conjugation to a fluorescent molecule, conjugation to a moiety that facilitates fluorescent detection, a sequence that allows for fluorescent detection, etc.), and/or a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like).

In some embodiments, a sgRNA comprises a spacer sequence that hybridizes to a sequence in a target polynucleotide. The spacer of a gRNA can interact with a target polynucleotide in a sequence-specific manner via hybridization (i.e., base pairing). The nucleotide sequence of the spacer can vary depending on the sequence of the target nucleic acid of interest.

In a CRISPR-endonuclease system, a spacer sequence can be designed to hybridize to a target polynucleotide that is located 5′ of a PAM of the endonuclease used in the system. The spacer may perfectly match the target sequence or may have mismatches. Each endonuclease, e.g., Cas9 nuclease, has a particular PAM sequence that it recognizes in a target DNA. For example, S. pyogenes Cas9 recognizes a PAM that comprises the sequence 5′-NRG-3′, where R comprises either A or G, where N is any nucleotide and N is immediately 3′ of the target nucleic acid sequence targeted by the spacer sequence.

A target polynucleotide sequence can comprise 20 nucleotides. The target polynucleotide can comprise less than 20 nucleotides. The target polynucleotide can comprise more than 20 nucleotides. The target polynucleotide can comprise at least: 5, 10, 15, 16, 17, 18, 19, 20, 21, 22, 23, 24, 25, 30 or more nucleotides. The target polynucleotide can comprise at most: 5, 10, 15, 16, 17, 18, 19, 20, 21, 22, 23, 24, 25, 30 or more nucleotides. The target polynucleotide sequence can comprise 20 bases immediately 5′ of the first nucleotide of the PAM.

A spacer sequence that hybridizes to a target polynucleotide can have a length of at least about 6 nucleotides (nt). The spacer sequence can be at least about 6 nt, at least about 10 nt, at least about 15 nt, at least about 18 nt, at least about 19 nt, at least about 20 nt, at least about 25 nt, at least about 30 nt, at least about 35 nt or at least about 40 nt, from about 6 nt to about 80 nt, from about 6 nt to about 50 nt, from about 6 nt to about 45 nt, from about 6 nt to about 40 nt, from about 6 nt to about 35 nt, from about 6 nt to about 30 nt, from about 6 nt to about 25 nt, from about 6 nt to about 20 nt, from about 6 nt to about 19 nt, from about 10 nt to about 50 nt, from about 10 nt to about 45 nt, from about 10 nt to about 40 nt, from about 10 nt to about 35 nt, from about 10 nt to about 30 nt, from about 10 nt to about 25 nt, from about 10 nt to about 20 nt, from about 10 nt to about 19 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt. In some examples, the spacer sequence can comprise 20 nucleotides. In some examples, the spacer can comprise 19 nucleotides. In some examples, the spacer can comprise 18 nucleotides. In some examples, the spacer can comprise 22 nucleotides.

In some examples, the percent complementarity between the spacer sequence and the target nucleic acid is at least about 30%, at least about 40%, at least about 50%, at least about 60%, at least about 65%, at least about 70%, at least about 75%, at least about 80%, at least about 85%, at least about 90%, at least about 95%, at least about 97%, at least about 98%, at least about 99%, or 100%. In some examples, the percent complementarity between the spacer sequence and the target nucleic acid is at most about 30%, at most about 40%, at most about 50%, at most about 60%, at most about 65%, at most about 70%, at most about 75%, at most about 80%, at most about 85%, at most about 90%, at most about 95%, at most about 97%, at most about 98%, at most about 99%, or 100%. In some examples, the percent complementarity between the spacer sequence and the target nucleic acid is 100% over the six contiguous 5′-most nucleotides of the target sequence of the complementary strand of the target nucleic acid. The percent complementarity between the spacer sequence and the target nucleic acid can be at least 60% over about 20 contiguous nucleotides. The length of the spacer sequence and the target nucleic acid can differ by 1 to 6 nucleotides, which may be thought of as a bulge or bulges.

A tracrRNA sequence can comprise nucleotides that hybridize to a minimum CRISPR repeat sequence in a cell. A minimum tracrRNA sequence and a minimum CRISPR repeat sequence may form a duplex, i.e. a base-paired double-stranded structure. Together, the minimum tracrRNA sequence and the minimum CRISPR repeat can bind to an RNA-guided endonuclease. At least a part of the minimum tracrRNA sequence can hybridize to the minimum CRISPR repeat sequence. The minimum tracrRNA sequence can be at least about 30%, about 40%, about 50%, about 60%, about 65%, about 70%, about 75%, about 80%, about 85%, about 90%, about 95%, or 100% complementary to the minimum CRISPR repeat sequence.

The minimum tracrRNA sequence can have a length from about 7 nucleotides to about 100 nucleotides. For example, the minimum tracrRNA sequence can be from about 7 nucleotides (nt) to about 50 nt, from about 7 nt to about 40 nt, from about 7 nt to about 30 nt, from about 7 nt to about 25 nt, from about 7 nt to about 20 nt, from about 7 nt to about 15 nt, from about 8 nt to about 40 nt, from about 8 nt to about 30 nt, from about 8 nt to about 25 nt, from about 8 nt to about 20 nt, from about 8 nt to about 15 nt, from about 15 nt to about 100 nt, from about 15 nt to about 80 nt, from about 15 nt to about 50 nt, from about 15 nt to about 40 nt, from about 15 nt to about 30 nt or from about 15 nt to about 25 nt long. The minimum tracrRNA sequence can be approximately 9 nucleotides in length. The minimum tracrRNA sequence can be approximately 12 nucleotides. The minimum tracrRNA can consist of tracrRNA nt 23-48 described in Jinek et al., supra.

The minimum tracrRNA sequence can be at least about 60% identical to a reference minimum tracrRNA (e.g., wild type, tracrRNA from S. pyogenes) sequence over a stretch of at least 6, 7, or 8 contiguous nucleotides. For example, the minimum tracrRNA sequence can be at least about 65% identical, about 70% identical, about 75% identical, about 80% identical, about 85% identical, about 90% identical, about 95% identical, about 98% identical, about 99% identical or 100% identical to a reference minimum tracrRNA sequence over a stretch of at least 6, 7, or 8 contiguous nucleotides.

The duplex between the minimum CRISPR RNA and the minimum tracrRNA can comprise a double helix. The duplex between the minimum CRISPR RNA and the minimum tracrRNA can comprise at least about 1, 2, 3, 4, 5, 6, 7, 8, 9, or 10 or more nucleotides. The duplex between the minimum CRISPR RNA and the minimum tracrRNA can comprise at most about 1, 2, 3, 4, 5, 6, 7, 8, 9, or 10 or more nucleotides.

The duplex can comprise a mismatch (i.e., the two strands of the duplex are not 100% complementary). The duplex can comprise at least about 1, 2, 3, 4, or 5 or mismatches. The duplex can comprise at most about 1, 2, 3, 4, or 5 or mismatches. The duplex can comprise no more than 2 mismatches.

In some embodiments, a tracrRNA may be a 3′ tracrRNA. In some embodiments, a 3′ tracrRNA sequence can comprise a sequence with at least about 30%, about 40%, about 50%, about 60%, about 65%, about 70%, about 75%, about 80%, about 85%, about 90%, about 95%, or 100% sequence identity to a reference tracrRNA sequence (e.g., a tracrRNA from S. pyogenes).

In some embodiments, a gRNA may comprise a tracrRNA extension sequence. A tracrRNA extension sequence can have a length from about 1 nucleotide to about 400 nucleotides. The tracrRNA extension sequence can have a length of more than 1, 5, 10, 15, 20, 25, 30, 35, 40, 45, 50, 60, 70, 80, 90, 100, 120, 140, 160, 180, or 200 nucleotides. The tracrRNA extension sequence can have a length from about 20 to about 5000 or more nucleotides. The tracrRNA extension sequence can have a length of less than 5, 10, 15, 20, 25, 30, 35, 40, 45, 50, 60, 70, 80, 90, or 100 nucleotides. The tracrRNA extension sequence can comprise less than 10 nucleotides in length. The tracrRNA extension sequence can be 10-30 nucleotides in length. The tracrRNA extension sequence can be 30-70 nucleotides in length.

The tracrRNA extension sequence can comprise a functional moiety (e.g., a stability control sequence, ribozyme, endoribonuclease binding sequence). The functional moiety can comprise a transcriptional terminator segment (i.e., a transcription termination sequence). The functional moiety can have a total length from about 10 nucleotides (nt) to about 100 nucleotides, from about 10 nt to about 20 nt, from about 20 nt to about 30 nt, from about 30 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt, from about 15 nt to about 80 nt, from about 15 nt to about 50 nt, from about 15 nt to about 40 nt, from about 15 nt to about 30 nt, or from about 15 nt to about 25 nt.

In some embodiments, a sgRNA may comprise a linker sequence with a length from about 3 nucleotides to about 100 nucleotides. In Jinek et al., supra, for example, a simple 4 nucleotide “tetraloop” (-GAAA-) was used (Jinek et al., Science, 2012, 337(6096):816-821). An illustrative linker has a length from about 3 nucleotides (nt) to about 90 nt, from about 3 nt to about 80 nt, from about 3 nt to about 70 nt, from about 3 nt to about 60 nt, from about 3 nt to about 50 nt, from about 3 nt to about 40 nt, from about 3 nt to about 30 nt, from about 3 nt to about 20 nt, from about 3 nt to about 10 nt. For example, the linker can have a length from about 3 nt to about 5 nt, from about 5 nt to about 10 nt, from about 10 nt to about 15 nt, from about 15 nt to about 20 nt, from about 20 nt to about 25 nt, from about 25 nt to about 30 nt, from about 30 nt to about 35 nt, from about 35 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt. The linker of a single-molecule guide nucleic acid can be between 4 and 40 nucleotides. The linker can be at least about 100, 500, 1000, 1500, 2000, 2500, 3000, 3500, 4000, 4500, 5000, 5500, 6000, 6500, or 7000 or more nucleotides. The linker can be at most about 100, 500, 1000, 1500, 2000, 2500, 3000, 3500, 4000, 4500, 5000, 5500, 6000, 6500, or 7000 or more nucleotides.

Linkers can comprise any of a variety of sequences, although in some examples the linker will not comprise sequences that have extensive regions of homology with other portions of the guide RNA, which might cause intramolecular binding that could interfere with other functional regions of the guide. In Jinek et al., supra, a simple 4 nucleotide sequence -GAAA- was used (Jinek et al., Science, 2012, 337(6096):816-821), but numerous other sequences, including longer sequences can likewise be used.

The linker sequence can comprise a functional moiety. For example, the linker sequence can comprise one or more features, including an aptamer, a ribozyme, a protein-interacting hairpin, a protein binding site, a CRISPR array, an intron, or an exon. The linker sequence can comprise at least about 1, 2, 3, 4, or 5 or more functional moieties. In some examples, the linker sequence can comprise at most about 1, 2, 3, 4, or 5 or more functional moieties.

In some embodiments, a sgRNA does not comprise a uracil, e.g., at the 3′ end of the sgRNA sequence. In some embodiments, a sgRNA does comprise one or more uracils, e.g., at the 3′ end of the sgRNA sequence. In some embodiments, a sgRNA comprises 1, 2, 3, 4, 5, 6, 7, 8, 9, or 10 uracils (U) at the 3′ end of the sgRNA sequence.

A sgRNA may be chemically modified. In some embodiments, a chemically modified gRNA is a gRNA that comprises at least one nucleotide with a chemical modification, e.g., a 2′-O-methyl sugar modification. In some embodiments, a chemically modified gRNA comprises a modified nucleic acid backbone. In some embodiments, a chemically modified gRNA comprises a 2′-O-methyl-phosphorothioate residue. In some embodiments, chemical modifications enhance stability, reduce the likelihood or degree of innate immune response, and/or enhance other attributes, as described in the art.

In some embodiments, a modified gRNA may comprise a modified backbones, for example, phosphorothioates, phosphotriesters, morpholinos, methyl phosphonates, short chain alkyl or cycloalkyl intersugar linkages or short chain heteroatomic or heterocyclic intersugar linkages.

Morpholino-based compounds are described in Braasch and David Corey, Biochemistry, 2002, 41(14): 4503-4510; Genesis, 2001, Volume 30, Issue 3; Heasman, Dev. Biol., 2002, 243: 209-214; Nasevicius et al., Nat. Genet., 2000, 26:216-220; Lacerra et al., Proc. Natl. Acad. Sci., 2000, 97: 9591-9596.; and U.S. Pat. No. 5,034,506, issued Jul. 23, 1991.

Cyclohexenyl nucleic acid oligonucleotide mimetics are described in Wang et al., J. Am. Chem. Soc., 2000, 122: 8595-8602.

In some embodiments, a modified gRNA may comprise one or more substituted sugar moieties, e.g., one of the following at the 2′ position: OH, SH, SCH₃, F, OCN, OCH₃, OCH₃O(CH₂)ₙ CH₃, O(CH₂)ₙNH₂, or O(CH₂)ₙCH₃, where n is from 1 to about 10; C1 to C10 lower alkyl, alkoxyalkoxy, substituted lower alkyl, alkaryl or aralkyl; Cl; Br; CN; CF3; OCF₃; O-, S-, or N-alkyl; O-, S-, or N-alkenyl; SOCH₃; SO₂CH₃; ONO₂; NO₂; N₃; NH₂; heterocycloalkyl; heterocycloalkaryl; aminoalkylamino; polyalkylamino; substituted silyl; an RNA cleaving group; a reporter group; an intercalator; 2′-O-(2-methoxyethyl); 2′-methoxy (2′-O—CH₃); 2′-propoxy (2′-OCH₂CH₂CH₃); and 2′-fluoro (2′-F). Similar modifications may also be made at other positions on the gRNA, particularly the 3′ position of the sugar on the 3′ terminal nucleotide and the 5′ position of 5′ terminal nucleotide. In some examples, both a sugar and an internucleoside linkage, i.e., the backbone, of the nucleotide units can be replaced with novel groups.

Guide RNAs can also include, additionally or alternatively, nucleobase (often referred to in the art simply as “base”) modifications or substitutions. As used herein, “unmodified” or “natural” nucleobases include adenine (A), guanine (G), thymine (T), cytosine (C), and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine, and 2,6-diaminopurine. Kornberg, A., DNA Replication, W. H. Freeman & Co., San Francisco, pp 75-77, 1980; Gebeyehu et al., Nucl. Acids Res. 1997, 15:4513. A “universal” base known in the art, e.g., inosine, can also be included. 5-Me-C substitutions have been shown to increase nucleic acid duplex stability by 0.6-1.2° C. (Sanghvi, Y. S., in Crooke, S. T. and Lebleu, B., eds., Antisense Research and Applications, CRC Press, Boca Raton, 1993, pp. 276-278) and are aspects of base substitutions.

Modified nucleobases can comprise other synthetic and natural nucleobases, such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine, and 3-deazaguanine and 3-deazaadenine.

### Complexes of a Genome-Targeting Nucleic Acid and an Endonuclease

A gRNA interacts with an endonuclease (e.g., an RNA-guided nuclease such as Cas9), thereby forming a complex. The gRNA guides the endonuclease to a target polynucleotide.

The endonuclease and gRNA can each be administered separately to a cell or a subject. In some embodiments, the endonuclease can be pre-complexed with one or more guide RNAs, or one or more crRNA together with a tracrRNA. The pre-complexed material can then be administered to a cell or a subject. Such pre-complexed material is known as a ribonucleoprotein particle (RNP). The endonuclease in the RNP can be, for example, a Cas9 endonuclease or a Cpf1 endonuclease. The endonuclease can be flanked at the N-terminus, the C-terminus, or both the N-terminus and C-terminus by one or more nuclear localization signals (NLSs). For example, a Cas9 endonuclease can be flanked by two NLSs, one NLS located at the N-terminus and the second NLS located at the C-terminus. The NLS can be any NLS known in the art, such as a SV40 NLS. The weight ratio of genome-targeting nucleic acid to endonuclease in the RNP can be 1:1. For example, the weight ratio of sgRNA to Cas9 endonuclease in the RNP can be 1:1.

### Cells

Provided herein are any of the cells described herein having any of the gene-edits described herein. In some embodiments, a cell (and corresponding unmodified cell) is a mammalian cell. In some embodiments, a cell (and corresponding unmodified cell) is a human cell. In some embodiments, a cell (and corresponding unmodified cell) is a stem cell. In some embodiments, a cell (and corresponding unmodified cell) is a pluripotent stem cell (PSC). In some embodiments, a cell (and corresponding unmodified cell) is an embryonic stem cell (ESC), an adult stem cell (ASC), an induced pluripotent stem cell (iPSC), or a hematopoietic stem or progenitor cell (HSPC). In some embodiments, a cell is an iPSC. In some embodiments, a cell may be a differentiated cell. In some embodiments, a cell is a somatic cell, e.g., an immune system cell or a contractile cell, e.g., a skeletal muscle cell.

In some embodiments, the stem cells described herein (e.g., iPSCs) are gene-edited as described herein and then differentiated into a cell type of interest. In some embodiments, the differentiated cell retains the gene-edits of the cell from which it is derived.

The cells described herein may be differentiated into relevant cell types. In general, differentiation comprises maintaining the cells of interest for a period time and under conditions sufficient for the cells to differentiate into the differentiated cells of interest. For example, the engineered stem cells disclosed herein may be differentiated into mesenchymal progenitor cells (MPCs), hypoimmunogenic cardiomyocytes, muscle progenitor cells, blast cells, endothelial cells (ECs), macrophages, natural killer cells, hepatoblasts, hepatocytes, beta cells (e.g., pancreatic beta cells), pancreatic endoderm progenitors, pancreatic endocrine progenitors, or neural progenitor cells (NPCs). In some embodiments, any of the stem cells described herein are differentiated after gene-editing. In some embodiments, a cell is differentiated into a natural killer (NK) cell. In some embodiments, a cell is differentiated into a hepatoblast or a hepatocyte.

Stem cells are capable of both proliferation and giving rise to more progenitor cells, these in turn having the ability to generate a large number of mother cells that can in turn give rise to differentiated or differentiable daughter cells. The daughter cells themselves can be induced to proliferate and produce progeny that subsequently differentiate into one or more mature cell types, while also retaining one or more cells with parental developmental potential. The term “stem cell” refers then, to a cell with the capacity or potential, under particular circumstances, to differentiate to a more specialized or differentiated phenotype, and which retains the capacity, under certain circumstances, to proliferate without substantially differentiating. In one aspect, the term progenitor or stem cell refers to a generalized mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell that itself is derived from a multipotent cell, and so on. While each of these multipotent cells may be considered stem cells, the range of cell types that each can give rise to may vary considerably. Some differentiated cells also have the capacity to give rise to cells of greater developmental potential. Such capacity may be natural or may be induced artificially upon treatment with various factors. In many biological instances, stem cells can also be “multipotent” because they can produce progeny of more than one distinct cell type, but this is not required for “stem-ness.”

A “differentiated cell” is a cell that has progressed further down the developmental pathway than the cell to which it is being compared. Thus, stem cells can differentiate into lineage-restricted precursor cells (such as a hematopoietic stem and progenitor cell (HSPC)), which in turn can differentiate into other types of precursor cells further down the pathway (such as a common lymphoid progenitor cell), and then to an end-stage differentiated cell, such as a natural killer cell, which plays a characteristic role in a certain tissue type, and may or may not retain the capacity to proliferate further.

In some embodiments, any of the gene-edited cells described herein have one of more of the following characteristics; increased persistence, immune evasiveness, lack of an alloimmune T cell response, increased cytotoxic activity, improved antibody-dependent cellular cytotoxicity (ADCC), or increased anti-tumor activity. In some embodiments, any of the gene-edited cells described herein have one of more of the following characteristics relative to an un-edited (wild-type) cell described herein; increased persistency, immune evasiveness, lack of an alloimmune T cell response, increased cytotoxic activity, improved antibody-dependent cellular cytotoxicity (ADCC), or increased anti-tumor activity. In some embodiments, any of the gene-edited cells described herein are capable of cell expansion in the absence of exogenous IL15.

### Embryonic Stem Cells

The cells described herein may be embryonic stem cells (ESCs). ESCs are derived from blastocytes of mammalian embryos and are able differentiate into any cell type and propagate rapidly. ESCs are also believed to have a normal karyotype, maintaining high telomerase activity, and exhibiting remarkable long-term proliferative potential, making these cells excellent candidates for use as gene-edited stem cells. In some embodiments, ESCs with one, two, three, four, five, six or all of the following edits: B2M null, CIITA null, SERPINB9 KI, HLA-E knock-in, IL15/IL15Rα fusion protein knock-in, CAR knock-in, XIAP KI, CISH KO, FAS KO are differentiated into NK cells. In some embodiments, ESCs with one, two, three, four, five, six or all of the following edits: B2M null, CIITA null, SERPINB9 KI, HLA-E knock-in, IL15/IL15Rα fusion protein knock-in, CAR knock-in, XIAP KI, CISH KO, FAS KO are differentiated into hepatoblasts or hepatocytes.

### Adult Stem Cells

The cells described herein may be adult stem cells (ASCs). ASCs are undifferentiated cells that may be found in mammals, e.g., humans. ASCs are defined by their ability to self-renew, e.g., be passaged through several rounds of cell replication while maintaining their undifferentiated state, and ability to differentiate into several distinct cell types, e.g., glial cells. Adult stem cells are a broad class of stem cells that may encompass hematopoietic stem cells, mammary stem cells, intestinal stem cells, mesenchymal stem cells, endothelial stem cells, neural stem cells, olfactory adult stem cells, neural crest stem cells, and testicular cells. In some embodiments, ASCs with one, two, three, four, five, six or all of the following edits: B2M null, CIITA null, SERPINB9 KI, HLA-E knock-in, IL15/IL15Rα fusion protein knock-in, CAR knock-in, XIAP KI, CISH KO, FAS KO are differentiated into NK cells. In some embodiments, ASCs with one, two, three, four, five, six or all of the following edits: B2M null, CIITA null, SERPINB9 KI, HLA-E knock-in, IL15/IL15Rα fusion protein knock-in, CAR knock-in, XIAP KI, CISH KO, FAS KO are differentiated into hepatoblasts or hepatocytes.

### Induced Pluripotent Stem Cells

The cells described herein may be induced pluripotent stem cells (iPSCs). An iPSC may be generated directly from an adult human cell by introducing genes that encode critical transcription factors involved in pluripotency, e.g., OCT4, SOX2, C-MYC, and KLF4. An iPSC may be derived from the same subject to which subsequent progenitor cells are to be administered. That is, a somatic cell can be obtained from a subject, reprogrammed to an induced pluripotent stem cell, and then re-differentiated into a progenitor cell to be administered to the subject (e.g., autologous cells). However, in the case of autologous cells, a risk of immune response and poor viability post-engraftment remain. In some embodiments, iPSC are generated from adult somatic cells using genetic reprogramming methods known in the art. In some embodiments, the iPSCs are derived from a commercial source. In some embodiments, the iPSCs are human iPSCs. In some embodiments, the cells described herein are iPSC or a derivative cell. In some embodiments, iPSCs with one, two, three, four, five, six or all of the following edits: B2M null, CIITA null, SERPINB9 KI, HLA-E knock-in, IL15/IL15Rα fusion protein knock-in, CAR knock-in, XIAP KI, CISH KO, FAS KO are differentiated into NK cells. In some embodiments, iPSCs with one, two, three, four, five, six or all of the following edits: B2M null, CIITA null, SERPINB9 KI, HLA-E knock-in, IL15/IL15Rα fusion protein knock-in, CAR knock-in, XIAP KI, CISH KO, FAS KO are differentiated into hepatoblasts or hepatocytes.

### Mesoderm

The cells described herein may be mesodermal cells. This cell type is one of the three germinal layers in embryonic development. The mesoderm eventually differentiates into, but is not limited to muscle, connective tissue, bone, red blood cells, white blood cells, and microglia. In some embodiments, the gene-edited cells described herein are mesodermal cells. In some embodiments, mesodermal cells are derived from any of the stem cells described herein. In some embodiments, mesodermal cells are derived from iPSC. In some embodiments, the mesodermal cells have any of the gene-edits described herein. In some embodiments, the mesodermal cells are differentiated into NK cells. In some embodiments, mesodermal cells with one, two, three, four, five, six or all of the following edits: B2M null, CIITA null, SERPINB9 KI, HLA-E KI, IL15/IL15Rα fusion protein KI, CAR KI, XIAP KI, CISH KO, FAS KO are differentiated into NK cells. In some embodiments, mesodermal cells with one, two, three, four, five, six or all of the following edits: B2M null, CIITA null, SERPINB9 KI, HLA-E KI, IL15/IL15Rα fusion protein KI, CAR KI, XIAP KI, CISH KO, FAS KO are differentiated into hepatoblasts or hepatocytes.

### Hemogenic Endothelium

The cells described herein may be hemogenic endothelium (HE) cells. This cell type is an intermediate precursor of hematopoietic progenitors. In some embodiments, the cells described herein are hemogenic endothelium cells. In some embodiments, the gene-edited cells described herein are hemogenic endothelium cells. In some embodiments, hemogenic endothelium cells are derived from any of the stem cells described herein. In some embodiments, hemogenic endothelium cells are derived from iPSC. In some embodiments, the hemogenic endothelial cells have any of the gene-edits described herein. In some embodiments, the hemogenic endothelial cells are differentiated into NK cells. In some embodiments, HE cells with one, two, three, four, five, six or all of the following edits: B2M null, CIITA null, SERPINB9 KI, HLA-E KI, IL15/IL15Rα fusion protein KI, CAR KI, XIAP KI, CISH KO, FAS KO, are differentiated into NK cells.

### Human Hematopoietic Stem and Progenitor Cells

The cells described herein may be human hematopoietic stem and progenitor cells (hHSPCs). This stem cell lineage gives rise to all blood cell types, including erythroid (erythrocytes or red blood cells (RBCs)), myeloid (monocytes and macrophages, neutrophils, basophils, eosinophils, megakaryocytes/platelets, and dendritic cells), and lymphoid (T-cells, B-cells, NK-cells). Blood cells are produced by the proliferation and differentiation of a very small population of pluripotent hematopoietic stem cells (HSCs) that also have the ability to replenish themselves by self-renewal. During differentiation, the progeny of HSCs progress through various intermediate maturational stages, generating multi-potential and lineage-committed progenitor cells prior to reaching maturity. Bone marrow (BM) is the major site of hematopoiesis in humans and, under normal conditions, only small numbers of hematopoietic stem and progenitor cells (HSPCs) can be found in the peripheral blood (PB). Treatment with cytokines, some myelosuppressive drugs used in cancer treatment, and compounds that disrupt the interaction between hematopoietic and BM stromal cells can rapidly mobilize large numbers of stem and progenitors into the circulation. In some embodiments, HSPCs are derived from any of the stem cells described herein. In some embodiments, HSPCs are derived from iPSCs. In some embodiments, the HSPCs have any of the gene-edits described herein. In some embodiments, the HSPCs cells are differentiated into NK cells. In some embodiments, HSPCs with one, two, three, four, five, six or all of the following edits: B2M null, CIITA null, SERPINB9 KI, HLA-E KI, IL15/IL15Rα fusion protein KI, CAR KI, XIAP KI, CISH KO, FAS KO, are differentiated into NK cells.

### Common Lymphoid Progenitor

The cells described herein may be common lymphoid progenitor (CLP) cells. CLPs are descendants of HSPCs. These cells differentiate into the lymphoid lineage of blood cells. Further differentiation yields B-cell progenitor cells, Natural Killer cells, and Thymocytes. In some embodiments, the cells described herein are common lymphoid progenitors. In some embodiments, the gene-edited cells described herein are common lymphoid progenitors. In some embodiments, CLP cells are derived from iPSCs. In some embodiments, the CLP cells have any of the gene-edits described herein. In some embodiments, the CLP cells are differentiated into NK cells. In some embodiments, CLP cells with one, two, three, four, five, six or all of the following edits: B2M null, CIITA null, SERPINB9 KI, HLA-E KI, IL15/IL15Rα fusion protein KI, CAR KI, XIAP KI, CISH KO, FAS KO, are differentiated into NK cells.

Differentiation of Cells into Other Cell Types

Another step of the methods of the present disclosure may comprise differentiating cells into differentiated cells. The differentiating step may be performed according to any method known in the art. For example, human iPSCs are differentiated into natural killer cells using methods known in the art. In some embodiments, the differentiating step may be performed according to Zhu and Kaufman, bioRxiv 2019; dx.doi.org/10.1101/614792. A differentiated cell may be any somatic cell of a mammal, e.g., a human. In some embodiments, a somatic cell may be an endocrine secretory epithelial cell (e.g., thyroid hormone secreting cells, adrenal cortical cells), an exocrine secretory epithelial cell (e.g., salivary gland mucous cell, prostate gland cell), a hormone-secreting cell (e.g., anterior pituitary cell, pancreatic islet cell), a keratinizing epithelial cell (e.g., epidermal keratinocyte), a wet stratified barrier epithelial cell, a sensory transducer cell (e.g., a photoreceptor), an autonomic neuron cells, a sense organ and peripheral neuron supporting cell (e.g., Schwann cell), a central nervous system neuron, a glial cell (e.g., astrocyte, oligodendrocyte), a lens cell, an adipocyte, a kidney cell, a barrier function cell (e.g., a duct cell), an extracellular matrix cell, a contractile cell (e.g., skeletal muscle cell, heart muscle cell, smooth muscle cell), a blood cell (e.g., erythrocyte), an immune system cell (e.g., megakaryocyte, microglial cell, neutrophil, mast cell, a T cell, a B cell, a Natural Killer cell), a germ cell (e.g., spermatid), a nurse cell, or an interstitial cell. In some embodiments, any of the stem cells described herein are differentiated into NK cells. In some embodiments, any of the derivative cell types described herein are differentiated into NK cells.

In some embodiments, engineered stem cells disclosed herein can be differentiated into definitive endoderm, hepatoblasts, or hepatocytes, wherein said cells have one, two, three, four, five, six or more of the following edits: SERPINB9 KI, B2M null, CIITA null, HLA-E KI, IL15/IL15Rα fusion protein KI, CAR KI, XIAP KI, CISH KO, FAS KO, CD16 KI, CD64 KI, ADAM17 KO, REGNASE-1 KO, TIGIT KO, PD-1 KO, NKG2A KO, CD70 KO, ALK4, type I activin receptor KO (e.g., a conditional KO), SOCS3 KO, tissue factor KO, and CD39 KI. In some embodiments, engineered stem cells disclosed herein can be differentiated into definitive endoderm, hepatoblasts, or hepatocytes, wherein said cells have one, two, three, four, five, six or all of the following edits: SERPINB9 KI, B2M null, CIITA null, HLA-E KI, IL15/IL15Rα fusion protein KI, CAR KI, XIAP KI, CISH KO, and/or FAS KO.

In some embodiments, the population of stem cells is a population of engineered cells, such as the engineered cells generated by the methods disclosed herein. In some embodiments, the population of engineered cells is differentiated by methods known in the art of generating natural killer (NK) cells. In some embodiments, the population of engineered cells is differentiated by methods known in the art of generating hepatoblasts or hepatocytes.

### Natural Killer Cells

Natural killer (NK) cells are a subpopulation of lymphocytes which play a critical role in the innate immune system. NK cells have cytotoxicity against a variety of cells including but not limited to tumor cells and virus-infected cells. In some embodiments, the stem cells described herein are differentiated to Natural Killer cells. In some embodiments, iPSCs are differentiated into NK cells. In some embodiments, the engineered NK cells (such as cells derived from gene-edited iPSCs by differentiation, i.e., iNK cells) have enhanced anti-tumor activity as compared to un-edited or wild type NK cells. In some embodiments, anti-tumor activity of the engineered NK cells is increased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, or at least 90% relative to control (e.g., un-edited or wild type) NK cells.

In some embodiments, the engineered NK cells exhibit increased cellular lysis capability relative to control cells. In some embodiments, the engineered NK cells of the present disclosure exhibit at least 10% increase in cellular lysis capability (kill at least 10% more target cells), or at least 20% increase in cellular lysis capability (kill at least 20% more target cells), relative to control (e.g., un-edited or wild type) cells. For example, the engineered NK cells of the present disclosure may exhibit an at least at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, or at least 90% increase in cellular lysis capability, relative to control (e.g., un-edited or wild type) cells. In some embodiments, the engineered NK cells of the present disclosure exhibit a 20%-100%, 20%-90%, 20%-80%, 20%-70%, 20%-60%, 20%-50%, 30%-100%, 30%-90%, 30%-80%, 30%-70%, 30%-60%, 30%-50%, 40%-100%, 40%-90%, 40%-80%, 40%-70%, 40%-60%, 40%-50%, 50%-100%, 50%-90%, 50%-80%, 50%-70%, or 50%-60% increase in cellular lysis capability, relative to control (e.g., un-edited or wild type) cells. In some embodiments, the target cells are T cells. In some embodiments, the target cells are cancer cells. In some embodiments, the target cells are leukemia cells. In some embodiments, this increase in cellular lysis capability is observed at E:T (effector:target cell) ratio of at or about 0.1:1. In some embodiments, this increase in cellular lysis capability is observed at E:T (effector:target cell) ratio of at or about 0.5:1. In some embodiments, this increase in cellular lysis capability is observed at E:T (effector:target cell) ratio of at or about 1:1. n some embodiments, this increase in cellular lysis capability is observed at E:T (effector:target cell) ratio of at or about 0.1:1, when the target cell is K562 and when the cells are co-cultured for, e.g., 24 hours. In some embodiments, this increase in cellular lysis capability is observed at E:T (effector:target cell) ratio of at or about 0.5:1, when the target cell is K562 and when the cells are co-cultured for, e.g., 24 hours. In some embodiments, this increase in cellular lysis capability is observed at E:T (effector:target cell) ratio of at or about 1:1, when the target cell is K562 and when the cells are co-cultured for, e.g., 24 hours. In some embodiments, this increase in cellular lysis capability is observed at E:T (effector:target cell) ratio of at or about 0.1:1, when the target cell is RPMI and when the cells are co-cultured for, e.g., 24 hours. In some embodiments, this increase in cellular lysis capability is observed at E:T (effector:target cell) ratio of at or about 0.5:1, when the target cell is RPMI and when the cells are co-cultured for, e.g., 24 hours. In some embodiments, this increase in cellular lysis capability is observed at E:T (effector:target cell) ratio of at or about 1:1, when the target cell is RPMI and when the cells are co-cultured for, e.g., 24 hours.

In some embodiments, the engineered NK cells express at least one, two, three, four, five, six, seven, eight or all of the following markers: CD45, CD56, CD94, NKG2A, CD16, NKp44, NKp46, KIR2DL4, and KIR3DL2, and optionally wherein the markers are expressed at least at 25%, 30%, 40%, 50%, 75%, 80%, 90%, 95% or 100% level or more relative to their expression in un-edited or wild type NK cells. In some embodiments, the engineered NK cells expresses at least one, two, three, four, five or all of the following markers: CD56, NKp44, NKp46, CD94, NKG2A and KIR2DL4, and optionally wherein the markers are expressed at least at 25%, 30%, 40%, 50%, 75%, 80%, 90%, 95% or 100% level or more relative to their expression in un-edited or wild type NK cells. In some embodiments, the engineered NK cells have at least 25%, at least 30%, at least 40%, at least 50%, at least 60%, at least 70%, at least 80%, at least 90%, at least 95% or at least 99% of the cell population expressing one, two, three, four, five, six, seven, eight or all of the following markers: CD45, CD56, CD94, NKG2A, CD16, NKp44, NKp46, KIR2DL4, and KIR3DL2. In some embodiments, the engineered NK cells have at least 25%, at least 30%, at least 40%, at least 50%, at least 60%, at least 70%, at least 80%, at least 90%, at least 95% or at least 99% of the cell population expressing one, two, three, four, five or all of the following markers: CD56, NKp44, NKp46, CD94, NKG2A and KIR2DL4.

In some embodiments, the engineered NK cells express at least one, two, three or all of the following markers: CD38, CD96, DNAM-1, and ICAM-1, and optionally wherein the markers are expressed at least at 25%, 30%, 40%, 50%, 75%, 80%, 90%, 95% or 100% level or more relative to their expression in un-edited or wild type NK cells. In some embodiments, the engineered NK cells express at least one, two, three or all of the following markers: CD38, CD96, DNAM-1, and ICAM-1, and optionally wherein the markers are expressed at least at 25%, 30%, 40%, 50%, 75%, 80%, 90%, 95% or 100% level or more relative to their expression in un-edited or wild type NK cells. In some embodiments, the engineered NK cells have at least 25%, at least 30%, at least 40%, at least 50%, at least 60%, at least 70%, at least 80%, at least 90%, at least 95% or at least 99% of the cell population expressing one, two, three or all of the following markers: CD38, CD96, DNAM-1, and ICAM-1. In some embodiments, the engineered NK cells have at least 25%, at least 30%, at least 40%, at least 50%, at least 60%, at least 70%, at least 80%, at least 90%, at least 95% or at least 99% of the cell population expressing one, two, three or all of the following markers: CD38, CD96, DNAM-1, and ICAM-1.

In some embodiments, the engineered NK cells express at least one, two, three or all of the following markers: NKG2D, TIM3, CD16, and CD25, and optionally wherein the markers are expressed at least at 25%, 30%, 40%, 50%, 75%, 80%, 90%, 95% or 100% level or more relative to their expression in un-edited or wild type NK cells. In some embodiments, the engineered NK cells express at least one, two, three or all of the following markers: NKG2D, TIM3, CD16, and CD25, and optionally wherein the markers are expressed at least at 25%, 30%, 40%, 50%, 75%, 80%, 90%, 95% or 100% level or more relative to their expression in un-edited or wild type NK cells. In some embodiments, the engineered NK cells have at least 10%, at least 15%, at least 20%, at least 25%, at least 30%, at least 40%, at least 50%, at least 60%, at least 70%, at least 80%, at least 90%, at least 95% or at least 99% of the cell population expressing one, two, three or all of the following markers: NKG2D, TIM3, CD16, and CD25. In some embodiments, the engineered NK cells have at least 10%, at least 15%, at least 20%, at least 25%, at least 30%, at least 40%, at least 50%, at least 60%, at least 70%, at least 80%, at least 90%, at least 95% or at least 99% of the cell population expressing one, two, three or all of the following markers: NKG2D, TIM3, CD16, and CD25.

In some embodiments, the engineered NK cells of the present disclosure exhibit an increased cytokine secretion relative to control (e.g., un-edited or wild type) cells. In some embodiments, the engineered NK cells of the present disclosure exhibit about the same cytokine secretion level relative to control (e.g., un-edited or wild type) cells. In some embodiments, the engineered NK cells of the present disclosure exhibit a reduced (e.g., reduced by less than 10%, less than 20%, less than 30%, less than 40%, or less than 50%) cytokine secretion level relative to control (e.g., un-edited or wild type) cells. In some embodiments, the engineered NK cells of the present disclosure exhibit a reduced (e.g., reduced by more than 20%, more than 30%, more than 40%, more than 50%, or more than 75%) cytokine secretion level relative to control (e.g., un-edited or wild type) cells. In some embodiments, the engineered NK cells of the present disclosure exhibit an increased (e.g., increased by at least 5%, at least 10%, at least 20%, at least 30%, at least 40%, at least 50%, or at least 75%) cytokine secretion level relative to control (e.g., un-edited or wild type) cells. The cytokine(s) being measured can be, without limitation any one or more of: TNFα, IFNγ and IL-7. In some embodiments, the level of cytokines (e.g., TNFα, IFNγ and IL-7) secreted by the engineered NK cells is about the same as the level in control (e.g., un-edited or wild type) cells, when cells are co-cultured with target cells at the E:T ratio of or about 0.1:1. In some embodiments, the level of cytokines (e.g., TNFα, IFNγ and IL-7) secreted by the engineered NK cells is reduced (by, e.g., at least 10%, 20%, 30%, 40%, 50%, 60% or 70%, and/or no more than 50%, 60%, 70%, 80%, or 90%) relative to the level in control (e.g., un-edited or wild type) cells, when cells are co-cultured with target cells at the E:T ratio of or about 0.1:1. In some embodiments, the level of cytokines (e.g., TNFα, IFNγ and IL-7) secreted by the engineered NK cells is increased (by, e.g., at least 5%, 10%, 20%, 30%, 40%, 50%, 60% or 70%) relative to the level in control (e.g., un-edited or wild type) cells, when cells are co-cultured with target cells at the E:T ratio of or about 0.1:1.

In some embodiments, the engineered NK cells of the present disclosure exhibit an increased expression or release of Granzyme B or perforin relative to control (e.g., un-edited or wild type) cells. In some embodiments, the engineered NK cells of the present disclosure exhibit about the same expression or release level of Granzyme B or perforin relative to control (e.g., un-edited or wild type) cells. In some embodiments, the engineered NK cells of the present disclosure exhibit a reduced (e.g., reduced by less than 10%, less than 20%, less than 30%, less than 40%, or less than 50%) Granzyme B or perforin expression or release level relative to control (e.g., un-edited or wild type) cells. In some embodiments, the engineered NK cells of the present disclosure exhibit a reduced (e.g., reduced by more than 20%, more than 30%, more than 40%, more than 50%, or more than 75%) Granzyme B or perforin expression or release level relative to control (e.g., un-edited or wild type) cells. In some embodiments, the engineered NK cells of the present disclosure exhibit an increased (e.g., increased by at least 5%, at least 10%, at least 20%, at least 30%, at least 40%, at least 50%, or at least 75%) Granzyme B or perforin expression or release level relative to control (e.g., un-edited or wild type) cells. In some embodiments, the level of Granzyme B or perforin secreted by the engineered NK cells is about the same as the level in control (e.g., un-edited or wild type) cells, when cells are co-cultured with target cells at the E:T ratio of or about 0.1:1. In some embodiments, the level of Granzyme B or perforin secreted by the engineered NK cells is reduced (by, e.g., at least 10%, 20%, 30%, 40%, 50%, 60% or 70%, and/or no more than 50%, 60%, 70%, 80%, or 90%) relative to the level in control (e.g., un-edited or wild type) cells, when cells are co-cultured with target cells at the E:T ratio of or about 0.1:1. In some embodiments, the level of Granzyme B or perforin secreted by the engineered NK cells is increased (by, e.g., at least 5%, 10%, 20%, 30%, 40%, 50%, 60% or 70%) relative to the level in control (e.g., un-edited or wild type) cells, when cells are co-cultured with target cells at the E:T ratio of or about 0.1:1.

In some embodiments, the engineered NK cells of the present disclosure exhibit an increased (e.g., increased by at least 5%, at least 10%, at least 20%, at least 30%, at least 40%, at least 50%, or at least 75%) expression level of CD107a relative to control (e.g., un-edited or wild type) cells. In some embodiments, the engineered NK cells of the present disclosure exhibit about the same expression level of CD107a relative to control (e.g., un-edited or wild type) cells. In some embodiments, engineered NK cells of the present disclosure exhibit a reduced (e.g., reduced by less than 10%, less than 20%, less than 30%, less than 40%, or less than 50%) CD107a expression level relative to control (e.g., un-edited or wild type) cells. In some embodiments, the engineered NK cells of the present disclosure exhibit a reduced (e.g., reduced by more than 20%, more than 30%, more than 40%, more than 50%, or more than 75%) CD107a expression level relative to control (e.g., un-edited or wild type) cells.

In some embodiments, the engineered NK cells have higher proliferative capacity as compared to un-edited or wild-type NK cells. In some embodiments, the engineered NK cells have approximately the same proliferative capacity compared to un-edited or wild-type NK cells.

In some embodiments, the engineered NK cells do not exhibit exhaustion or exhibit a low level of exhaustion (e.g., a level of exhaustion markers associated with a functional NK cell). In some embodiments, exhaustion is detected by detecting a reduced expression of IFNγ, granzyme B, perforin, CD107a, and/or TNFα in cells. In some embodiments, exhaustion is detected by detecting increased expression (e.g., on the surface of the cell) of an exhaustion marker, e.g., PD-1, LAG-3, TIGIT and/or TIM-3. In some embodiments, the engineered NK cells have normal or higher than normal expression of perforin, granzyme B, CD107a, IFNγ and/or TNFα (relative to un-edited or wild-type cells). In some embodiments, the engineered NK cells have lower than normal or no expression of PD-1, LAG-3, TIGIT and/or TIM-3 (relative to un-edited or wild-type cells). In some embodiments, engineered NK cells of the present disclosure exhibit reduced exhaustion, relative to control (e.g., un-edited cells or wild type) NK cells.

In some embodiments, the engineered NK cells of the present disclosure exhibit about the same cellular viability as control (e.g., un-edited or wild-type) cells. In some embodiments, the engineered NK cells of the present disclosure exhibit increased cellular viability relative to control (e.g., un-edited or wild-type) cells. In some embodiments, the engineered NK cells of the present disclosure exhibit at least 10% or at least 20% increase in cellular viability, relative to control cells. For example, the engineered NK cells of the present disclosure may exhibit at least 25%, at least 30%, at least 35%, at least 40%, at least 45%, at least 50%, at least 55%, at least 60%, at least 65%, at least 70%, at least 75%, at least 80%, or at least 90% increase in cellular viability, relative to control cells. In some embodiments, the engineered NK cells of the present disclosure exhibit a 20%-100%, 20%-90%, 20%-80%, 20%-70%, 20%-60%, 20%-50%, 30%-100%, 30%-90%, 30%-80%, 30%-70%, 30%-60%, 30%-50%, 40%-100%, 40%-90%, 40%-80%, 40%-70%, 40%-60%, 40%-50%, 50%-100%, 50%-90%, 50%-80%, 50%-70%, or 50%-60% increase in cellular viability, relative to control cells. Methods of measuring cell viability are known to those of skill in the art and described herein.

In some embodiments, the engineered NK cells have higher expression of one or more cell cycle genes, one or more cell division genes, and/or one or more DNA replication genes, as compared to un-edited or wild type NK cells. In some embodiments, the engineered NK cells have approximately the same expression of one or more cell cycle genes, one or more cell division genes, and/or one or more DNA replication genes, as compared to un-edited or wild type NK cells.

In some embodiments, any of the engineered NK cells described herein have one of more of the following characteristics relative to an un-edited (wild-type) NK cell described herein: increased persistence, increased immune evasiveness, lack of an alloimmune T cell response, increased cytotoxic activity, improved antibody-dependent cellular cytotoxicity (ADCC), or increased anti-tumor activity.

In some embodiments, the engineered hepatoblasts express at least one two, or all of the following markers: AFP, ALB, and/or HNF-4a, and optionally wherein the markers are expressed at least at 25%, 30%, 40%, 50%, 75%, 80%, 90%, 95% or 100% level or more relative to their expression in un-edited or wild type hepatoblasts. In some embodiments, the engineered hepatoblasts have at least 10%, at least 15%, at least 20%, at least 25%, at least 30%, at least 40%, at least 50%, at least 60%, at least 70%, at least 80%, at least 90%, at least 95% or at least 99% of the cell population expressing one, two, three or all of the following markers: AFP, ALB, and/or HNF-4a.

In some embodiments, the engineered hepatocytes express at least one, two, three or all of the following markers: ALB, G6PC, CPS1, ABCC2, UGT2B4, CYP1A2, and/or CYP3A4, and optionally wherein the markers are expressed at least at 25%, 30%, 40%, 50%, 75%, 80%, 90%, 95% or 100% level or more relative to their expression in un-edited or wild type hepatocytes. In some embodiments, the engineered hepatocytes cells have at least 10%, at least 15%, at least 20%, at least 25%, at least 30%, at least 40%, at least 50%, at least 60%, at least 70%, at least 80%, at least 90%, at least 95% or at least 99% of the cell population expressing one, two, three or all of the following markers: ALB, G6PC, CPS1, ABCC2, UGT2B4, CYP1A2, and/or CYP3A4.

In some embodiments, the population of engineered cells of the present disclosure is engineered (e.g., by use of CRISPR-Cas9 gene-editing) to induce a site-specific disruption in a target gene sequence that eliminates the expression of an allogeneic antigen. In some embodiments, an allogeneic antigen is a major histocompatibility antigen. In some embodiments, a major histocompatibility antigen is a MHC I complex. In some embodiments, the target gene sequence is found in the B2M gene that encodes a protein component of the MHC I complex.

In some embodiments, persistence of the engineered cells is assessed by analyzing their presence and quantity in one or more tissue samples that are collected from a subject following administration of the engineered cells to the subject. In some embodiments, persistence is defined as the longest duration of time from administration to a time wherein a detectable level of the engineered cells is present in a given tissue type (e.g., peripheral blood). In some embodiments, persistence is defined as the continued absence of disease (e.g., complete response or partial response). Determination of the absence of disease and response to treatment are known to those of skill in the art and described herein.

Methods of appropriate tissue collection, preparation, and storage are known to one skilled in the art. In some embodiments, persistence of cells is assessed in one or more tissue samples from a group comprised of peripheral blood, cerebrospinal fluid, tumor, skin, bone, bone marrow, breast, kidney, liver, lung, lymph node, spleen, gastrointestinal tract, tonsils, thymus and prostate. In some embodiments, a quantity of cells is measured in a single type of tissue sample (e.g., peripheral blood). In some embodiments, a quantity of cells is measured in multiple tissue types (e.g., peripheral blood in addition to bone marrow and cerebrospinal fluid). By measuring quantity of cells in multiple tissue types, the distribution of cells throughout different tissues of the body can be determined. In some embodiments, a quantity of cells is measured in one or more tissue samples at a single time point following administration. In some embodiments, a quantity of cells is measured in one or more tissue samples at multiple time points following administration.

A detectable level of the engineered cells in a given tissue can be measured by known methodologies. Methods for assessing the presence or quantity of cells in a tissue of interest are known to those of skill in the art. Such methods include, but are not limited to, reverse transcription polymerase chain reaction (RT-PCR), competitive RT-PCR, real-time RT-PCR, droplet digital PCR (ddPCR), RNase protection assay (RPA), quantitative immunofluorescence (QIF), flow cytometry, northern blotting, nucleic acid microarray using DNA, western blotting, enzyme-linked immunosorbent assay (ELISA), radioimmunoassay (RIA), tissue immunostaining, immunoprecipitation assay, complement fixation assay, fluorescence-activated cell sorting (FACS), mass spectrometry, magnetic bead-antibody immunoprecipitation, or protein chip.

As used herein, in some embodiments, persistence is the longest period from the time of administration to a time wherein a detectable level of the engineered cells is measured. In some embodiments, a detectable level of cells is defined in terms of the limit of detection of a method of analysis. The limit of detection can be defined as the lowest quantity of a component or substance that can be reliably and reproducibly measured by an analytical procedure when compared to a tissue sample expected to have no quantity of the component or substance of interest. A non-limiting exemplary method to determine a reproducible limit of detection is to measure the analytical signal for replicates of a zero calibrator relative to a blank sample (Armbruster, D. et al. (2008) Clin Biochem Rev. 29:S49-S52). A blank sample is known to be devoid of an analyte of interest. A zero calibrator is the highest dilution of a test sample of known concentration or quantity that gives analytical signal above that measured for the blank sample. By quantifying the analytical signal for at least 5, 6, 7, 8, 9, 10, 11, 12, 13, 14, 15, 16, 17, 18, 19, 20, 21, 22, 23, 24, 25, 26, 27, 28, 29, or 30 replicates of a zero calibrator, one can determine an average and standard deviation (SD) for the limit of detection of an analytical method of interest. Selection of a method with a suitable limit of detection for quantifying donor T cells in a given tissue can be ascertained by one skilled in the art. In some embodiments, a detectable level of cells is any quantity of cells in a tissue sample that gives an analytical signal above the limit of detection for a method of analysis. In some embodiments, a detectable level of cells is any quantity of cells in a tissue sample that gives an analytical signal that is at least 2 SDs, 3 SDs, 4 SDs, 5 SDs, 6 SDs, 7 SDs, 8 SDs, 9 SDs, or 10 SDs, above the limit of detection for the method of analysis.

Differentiated and/or engineered cells can undergo expansion following administration to a recipient. Expansion is a response to antigen recognition and signal activation. In some embodiments, following expansion, engineered cells can undergo a contraction period, wherein a portion of the cell population that are short-lived effector cells are eliminated and what remains is a portion of the cell population that are long-lived memory cells. In some embodiments, persistence is a measure of the longevity of the engineered cell population following expansion and contraction. The duration of the expansion, contraction and persistence phases are evaluated using a pharmacokinetic profile. In some embodiments, a pharmacokinetic (PK) profile is a description of the cells measured in a given tissue over time and is readily ascertained by one skilled in the art by measuring the cells in a given tissue (e.g., peripheral blood) at multiple time points. In some embodiments, a measure of a PK profile provides a method of evaluating or monitoring the effectiveness of the engineered cell therapy in a subject (e.g., having cancer). In some embodiments, a measure of a PK profile provides a method of evaluating the persistence of the engineered cells in a subject. In some embodiments, a PK profile provides a method of evaluating the expansion of the engineered cells in a subject. In some embodiments, a measure of persistence of engineered cells in a subject is used to evaluate the effectiveness of engineered cell therapy in a subject. In some embodiments, a measure of expansion of engineered cells in a subject is used to evaluate the effectiveness of engineered cell therapy in a subject.

In some embodiments, a PK profile is prepared by measuring a quantity of engineered cells in a sample of a given tissue type (e.g., peripheral blood) collected from a recipient and repeating the assessment at different time points. In some embodiments, a baseline tissue sample is collected from a recipient no more than 1 day, 2 days, 3 days, 4 days, 5 days, 6 days, 7 days, 8 days, 9 days, 10 days, 12 days, 13 days, 14 days, or 15 days prior to administration. In some embodiments, tissue collection from a recipient is performed within 0.25-2 hours, within 1-3 hours, within 2-6 hours, within 3-11 hours, within 4-20 hours, within 5-48 hours of the time of administration of engineered cells. In some embodiments, tissue collection from a recipient is performed on a daily basis starting on day 1, day 2, day 3, or day 4 and continuing through at least day 5, day 6, day 7, day 8, day 9, day 10, day 11, day 12, day 13, day 14, day 15, day 16, day 17, day 18, day 19, or day 20. In some embodiments, tissue collection from a recipient is performed at least 1 time, 2 times, 3 times, 4 times, 5 times, or 6 times per week for up to 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, or 16 weeks following administration of cells. In some embodiments, tissue collection from a recipient is performed at least 1 time, 2 times, 3 times, 4 times, 5 times, or 6 times per month for up to 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, or 24 months following administration of cells. In some embodiments, tissue collection from a recipient is performed at least 1 time, 2 times, 3 times, 4 times, 5 times, or 6 times per year for up to 1 year, 2 years, 3 years, 4 years, 5 years, 6 year, 7 years, 8 years, 9 years, or 10 years following administration of cells.

In some embodiments, engineered cell persistence is defined as the duration of time from administration wherein a quantity of engineered cells is present that is at least 0.005-0.05%, 0.01-0.1%, 0.05-0.5%, 0.1-1%, 0.5%-5%, 1-10%, 5%-10%, or 10%-15% (e.g., at least 1%, 5%, 10%, or 15%) of the peak quantity of engineered cells. In some embodiments, a persistence of cells is determined by comparing the quantity of cells measured in a given tissue type (e.g., peripheral blood) to the peak quantity of cells that is measured in the same tissue type. In some embodiments, a persistence of cells is determined by comparing the quantity of cells measured in a given subject (e.g., peripheral blood) to the peak quantity of cells that is measured in the same subject. In some embodiments, a persistence of cells is determined by comparing the quantity of cells measured in a given subject (e.g., peripheral blood) to the peak quantity of cells that is measured in a different subject (i.e., a subject with partial response, a subject with complete response).

In some embodiments, a persistence of engineered cells is present in one or more tissue types following administration wherein engineered cells are administered on day 1. In some embodiments, a persistence of engineered cells is present in one or more tissue types up to 1 day, 2 days, 3 days, 4, days, 5 days, 6 days, 7 days, 8 days, 9 days, 10 days, 11 days, 12 days, 13 days, 14 days, 15 days, 16 days, 17 days, 18 days, 19 days, 21 days, 21 days, 22 days, 23 days, 24 days, 25 days, 26 days, 27 days, 28 days, 29 days, 30 days, 31 days, 32 days, 33 days, 34 days, or 35 days following administration wherein engineered cells are administered on day 1. In some embodiments, a persistence of engineered cells is present in one or more tissue types up to 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 21 months, 22 months, 23 months, or 24 months following administration of engineered cells). In some embodiments, a persistence of engineered cells is measured in one or more tissue types up to 1 year, 2 years, 3 years, 4 years, 5 years, 6 years, 7 years, 8 years, 9 years, and 10 years following administration of engineered cells. In some embodiments, a persistence of engineered cells that is at least 10-25 days, at least 25-50 days, at least 50-100 days, at least 100-364 days, at least one year, at least two years, at least three years, at least four years or at least five years from administration wherein engineered cells are administered on day 1 is indicative of a response in a recipient (e.g. complete response or partial response).

### Isolation and Purification of Cells

### Purification

In some embodiments, the population of gene-edited cells (e.g., iPSC, iNK or NK cells, hepatoblasts and/or hepatocytes) described herein are activated and/or expanded before or after genome editing. In some embodiments, iPSC cells are differentiated after gene-editing. In some embodiments, iNK cells or heapatoblasts/hepatocytes are activated and expanded for about 1 day to about 4 days, about 1 day to about 3 days, about 1 day to about 2 days, about 2 days to about 3 days, about 2 days to about 4 days, about 3 days to about 4 days, or about 1 day, about 2 days, about 3 days, or about 4 days prior to genome editing.

In some embodiments, the disclosure provides a method for substantially isolating cells that express a detectable level of a protein (e.g., HLA-E) from a population of cells comprising engineered cells (e.g., SERPINB9 KI, IL15/IL15Rα fusion protein KI, HLA-E KI, B2M null, CIITA null, CAR KI, XIAP KI, CISH null, and/or FAS null cells).

In some embodiments, the disclosure provides a method for isolating a population of cells comprising engineered cells (e.g., SERPINB9 KI, IL15/IL15Rα fusion protein KI, HLA-E KI, B2M null, CIITA null, CAR KI, XIAP KI, CISH null, and/or FAS null cells), comprising: providing the population of cells wherein the engineered cells comprise a disrupted CIITA gene and a disrupted B2M gene; and isolating the population of cells expressing SERPINB9 (e.g., such that >99% of the population comprises the SERPINB9 expressing cells).

Removal of a subset of cells from a population can be performed using conventional cell purification methods. Non-limiting examples of cell sorting methods include fluorescence-activated cell sorting, immunomagnetic separation, chromatography, and microfluidic cell sorting. In some embodiments, SERPINB9 expressing cells are removed from a population of engineered cells by immunomagnetic separation. In some embodiments, HLA-E-expressing cells are removed from a population of engineered cells by immunomagnetic separation.

In some embodiments, genome edited cells are sorted into single cells. In some embodiments, single cell isolates of gene-edited cells are grown into single cell clonal populations. In some embodiments, multiple single-cell clones are generated. In some embodiments, an edited clone is expanded to generate a master cell bank (MCB).

### Formulations and Administrations

### Formulation and Delivery for Gene Editing

Guide RNAs, polynucleotides, e.g., polynucleotides that encode any protein described herein or polynucleotides that encode an endonuclease, and endonucleases as described herein may be formulated and delivered to cells in any manner known in the art.

Guide RNAs and/or polynucleotides may be formulated with pharmaceutically acceptable excipients such as carriers, solvents, stabilizers, adjuvants, diluents, etc., depending upon the particular mode of administration and dosage form. Guide RNAs and/or polynucleotides compositions can be formulated to achieve a physiologically compatible pH, and range from a pH of about 3 to a pH of about 11, about pH 3 to about pH 7, depending on the formulation and route of administration. In some cases, the pH can be adjusted to a range from about pH 5.0 to about pH 8. In some cases, the compositions can comprise a therapeutically effective amount of at least one compound as described herein, together with one or more pharmaceutically acceptable excipients. Optionally, the compositions can comprise a combination of the compounds described herein, or can include a second active ingredient useful in the treatment or prevention of bacterial growth (for example and without limitation, anti-bacterial or anti-microbial agents), or can include a combination of reagents of the present disclosure.

Suitable excipients include, for example, carrier molecules that include large, slowly metabolized macromolecules such as proteins, polysaccharides, polylactic acids, polyglycolic acids, polymeric amino acids, amino acid copolymers, and inactive virus particles. Other exemplary excipients can include antioxidants (for example and without limitation, ascorbic acid), chelating agents (for example and without limitation, EDTA), carbohydrates (for example and without limitation, dextrin, hydroxyalkylcellulose, and hydroxyalkylmethylcellulose), stearic acid, liquids (for example and without limitation, oils, water, saline, glycerol and ethanol), wetting or emulsifying agents, pH buffering substances, and the like.

Guide RNA polynucleotides (RNA or DNA) and/or endonuclease polynucleotide(s) (RNA or DNA) can be delivered by viral or non-viral delivery vehicles known in the art. Alternatively, endonuclease polypeptide(s) can be delivered by viral or non-viral delivery vehicles known in the art, such as electroporation or lipid nanoparticles. In further alternative aspects, the DNA endonuclease can be delivered as one or more polypeptides, either alone or pre-complexed with one or more guide RNAs, or one or more crRNA together with a tracrRNA.

Polynucleotides can be delivered by non-viral delivery vehicles including, but not limited to, nanoparticles, liposomes, ribonucleoproteins, positively charged peptides, small molecule RNA-conjugates, aptamer-RNA chimeras, and RNA-fusion protein complexes. Some exemplary non-viral delivery vehicles are described in Peer and Lieberman, Gene Therapy, 2011, 18: 1127-1133 (which focuses on non-viral delivery vehicles for siRNA that are also useful for delivery of other polynucleotides).

For polynucleotides of the disclosure, the formulation may be selected from any of those taught, for example, in International Application PCT/US2012/069610.

Polynucleotides, such as guide RNA, sgRNA, and mRNA encoding an endonuclease, may be delivered to a cell or a subject by a lipid nanoparticle (LNP).

A LNP refers to any particle having a diameter of less than 1000 nm, 500 nm, 250 nm, 200 nm, 150 nm, 100 nm, 75 nm, 50 nm, or 25 nm. Alternatively, a nanoparticle may range in size from 1-1000 nm, 1-500 nm, 1-250 nm, 25-200 nm, 25-100 nm, 35-75 nm, or 25-60 nm.

LNPs may be made from cationic, anionic, or neutral lipids. Neutral lipids, such as the fusogenic phospholipid DOPE or the membrane component cholesterol, may be included in LNPs as ‘helper lipids’ to enhance transfection activity and nanoparticle stability. Limitations of cationic lipids include low efficacy owing to poor stability and rapid clearance, as well as the generation of inflammatory or anti-inflammatory responses.

LNPs may also be comprised of hydrophobic lipids, hydrophilic lipids, or both hydrophobic and hydrophilic lipids.

Any lipid or combination of lipids that are known in the art can be used to produce a LNP. Examples of lipids used to produce LNPs are: DOTMA, DOSPA, DOTAP, DMRIE, DC-cholesterol, DOTAP-cholesterol, GAP-DMORIE-DPyPE, and GL67A-DOPE-DMPE-polyethylene glycol (PEG). Examples of cationic lipids are: 98N12-5, C12-200, DLin-KC2-DMA (KC2), DLin-MC3-DMA (MC3), XTC, MID1, and 7C1. Examples of neutral lipids are: DPSC, DPPC, POPC, DOPE, and SM. Examples of PEG-modified lipids are: PEG-DMG, PEG-CerC14, and PEG-CerC20.

The lipids can be combined in any number of molar ratios to produce a LNP. In addition, the polynucleotide(s) can be combined with lipid(s) in a wide range of molar ratios to produce a LNP.

A recombinant adeno-associated virus (AAV) vector can be used for delivery. Techniques to produce rAAV particles, in which an AAV genome to be packaged that includes the polynucleotide to be delivered, rep and cap genes, and helper virus functions are provided to a cell are standard in the art. Production of rAAV typically requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived, and may be from a different AAV serotype than the rAAV genome ITRs, including, but not limited to, AAV serotypes described herein. Production of pseudotyped rAAV is disclosed in, for example, international patent application publication number WO 01/83692.

### Formulation and Administration of Cells

Genetically modified cells, as described herein may be formulated and administered to a subject by any manner known in the art.

The terms “administering,” “introducing”, “implanting”, “engrafting” and “transplanting” are used interchangeably in the context of the placement of cells, e.g., progenitor cells, into a subject, by a method or route that results in at least partial localization of the introduced cells at a desired site. The cells e.g., progenitor cells, or their differentiated progeny can be administered by any appropriate route that results in delivery to a desired location in the subject where at least a portion of the implanted cells or components of the cells remain viable. The period of viability of the cells after administration to a subject can be as short as a few hours, e.g., twenty-four hours, to a few days, to as long as several years, or even the life-time of the subject, i.e., long-term engraftment.

In some embodiments, a genetically modified cell as described herein is viable after administration to a subject for a period that is longer than that of an unmodified cell.

In some embodiments, a composition comprising cells as described herein are administered by a suitable route, which may include intravenous administration, e.g., as a bolus or by continuous infusion over a period of time. In some embodiments, intravenous administration may be performed by intramuscular, intraperitoneal, intracerebrospinal, subcutaneous, intra-articular, intrasynovial, or intrathecal routes. In some embodiments, a composition may be in solid form, aqueous form, or a liquid form. In some embodiments, an aqueous or liquid form may be nebulized or lyophilized. In some embodiments, a nebulized or lyophilized form may be reconstituted with an aqueous or liquid solution.

A cell composition can also be emulsified or presented as a liposome composition, provided that the emulsification procedure does not adversely affect cell viability. The cells and any other active ingredient can be mixed with excipients that are pharmaceutically acceptable and compatible with the active ingredient, and in amounts suitable for use in the therapeutic methods described herein.

Additional agents included in a cell composition can include pharmaceutically acceptable salts of the components therein. Pharmaceutically acceptable salts include the acid addition salts (formed with the free amino groups of the polypeptide) that are formed with inorganic acids, such as, for example, hydrochloric or phosphoric acids, or such organic acids as acetic, tartaric, mandelic and the like. Salts formed with the free carboxyl groups can also be derived from inorganic bases, such as, for example, sodium, potassium, ammonium, calcium or ferric hydroxides, and such organic bases as isopropylamine, trimethylamine, 2-ethylamino ethanol, histidine, procaine and the like.

Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active compound used in the cell compositions that is effective in the treatment of a particular disorder or condition can depend on the nature of the disorder or condition, and can be determined by standard clinical techniques.

In some embodiments, a composition comprising cells may be administered to a subject, e.g., a human subject, who has, is suspected of having, or is at risk for a disease. In some embodiments, a composition may be administered to a subject who does not have, is not suspected of having or is not at risk for a disease. In some embodiments, a subject is a healthy human. In some embodiments, a subject e.g., a human subject, who has, is suspected of having, or is at risk for a genetically inheritable disease. In some embodiments, the subject is suffering or is at risk of developing symptoms indicative of a disease.

### Treatment Methods

Provided herein, in some embodiments, are methods for treating a subject in need thereof. In some embodiment, methods are provided for treating cancer (e.g., leukemias, e.g., acute myeloid leukemia) using any engineered cells described herein (or any population of cells described herein).

In some embodiments, the cancer being treated can be multiple myeloma, Hodgkin's lymphoma, lung cancer, leukemia, B-cell acute lymphoblastic leukemia (B-ALL), B-cell non-Hodgkin's lymphoma (B-NL), acute lymphoblastic leukemia (ALL), T cell lymphoma, T cell leukemia, clear cell renal cell carcinoma (ccRCC), thyroid cancer, nasopharyngeal cancer, non-small cell lung (NSCLC), pancreatic cancer, melanoma, ovarian cancer, glioblastoma, liver cancer, or cervical cancer. In some embodiments, provided herein is a method of treating cancer in a subject (e.g., human) in need thereof, comprising administering any engineered cell described herein to the subject (e.g., wherein the subject has or has been diagnosed with cancer). In some embodiments, the method is not a method for treatment of the human or animal body by therapy.

In some embodiments, provided herein is a method of treating Hodgkin's lymphoma, a non-Hodgkin lymphoma (e.g., diffuse large B-cell lymphoma (DLBCL), high grade B-cell lymphoma, transformed follicular lymphoma (FL), grade 3B FL, and Richter's transformation of CLL) in a subject (e.g., human) in need thereof, comprising administering any engineered cell described herein to the subject (e.g., wherein the subject has or has been diagnosed with a Hodgkin's lymphoma or non-Hodgkin lymphoma, or is at risk of a Hodgkin's lymphoma or non-Hodgkin lymphoma). In some embodiments, the subject (e.g., a human) has (e.g., has been diagnosed with) a relapsed and/or refractory Hodgkin lymphoma. In some embodiments, the subject (e.g., a human) has (e.g., has been diagnosed with) a non-relapsed or early stage Hodgkin lymphoma. In some embodiments, the subject (e.g., a human) has (e.g., has been diagnosed with) a relapsed and/or refractory non-Hodgkin lymphoma. In some embodiments, the subject (e.g., a human) has (e.g., has been diagnosed with) a non-relapsed or early stage non-Hodgkin lymphoma. In some embodiments, provided herein is a method of treating chronic lymphocytic leukemia (CLL) or acute lymphoblastic leukemia (ALL) in a subject (e.g., human) in need thereof, comprising administering any engineered cell described herein to the subject (e.g., wherein the subject has or has been diagnosed with CLL or ALL). In some embodiments, the subject (e.g., a human) has (e.g., has been diagnosed with) a relapsed and/or refractory CLL or ALL. In some embodiments, the subject (e.g., a human) has (e.g., has been diagnosed with) a non-relapsed or early stage CLL or ALL. The engineered cell can be administered at any dose described herein, in particular, in a therapeutically effective amount. In some embodiments, a human being treated is an adult, e.g., a human over 18 years of age. In some embodiments, a human being treated is under 18 years of age.

In some embodiment, methods are provided for treating hepatic diseases or disorders using any engineered cells described herein (or any population of cells described herein). Non-limiting examples of hepatic diseases or disorders that may be treated as provided herein include fatty liver disease, non-alcoholic fatty liver disease, autoimmune hepatitis, alcoholic hepatitis, viral hepatitis, ischemic hepatitis, metabolic disorder hepatitis, chronic liver inflammation, hepatic fibrosis, cholestasis, primary sclerosing cholangitis, cirrhosis, primary biliary cirrhosis, zonal necrosis, hemochromatosis, Wilson's disease, alpha 1-antitrypsin deficiency, glycogen storage disease type II, Gilbert's syndrome, portal hypertension, portal vein thrombosis, ascites, hepatic steatosis post-liver transplantation, acute or chronic liver transplant rejection and metabolic conditions. Methods for treating hepatic diseases or disorders comprise administering a population of hepatoblasts/hepatocytes differentiated from engineered stem cells (e.g., SERPINB9 KI, IL15/IL15Rα fusion protein KI, HLA-E KI, B2M null, CIITA null, CAR KI, XIAP KI, CISH null, and/or FAS null), wherein the differentiated cells maintain all the edits introduced in the engineered stem cells.

In some embodiments, the methods comprise delivering the engineered cells (e.g., SERPINB9 expressing, anti-BCMA CAR NK cells) of the present disclosure to a subject having a cancer (e.g., leukemia), wherein cancer cells express BCMA. In some embodiments, the methods comprise delivering the engineered cells (e.g., SERPINB9 expressing, anti-CD30 CAR NK cells) of the present disclosure to a subject having a cancer (e.g., leukemia), wherein cancer cells express CD30. In some embodiments where the disease being treated is a Hodgkin's lymphoma, the cells used express a CD30 CAR (e.g., anti-CD30 CAR NK cells).

The step of administering may include the placement (e.g., transplantation) of cells, e.g., engineered NK cells or engineered hepatoblasts/hepatocytes, into a subject, by a method or route that results in at least partial localization of the introduced cells at a desired site, such as tumor, such that a desired effect(s) is produced. Engineered cells can be administered by any appropriate route that results in delivery to a desired location in the subject where at least a portion of the implanted cells or components of the cells remain viable. The period of viability of the cells after administration to a subject can be as short as a few hours, e.g., twenty-four hours, to a few days, to as long as several years, or even the life-time of the subject, i.e., long-term engraftment. For example, in some embodiments, an effective amount of engineered cell is administered via a systemic route of administration, such as an intraperitoneal or intravenous route.

A subject may be any subject for whom diagnosis, treatment, or therapy is desired. In some embodiments, the subject is a mammal. In some embodiments, the subject is a human.

In some embodiments, an engineered cell population being administered according to the methods described herein comprises gene edited hematopoietic cells (e.g., NK cells) differentiated from gene-edited stem cells (e.g., iPSC cells). In some embodiments, an engineered cell population being administered according to the methods described herein comprises gene edited hepatoblasts/hepatocytes differentiated from gene-edited stem cells (e.g., iPSC cells).

In some embodiments, an engineered cell population being administered according to the methods described herein does not induce toxicity in the subject, e.g., the engineered NK cells do not induce toxicity in non-cancer cells. In some embodiments, an engineered cell population (e.g., NK cells) being administered does not trigger complement mediated lysis, or does not stimulate antibody-dependent cell mediated cytotoxicity (ADCC).

In some embodiments, the subject being treated has no chronic immune suppression.

An effective amount refers to the amount of a population of engineered cells (needed to prevent or alleviate at least one or more signs or symptoms of a medical condition, and relates to a sufficient amount of a composition to provide the desired effect, e.g., to treat a subject having a medical condition. An effective amount also includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

In some embodiments, a subject is administered a population of cells comprising engineered cells (e.g., SERPINB9 KI, IL15/IL15Rα fusion protein KI, HLA-E KI, B2M null, CIITA null, CAR KI, XIAP KI, CISH null, and/or FAS null cells) at a dose in the range of about 1×10⁷ to 1×10⁹ engineered cells. In some embodiments, a subject is administered a population of cells comprising engineered cells (e.g., SERPINB9 KI, IL15/IL15Rα fusion protein KI, HLA-E KI, B2M null, CIITA null, CAR KI, XIAP KI, CISH null, and/or FAS null cells) at a dose in the range of about 1×10⁷ to 3×10⁸ engineered cells. In some embodiments, a subject is administered a population of cells comprising engineered cells (e.g., engineered SERPINB9 KI, IL15/IL15Rα fusion protein KI, HLA-E KI, B2M null, CIITA null, CAR KI, XIAP KI, CISH null, and/or FAS null cells) at a dose in the range of about 3×10⁷ to 3×10⁸ engineered cells.

In some embodiments, the cells are derived from iPSCs. In some embodiments, the cells are expanded in culture prior to administration to a subject in need thereof.

Modes of administration include but are not limited to injection and infusion. In some embodiments, injection includes, without limitation, intravenous, intrathecal, intraperitoneal, intraspinal, intracerebrospinal, and intrasternal infusion. In some embodiments, the route is intravenous. In some embodiments, cells described herein are administered as a bolus or by continuous infusion (e.g., intravenous infusion) over a period of time. In some embodiments, cells described herein are administered in several doses over a period of time (e.g., several infusions over a period of time). The cells described herein can be administered in a single dose or in 2, 3, 4, 5, 6 or more doses (or infusions). In some embodiments, the subject being treated is dosed (e.g., with an infusion) about every 1, 2, 3, 4, 5, 6, 7 or 8 weeks. In some embodiments, the subject being treated is dosed (e.g., with an infusion) every 2-4 weeks (e.g., every 2 weeks, 3 weeks or 4 weeks).

In some embodiments, engineered cells are administered systemically, which refers to the administration of a population of cells other than directly into a target site, tissue, or organ, such that it enters, instead, the subject's circulatory system and, thus, is subject to metabolism and other like processes.

The efficacy of a treatment comprising a composition for the treatment of a medical condition can be determined by the skilled clinician. A treatment is considered “effective treatment,” if any one or all of the signs or symptoms of, as but one example, levels of functional target are altered in a beneficial manner (e.g., increased by at least 10%), or other clinically accepted symptoms or markers of disease are improved or ameliorated. Efficacy can also be measured by failure of a subject to worsen as assessed by hospitalization or need for medical interventions (e.g., progression of the disease is halted or at least slowed). Methods of measuring these indicators are known to those of skill in the art and/or described herein. Treatment includes any treatment of a disease in subject and includes: (1) inhibiting the disease, e.g., arresting, or slowing the progression of symptoms; or (2) relieving the disease, e.g., causing regression of symptoms; and (3) preventing or reducing the likelihood of the development of symptoms.

### Lymphodepletion Conditioning Therapy

In some embodiments, any engineered cells described herein (or any population of cells described herein) are administered to a subject (e.g., a human patient having a cancer, e.g., a Hodgkin's lymphoma) after a subject has received a lymphodepleting regimen.

In some embodiments, the lymphodepleting regimen comprises administering at least one chemotherapeutic agent. In some embodiments, at least one chemotherapeutic agent is cyclophosphamide. In some embodiments, the lymphodepleting regimen comprises administering at least two chemotherapeutic agents. In some embodiments, at least two chemotherapeutic agents are cyclophosphamide and fludarabine.

In some embodiments, the first dose (e.g., infusion) of the engineered cells described herein is administered to a subject after lymphodepletion.

### VI. Specific Compositions and Methods of the Disclosure

Accordingly, the present disclosure relates, in particular, to the following non-limiting compositions and methods.

In a first composition, Composition 1, the present disclosure provides a composition comprising an engineered cell comprising (a) an insertion of a polynucleotide encoding a SERPINB9 and (b) a disruption of at least one gene encoding a MHC-I or MHC-II human leukocyte antigen, a component of a MHC-I or MHC-II complex, or a transcriptional regulator of a MHC-I or MHC-II complex, wherein the engineered cell expresses SERPINB9 and has disrupted expression of one or more of the MHC-I or MHC-II human leukocyte antigens, the component of the MHC-I or MHC-II complex, or the transcriptional regulator of the MHC-I or MHC-II complex.

In another composition, Composition 2, the present disclosure provides a composition according to composition 1, wherein the polynucleotide encoding the SERPINB9 is inserted into a targeted chromosomal location.

In another composition, Composition 3, the present disclosure provides a composition according to composition 1 or 2, wherein the gene encoding the MHC-I or MHC-II human leukocyte antigen, the component of the MHC-I or MHC-II complex, or the transcriptional regulator of the MHC-I or MHC-II complex is a MHC-I gene chosen from HLA-A, HLA-B, or HLA-C, a MHC-II gene chosen from HLA-DP, HLA-DM, HLA-DOA, HLA-DOB, HLA-DQ, or HLA-DR, or a gene chosen from B2M, NLRC5, CIITA, RFX5, RFXAP, or RFXANK.

In another composition, Composition 4, the present disclosure provides a composition according to any one of compositions 1 to 3, wherein the disruption comprises a disrupted B2M gene, and the cell has disrupted expression of B2M.

In another composition, Composition 5, the present disclosure provides a composition according to composition 4, wherein disrupted expression of B2M comprises reduced or eliminated expression of B2M.

In another composition, Composition 6, the present disclosure provides a composition according to composition 4 or 5, wherein the polynucleotide encoding SERPINB9 is inserted within or near the B2M gene, thereby disrupting the B2M gene.

In another composition, Composition 7, the present disclosure provides a composition according to any one of compositions 1 to 6, wherein the polynucleotide encoding the SERPINB9 is linked to a polynucleotide encoding an IL15/IL15Rα fusion protein, and the cell further expresses the IL15/IL15Rα fusion protein.

In another composition, Composition 8, the present disclosure provides a composition according to composition 7, wherein the polynucleotide encoding the SERPINB9 is linked to the polynucleotide encoding the Il15/IL15Rα fusion protein by a 2A peptide coding sequence (SERPINB9-P2A-IL15/IL15Rα).

In another composition, Composition 9, the present disclosure provides a composition according to composition 8, wherein SERPINB9-P2A-IL15/IL15Rα consists essentially of SEQ ID NO: 37.

In another composition, Composition 10, the present disclosure provides a composition according to composition 8 or 9, wherein SERPINB9-P2A-IL15/IL15Rα is operably linked to an exogenous promoter.

In another composition, Composition 11, the present disclosure provides a composition according to composition 10, wherein the exogenous promoter is a CAG, CMV, EF1α, PGK, or UBC promoter.

In another composition, Composition 12, the present disclosure provides a composition according to composition 10 or 11, wherein the exogenous promoter is CAG and CAG-SERPINB9-P2A-IL15/IL15Rα consists essentially of SEQ ID NO: 38.

In another composition, Composition 13, the present disclosure provides a composition according to any one of compositions 1 to 12, further comprising an insertion of a first polynucleotide encoding a chimeric antigen receptor (CAR) and/or an insertion of a second polynucleotide encoding an HLA-E, and the cell expresses the CAR and/or HLA-E.

In another composition, Composition 14, the present disclosure provides a composition according to composition 13, wherein the CAR is a CD30 CAR, a BCMA CAR, a GPC3 CAR, a CD19 CAR, a CD33 CAR, a NKG2D CAR, a CD70 CAR, an NKp30 CAR, a CD73 CAR, a GPR87 CAR, a L1V1A CAR, a A33 CAR, a EGFR CAR, a CD20 CAR, or a SLC7A11 CAR.

In another composition, Composition 15, the present disclosure provides a composition according to composition 13 or 14, wherein the first polynucleotide encoding the CAR is linked to the second polynucleotide encoding the HLA-E by a 2A peptide coding sequence (CAR-P2A-HLA-E).

In another composition, Composition 16, the present disclosure provides a composition according to composition 15, wherein the HLA-E is an HLA-E trimer comprising a B2M signal peptide fused to an HLA-G presentation peptide fused to the B2M membrane protein fused to the HLA-E protein without a signal peptide.

In another composition, Composition 17, the present disclosure provides a composition according to composition 15 or 16, wherein CAR-P2A-HLA-E is operably linked to an exogenous promoter.

In another composition, Composition 18, the present disclosure provides a composition according to composition 17, wherein the exogenous promoter is a CAG, CMV, EF1α, PGK, or UBC promoter.

In another composition, Composition 19, the present disclosure provides a composition according to any one of compositions 1 to 18, wherein the cell further comprises a disrupted CIITA gene, and the cell has disrupted expression of CIITA.

In another composition, Composition 20, the present disclosure provides a composition according to composition 19, wherein disrupted expression of CIITA comprises reduced or eliminated expression of CIITA.

In another composition, Composition 21, the present disclosure provides a composition according to any one of compositions 15 to 20, wherein CAR-P2A-HLA-E is inserted within or near the CIITA gene, thereby disrupting expression of CIITA.

In another composition, Composition 22, the present disclosure provides a composition according to any one of compositions 1 to 6, wherein the polynucleotide encoding the SERPINB9 is linked to a polynucleotide encoding an HLA-E protein by a 2A peptide coding sequence (SERPINB9-P2A-HLA-E), and the cell further expresses the HLA-E.

In another composition, Composition 23, the present disclosure provides a composition according to composition 22, wherein the HLA-E is an HLA-E trimer comprising a B2M signal peptide fused to an HLA-G presentation peptide fused to the B2M membrane protein fused to the HLA-E protein without a signal peptide.

In another composition, Composition 24, the present disclosure provides a composition according to composition 22 or 23, wherein SERPINB9-P2A-HLA-E consists essentially of SEQ ID NO: 21.

In another composition, Composition 25, the present disclosure provides a composition according to any one of compositions 22 to 24, wherein SERPINB9-P2A-HLA-E is operably linked to an exogenous promoter.

In another composition, Composition 26, the present disclosure provides a composition according to composition 25, wherein the exogenous promoter is a CAG, CMV, EF1α, PGK, or UBC promoter.

In another composition, Composition 27, the present disclosure provides a composition according to composition 25 or 26, wherein the exogenous promoter is CAG and CAG-SERPINB9-P2A-HLA-E consists essentially of SEQ ID NO: 22.

In another composition, Composition 28, the present disclosure provides a composition according to any one of compositions 1 to 3, wherein the disruption comprises a disrupted a CIITA gene, and the cell has disrupted expression of CIITA.

In another composition, Composition 29, the present disclosure provides a composition according to composition 28, wherein disrupted expression of CIITA comprises reduced or eliminated expression of CIITA.

In another composition, Composition 30, the present disclosure provides a composition according to composition 28 or 29, wherein the polynucleotide encoding the SERPINB9 is inserted within or near the CIITA gene, thereby disrupting expression of CIITA.

In another composition, Composition 31, the present disclosure provides a composition according to composition 30, wherein the polynucleotide encoding the SERPINB9 is linked to a polynucleotide encoding an HLA-E protein by a 2A peptide coding sequence (SERPINB9-P2A-HLA-E), and the cell further expresses the HLA-E.

In another composition, Composition 32, the present disclosure provides a composition according to composition 31, wherein the HLA-E is an HLA-E trimer comprising a B2M signal peptide fused to an HLA-G presentation peptide fused to the B2M membrane protein fused to the HLA-E protein without a signal peptide.

In another composition, Composition 33, the present disclosure provides a composition according to composition 31 or 32, wherein SERPINB9-P2A-HLA-E consists essentially of SEQ ID NO: 21.

In another composition, Composition 34, the present disclosure provides a composition according to any one of compositions 31 to 33, wherein SERPINB9-P2A-HLA-E is operably linked to an exogenous promoter.

In another composition, Composition 35, the present disclosure provides a composition according to composition 34, wherein the exogenous promoter is a CAG, CMV, EF1α, PGK, or UBC promoter.

In another composition, Composition 36, the present disclosure provides a composition according to composition 34 or 35, wherein the exogenous promoter is CAG and CAG-SERPINB9-P2A-HLA-E consists essentially of SEQ ID NO: 22.

In another composition, Composition 37, the present disclosure provides a composition according to composition 34 or 35, wherein the cell further comprises an insertion of a first polynucleotide encoding a XIAP and/or an insertion of a second polynucleotide encoding an IL15/IL15Rα fusion protein, and the cells expresses the XIAP and/or the IL15/IL15Rα fusion protein.

In another composition, Composition 38, the present disclosure provides a composition according to composition 37, wherein the first polynucleotide encoding the XIAP is linked to the second polynucleotide encoding the IL15/IL15Rα fusion protein by a 2A peptide coding sequence (XIAP-P2A-IL15/IL15Rα).

In another composition, Composition 39, the present disclosure provides a composition according to composition 38, wherein XIAP-P2A-IL15/IL15Rα consists essentially of SEQ ID NO: 46.

In another composition, Composition 40, the present disclosure provides a composition according to composition 38 or 39, wherein XIAP-P2A-IL15/IL15Rα is operably linked to an exogenous promoter.

In another composition, Composition 41, the present disclosure provides a composition according to composition 40, wherein the exogenous promoter is a CAG, CMV, EF1α, PGK, or UBC promoter.

In another composition, Composition 42, the present disclosure provides a composition according to composition 40 or 41, wherein the exogenous promoter is CAG and CAG-XIAP-P2A-IL15/IL15Rα consists essentially of SEQ ID NO: 47.

In another composition, Composition 43, the present disclosure provides a composition according to any one of compositions 28 to 42, wherein the cell further comprises a disrupted B2M gene, and the cell has disrupted expression of B2M.

In another composition, Composition 44, the present disclosure provides a composition according to composition 43, wherein disrupted expression of B2M comprises reduced or eliminated expression of B2M.

In another composition, Composition 45, the present disclosure provides a composition according to any one of compositions 38 to 44, wherein XIAP-P2A-IL15/IL15Rα is inserted within or near the B2M gene, thereby disrupting expression of B2M.

In another composition, Composition 46, the present disclosure provides a composition according to any one of compositions 1 to 45, wherein the cell further comprises a disrupted FAS gene, and the cell has disrupted expression of FAS.

In another composition, Composition 47, the present disclosure provides a composition according to composition 46, wherein disrupted expression of FAS comprises reduced or eliminated expression of FAS.

In another composition, Composition 48, the present disclosure provides a composition according to any one of compositions 1 to 47, wherein the cell further comprises a disrupted CISH gene, and the cell has disrupted expression of CISH.

In another composition, Composition 49, the present disclosure provides a composition according to composition 48, wherein disrupted expression of CISH comprises reduced or eliminated expression of CISH.

In another composition, Composition 50, the present disclosure provides a composition comprising an engineered cell comprising an insertion of a first polynucleotide encoding a SERPINB9 and an insertion of a second polynucleotide encoding an HLA-E, wherein the cell expresses the SERPINB9 and HLA-E.

In another composition, Composition 51, the present disclosure provides a composition according to composition 50, wherein the polynucleotide encoding the SERPINB9 is linked to the polynucleotide encoding an HLA-E protein by a 2A peptide coding sequence (SERPINB9-P2A-HLA-E).

In another composition, Composition 52, the present disclosure provides a composition according to composition 50 or 51, wherein the HLA-E is an HLA-E trimer comprising a B2M signal peptide fused to an HLA-G presentation peptide fused to the B2M membrane protein fused to the HLA-E protein without a signal peptide.

In another composition, Composition 53, the present disclosure provides a composition according to composition 51 or 52, wherein SERPINB9-P2A-HLA-E consists essentially of SEQ ID NO: 21.

In another composition, Composition 54, the present disclosure provides a composition according to any one of compositions 51 to 53, wherein SERPINB9-P2A-HLA-E is operably linked to an exogenous promoter.

In another composition, Composition 55, the present disclosure provides a composition according to composition 54, wherein the exogenous promoter is a CAG, CMV, EF1α, PGK, or UBC promoter.

In another composition, Composition 56, the present disclosure provides a composition according to composition 54 or 55, wherein the exogenous promoter is CAG and CAG-SERPINB9-P2A-HLA-E consists essentially of SEQ ID NO: 22.

In another composition, Composition 57, the present disclosure provides a composition according to any one of compositions 50 to 56, wherein the first polynucleotide encoding a SERPINB9 and the second polynucleotide encoding an HLA-E are inserted within or near a B2M gene locus, and the cell has disrupted expression of B2M.

In another composition, Composition 58, the present disclosure provides a composition according to composition 57, wherein disrupted expression of B2M comprises reduced or eliminated expression of B2M.

In another composition, Composition 59, the present disclosure provides a composition according to any one of compositions 50 to 56, wherein the first polynucleotide encoding a SERPINB9 and the second polynucleotide encoding an HLA-E are inserted within or near a CIITA gene locus, and the cell has disrupted expression of CIITA.

In another composition, Composition 60, the present disclosure provides a composition according to composition 59, wherein disrupted expression of CIITA comprises reduced or eliminated expression of CIITA.

In another composition, Composition 61, the present disclosure provides a composition according to any one of compositions 1 to 60, wherein the engineered cell is a stem cell.

In another composition, Composition 62, the present disclosure provides a composition according to composition 61, wherein the stem cell is an embryonic stem cell, an adult stem cell, an induced pluripotent stem cell, or a hematopoietic stem cell.

In another composition, Composition 63, the present disclosure provides a composition according to any one of compositions 1 to 60, wherein the engineered cell is a differentiated cell or a somatic cell.

In another composition, Composition 64, the present disclosure provides a composition according to any one of compositions 1 to 60, wherein the engineered cell is capable of being differentiated into a lineage-restricted progenitor cell or a fully differentiated somatic cell.

In another composition, Composition 65, the present disclosure provides a composition according to composition 64, wherein the lineage-restricted progenitor cell is a hematopoietic progenitor cell, mesodermal cell, definitive hemogenic endothelium, definitive hematopoietic stem or progenitor cell, CD34+ cell, multipotent progenitor (MPP), common lymphoid progenitor cell, T cell progenitor, NK cell progenitor, definitive endoderm, hepatoblast, pancreatic endoderm progenitor, pancreatic endocrine progenitor, mesenchymal progenitor cell, muscle progenitor cell, blast cell, or neural progenitor cell, and the fully differentiated somatic cell is a hematopoietic cell, hepatocyte, pancreatic beta cell, epithelial cell, endodermal cell, macrophage, adipocyte, kidney cell, blood cell, cardiomyocyte, or immune system cell.

In another composition, Composition 66, the present disclosure provides a composition according to any one of compositions 1 to 60, wherein the engineered cell is a natural killer (NK) cell.

In another composition, Composition 67, the present disclosure provides a composition according to compositions 66, wherein the NK cell has been differentiated from a genome-edited iPSC, wherein the NK cell comprises the genome edits of the genome-edited iPSC, wherein the NK cell has not been genome-edited after the differentiation.

In another composition, Composition 68, the present disclosure provides a composition according to any one of compositions 1 to 60, wherein the engineered cell is a hepatoblast or a hepatocyte.

In another composition, Composition 69, the present disclosure provides a composition according to composition 68, wherein the hepatoblast or hepatocyte has been differentiated from a genome-edited iPSC, wherein the hepatoblast or hepatocyte comprises the genome edits of the genome-edited iPSC, wherein the hepatoblast or hepatocyte has not been genome-edited after the differentiation.

In another composition, Composition 70, the present disclosure provides a composition comprising a population of engineered cells according to compositions 61 or 62.

In another composition, Composition 71, the present disclosure provides a composition comprising a population of engineered cells according to any one of compositions 63 to 69.

In another composition, Composition 72, the present disclosure provides a composition comprising a population of engineered cells according to composition 66 or 67.

In another composition, Composition 73, the present disclosure provides a composition comprising a population of engineered cells according to composition 68 or 69.

In another composition, Composition 74, the present disclosure provides a composition comprising a population of engineered cells according to any one of compositions 70 to 73 and at least one pharmaceutically acceptable excipient.

In another composition, Composition 75, the present disclosure provides a composition according to composition 74, for use in treating a subject in need thereof.

In another composition, Composition 76, the present disclosure provides a composition for use according to composition 74, for use in treating cancer in a subject in need thereof, wherein the composition comprises the population of cells of composition 72.

In another composition, Composition 77, the present disclosure provides a composition for use according to composition 75 or 76, wherein the subject has multiple myeloma, Hodgkin's lymphoma, lung cancer, leukemia, B-cell acute lymphoblastic leukemia (B-ALL), B-cell non-Hodgkin's lymphoma (B-NL), Chronic lymphocytic leukemia (C-CLL), T cell lymphoma, T cell leukemia, clear cell renal cell carcinoma (ccRCC), thyroid cancer, nasopharyngeal cancer, non-small cell lung (NSCLC), pancreatic cancer, melanoma, ovarian cancer, glioblastoma, liver cancer, or cervical cancer.

In another composition, Composition 78, the present disclosure provides a composition according to composition 74, for use in treating a hepatic disease or disorder in a subject in need thereof, wherein the composition comprises the population of cells of composition 73.

In another composition, Composition 79, the present disclosure provides a composition according to composition 78, wherein the subject has fatty liver disease, non-alcoholic fatty liver disease, autoimmune hepatitis, alcoholic hepatitis, viral hepatitis, ischemic hepatitis, metabolic disorder hepatitis, chronic liver inflammation, hepatic fibrosis, cholestasis, primary sclerosing cholangitis, cirrhosis, primary biliary cirrhosis, zonal necrosis, hemochromatosis, Wilson's disease, alpha 1-antitrypsin deficiency, glycogen storage disease type II, Gilbert's syndrome, portal hypertension, portal vein thrombosis, ascites, hepatic steatosis post-liver transplantation, or acute or chronic liver transplant rejection and metabolic conditions.

In another composition, Composition 80, the present disclosure provides a composition according to any one of compositions 75 to 79, wherein the subject is human.

In a first method, Method 81, the present disclosure provides a method of obtaining cells for administration to a subject in need thereof, the method comprising: (a) obtaining or having obtained the population of engineered cells according to composition 70, and (b) maintaining the population of engineered cells for a time and under conditions sufficient for the cells to differentiate into lineage-restricted progenitor cells or fully differentiated somatic cells.

In another method, Method 82, the present disclosure provides a method for treating of a subject in need thereof, the method comprising: (a) obtaining or having obtained the population of engineered cells according to composition 70 following differentiation into lineage-restricted progenitor cells or fully differentiated somatic cells; and (b) administering the lineage-restricted progenitor cells or fully differentiated somatic cells to the subject.

In another method, Method 83, the present disclosure provides a method according to method 81 or 82, wherein the lineage-restricted progenitor cells are hematopoietic progenitor cells, mesodermal cells, definitive hemogenic endothelium, definitive hematopoietic stem or progenitor cells, CD34+ cells, multipotent progenitors (MPP), common lymphoid progenitor cells, T cell progenitors, NK cell progenitors, definitive endoderm, hepatoblasts, pancreatic endoderm progenitors, pancreatic endocrine progenitors, mesenchymal progenitor cells, muscle progenitor cells, blast cells, or neural progenitor cells, and the fully differentiated somatic cells are hematopoietic cells, hepatocytes, pancreatic beta cells, epithelial cells, endodermal cells, macrophages, hepatocytes, adipocytes, kidney cells, blood cells, cardiomyocytes, or immune system cells.

In another method, Method 84, the present disclosure provides a method according to any one of methods 81 to 83, wherein the subject has, is suspected of having, or is at risk for a cancer, or the subject has, is suspected of having, or is at risk for a hepatic disease or disorder.

In another method, Method 85, the present disclosure provides a method according to any one of methods 81 or 84, wherein the subject is human.

In another method, Method 86, the present disclosure provides an in vitro method for generating an engineered cell, the method comprising delivering to a cell: (a) a first RNP complex comprising an RNA-guided nuclease and a gRNA targeting a target site in a B2M gene locus or a first RNA-guided nuclease and a first gRNA targeting a target site in a B2M gene locus; and (b) a first vector comprising a nucleic acid, the nucleic acid comprising: (i) nucleotide sequence encoding a SERPINB9 and a nucleotide sequence encoding an IL15/IL15Rα fusion protein; (ii) a nucleotide sequence having sequence homology with a genomic region located left of the target site in the B2M gene locus; and (iii) a nucleotide sequence having sequence homology with a genomic region located right of the target site in the B2M gene locus, wherein (i) is flanked by (ii) and (iii); wherein the B2M gene locus is cleaved at the target site and the nucleotide sequences encoding the SERPINB9 and the IL15/IL15Rα fusion protein are inserted into the B2M gene locus, thereby disrupting the B2M gene.

In another method, Method 87, the present disclosure provides an in vitro method according to method 86, wherein the gRNA of the first RNP complex comprises a spacer sequence corresponding to a sequence consisting of SEQ ID NO: 1.

In another method, Method 88, the present disclosure provides an in vitro method according to method 86 or 87, wherein the nucleotide sequence of (b)(i) comprises the nucleotide sequence encoding the SERPINB9 linked to a nucleotide sequence encoding a P2A peptide sequence linked to the nucleotide sequence encoding the IL15/IL15Rα fusion protein (SERPINB9-P2A-IL15/IL15Rα).

In another method, Method 89, the present disclosure provides an in vitro method according to method 88, wherein SERPINB9-P2A-IL15/IL15Rα consists essentially of SEQ ID NO: 37.

In another method, Method 90, the present disclosure provides an in vitro method according to method 88 or 89, wherein SERPINB9-P2A-IL15/IL15Rα is operably linked to an exogenous promoter.

In another method, Method 91, the present disclosure provides an in vitro method according to method 90, wherein the exogenous promoter is CAG (CAG-SERPINB9-P2A-IL15/IL15Rα), and CAG-SERPINB9-P2A-IL15/IL15Rα consists essentially of SEQ ID NO: 38.

In another method, Method 92, the present disclosure provides an in vitro method according to any one of methods 86 to 91, wherein the nucleotide sequence of (b)(ii) consists essentially of SEQ ID NO: 3, and the nucleotide sequence of (b)(iii) consists essentially of SEQ ID NO: 19.

In another method, Method 93, the present disclosure provides an in vitro method according to any one of methods 86 to 92, wherein the first vector consists essentially of SEQ ID NO: 39.

In another method, Method 94, the present disclosure provides an in vitro method according to any one of methods 86 to 93, further comprising delivering to the cell: (c) a second RNP complex comprising an RNA-guided nuclease and a gRNA targeting a target site in a CIITA gene locus or a second RNA-guided nuclease and a second gRNA targeting a target site in a B2M gene locus; (d) a second vector comprising a nucleic acid, the nucleic acid comprising: (i) a nucleotide sequence encoding a CAR and a nucleotide sequence encoding a HLA-E trimer; (ii) a nucleotide sequence having sequence homology with a genomic region located left of the target site in the CIITA gene locus; and (iii) a nucleotide sequence having sequence homology with a genomic region located right of the target site in the CIITA gene locus, wherein (i) is flanked by (ii) and (iii); and wherein the CIITA gene locus is cleaved at the target site and the nucleotide sequences encoding the CAR and the HLA-E trimer are inserted into the CIITA gene locus, thereby disrupting the CIITA gene.

In another method, Method 95, the present disclosure provides an in vitro method according to method 94, wherein the gRNA of the second RNP complex comprises a spacer sequence corresponding to a sequence consisting of SEQ ID NO: 41.

In another method, Method 96, the present disclosure provides an in vitro method according to method 94 or 95, wherein the nucleotide sequence of (d)(i) comprises the nucleotide sequence encoding the CAR linked to a nucleotide sequence encoding a P2A peptide sequence linked to the nucleotide sequence encoding the HLA-E trimer.

In another method, Method 97, the present disclosure provides an in vitro method according to any one of methods 94 to 96, wherein the nucleotide sequence of (d)(ii) consists essentially of SEQ ID NO: 42, and the nucleotide sequence of (d)(iii) consists essentially of SEQ ID NO: 43.

In another method, Method 98, the present disclosure provides an in vitro method according to any one of methods 94 to 97, wherein the engineered cell has reduced or eliminated expression of CIITA.

In another method, Method 99, the present disclosure provides an in vitro method for generating an engineered cell, the method comprising delivering to a cell: (a) a RNP complex comprising an RNA-guided nuclease and a gRNA targeting a target site in a B2M gene locus or a first RNA-guided nuclease and a first gRNA targeting a target site in a B2M gene locus; (b) a vector comprising a nucleic acid, the nucleic acid comprising: (i) nucleotide sequence encoding a SERPINB9 and a nucleotide sequence encoding an HLA-E trimer; (ii) a nucleotide sequence having sequence homology with a genomic region located left of the target site in the B2M gene locus; and (iii) a nucleotide sequence having sequence homology with a genomic region located right of the target site in the B2M gene locus, wherein (i) is flanked by (ii) and (iii); wherein the B2M gene locus is cleaved at the target site and the nucleotide sequences encoding the SERPINB9 and the HLA-E trimer are inserted into the B2M gene locus, thereby disrupting the B2M gene.

In another method, Method 100, the present disclosure provides an in vitro method according to method 99, wherein the gRNA comprises a spacer sequence corresponding to a sequence consisting essentially of SEQ ID NO: 1.

In another method, Method 101, the present disclosure provides an in vitro method according to method 99 or 100, wherein the nucleotide sequence of (b)(i) comprises the nucleotide sequence encoding the SERPINB9 linked to a nucleotide sequence encoding a P2A peptide sequence linked to the nucleotide sequence encoding the HLA-E trimer (SERPINB9-P2A-HLA-E).

In another method, Method 102, the present disclosure provides an in vitro method according to method 101, wherein SERPINB9-P2A-HLA-E consists essentially of SEQ ID NO: 21.

In another method, Method 103, the present disclosure provides an in vitro method according to method 101 or 102, wherein SERPINB9-P2A-HLA-E is operably linked to an exogenous promoter.

In another method, Method 104, the present disclosure provides an in vitro method according to method 103, wherein the exogenous promoter is CAG (CAG-SERPINB9-P2A-HLA-E), and CAG-SERPINB9-P2A-HLA-E consists essentially of SEQ ID NO: 22.

In another method, Method 105, the present disclosure provides an in vitro method according to any one of methods 99 to 104, wherein the nucleotide sequence of (b)(ii) consists essentially of SEQ ID NO: 3, and the nucleotide sequence of (b)(iii) consists essentially of SEQ ID NO: 19.

In another method, Method 106, the present disclosure provides an in vitro method according to any one of methods 99 to 105, wherein the vector consists essentially of SEQ ID NO: 23.

In another method, Method 107, the present disclosure provides an in vitro method according to any one of methods 86 to 106, wherein the engineered cell has reduced or eliminated expression of B2M.

In another method, Method 108, the present disclosure provides an in vitro method for generating an engineered cell, the method comprising delivering to a cell: (a) a first RNP complex comprising an RNA-guided nuclease and a gRNA targeting a target site in a CIITA gene locus or a first RNA-guided nuclease and a first gRNA targeting a target site in a CIITA gene locus; (b) a first vector comprising a nucleic acid, the nucleic acid comprising: (i) nucleotide sequence encoding a SERPINB9 and a nucleotide sequence encoding an HLA-E trimer; (ii) a nucleotide sequence having sequence homology with a genomic region located left of the target site in the CIITA gene locus; and (iii) a nucleotide sequence having sequence homology with a genomic region located right of the target site in the CIITA gene locus, wherein (i) is flanked by (ii) and (iii); wherein the CIITA gene locus is cleaved at the target site and the nucleotide sequences encoding the SERPINB9 and the HLA-E trimer are inserted into the CIITA gene locus, thereby disrupting the CIITA gene.

In another method, Method 109, the present disclosure provides an in vitro method according to method 108, wherein the gRNA of the first RNP complex comprises a spacer sequence corresponding to a sequence consisting of SEQ ID NO: 41.

In another method, Method 110, the present disclosure provides an in vitro method according to method 108 or 109, wherein the nucleotide sequence of (b)(i) comprises the nucleotide sequence encoding the SERPINB9 linked to a nucleotide sequence encoding a P2A peptide sequence linked to the nucleotide sequence encoding the HLA-E trimer (SERPINB9-P2A-HLA-E).

In another method, Method 111, the present disclosure provides an in vitro method according to method 110, wherein SERPINB9-P2A-HLA-E consists essentially of SEQ ID NO: 21.

In another method, Method 112, the present disclosure provides an in vitro method according to method 110 or 111, wherein SERPINB9-P2A-HLA-E is operably linked to an exogenous promoter.

In another method, Method 113, the present disclosure provides an in vitro method according to method 112, wherein the exogenous promoter is CAG (CAG-SERPINB9-P2A-HLA-E), and CAG-SERPINB9-P2A-HLA-E consists essentially of SEQ ID NO: 22.

In another method, Method 114, the present disclosure provides an in vitro method according to any one of methods 108 to 113, wherein the nucleotide sequence of (b)(ii) consists essentially of SEQ ID NO: 42, and the nucleotide sequence of (b)(iii) consists essentially of SEQ ID NO: 43.

In another method, Method 115, the present disclosure provides an in vitro method according to any one of methods 108 to 114, wherein the first vector consists essentially of SEQ ID NO: 44.

In another method, Method 116, the present disclosure provides an in vitro method according to any one of methods 108 to 115, further comprising delivering to the cell: (c) a second RNP complex comprising an RNA-guided nuclease and a gRNA targeting a target site in a B2M gene locus or a second RNA-guided nuclease and a second gRNA targeting a target site in a B2M gene locus; (d) a second vector comprising a nucleic acid, the nucleic acid comprising: (i) a nucleotide sequence encoding XIAP and a nucleotide sequence encoding a IL15/IL15Rα fusion protein; (ii) a nucleotide sequence having sequence homology with a genomic region located left of the target site in the B2M gene locus; and (iii) a nucleotide sequence having sequence homology with a genomic region located right of the target site in the B2M gene locus, wherein (i) is flanked by (ii) and (iii); and wherein the B2M gene locus is cleaved at the target site and the nucleotide sequences encoding XIAP and IL15/IL15Rα are inserted into the B2M gene locus, thereby disrupting the B2M gene.

In another method, Method 117, the present disclosure provides an in vitro method according to method 116, wherein the gRNA of the second RNP complex comprises a spacer sequence corresponding to a sequence consisting of SEQ ID NO: 1.

In another method, Method 118, the present disclosure provides an in vitro method according to method 116 or 117, wherein the nucleotide sequence of (d)(i) comprises the nucleotide sequence encoding XIAP linked to a nucleotide sequence encoding a P2A peptide sequence linked to the nucleotide sequence encoding the IL15/IL15Rα fusion protein (XIAP-P2A-IL15/IL15Rα).

In another method, Method 119, the present disclosure provides an in vitro method according to method 118, wherein XIAP-P2A-IL15/IL15Rα consists essentially of SEQ ID NO: 46.

In another method, Method 120, the present disclosure provides an in vitro method according to method 118 or 119, wherein XIAP-P2A-IL15/IL15Rα is operably linked to an exogenous promoter.

In another method, Method 121, the present disclosure provides an in vitro method according to method 120, wherein the exogenous promoter is CAG (CAG-XIAP-P2A-IL15/IL15Rα), and CAG-XIAP-P2A-IL15/IL15Rα consists essentially of SEQ ID NO: 47.

In another method, Method 122, the present disclosure provides an in vitro method according to any one of methods 116 to 121, wherein the nucleotide sequence of (d)(ii) consists essentially of SEQ ID NO: 3, and the nucleotide sequence of (d)(iii) consists essentially of SEQ ID NO: 19.

In another method, Method 123, the present disclosure provides an in vitro method according to any one of methods 116 to 122, wherein the second vector consists essentially of SEQ ID NO: 48.

In another method, Method 124, the present disclosure provides an in vitro method according to any one of methods 116 to 123, wherein the engineered cell has reduced or eliminated expression of B2M.

In another method, Method 125, the present disclosure provides an in vitro method according to any one of methods 108 to 124, wherein the engineered cell has reduced or eliminated expression of CIITA.

In another method, Method 126, the present disclosure provides an in vitro method according to any one of methods 86 to 124, further comprising delivering to the cell an RNP complex comprising an RNA-guided nuclease and a gRNA targeting a target site in a CISH gene locus.

In another method, Method 127, the present disclosure provides an in vitro method according to method 126, wherein the gRNA targeting a target site in a CISH gene locus comprises a spacer sequence corresponding to a sequence consisting of any one of SEQ ID NOS: 49-60.

In another method, Method 128, the present disclosure provides an in vitro method according to any one of methods 86 to 127, further comprising delivering to the cell an RNP complex comprising an RNA-guided nuclease and a gRNA targeting a target site in a FAS gene locus.

In another method, Method 129, the present disclosure provides an in vitro method according to method 128, wherein the gRNA targeting a target site in a FAS gene locus comprises a spacer sequence corresponding to a sequence consisting of any one of SEQ ID NOS: 61-67.

In another method, Method 130, the present disclosure provides an in vitro method according to any one of methods 86 to 129, wherein the RNA-guided nuclease is a Cas9 nuclease.

In another method, Method 131, the present disclosure provides an in vitro method according to method 130, wherein the Cas9 nuclease is linked to at least one nuclear localization signal.

In another method, Method 132, the present disclosure provides an in vitro method according to any one of methods 86 to 131, wherein the cell is a stem cell.

In another method, Method 133, the present disclosure provides an in vitro method according to method 132, wherein the stem cell is an embryonic stem cell, an adult stem cell, an induced pluripotent stem cell, or a hematopoietic stem cell.

In another method, Method 134, the present disclosure provides an in vitro method according to method 132 or 133, wherein the stem cell is a human stem cell.

In another composition, Composition 135, the present disclosure provides a population of engineered cells generated by the method according to any one of methods 86 to 134.

In another composition, Composition 136, the present disclosure provides a population of cells according to Composition 135, wherein the population is maintained for a time and under conditions sufficient for the cells to undergo differentiation.

In another composition, Composition 137, the present disclosure provides a population of cells according to Composition 135 or 136, for use in treating a subject in need thereof.

In another composition, Composition 138, the present disclosure provides a population of cells for use according to Composition 137, wherein the subject is a human who has, is suspected of having, or is at risk for a cancer, or a human who has, is suspected of having, or is at risk for a hepatic disease or disorder.

In another method, Method 139, the present disclosure provides a method comprising administering to a subject the population of engineered cells according to Composition 135 or 136.

In another method, Method 140, the present disclosure provides a method for treating of a subject in need thereof, the method comprising: (a) obtaining or having obtained the population of engineered cells according to Composition 135 following differentiation into lineage-restricted progenitor cells or fully differentiated somatic cells; and (b) administering the lineage-restricted progenitor cells or fully differentiated somatic cells to the subject.

In another method, Method 141, the present disclosure provides a method of obtaining cells for administration to a subject in need thereof, the method comprising: (a) obtaining or having obtained the population engineered cells according to Composition 135; and (b) maintaining the engineered cells for a time and under conditions sufficient for the cells to differentiate into lineage-restricted progenitor cells or fully differentiated somatic cells.

In another method, Method 142, the present disclosure provides a method according to method 140 or 141, wherein the lineage-restricted progenitor cells are hematopoietic progenitor cells, mesodermal cells, definitive hemogenic endothelium, definitive hematopoietic stem or progenitor cells, CD34+ cells, multipotent progenitors (MPP), common lymphoid progenitor cells, T cell progenitors, NK cell progenitors, definitive endoderm, hepatoblasts, pancreatic endoderm progenitors, pancreatic endocrine progenitors, mesenchymal progenitor cells, muscle progenitor cells, blast cells, or neural progenitor cells, and the fully differentiated somatic cells are hematopoietic cells, hepatocytes, pancreatic beta cells, epithelial cells, endodermal cells, macrophages, hepatocytes, adipocytes, kidney cells, blood cells, cardiomyocytes, or immune system cells.

In another method, Method 143, the present disclosure provides a method according to any one of methods 139-142, wherein the subject is a human who has, is suspected of having, or is at risk for a cancer.

In another method, Method 144, the present disclosure provides a method according to method 143, wherein the subject has multiple myeloma. Hodgkin's lymphoma, lung cancer, leukemia, B-cell acute lymphoblastic leukemia (B-ALL), B-cell non-Hodgkin's lymphoma (B-NL), Chronic lymphocytic leukemia (C-CLL), T cell lymphoma, T cell leukemia, clear cell renal cell carcinoma (ccRCC), thyroid cancer, nasopharyngeal cancer, non-small cell lung (NSCLC), pancreatic cancer, liver cancer, melanoma, ovarian cancer, glioblastoma, or cervical cancer.

In another method, Method 145, the present disclosure provides a method according to any one of the methods 139 to 142, wherein the subject is a human who has, is suspected of having, or is at risk for a hepatic disease or disorder.

In another method, Method 146, the present disclosure provides a method according to method 145, wherein the subject has fatty liver disease, non-alcoholic fatty liver disease, autoimmune hepatitis, alcoholic hepatitis, viral hepatitis, ischemic hepatitis, metabolic disorder hepatitis, chronic liver inflammation, hepatic fibrosis, cholestasis, primary sclerosing cholangitis, cirrhosis, primary biliary cirrhosis, zonal necrosis, hemochromatosis, Wilson's disease, alpha 1-antitrypsin deficiency, glycogen storage disease type II, Gilbert's syndrome, portal hypertension, portal vein thrombosis, ascites, hepatic steatosis post-liver transplantation, or acute or chronic liver transplant rejection and metabolic conditions.

In another composition, Composition 147, the present disclosure provides a composition comprising a gRNA comprising a spacer sequence corresponding to a sequence consisting of any one of SEQ ID NOS: 49, 50, 51, 52, 53, 54, 55, 56, 57, 58, 59, or 60.

In another composition, Composition 148, the present disclosure provides a composition comprising a gRNA comprising a spacer sequence corresponding to a sequence consisting of any one of SEQ ID NOS: 61, 62, 63, 64, 65, 66, or 67.

In another composition, Composition 149, the present disclosure provides a composition comprising an engineered cell comprising an insertion of a polynucleotide encoding a SERPINB9 and (a) a disrupted B2M gene and/or (b) a disrupted CIITA gene, wherein the cell expresses SERPINB9 and has disrupted expression of B2M and/or CIITA.

In another composition, Composition 150, the present disclosure provides a composition according to Composition 149, wherein the polynucleotide encoding SERPINB9 is inserted within or near the B2M gene, thereby disrupting the B2M gene.

In another composition, Composition 151, the present disclosure provides a composition according to Composition 149, wherein the polynucleotide encoding SERPINB9 is inserted within or near the CIITA gene, thereby disrupting the CIITA gene.

In another composition, Composition 152, the present disclosure provides a composition according to Composition 149, wherein the polynucleotide encoding the SERPINB9 is linked to a polynucleotide encoding an IL15/IL15Rα fusion protein, and the cell further expresses the IL15/IL15Rα fusion protein.

In another composition, Composition 153, the present disclosure provides a composition according to Composition 152, wherein the polynucleotide encoding the SERPINB9 is linked to the polynucleotide encoding the Il15/IL15Rα fusion protein by a 2A peptide coding sequence to form a SERPINB9-P2A-IL15/IL15Rα construct.

In another composition, Composition 154, the present disclosure provides a composition according to Composition 153, wherein the SERPINB9-P2A-IL15/IL15Rα construct consists essentially of SEQ ID NO: 37.

In another composition, Composition 155, the present disclosure provides a composition according to Composition 153 or 154, wherein the SERPINB9-P2A-IL15/IL15Rα construct is operably linked to an exogenous promoter.

In another composition, Composition 156, the present disclosure provides a composition according to Composition 155, wherein the exogenous promoter is a CAG, CMV, EF1α, PGK, or UBC promoter.

In another composition, Composition 157, the present disclosure provides a composition according to Composition 156, wherein the exogenous promoter is CAG and CAG-SERPINB9-P2A-IL15/IL15Rα consists essentially of SEQ ID NO: 38.

In another composition, Composition 158, the present disclosure provides a composition according to Composition 149, wherein the polynucleotide encoding the SERPINB9 is linked to a polynucleotide encoding an HLA-E protein by a 2A peptide coding sequence to form a SERPINB9-P2A-HLA-E construct, and the cell further expresses the HLA-E.

In another composition, Composition 159, the present disclosure provides a composition according to Composition 158, wherein the HLA-E is an HLA-E trimer comprising a B2M signal peptide fused to an HLA-G presentation peptide fused to the B2M membrane protein fused to the HLA-E protein without a signal peptide.

In another composition, Composition 160, the present disclosure provides a composition according to Composition 158 wherein the SERPINB9-P2A-HLA-E construct consists essentially of SEQ ID NO: 21.

In another composition, Composition 161, the present disclosure provides a composition according to any one of Compositions 158 to 160, wherein the SERPINB9-P2A-HLA-E construct is operably linked to an exogenous promoter.

In another composition, Composition 162, the present disclosure provides a composition according to Composition 161, wherein the exogenous promoter is a CAG, CMV, EF1α, PGK, or UBC promoter.

In another composition, Composition 163, the present disclosure provides a composition according to Composition 162, wherein the exogenous promoter is CAG and CAG-SERPINB9-P2A-HLA-E consists essentially of SEQ ID NO: 22.

In another composition, Composition 164, the present disclosure provides a composition according to any one of Compositions 149 to 163, wherein the cell further comprises a disrupted FAS gene, and the cell has disrupted expression of FAS.

In another composition, Composition 165, the present disclosure provides a composition according to Composition 164, wherein disrupted expression of FAS comprises reduced or eliminated expression of FAS.

In another composition, Composition 166, the present disclosure provides a composition according to any one of Compositions 149 to 165, wherein the cell further comprises a disrupted CISH gene, and the cell has disrupted expression of CISH.

In another composition, Composition 167, the present disclosure provides a composition according to Composition 166, wherein disrupted expression of CISH comprises reduced or eliminated expression of CISH.

In another composition, Composition 168, the present disclosure provides a composition according to any one of Compositions 149 to 167, wherein the engineered cell is a stem cell.

In another composition, Composition 169, the present disclosure provides a composition according to Composition 168, wherein the stem cell is an a pluripotent stem cell, an adult stem cell, or a hematopoietic stem cell.

In another composition, Composition 170, the present disclosure provides a composition according to Composition 169, wherein the stem cell is an embryonic stem cell or an induced pluripotent stem cell.

In another composition, Composition 171, the present disclosure provides a composition according to any one of Compositions 149 to 167, wherein engineered cell is a differentiated cell or a somatic cell.

In another composition, Composition 172, the present disclosure provides a composition according to any one of Compositions 149 to 167, wherein the cell is capable of being differentiated into lineage-restricted progenitor cells or fully differentiated somatic cells.

In another composition, Composition 173, the present disclosure provides a composition according to Composition 172, wherein the lineage-restricted progenitor cell is a hematopoietic progenitor cell, mesodermal cell, definitive hemogenic endothelium, definitive hematopoietic stem or progenitor cell, CD34+ cell, multipotent progenitor (MPP), common lymphoid progenitor cell, T cell progenitor, NK cell progenitor, definitive endoderm, hepatoblast, pancreatic endoderm progenitor, pancreatic endocrine progenitor, mesenchymal progenitor cell, muscle progenitor cell, blast cell, or neural progenitor cell, and the fully differentiated somatic cell is a hematopoietic cell, hepatocyte, pancreatic beta cell, epithelial cell, endodermal cell, macrophage, adipocyte, kidney cell, blood cell, cardiomyocyte, or immune system cell.

In another composition, Composition 174, the present disclosure provides a population of cells comprising one or more engineered cell according to Composition 149.

In another composition, Composition 174, the present disclosure provides a population of cells comprising lineage-restricted progenitor cells or fully differentiated somatic cells derived from one or more engineered cells according to Composition 149.

In another composition, Composition 175, the present disclosure provides a population of cells according to Composition 174, wherein lineage-restricted progenitor cells comprise at least one hematopoietic progenitor cell, mesodermal cell, definitive hemogenic endothelium, definitive hematopoietic stem or progenitor cell, CD34+ cell, multipotent progenitor (MPP), common lymphoid progenitor cell, T cell progenitor, NK cell progenitor, definitive endoderm, hepatoblast, pancreatic endoderm progenitor, pancreatic endocrine progenitor, mesenchymal progenitor cell, muscle progenitor cell, blast cell, or neural progenitor cell, and the fully differentiated somatic cells comprise at least one hematopoietic cell, hepatocyte, pancreatic beta cell, epithelial cell, endodermal cell, macrophage, adipocyte, kidney cell, blood cell, cardiomyocyte, or immune system cell.

In another method, Method 176, the present disclosure provides an in vitro method for generating an engineered cell, the method comprising delivering to a cell: (a) an RNA-guided nuclease and a gRNA targeting a target site in a B2M gene locus or a first RNP complex comprising an RNA-guided nuclease and a gRNA targeting a target site in a B2M gene locus; and (b) a vector comprising a nucleic acid, the nucleic acid comprising: (i) nucleotide sequence encoding a SERPINB9 protein; (ii) a nucleotide sequence having sequence homology with a genomic region located left of the target site in the B2M gene locus; and (iii) a nucleotide sequence having sequence homology with a genomic region located right of the target site in the B2M gene locus, wherein (i) is flanked by (ii) and (iii); wherein the B2M gene locus is cleaved at the target site and the nucleotide sequences encoding the SERPINB9 protein are inserted into the B2M gene locus, thereby disrupting the B2M gene.

In another method, Method 177, the present disclosure provides an in vitro method according to Method 176 wherein the gRNA comprises a spacer sequence corresponding to a sequence consisting of SEQ ID NO: 1.

In another method, Method 178, the present disclosure provides an in vitro method according to Method 176 or 177, wherein the nucleotide sequence of (b)(i) further comprises a nucleotide sequence encoding a IL15/IL15Rα fusion protein.

In another method, Method 179, the present disclosure provides an in vitro method according to any one of Method 176 to 178, wherein the nucleotide sequence of (b)(i) comprises the nucleotide sequence encoding the SERPINB9 protein linked to a nucleotide sequence encoding a P2A peptide sequence linked to the nucleotide sequence encoding the IL15/IL15Rα fusion protein to form a SERPINB9-P2A-IL15/IL15Rα construct.

In another method, Method 180, the present disclosure provides an in vitro method according to Method 179, wherein the SERPINB9-P2A-IL15/IL15Rα construct consists essentially of SEQ ID NO: 37.

In another method, Method 181, the present disclosure provides an in vitro method according to Method 179 or 180, wherein the SERPINB9-P2A-IL15/IL15Rα construct is operably linked to an exogenous promoter.

In another method, Method 182, the present disclosure provides an in vitro method according to Method 176 or 177 wherein the nucleotide sequence of (b)(i) further comprises a nucleotide sequence encoding an HLA-E trimer.

In another method, Method 183, the present disclosure provides an in vitro method according to Method 182, wherein the nucleotide sequence of (b)(i) comprises the nucleotide sequence encoding the SERPINB9 linked to a nucleotide sequence encoding a P2A peptide sequence linked to the nucleotide sequence encoding the HLA-E trimer to form a SERPINB9-P2A-HLA-E construct.

In another method, Method 184, the present disclosure provides an in vitro method according to Method 183, wherein the SERPINB9-P2A-HLA-E construct consists essentially of SEQ ID NO: 21.

In another method, Method 185 the present disclosure provides an in vitro method according to Method 183 or 184, wherein the SERPINB9-P2A-HLA-E construct is operably linked to an exogenous promoter.

In another method, Method 185.1 the present disclosure provides an in vitro method according to Method 176, wherein the nucleotide sequence of (b)(ii) consists essentially of SEQ ID NO: 3, and the nucleotide sequence of (b)(iii) consists essentially of SEQ ID NO: 19.

In another method, Method 186 the present disclosure provides an in vitro method according to Method 176, wherein the first vector consists essentially of SEQ ID NO: 23 or 39.

In another method, Method 187 the present disclosure provides an in vitro method according to any one of Methods 176 to 186, further comprising delivering to the cell an RNA-guided nuclease and a gRNA targeting a target site in a CISH gene locus or a second RNP complex comprising an RNA-guided nuclease and a gRNA targeting a target site in a CISH gene locus.

In another method, Method 188 the present disclosure provides an in vitro method according to Method 187, wherein the gRNA targeting a target site in a CISH gene locus comprises a spacer sequence corresponding to a sequence consisting of any one of SEQ ID NOS: 49-60.

In another method, Method 189 the present disclosure provides an in vitro method according to any one of Methods 176 to 188, further comprising delivering to the cell an RNA-guided nuclease and a gRNA targeting a target site in a FAS gene locus or a second RNP complex comprising an RNA-guided nuclease and a gRNA targeting a target site in a FAS gene locus.

In another method, Method 190 the present disclosure provides an in vitro method according to Method 189, wherein the gRNA targeting a target site in a FAS gene locus comprises a spacer sequence corresponding to a sequence consisting of any one of SEQ ID NOS: 61-67.

In another method, Method 191 the present disclosure provides an in vitro method according to any one of Methods 176 to 190, wherein the cell is a pluripotent stem cell or an adult stem cell.

In another method, Method 192 the present disclosure provides an in vitro method according to Method 191, wherein the cell is an induced pluripotent stem cell or an embryonic stem cell.

In another method, Method 193, the present disclosure provides an in vitro method for generating an engineered cell, the method comprising delivering to a cell: (a) an RNA-guided nuclease and a gRNA targeting a target site in a CIITA gene locus or a first RNP complex comprising an RNA-guided nuclease and a gRNA targeting a target site in a CIITA gene locus; and (b) a vector comprising a nucleic acid, the nucleic acid comprising: (i) nucleotide sequence encoding a SERPINB9 protein and a nucleotide sequence encoding an HLA-E trimer; (ii) a nucleotide sequence having sequence homology with a genomic region located left of the target site in the CIITA gene locus; and (iii) a nucleotide sequence having sequence homology with a genomic region located right of the target site in the CIITA gene locus, wherein (i) is flanked by (ii) and (iii); wherein the CIITA gene locus is cleaved at the target site and the nucleotide sequences encoding the SERPINB9 protein are inserted into the CIITA gene locus, thereby disrupting the CIITA gene.

In another method, Method 194, the present disclosure provides an in vitro method according to Method 193, wherein the gRNA comprises a spacer sequence corresponding to a sequence consisting of SEQ ID NO: 41.

In another method, Method 195, the present disclosure provides an in vitro method according to Method 193 or 194, wherein the nucleotide sequence of (b)(i) comprises the nucleotide sequence encoding the SERPINB9 linked to a nucleotide sequence encoding a P2A peptide sequence linked to the nucleotide sequence encoding the HLA-E trimer to form a SERPINB9-P2A-HLA-E construct.

In another method, Method 196, the present disclosure provides an in vitro method according to Method 195, wherein the SERPINB9-P2A-HLA-E construct consists essentially of SEQ ID NO: 21.

In another method, Method 197, the present disclosure provides an in vitro method according to Method 195 or 196, wherein the SERPINB9-P2A-HLA-E construct is operably linked to an exogenous promoter.

In another method, Method 198, the present disclosure provides an in vitro method according to any one of Methods 193 to 197, wherein the nucleotide sequence of (b)(ii) consists essentially of SEQ ID NO: 42, and the nucleotide sequence of (b)(iii) consists essentially of SEQ ID NO: 43.

In another method, Method 199, the present disclosure provides an in vitro method according to any one of Methods 193 to 198, wherein the first vector consists essentially of SEQ ID NO: 44.

In another method, Method 200, the present disclosure provides an in vitro method according to any one of Methods 193 to 199, further comprising delivering to the cell an RNA-guided nuclease and a gRNA targeting a target site in a CISH gene locus.

In another method, Method 201, the present disclosure provides an in vitro method according to Method 200, wherein the gRNA targeting a target site in a CISH gene locus comprises a spacer sequence corresponding to a sequence consisting of any one of SEQ ID NOS: 49-60.

In another method, Method 202, the present disclosure provides an in vitro method according to any one of Methods 193 to 201, further comprising delivering to the cell an RNA-guided nuclease and a gRNA targeting a target site in a FAS gene locus.

In another method, Method 203, the present disclosure provides an in vitro method according to Method 203, wherein the gRNA targeting a target site in a FAS gene locus comprises a spacer sequence corresponding to a sequence consisting of any one of SEQ ID NOS: 61-67.

In another method, Method 204, the present disclosure provides an in vitro method according to any one of Methods 193 to 203, wherein the cell is a pluripotent stem cell or an adult stem cell.

In another method, Method 205, the present disclosure provides an in vitro method according to Method 204, wherein the cell is induced pluripotent stem cell or an embryonic stem cell.

In another method, Method 205.1, the present disclosure provides an in vitro method for generating an engineered cell, the method comprising delivering to a cell: (a) an RNA-guided nuclease and a gRNA targeting a target site in a B2M gene locus; and (b) a vector comprising a nucleic acid, the nucleic acid comprising: (i) nucleotide sequence encoding a SERPINB9 protein; (ii) a nucleotide sequence having sequence homology with a genomic region located left of the target site in the B2M gene locus; and (iii) a nucleotide sequence having sequence homology with a genomic region located right of the target site in the B2M gene locus, wherein (i) is flanked by (ii) and (iii); wherein the B2M gene locus is cleaved at the target site and the nucleotide sequences encoding the SERPINB9 protein are inserted into the B2M gene locus, thereby disrupting the B2M gene.

In another method, Method 206, the present disclosure provides an in vitro method according to Method 205.1, wherein the gRNA comprises a spacer sequence corresponding to a sequence consisting of SEQ ID NO: 1.

In another method, Method 207, the present disclosure provides an in vitro method of according to Method 205.1, wherein the RNA-guided nuclease and the gRNA targeting a target site in a B2M gene locus are delivered as a ribonucleoprotein (RNP). complex

In another method, Method 208, the present disclosure provides an in vitro method of according to Method 205.1, wherein the nucleotide sequence of (b)(i) further comprises a nucleotide sequence encoding a IL15/IL15Rα fusion protein.

In another method, Method 209, the present disclosure provides an in vitro method of according to Method 205.1, wherein the nucleotide sequence of (b)(i) comprises the nucleotide sequence encoding the SERPINB9 protein linked to a nucleotide sequence encoding a P2A peptide sequence linked to the nucleotide sequence encoding the IL15/IL15Rα fusion protein to form a SERPINB9-P2A-IL15/IL15Rα construct.

In another method, Method 210, the present disclosure provides an in vitro method of according to Method 209, wherein the SERPINB9-P2A-IL15/IL15Rα construct consists essentially of SEQ ID NO: 37.

In another method, Method 211, the present disclosure provides an in vitro method of according to Method 209, wherein the SERPINB9-P2A-IL15/IL15Rα construct is operably linked to an exogenous promoter.

In another method, Method 212, the present disclosure provides an in vitro method of according to Method 211, wherein the exogenous promoter is a CAG, CMV, EF1α, PGK, or UBC promoter.

In another method, Method 213, the present disclosure provides an in vitro method of according to Method 212, wherein the exogenous promoter is CAG and CAG-SERPINB9-P2A-IL15/IL15Rα consists essentially of SEQ ID NO: 38.

In another method, Method 214, the present disclosure provides an in vitro method of according to Method 205.1, wherein the nucleotide sequence of (b)(i) further comprises a nucleotide sequence encoding an HLA-E trimer.

In another method, Method 215, the present disclosure provides an in vitro method of according to Method 214, wherein the nucleotide sequence of (b)(i) comprises the nucleotide sequence encoding the SERPINB9 linked to a nucleotide sequence encoding a P2A peptide sequence linked to the nucleotide sequence encoding the HLA-E trimer to form a SERPINB9-P2A-HLA-E construct.

In another method, Method 216, the present disclosure provides an in vitro method of according to Method 215, wherein the SERPINB9-P2A-HLA-E construct consists essentially of SEQ ID NO: 21.

In another method, Method 217, the present disclosure provides an in vitro method of according to Method 215, wherein the SERPINB9-P2A-HLA-E construct is operably linked to an exogenous promoter.

In another method, Method 218, the present disclosure provides an in vitro method of according to Method 217, wherein the exogenous promoter is a CAG, CMV, EF1α, PGK, or UBC promoter.

In another method, Method 219, the present disclosure provides an in vitro method of according to Method 218, wherein the exogenous promoter is CAG and CAG-SERPINB9-P2A-HLA-E consists essentially of SEQ ID NO: 22

In another method, Method 220, the present disclosure provides an in vitro method of according to Method 205.1, wherein the nucleotide sequence of (b)(ii) consists essentially of SEQ ID NO: 3, and the nucleotide sequence of (b)(iii) consists essentially of SEQ ID NO: 19.

In another method, Method 221, the present disclosure provides an in vitro method of according to Method 205.1, wherein the first vector consists essentially of SEQ ID NO: 39.

In another method, Method 222, the present disclosure provides an in vitro method of according to Method 205.1, wherein the first vector consists essentially of SEQ ID NO: 23.

In another method, Method 223, the present disclosure provides an in vitro method of according to Method 205.1, further comprising delivering to the cell an RNA-guided nuclease and a gRNA targeting a target site in a CISH gene locus.

In another method, Method 224, the present disclosure provides an in vitro method of according to Method 223, wherein the RNA-guided nuclease and gRNA are delivered as a ribonucleoprotein (RNP) complex.

In another method, Method 225, the present disclosure provides an in vitro method of according to Method 223, wherein the gRNA targeting a target site in a CISH gene locus comprises a spacer sequence corresponding to a sequence consisting of any one of SEQ ID NOS: 49-60.

In another method, Method 226, the present disclosure provides an in vitro method of according to Method 205.1, further comprising delivering to the cell an RNA-guided nuclease and a gRNA targeting a target site in a FAS gene locus.

In another method, Method 227, the present disclosure provides an in vitro method of according to Method 226, wherein the RNA-guided nuclease and gRNA are delivered as a ribonucleoprotein (RNP) complex.

In another method, Method 228, the present disclosure provides an in vitro method of according to Method 226, wherein the gRNA targeting a target site in a FAS gene locus comprises a spacer sequence corresponding to a sequence consisting of any one of SEQ ID NOS: 61-67.

In another method, Method 229, the present disclosure provides an in vitro method of according to Method 205.1, wherein the cell is a pluripotent stem cell or an adult stem cell.

In another method, Method 230, the present disclosure provides an in vitro method of according to Method 229, wherein the cell is an induced pluripotent stem cell, or an embryonic stem cell.

In another method, Method 231, the present disclosure provides an in vitro method of according to Method 205.1, wherein the cell is a terminally differentiated somatic cell or a lineage restricted progenitor cell.

In another method, Method 232, the present disclosure provides an in vitro method of according to Method 231, wherein the lineage restricted progenitor cell is hematopoietic progenitor cells, mesodermal cells, definitive hemogenic endothelium, definitive hematopoietic stem or progenitor cells, CD34+ cells, multipotent progenitors (MPP), common lymphoid progenitor cells, T cell progenitors, NK cell progenitors, pancreatic endoderm progenitors, pancreatic endocrine progenitors, mesenchymal progenitor cells, muscle progenitor cells, blast cells, or neural progenitor cells, and the fully differentiated somatic cell is selected from a hematopoietic cell, a pancreatic beta cell, an epithelial cell, an endodermal cell, a macrophages, a hepatocyte, an adipocyte, a kidney cell, a blood cell, a cardiomyocyte, or an immune system cell.

In another method, Method 233, the present disclosure provides an in vitro method of according to Method 205.1, wherein the cell is a mammalian cell.

### EXAMPLES

### Example 1: Cell Maintenance and Expansion

Maintenance of hiPSCs. Human induced pluripotent stem cells (hiPSCs) were maintained in StemFlex Complete (Life Technologies, A3349401) on BIOLAMININ 521 CTG (BioLamina Cat #CT521) or laminin 511 coated tissue culture plates. The cells were fed daily with StemFlex media. For plating of the cells as single cells, the cells were plated with 1% RevitaCell™ Supplement (100×) (ThermoFisher Cat #A2644501) in StemFlex on BIOLAMININ or laminin 511 coated plates. For passaging, 1% REVITACELL™ Supplement (100×) was added.

Single cell cloning of hiPSCs. For single cell cloning, hiPSCs were fed with STEMFLEX™ Complete media (Life Technologies, A3349401) with 1% REVITACELL™ Supplement (100×) (ThermoFisher Cat #A2644501). Following dissociation with ACCUTASE®, the cells were sorted as a single cell per well of a pre-coated plate. The 96 well plates were pre-coated with a 1:10 or a 1:20 dilution of BIOLAMININ 521 LN (LN521) in DPBS, calcium, magnesium (Life Technologies, 14040133) for 2 hours at 37° C. The WOLF FACS-sorter (Nanocellect) was used to sort single cells into the wells. Three days post cell seeding, the cells were fed with fresh STEMFLEX™ and continued to be fed every other day with 100-200 μL of media. After 10 days of growth, the cells were fed daily with STEMFLEX™ until day 12-16. At this time, the plates were dissociated with ACCUTASE® and the collected cell suspensions were split 1:2 with half going into a new 96 well plate for maintenance and half going into a DNA extraction solution QuickExtract™ DNA Extraction Solution (Lucigen). Following DNA extraction, PCR was performed to assess presence or absence of desired gene edits at the targeted DNA locus. Sanger sequencing was used to verify desired knock-out (KO) edits.

Expansion of single cell derived hiPSCs clones. Successfully targeted clones were passaged from 96-well plates to 24-well plates using STEMFLEX™ and BIOLAMININ 521 or Recombinant Laminin iMatrix-511 E8 and 1% REVITACELL™ Supplement (100×). Following expansion in 24-well plates, the cells were passaged onto 6-well plates and then T25 and larger flask formats.

### Example 2: Generation of Human Pluripotent Stem Cells with SERPINB9-P2A-HLA-E Trimer Knock-In and B2M Knock-Out

The SERPINB9-P2A-HLA-E trimer sequence was inserted into a human iPSCs cell line. B2M-2 gRNA (Table 1) was used to facilitate the insertion of the SERPINB9-P2A-HLA-E trimer transgene at the targeted B2M locus.

TABLE 1
B2M gRNA Target Sequence

| | Target Sequence | SEQ | |
|---|---|---|---|
| Name | (5′-3′) | ID NO: | PAM |
| B2M-2 gRNA | GGCCGAGATGTCTCGCTCCG | 1 | TGG |
| (Exon 1_T2) | | | |

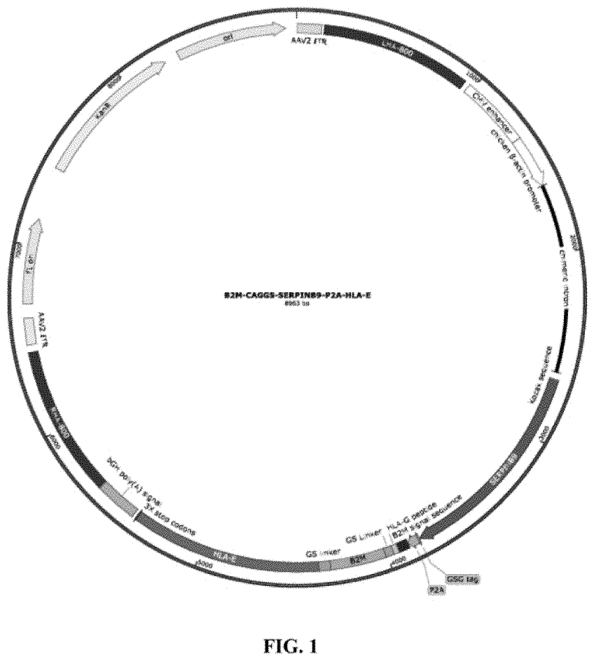

A donor plasmid was designed to insert the SERPINB9-P2A-HLA-E trimer transgene into the B2M locus such that the starting codon of B2M was removed after undergoing homology directed repair (HDR) to insert the transgene, nullifying any chance of partial B2M expression. The SERPINB9 and HLA-E trimer sequences were linked by P2A peptide sequences to allow for expression of two separate proteins encoded from a single transcript. FIG. 1 presents a schematic of the donor plasmid (SEQ ID NO: 23) and Table 2 identifies the elements and locations therein. The donor plasmid comprises the SERPINB9-P2A-HLA-E trimer transgene operably linked to a CAGGS promoter (comprising a CMV enhancer, a chicken 3-actin promoter, and a chimeric intron) flanked by 800 base pair homology arms with sequence identity to the B2M locus around the target site in exon 1. The HLA-E trimer cDNA was composed of a B2M signal peptide fused to an HLA-G presentation peptide fused to the B2M membrane protein fused to the HLA-E protein without its signal peptide. The HLA-E trimer coding sequence (including linkers) is SEQ ID NO: 24 (i.e., SEQ ID NOs: 11, 12, 13, 14, 15, and 16). This HLA-E trimer design has been previously published (Gornalusse et al. (2017) Nat. Biotechnol. 35(8): 765-772). The SERPINB9-P2A-HLA-E coding sequence is SEQ ID NO: 95 (i.e., SEQ ID NOS: 93, 10, 11, 12, 13, 14, 14, and 16).

TABLE 2
Elements of (B2M) SERPINB9-P2A-
HLA-E Trimer Donor Plasmid

| | | |
|---|---|---|
| | Location | SEQ |
| Element | (size in bp) | ID NO: |

| | | | |
|---|---|---|---|
| Left ITR | 1-130 | (130) | 2 |
| LHA-B2M | 145-944 | (800) | 3 |
| CMV enhancer | 973-1352 | (380) | 4 |
| chicken β-actin promoter | 1355-1630 | (276) | 5 |
| chimeric intron | 1631-2639 | (1009) | 6 |
| SERP1NB9 CDS | 2684-3811 | (1128) | 8 |

| | | |
|---|---|---|
| SERPINB9-GSG tag | 2684-3920 | 93 |

| | | | |
|---|---|---|---|
| P2A | 3821-3877 | (57) | 10 |
| B2M signal sequence | 3878-3937 | (60) | 11 |
| HLA-G peptide | 3938-3964 | (27) | 12 |
| GS linker 1 | 3965-4009 | (45) | 13 |
| B2M membrane protein | 4010-4306 | (297) | 14 |
| GS linker 2 | 4307-4366 | (60) | 15 |
| HLA-E CDS | 4367-5377 | (1011) | 16 |
| bGH poly(A) signal | 5404-5628 | (225) | 18 |
| RHA-B2M | 5635-6434 | (800) | 19 |
| Right ITR | 6476-6616 | (141) | 20 |

| | | |
|---|---|---|
| SERPINB9-P2A-HLA-E | 2684-5377 | 95 |
| coding sequence | | |
| SERPINB9-P2A-HLA-E | 2684-5628 | 21 |
| (transgene) | | |
| CAGGS-SERPINB9-P2A- | 973-5628 | 22 |
| HLA-E (promoter-transgene) | | |
| Entire plasmid | (8963) | 23 |

The SERPINB9-P2A-HLA-E trimer donor plasmid was introduced along with a ribonucleoprotein (RNP) complex made up of the B2M targeting gRNA and Cas9 protein. Per 1 million of hiPSC cells, 4 μg of plasmid DNA was delivered along with the RNP via electroporation. Electroporation was carried out in hiPSC cells using the Neon Electroporator with the RNP mixture of Cas9 protein (Biomay) and guide RNA (Biospring) at a molar ratio of 5:1 (gRNA:Cas9) with absolute values of 125 pmol Cas9 and 625 pmol gRNA per 1 million cells. To form the RNP complex, gRNA and Cas9 were combined in one vessel with R-buffer (Neon Transfection Kit) to a total volume of 25-50 μL and incubated for 15 min at room temperature (RT). Cells were dissociated using ACCUTASE®, then resuspended in StemFlex media, counted using an NC-200 (Chemometec) and centrifuged. A total of 2×10⁶ cells were resuspended with the RNP complex and R-buffer was added to a total volume of ˜115 μL. This mixture was then electroporated with 3 pulses for 30 ms at 1100 V. Two electroporations was performed. Following electroporation, the cells were pipetted out into a well of a 6 well plate filled with StemFlex media with RevitaCell and laminin 511. The plates were pre-coated with BIOLAMININ 521 CTG at 1:10 dilution. Cells were cultured in a normoxia incubator (37° C., 8% CO₂).

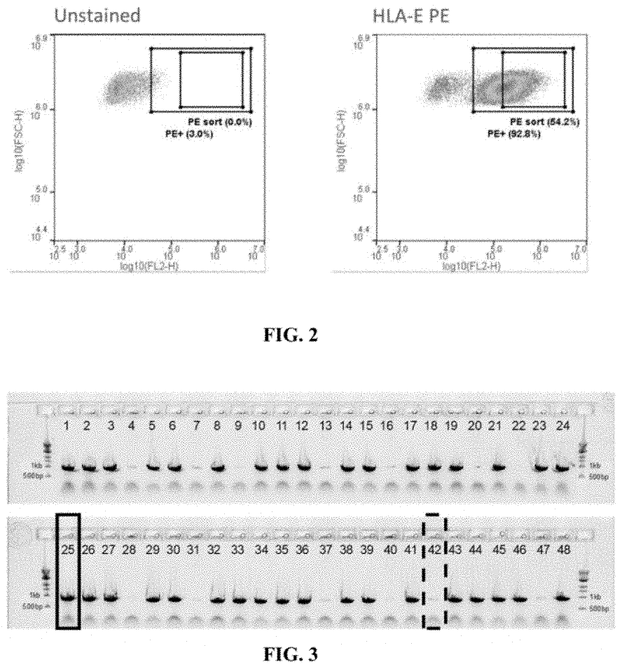

Seven to ten days post electroporation, the cells were enriched for HLA-E trimer expressing cells using an antibody against HLA-E (Table 3) via magnetic assisted cell sorting (MACS) using anti-mouse IgG Dynabeads (ThermoFisher, CELLection™ Pan Mouse IgG Kit, 11531D). These enriched cells represent a bulk KI population of SERPINB9-P2A-HLA-E trimer positive cells. This population was assessed for HLA-E expression by flow cytometry, showing >90% HLA-E expression (FIG. 2).

TABLE 3
Antibodies for Flow Cytometry

| Antigen | Clone | Fluorophore | Manufacturer | Catalog # |
|---|---|---|---|---|
| IL15 | 34559 | PE | ThermoFisher | MA5-23561 |
| B2M | 2M2 | PE | Biolegend | 316305 |
| HLA-ABC | W6/32 | Alexa 488 | Biolegend | 311415 |
| mIgG1 kappa | N/A | PE | BD Bioscience | 555749 |
| PD-L1 | B7-H1 | Alexa-488 | ThermoFisher | 53-5983-42 |
| HLA-E | 3D12 | PE | ThermoFisher | 12-9953-42 |
| HLA-E | 3D12 | APC | ThermoFisher | 17-9953-42 |

Following MACS-enrichment, the cells were single-cell sorted as described in Example 1. The anti-SLA-E-PE antibody (Table 3) was used for FACS-sorting into 96-well plates. For FACS-sorting, unedited cells served as a negative control. After sorting, the cells were expanded as described in Example 1 and when confluent, samples were split for maintenance and genomic DNA extraction.

PCR for the genotyping of the edited clones (SERPINB9-P2A-HLA-E trimer knock-in, B2M Null Human Pluripotent Stem Cells (hPSCs)) was performed and the resulting amplified DNA was assessed for cutting efficiency by TIDE analysis.

For determining SERPINB9-P2A-HLA-E trimer knock-in genotyping in the target B2M sequence, PCR for relevant regions was performed using a 2-step protocol with Platinum Taq Supermix (Invitrogen, cat #125320176 and Cat #11495017). The sequence of the PCR primers are presented in Table 4; and the cycling conditions provided in Table 5.

TABLE 4
B2M KI Primers

| | | Sequence | SEQ |
|---|---|---|---|
| Name | Type | (5′-3′) | ID NO: |
| Poly-A-F | forward | AGGATTGGGAAGACAA | 25 |
| | | TAGCAGGCATGCTGGG | |
| | | GATGCGGTGG | |
| B2M-geno- | reverse | GCTCTGGAGAATCTCA | 26 |
| R1 | | CGCAGAAGGCAGGCGT | |
| | | TTTTCTTAAAAAAAAA | |
| | | TGCACGAATTA | |

TABLE 5
B2M KI PCR Cycling Parameters

| | | | |
|---|---|---|---|
| Step | Temperature | Time | Cycles |

| | | | | | |
|---|---|---|---|---|---|
| Denaturation | 98° | C. | 30 | sec | 1 |
| Denaturation | 98° | C. | 10 | sec | 30 |
| Extension | 72° | C. | 25 | sec | |
| Elongation | 72° | C. | 1 | min | 1 |

| | | | |
|---|---|---|---|
| Hold | 4° | C. | hold |

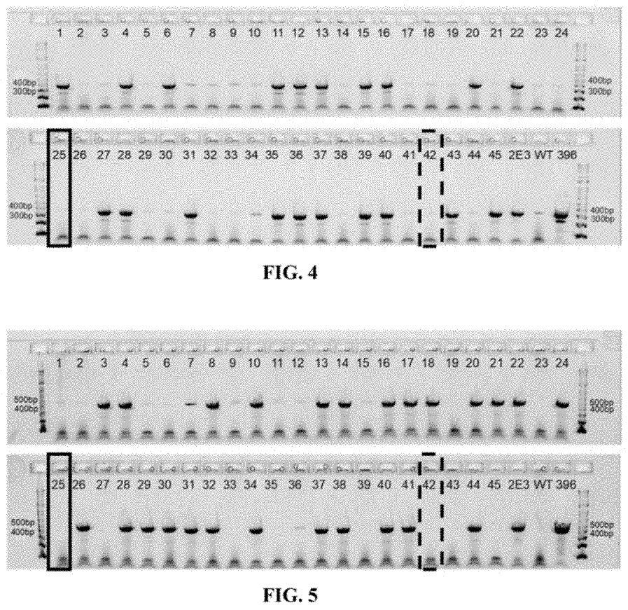

FIG. 3 shows genotyping results of the transgene KI into B2M locus for various edited clones. The presence of a 1.1 kb band indicated successful integration of the KI construct into the B2M locus, while the absence of a band indicated a WT genotype.

For determining the presence of any unwanted bacterial plasmid elements from the KI plasmid, two PCRs were performed using Platinum Taq Supermix (Invitrogen, cat #125320176 and Cat #11495017). The sequences of the PCR primers are presented in Tables 6 and 8; and the cycling conditions provided in Tables 7 and 9.

TABLE 6
Plasmid #1 Primers

| | | Sequence | SEQ |
|---|---|---|---|
| Name | Type | (5′-3′) | ID NO: |
| Ori-F2 | forward | CCCTTAACGTGAGTTT | 27 |
| | | TCGTTCCACTGAGCGT | |
| | | CAGACCCCGTAGAAAA | |
| | | GATCAAAGG | |
| Ori-R | reverse | GTCCAACCCGGTAAGA | 28 |
| | | CACGACTTATCGCCAC | |
| | | TGGCAGCAGCCACTGG | |
| | | TAACAG | |

TABLE 7
Plasmid #1 PCR Cycling Parameters

| | | | |
|---|---|---|---|
| Step | Temperature | Time | Cycles |

| | | | | | |
|---|---|---|---|---|---|
| Denaturation | 98° | C. | 30 | sec | 1 |
| Denaturation | 98° | C. | 10 | sec | 30 |
| Extension | 72° | C. | 10 | sec | |
| Elongation | 72° | C. | 1 | min | 1 |

| | | | |
|---|---|---|---|
| Hold | 4° | C. | hold |

TABLE 8
Plasmid #2 Primers

| | | Sequence | SEQ |
|---|---|---|---|
| Name | Type | (5′-3′) | ID NO: |
| F1-Ori-F | forward | CACTTGCCAGCGCCCTA | 29 |
| | | GCGCCCGCTCCTTTCGC | |
| | | TTTCTTCCCTTCCTTTC | |
| | | TC | |
| F1-Ori-R2 | reverse | GGGCGCGTCAGCGGGTG | 30 |
| | | TTGGCGGGTGTCGGGG | |

TABLE 9
Plasmid #2 PCR Cycling Parameters

| Step | Temperature | Time | | Cycles |
|---|---|---|---|---|
| Denaturation | 98° C. | 30 | sec | 1 |
| Denaturation | 98° C. | 10 | sec | 30 |
| Extension | 72° C. | 10 | sec | |
| Elongation | 72° C. | 1 | min | 1 |

| | | |
|---|---|---|
| Hold | 4 | hold |

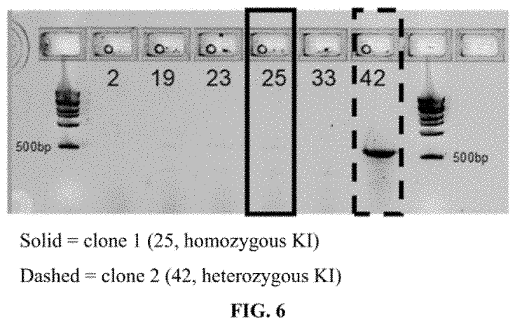

FIG. 4 shows the first PCR amplifying the bacterial plasmid elements that are not supposed to integrate into the genome by HDR because they are outside the homology arms. Both the 5′ and 3′ primers bind outside of the homology arms within the KI plasmid. The presence of a 340 bp band indicates that there is random integration of the plasmid backbone within the genome, clones without bands do not have plasmid insertion.

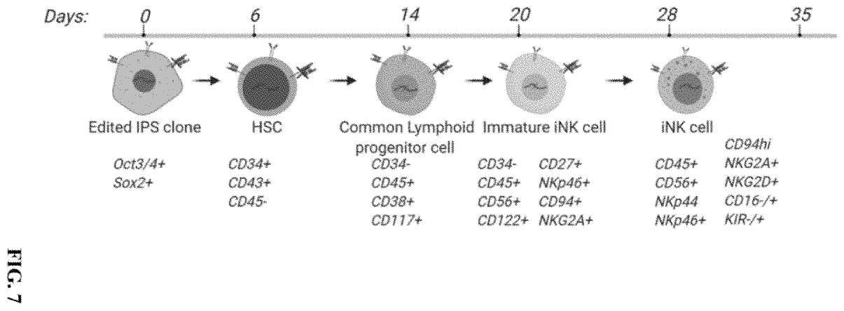

FIG. 5 shows the second PCR amplifying the bacterial plasmid elements outside of the homology arms. The presence of a 476 bp band indicates that there is random integration of the plasmid backbone within the genome, clones without bands do not have plasmid insertion.

For determining indels in the target B2M sequence, PCR for relevant regions was performed using Platinum Taq Supermix (Invitrogen, cat #125320176 and Cat #11495017). The sequences of the PCR primers are presented in Table 10; and the cycling conditions provided in Table 11.

TABLE 10
B2M Indel Primers

| Name | Type | Sequence (5′-3′) | SEQ ID NO: |
|---|---|---|---|
| B2MF2 | Forward | CAGACAGCAAACTCACCCAG | 31 |
| B2MR2 | Reverse | AAACTTTGTCCCGACCCTCC | 32 |

TABLE 11
B2M Indel PCR Cycling Parameters

| | | | |
|---|---|---|---|
| Step | Temperature | Time | Cycles |

| | | | | | |
|---|---|---|---|---|---|
| Denaturation | 94° | C. | 2 | min | 1 |
| Denaturation | 94° | C. | 15 | sec | 30 |
| Annealing | 57° | C. | 30 | sec | |
| Extension | 68° | C. | 45 | sec | |
| Elongation | 68° | C. | 5 | min | 1 |

| | | | |
|---|---|---|---|
| Hold | 4° | C. | hold |

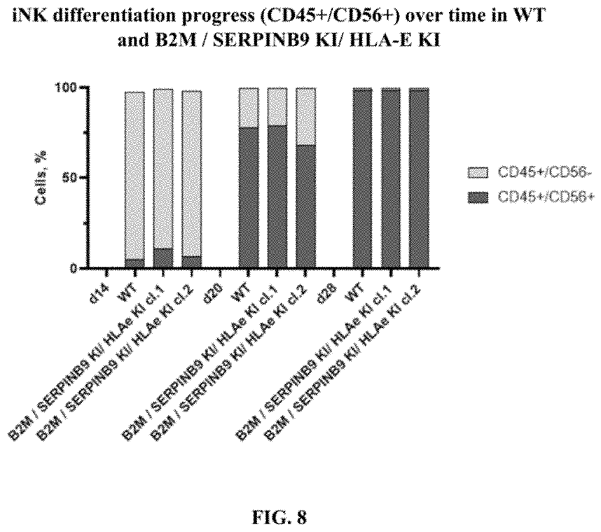

FIG. 6 shows the B2M indel results for various edited clones. The presence of a 573 bp band indicated a WT genotype which would be found in clones that are unedited or are heterozygous for the KI construct, as homozygous clones will not have a band. The B2M KO state of clones was confirmed via PCR and Sanger sequencing. The resulting DNA sequences of the target B2M region were aligned in Snapgene software to determine indel identity and homo- or heterozygosity.

Based on the PCR and Sanger sequencing analysis of the edited clones, the clone shown in lane 25 in FIGS. 3-6 was chosen as “clone 1” and the clone shown in lane 42 was chosen as “clone 2,” which were shown to have the SERPINB9-P2A-HLA-E KI and no bacterial plasmid elements, while the sequencing data confirmed that B2M was completely knocked-out. Clone 1 was homozygous for the KI into B2M while clone 2 was heterozygous for the KI and had an indel of +1T in the B2M WT band. Clones in lanes 2, 19, 23, and 33 were also chosen as “clones 3-6,” respectively, and were confirmed homozygous for the SERPINB9-P2A-HLA-E KI into B2M.

### Example 3: Differentiation of Stem Cells into Natural Killer Cells

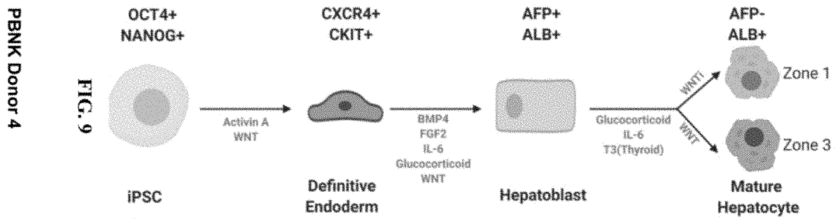

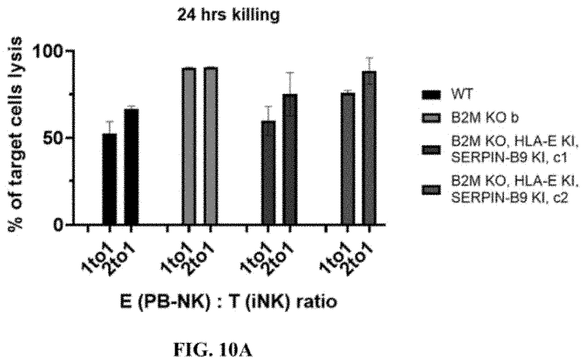

The SERPINB9 KI/HLA-E KI/B2M KO stem cells (clones 1-4) prepared in Example 2, were differentiated into natural killer (NK) cells (iNK cells). FIG. 7 provides a schematic timeline and cell stages of iNK differentiation, as well as the characteristic cell markers at each stage. The iNK differentiation protocol was developed and based on published protocols (see e.g., Ng et al., Nat Protocols 3:768:776 (2008) and U.S. Pat. No. 9,260,696). The iNK cells expressed NK cell markers. FIG. 8 presents an example of CD45⁺/CD56⁺ iNK cells development during IPSC WT and SERPINB9 KI/HLA-E KI/B2M KO lines differentiation to iNK using the iNK differentiation protocol. Listed edits introduced into IPSC did not affect iNK differentiation.

### Example 4: Differentiation of Stem Cells to Hepatocytes

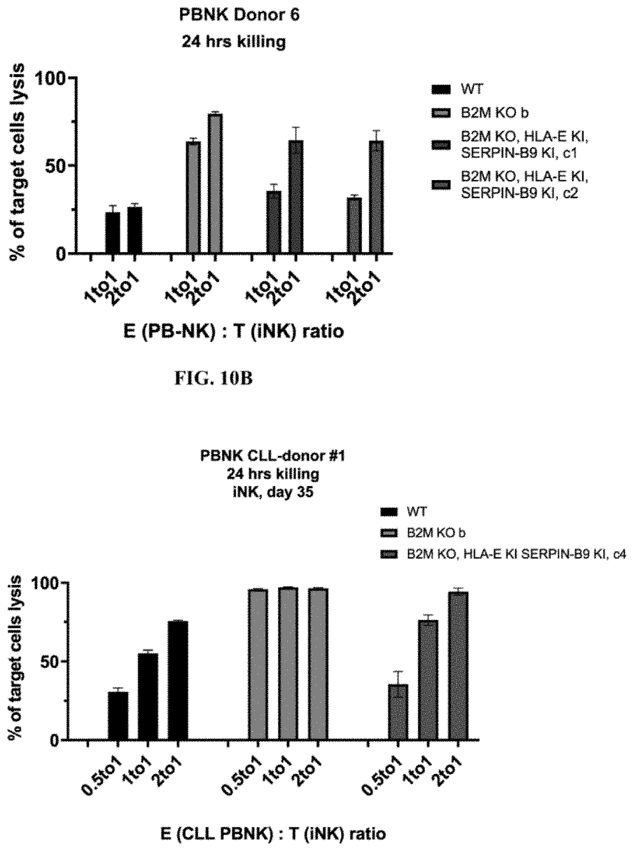

The SERPINB9 KI/HLA-E KI/B2M KO stem cells (clones 1-6) prepared in Example 2, were differentiated into hepatoblasts or hepatocytes using a protocol based upon US 2016/0002595 and WO 2020245747. FIG. 9 presents an overview of the differentiation protocol. The hepatoblasts differentiated from various stem cells expressed AFP and ALB.

### Example 5: SERPINB9 Protects Differentiated Cells from NK Cell Killing

The ability of cells differentiated from the SERPINB9 KI stem cells to survive attack from peripheral blood NK (PB-NK) cells was determined using a luminescent cell viability assay (CellTiter-Glo®, Promega). This assay determines the number of viable cells based on quantitation of the ATP present, which signals the presence of metabolically active cells. After incubation with effector cells, the CellTiter-Glo reagent was added to the target cells and luminescence was measured. The light intensity is linearly related to ATP concentration.

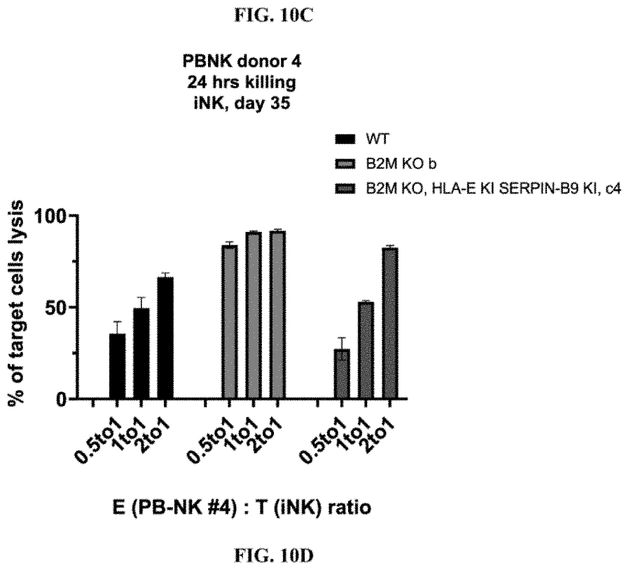

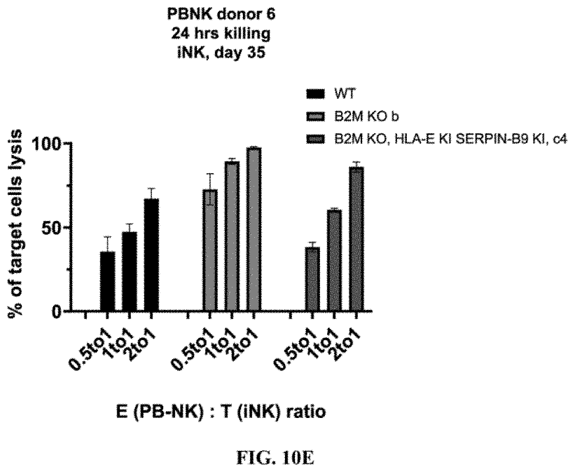

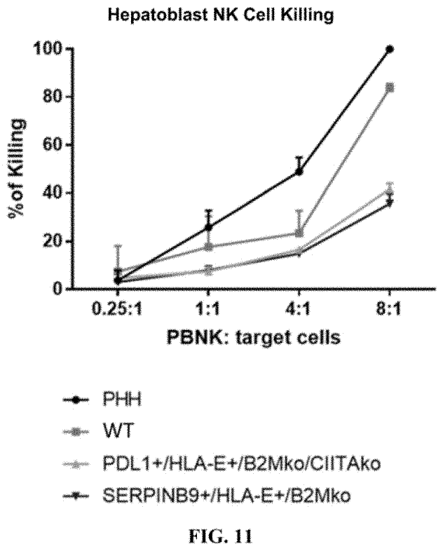

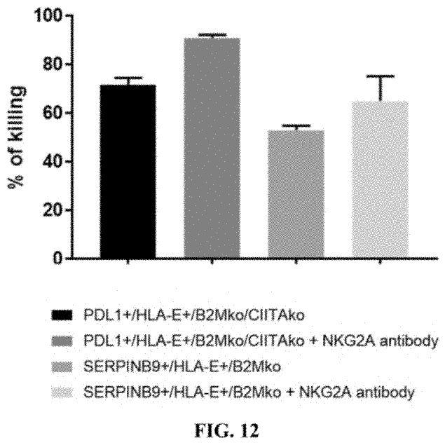

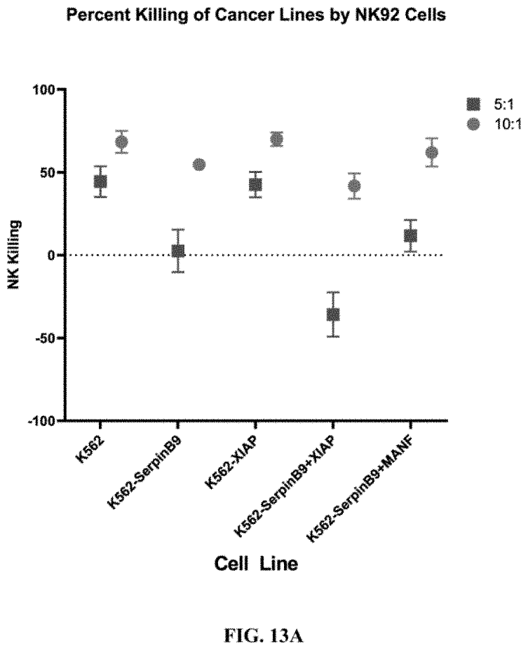

First, the cytotoxicity of PB-NK cells toward iNK cells differentiated from edited iPSCs was examined. PB-NK effector cells derived from several donors were incubated with day 31 iNK target cells (derived from clones 1 and 2) prepared above in Example 3 at E:T ratios of 1:1 or 2:1 for 18-24 hour. Control target cells included iNK derived from wildtype iPSC cells and B2M KO iPSC cells. FIG. 10A and FIG. 10B present the percent of target cell lysis in the presence of PB-NK cells from two different donors, PBNK donor 4 (FIG. 10A) and PBNK donor 6 (FIG. 10B), respectively. The B2M KO/SERPINB9 KI/HLA-E KI provided protection from NK killing as compared to B2M KO alone. FIGS. 10C-10E show the percent of target cell lysis (i.e., day 35 iNK target cells (derived from clone 4) prepared above in Example 3) in the presence of PB-NK cells from 3 different donors, PBNK-CLL-donor #1 (FIG. 10C), PBNK donor 4 (FIG. 10D), and PBNK donor 6 (FIG. 10E), respectively, at E:T ratios of 0.5:1, 1:1 or 2:1 for 24 hours.

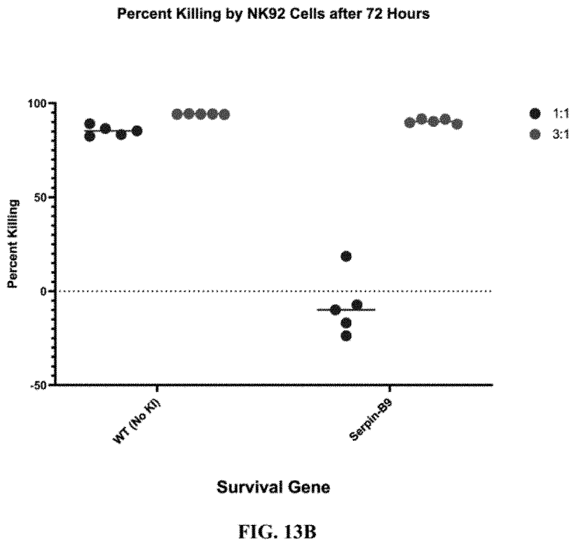

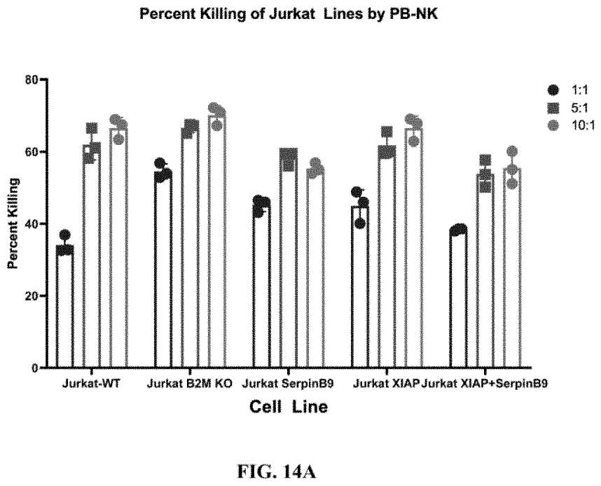

Second, the cytotoxicity of PB-NK cells toward hepatoblasts differentiated from edited iPSCs was examined. PB-NK cells were incubated with hepatoblasts prepared above in Example 4 (i.e., differentiated from the SERPINB9 KI/HLA-E KI/B2M KO cells) at various E:T ratios in a Cell Titer Glo assay, as described above. Control cells included primary human hepatocytes (PHH) and hepatoblasts differentiated from wild type iPSCs. As shown in FIG. 11, the SERPINB9 KI/HLA-E KI/B2M KO hepatoblasts have reduced killing compared to those derived from WT iPSCs. At the highest E:T ratio (8:1) the killing was reduced to 30% as compared to 80% in those derived from WT iPSCs. To determine how much the SERPINB9 KI protected the hepatoblasts from NK killing, the assay was repeated with 1 μL of NKG2A antibodies during the incubation of the target hepatoblasts with the effector PB-NK cells (FIG. 12). The NKG2A antibody was used to block the HLA-E binding to the NKG2A receptor and thus block HLA-E's effectiveness.

### Example 6: SERPINB9 Protects Cancer Cells from NK Cell Killing

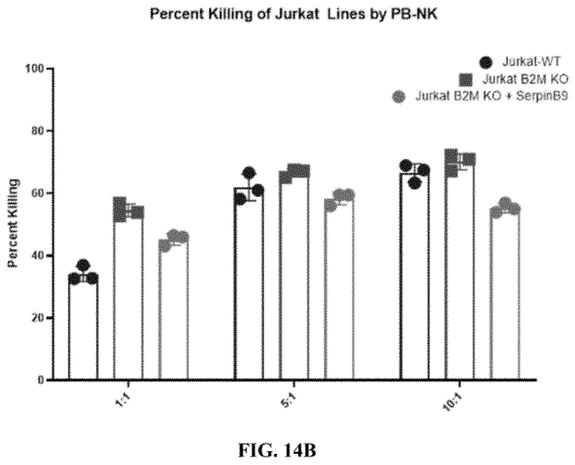

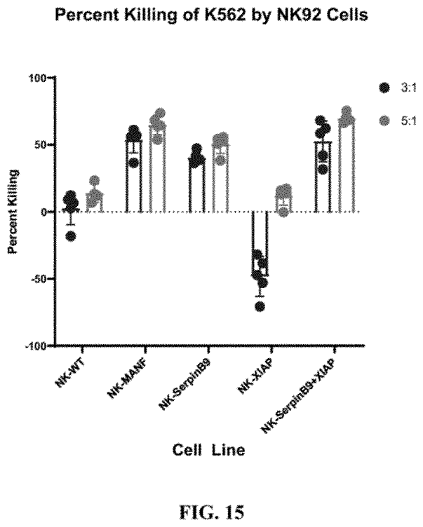

SERPINB9 (alone or in combination with XIAP or MANF) was introduced into K562-luciferase cells, a myelogenous leukemia cell line, using a lentiviral delivery system. Control and modified K562 cells were incubated with NK92 cells for 24 hours and/or 72 hrs, after which luciferase activity was measured. FIG. 13A shows that SERPINB9 and, in particular, the combination of SERPINB9 and XIAP protected the cancer cells from NK-mediated cell killing after 24 hrs. FIG. 13B shows that SERPINB9 protected the K562 cells from NK92 mediated cell killing after 72 hrs.

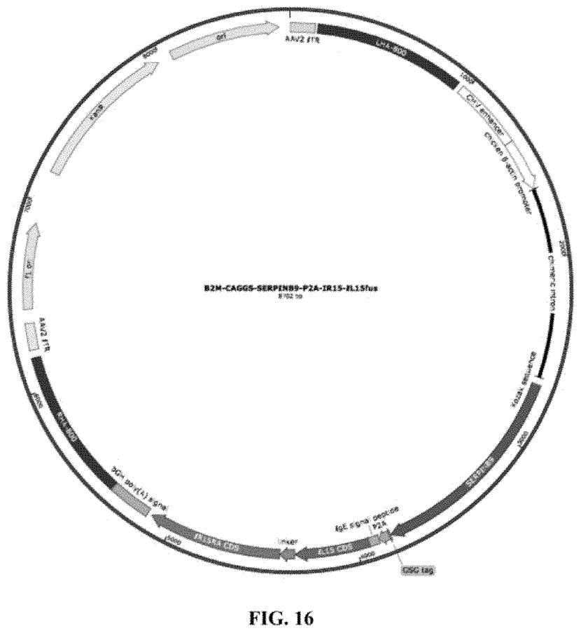

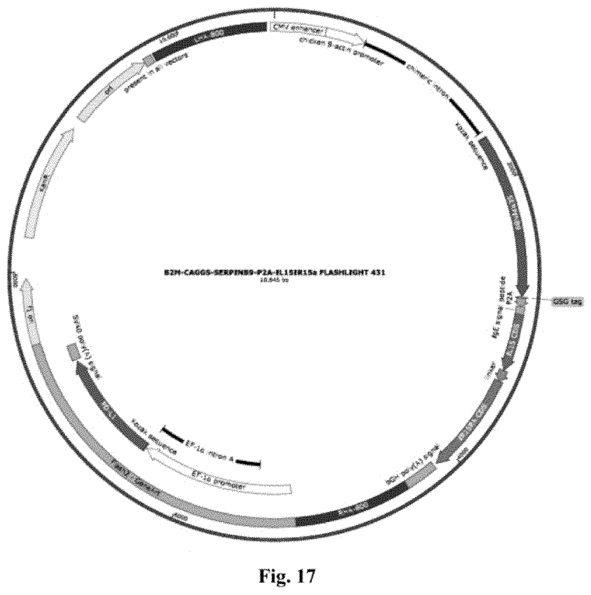

Jurkat cells, an immortalized T cell line, were gene edited to knock-out B2M and then modified further as described above to integrate SERPINB9 and/or XIAP genes into the cells via a lentivirus. Killing by PB-NK cells was evaluated using a cell suspension cell killing assay with WT and the parental B2M KO as controls. As shown in FIGS. 14A and 14B, Jurkat cells containing SERPINB9 (especially in combination with XIAP) were more resistant to PB-NK killing at the higher E:T ratios as compared to the parental B2M KO Jurkat cell line.

### Example 7. SERPINB9 does not Affect Killing Ability of NK Cells

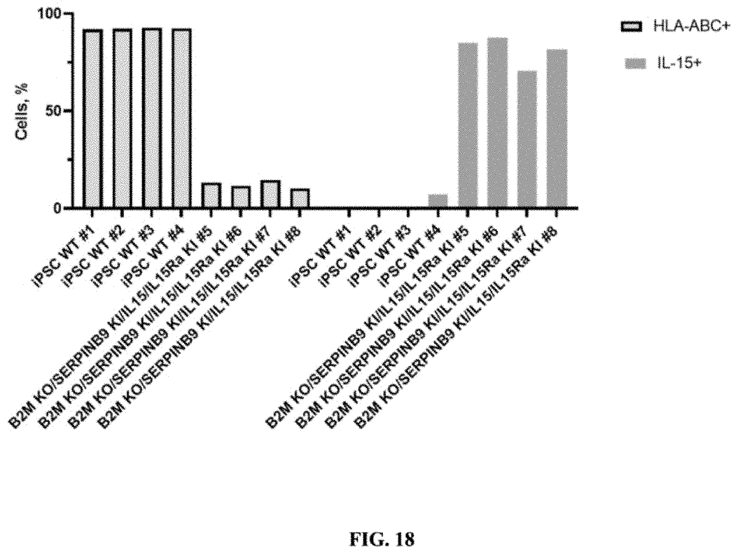

SERPINB9 (and/or XIAP) was transduced into NK92 cells using a lentiviral system. The killing activity of these modified NK cells against K562 cells was monitored with the cell suspension cell killing assay. FIG. 15 shows that SERPINB9 did not affect the killing activity of NK cells.

### Example 8: Generation of Human Pluripotent Stem Cells with SERPINB9-P2A-IL15/IL15Rα Fusion Knock-In and B2M Knock-Out

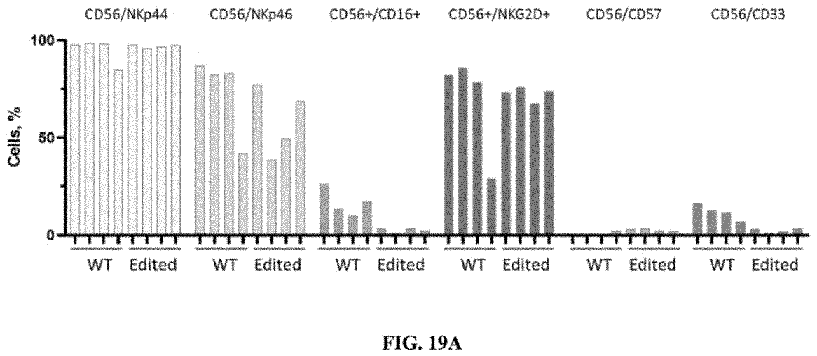

A transgene comprising SERPINB9-P2A-IL15/IL15Rα fusion protein will be inserted in the B2M gene locus of human iPSCs essentially as described above in Example 2. The B2M-2 gRNA (SEQ ID NO: 1) shown in Table 1 will be used. The donor plasmid was designed such that the starting codon of B2M was removed after undergoing homology directed repair to insert the SERPINB9-P2A-IL15/IL15Rα sequence, nullifying any chance of partial B2M expression. FIG. 16 presents a schematic of the plasmid (SEQ ID NO: 39) and Table 12 identifies the elements and locations therein. The donor plasmid contained a CAGGS promoter driven cDNA of SERPINB9-P2A-L15/IL15Rα flanked by 800 base pair homology arms with identical sequence to the B2M locus around exon 1. The IL5/IR1-t fusion protein sequence was designed as previously published (Hurton et al. (2016) Proc Natl Acad Sci USA.; 113(48):E7788-E7797. doi: 10.1073/pnas.1610544113). The IL15/IR15α fusion protein coding sequence (including linkers) is SEQ ID NO: 40 (i.e., SEQ ID NOs: 33, 34, 35, and 36). The SERPINB9-P2A-IL15/IR15α coding sequence is SEQ ID NO: 96 (i.e., SEQ ID NOS: 93, 10, 33, 34, 35, and 35).

TABLE 12
Elements of (B2M) SERPINB9-P2A-IL15/IL15Rα Donor Plasmid

| | Location | | SEQ |
|---|---|---|---|
| Element | (size in bp) | | ID NO: |
| Left ITR | 1-130 | (130) | 2 |
| LHA-B2M | 145-944 | (800) | 3 |
| CMV enhancer | 973-1352 | (380) | 4 |
| chicken β-actin promoter | 1355-1630 | (276) | 5 |
| chimeric intron | 1631-2639 | (1009) | 6 |
| SERPINB9 CDS | 2684-3811 | (1128) | 8 |
| SERPINB9-GSG tag | 2684-3820 | | 93 |
| P2A | 3821-3877 | (57) | 10 |
| IgE signal peptide | 3878-3931 | (54) | 33 |
| IL15 CDS | 3032-4330 | (399) | 34 |
| linker | 4331-4408 | (78) | 35 |
| IR15RA CDS | 4409-5119 | (711) | 36 |
| bGH poly(A) signal | 5143-5367 | (225) | 18 |
| RHA-B2M | 5379-6173 | (800) | 19 |
| Right ITR | 6215-6355 | (141) | 20 |

| | | |
|---|---|---|
| SERPINB9-P2A-IL15/IL15Rα | 2684-5119 | 96 |
| coding sequence | | |
| SERPINB9-P2A-IL15/IL15Rα | 2684-5367 | 37 |
| (transgene) | | |
| CAGGS- SERPINB9-P2A- | 973-5367 | 38 |
| IL15/IL15Rα (promoter-transgene) | | |
| Entire plasmid | (8702) | 39 |

The cells will be electroporated with an RNP comprising Cas9 and B2M-2 gRNA and the donor plasmid, cultured, and characterized as described above in Example 2. After confirmation of the transgene KI and B2M KO, the cells can be edited further, and/or differentiated as described above in Examples 3 and 4.

### Example 9: Generation of Human Pluripotent Stem Cells with SERPINB9-P2A-IL15/IL15Rα Fusion Knock-In and B2M Knock-Out

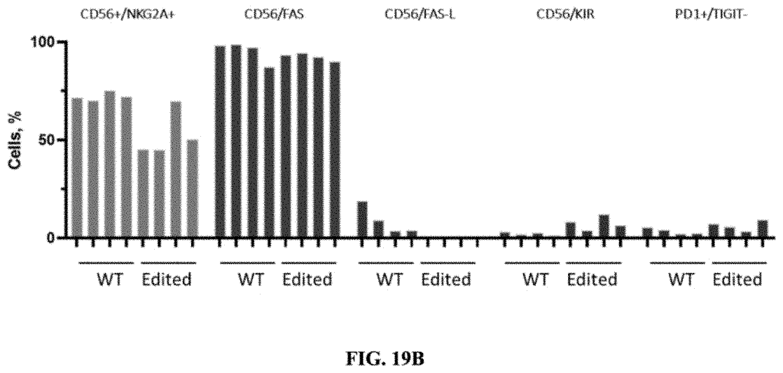

A transgene comprising SERPINB9-P2A-IL15/IL15Rα fusion protein was inserted in the B2M gene locus of human iPSCs essentially as described above. The B2M-2 gRNA (SEQ ID NO: 1) shown in Table 1 was used. The donor plasmid was designed such that the starting codon of B2M was removed after undergoing homology directed repair to insert the SERPINB9-P2A-IL15/IL15Rα sequence, nullifying any chance of partial B2M expression. FIG. 17 presents a schematic of the (flashlight) plasmid (SEQ ID NO: 68) and Table 13 identifies the elements and locations therein. The donor plasmid contained a CAGGS promoter driven cDNA of SERPINB9-P2A-IL15/IL15Rα flanked by 800 base pair homology arms with identical sequence to the B2M locus around exon 1. The IL15/IR15α fusion protein sequence was designed as previously published (Hurton et al. (2016) Proc Natl Acad Sci USA.; 113(48):E7788-E7797. doi: 10.1073/pnas.1610544113). The IL15/IR15α fusion protein coding sequence (including linkers) is SEQ ID NO: 40 (i.e., SEQ ID NOs: 33, 34, 35, and 36). The SERPINB9-P2A-IL15/IR15α coding sequence is SEQ ID NO: 96 (i.e., SEQ ID NOS: 93, 10, 33, 34, 35, and 35). The donor plasmid (SEQ ID NO: 68) also contained sequence encoding PD-L1 (SEQ ID NO: 9) driven by an EF-1 alpha promoter (SEQ ID NO: 7) downstream of the right homology arm for screening and removing cell clones in which the donor plasmid erroneously integrated into the genome.

TABLE 13
Elements of (B2M) SERPINB9-P2A-IL15/IL15Rα
Flashlight Donor Plasmid

| | | |
|---|---|---|
| | Location | SEQ |
| Element | (size in bp) | ID NO: |

| | | | |
|---|---|---|---|
| LHA-B2M | 9791-10590 | (800) | 3 |
| CMV enhancer | 10619-353 | (380) | 4 |
| chicken β-actin promoter | 356-631 | (276) | 5 |
| chimeric intron | 632-1640 | (1009) | 6 |
| SERPINB9 CDS | 1685-2812 | (1128) | 8 |

| | | |
|---|---|---|
| SERPINB9-GSG tag | 1685-2821 | 93 |

| | | | |
|---|---|---|---|
| P2A | 2822-2878 | (57) | 10 |
| IgE signal peptide | 2879-2932 | (54) | 33 |
| IL-15 CDS | 2933-3331 | (399) | 34 |
| linker | 3332-3409 | (78) | 35 |
| IL15Rα CDS | 3410-4120 | (711) | 36 |
| bGH poly(A) signal | 4144-4368 | (225) | 18 |
| RHA-B2M | 4375-5174 | (800) | 19 |
| EF-1α promoter | 5194-6396 | (1203) | 7 |
| PD-L1 | 6412-7284 | (873) | 9 |
| SV40 poly(A) signal | 7302-7423 | (122) | 17 |

| | | |
|---|---|---|
| SERPINB9-P2A- | 1685-4120 | 96 |
| IL15/IL15Rα coding | | |
| sequence | | |
| SERPINB9-P2A- | 1685-4368 | 37 |
| IL15/IL15Rα (transgene) | | |
| CAGGS- SERPINB9- | 10619-4368 | 38 |
| P2A-IL15/IL15Rα | | |
| (promoter-transgene) | | |
| Entire plasmid | 10,645 bp | 68 |

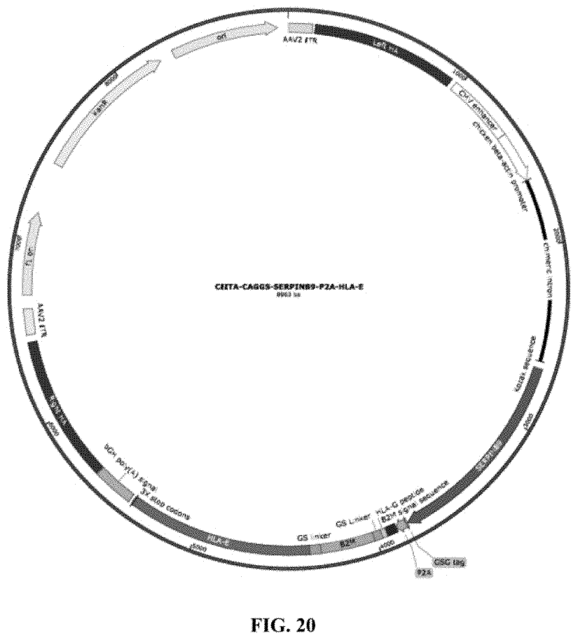

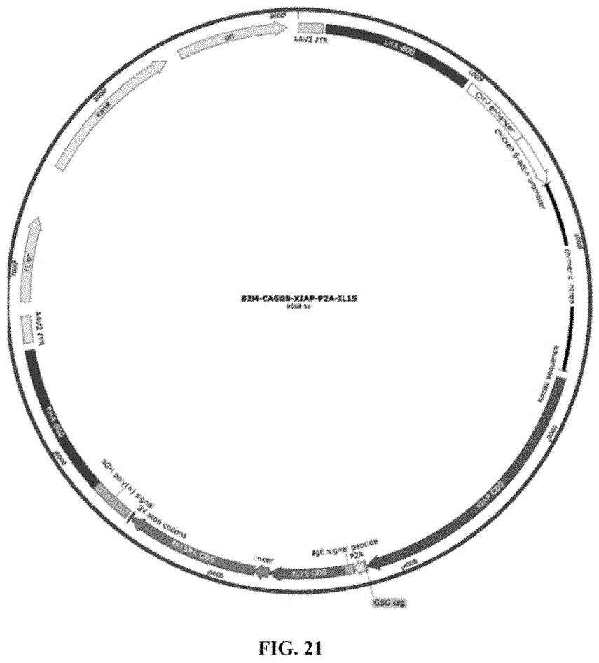

The cells were electroporated with an RNP comprising Cas9 and B32M-2 gRNA and the donor plasmid, cultured, and characterized as described above in Example 2. FIG. 18 shows that the edited cells were effectively edited and maintained in bulk populations. The bulk population of edited cells were differentiated, essentially as described in Example 3. iNK biomarkers were measured on Day 28 (FIGS. 19A and 19B). In a cell killing assay, day 28 and 35 iNK cells had high level of cytotoxicity against K562 cells (4 hr incubation).

After confirmation of the transgene KI and B2M KO, the cells were further edited to have CISH KO (CISH Ex1 T18; SEQ ID NO: 50) and FAS KO (FAS Ex 1 T9; SEQ ID NO: 62), and/or differentiated as described above in Examples 3 and 4.

### Example 10: Generation of Human Pluripotent Stem Cells with SERPINB9-P2A-HLA-E Trimer Knock-In and CIITA Knock-Out

Human iPSCs will be edited to insert a transgene comprising SERPINB9-P2A-HLA-E trimer into the CIITA gene locus, thereby eliminating expression of the CIITA gene. The protocol will be similar to that described above in Example 2. CIITA Ex3_T6 gRNA (Table 14) will be used to target the CIITA gene for insertion of the SERPINB9-P2A-HLA-E trimer transgene. This gRNA was chosen because if its high on-target activity and undetectable off-target activity.

TABLE 14
CIITA gRNA Target Sequence

| | Target Sequence | SEQ | |
|---|---|---|---|
| Nam | (5′-3′) | ID NO: | PAM |
| CIITA Ex3_T6 | GGTCCATCTGGTCATAGAAG | 41 | TGG |

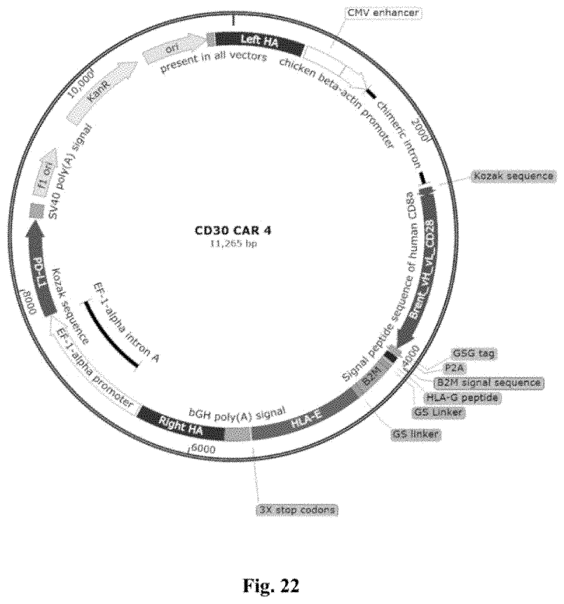

The donor plasmid comprising SERPINB9-P2A-HLA-E was similar to that detailed above in Table 2, except the 800 bp homology arms flanking the transgene are identical to sequence at the CIITA target site. A schematic of the plasmid (SEQ ID NO: 44) and is presented in FIG. 20 and elements and locations therein of the plasmid are shown in Table 15. The SERPINB9-P2A-HLA-E coding sequence is SEQ ID NO: 95 (i.e., SEQ ID NOS: 93, 10, 11, 12, 13, 14, 15, and 16).

TABLE 15
Elements of (CIITA) SERPINB9-
P2A-HLA-E Trimer Donor Plasmid

| | | |
|---|---|---|
| | Location | SEQ |
| Element | (size in bp) | ID NO: |

| | | | |
|---|---|---|---|
| Left ITR | 1-130 | (130) | 2 |
| LHA-CIITA | 145-944 | (800) | 42 |
| CMV enhancer | 973-1352 | (380) | 4 |
| chicken β-actin promoter | 1355-1630 | (276) | 5 |
| chimeric intron | 1631-2639 | (1009) | 6 |
| SERPINB9 CDS | 2684-3811 | (1128) | 8 |

| | | |
|---|---|---|
| SERPINB9-GSG tag | 2684-3820 | 93 |

| | | | |
|---|---|---|---|
| P2A | 3821-3877 | (57) | 10 |
| B2M signal sequence | 3878-3937 | (60) | 11 |
| HLA-G peptide | 3938-3964 | (27) | 12 |
| GS linker 1 | 3965-4009 | (45) | 13 |
| B2M | 4010-4306 | (297) | 14 |
| GS linker 2 | 4307-4366 | (60) | 15 |
| HLA-E CDS | 4367-5377 | (1011) | 16 |
| bGH poly(A) signal | 5404-5628 | (225) | 18 |
| RHA-CIITA | 5635-6434 | (800) | 43 |
| Right ITR | 6476-6616 | (141) | 20 |

| | | |
|---|---|---|
| SERPINB9-P2A-HLA-E | 2684-6377 | 95 |
| coding sequence | | |
| SERPINB9-P2A-HLA-E | 2684-5628 | 21 |
| (transgene) | | |
| CAGGS- SERPINB9-P2A- | 973-5628 | 22 |
| HLA-E (promoter- | | |
| transgene) | | |
| Entire plasmid | (8963) | 44 |

The cells will be electroporated with an RNP comprising Cas9 and CIITA Ex3_T6 gRNA (SEQ ID NO: 41) and the donor plasmid, cultured, and characterized as described above in Example 2. After confirmation of the transgene KI and B32M KG, the cells can be edited further (e.g., see next paragraph), and/or differentiated as described above in Examples 3 and 4.

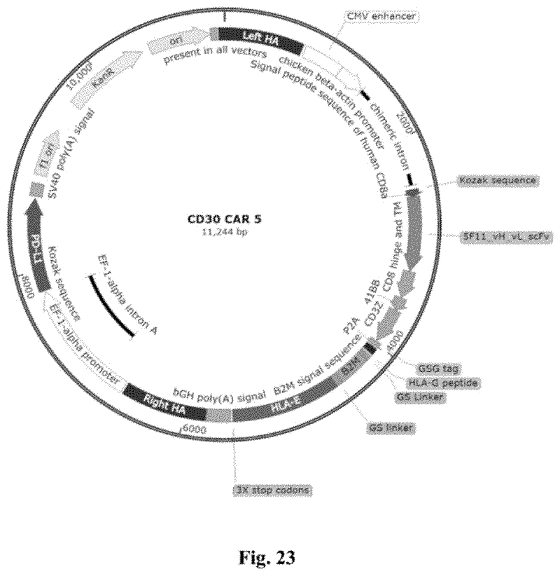

In some instances, the SERPINB9 KI/HLA-E KI/CIITA KG cells will be edited to insert a XIAP-IL15/IL15Rα transgene into the B32M gene locus. For this, the B32M-2 gRNA (SEQ ID NO: 1) detailed above in Table 1 will be used along with a donor plasmid comprising the XIAP-P2A-IL15/IL15Rα transgene. As detailed above in Example 8, the IL15/IR15α coding sequence is SEQ ID NO: 40. The XIAP-P2A-IL15/IL15Rα coding sequence is SEQ ID NO: 97 (i.e, SEQ ID NOS: 94, 10, 33, 34, 35, and 36). The coding sequence is operably linked to a CAGGS promoter and is flanked by 800 base pair homology arms that are identical to sequence around the B2M target site. FIG. 21 presents a schematic of the plasmid (SEQ ID NO: 48) and Table 16 identifies the elements and locations therein.

TABLE 16
Elements of (B2M) XIAP-P2A-IL15/IL15Rα Donor Plasmid

| | | |
|---|---|---|
| | Location | SEQ |
| Element | (size in bp) | ID NO: |

| | | | |
|---|---|---|---|
| Left ITR | 1-130 | (130) | 2 |
| LHA-B2M | 145-944 | (800) | 3 |
| CMV enhancer | 973-1352 | (380) | 4 |
| chicken β-actin promoter | 1355-1630 | (276) | 5 |
| chimeric intron | 1631-2639 | (1009) | 6 |
| XIAP CDS | 2684-4174 | (1491) | 45 |

| | | |
|---|---|---|
| XIAP-GSG tag | 2684-4183 | 94 |

| | | | |
|---|---|---|---|
| P2A | 4184-4240 | (57) | 10 |
| IgE signal peptide | 4241-4294 | (54) | 33 |
| IL15 CDS | 4295-4693 | (399) | 34 |
| linker | 4694-4771 | (78) | 35 |
| IL15Rα CDS | 4772-5482 | (711) | 36 |
| bGH poly(A) signal | 5509-5733 | (225) | 18 |
| RHA-B2M | 5740-6539 | (800) | 19 |
| Right ITR | 6581-6721 | (141) | 20 |

| | | |
|---|---|---|
| XIAP-P2A-IL15/IL15Rα | 2684-5483 | 97 |
| coding sequence | | |
| XIAP-P2A-IL15/IL15Rα | 2684-5733 | 46 |
| (transgene) | | |
| CAGGS- XIAP-P2A- | 973-5733 | 47 |
| IL15/IL15Rα (promoter- | | |
| transgene) | | |
| Entire plasmid | (9068) | 48 |

### Example 11: Generation of Anti-CD30 CAR-P2A-HLA-E Trimer Knock-In, CIITA Null Human Pluripotent Stem Cells

Plasmids were designed to insert an anti-CD30 CAR-P2A-HLA-E trimer into the CIITA gene locus essentially as described above in Example 9 (i.e., 86 bp of the CIITA exon 2 would be removed after undergoing HDR). Each donor plasmid contained a CAGGS promoter operably linked to a cDNA of an anti-CD30 CAR-P2A-HLA-E trimer flanked by 800 base pair homology arms with identical sequence to the CIITA gene locus around exon 2. The HLA-E trimer cDNA was composed of a B2M signal peptide fused to an HLA-G presentation peptide fused to the B2M membrane protein fused to the HLA-E protein without its signal peptide. The HLA-E trimer coding sequence (including linkers) is SEQ ID NO: 24 (i.e., SEQ ID NOs: 11, 12, 13, 14, 15, and 16). The P2A peptide sequence (SEQ ID NO: 10) connecting the anti-CD30 CAR and the HLA-E trimer allows for the separate expression of both proteins from the single mRNA. Each donor plasmid also contained a PD-L1 coding sequence (SEQ ID NO: 9) operably linked to an EF-1 alpha promoter (SEQ ID NO: 92) downstream of the right homology arm sequence (SEQ ID NO: 43) such that PD-L1 would be expressed if the plasmid integrated into the genome. Probes spanning the plasmid backbone can be used to detect plasmid integration using ddPCR. FACS with an anti-PD-L1 antibody can be used to remove PD-L1 positive cells.

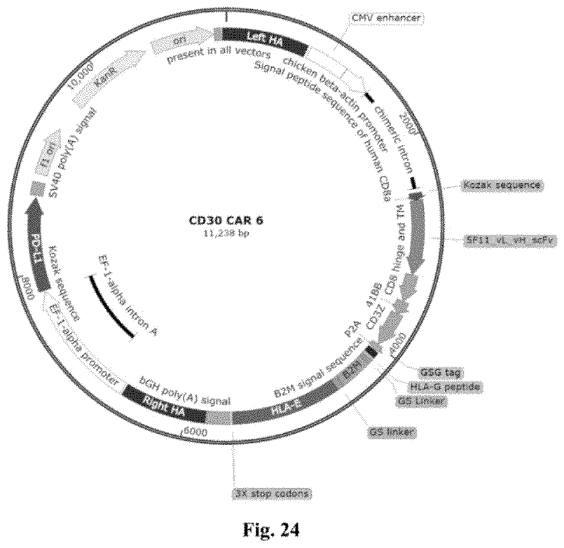

FIG. 22 presents a schematic of an anti-CD30 CAR 4-P2A-HLA-E encoding plasmid (SEQ ID NO: 77) and Table 17 identifies the elements and locations therein. The anti-CD30 CAR 4 coding sequence is SEQ ID NO: 74 (i.e., SEQ ID NOS: 69, 70, 71, 72, and 73) and the anti-CD30 CAR 4 amino acid sequence is SEQ ID NO: 78. The anti-CD30 CAR 4-P2A-HLA-E coding sequence is SEQ ID NO:98 (i.e., SEQ ID NOS: 69, 70, 71, 72, 73, 10, 11, 12, 13, 14, 15, and 16).

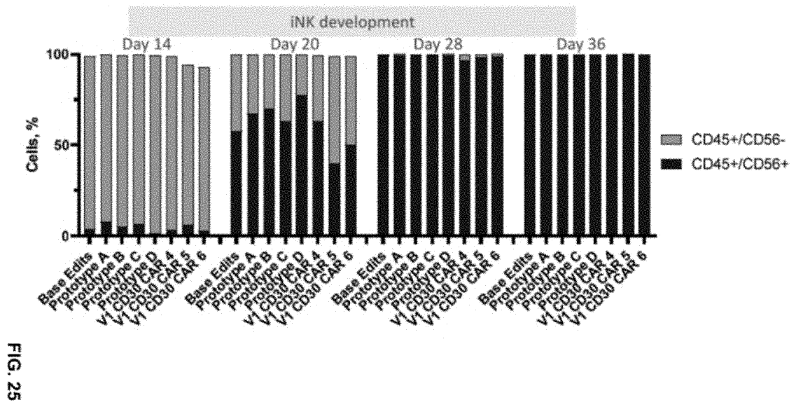

FIG. 23 presents a schematic of an anti-CD30 CAR 5-P2A-HLA-E encoding plasmid (SEQ ID NO:84) and Table 18 identifies the elements and locations therein. The anti-CD30 CAR 5 coding sequence is SEQ ID NO: 81 (i.e., SEQ ID NOS: 69, 79, 71, 80, and 73) and the anti-CD30 CAR 4 amino acid sequence is SEQ ID NO: 85. The anti-CD30 CAR 5-P2A-HLA-E coding sequence is SEQ ID NO: 99 (i.e., SEQ ID NOS: 69, 79, 71, 80, 73, 10, 11, 12, 13, 14, 15, and 16).

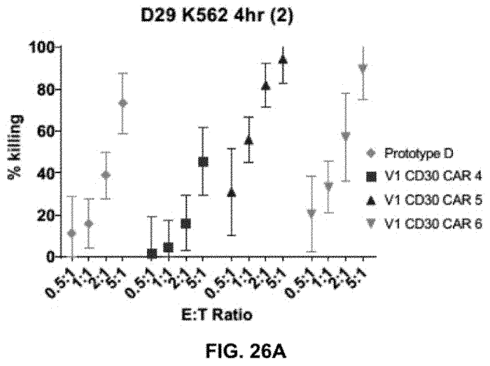

FIG. 24 presents a schematic of an anti-CD30 CAR 6-P2A-HLA-E encoding plasmid (SEQ ID NO: 90) and Table 19 identifies the elements and locations therein. The anti-CD30 CAR 6 coding sequence is SEQ ID NO: 87 (i.e., SEQ ID NOS: 69, 86, 71, 80, and 73) and the anti-CD30 CAR 4 amino acid sequence is SEQ ID NO: 91. The anti-CD30 CAR 5-P2A-LA-E coding sequence is SEQ ID NO: 99 (i.e., SEQ ID NOS: 69, 79, 71, 80, 73, 10, 11, 12, 13, 14, 15, and 16). The anti-CD3 CAR 6-P2A-LA-E coding sequence is SEQ ID NO: 100 (i.e., SEQ ID NOS: 69, 86, 71, 80, 73, 10, 11, 12, 13, 14, 15, and 16).

TABLE 17
Elements of anti-CD30 CAR 4-P2A-HLA-E Donor Plasmid

| | | |
|---|---|---|
| | Location | SEQ |
| Element | (size in bp) | ID NO: |

| | | | |
|---|---|---|---|
| LHA-CIITA | 11,107-641 | (800) | 42 |
| CMV enhancer | 670-1049 | (380) | 4 |
| chicken β-actin promoter | 1052-1327 | (276) | 5 |
| chimeric intron | 1328-2336 | (1009) | 6 |
| CD8a signal peptide | 2381-2443 | (63) | 69 |
| Brent_vH_vL | 2444-3172 | (729) | 70 |
| CD8TM | 3173-3436 | (264) | 71 |
| CD28 domain | 3437-3556 | (120) | 72 |
| CD3Z domain | 3557-3892 | (336) | 73 |
| P2A | 3902-3958 | (57) | 10 |
| B2M signal sequence | 3959-4018 | (60) | 11 |
| HLA-G peptide | 4019-4045 | (27) | 12 |
| GS linker | 4046-4090 | (45) | 13 |
| B2M | 4091-4387 | (297) | 14 |
| GS linker 2 | 4388-4447 | (60) | 15 |
| HLA-E CDS | 4448-5458 | (1011) | 16 |
| bGH poly(A) signal | 5485-5709 | (225) | 18 |
| RHA-CIITA | 5716-6515 | (800) | 43 |
| EF-1 alpha promoter | 6535-7712 | (1178) | 92 |
| PD-L1 CDS | 7728-8600 | (873) | 9 |
| SV40 poly(A) sequence | 8618-8739 | (122) | 17 |

| | | |
|---|---|---|
| anti-CD30 CAR 4 coding | 2381-3892 | 74 |
| sequence | | |
| anti-CD30-CAR-4-P2A- | 2381-5458 | 98 |
| HLA-E | | |
| anti-CD30-CAR-4-P2A- | 2381-5709 | 75 |
| HLA-E (transgene | | |
| CAGGS-CAR-4-P2A-HLA | 670-5709 | 76 |
| (promoter-transgene) | | |
| Total plasmid | 11,265 bp | 77 |

TABLE 18
Elements of anti-CD30 CAR 5-P2A-HLA-E Donor Plasmid

| | | |
|---|---|---|
| | Location | SEQ |
| Element | (size in bp) | ID NO: |

| | | | |
|---|---|---|---|
| LHA-CIITA | 11,205-766 | (800) | 42 |
| CMV enhancer | 774-1153 | (380) | 4 |
| chicken β-actin promoter | 1156-1431 | (276) | 5 |
| chimeric intron | 1432-2440 | (1009) | 6 |
| CD8a signal peptide | 2485-2547 | (63) | 69 |
| 5F11_vH_vL | 2548-3249 | (702) | 79 |
| CD8TM | 3250-3513 | (264) | 71 |
| 41BB co-stim domain | 3514-3639 | (126) | 80 |
| CD3Z domain | 3640-3975 | (336) | 73 |
| P2A | 3985-4041 | (57) | 10 |
| B2M signal sequence | 4042-4101 | (60) | 11 |
| HLA-G peptide | 4102-4128 | (27) | 12 |
| GS linker | 4129-4173 | (45) | 13 |
| B2M | 4174-4470 | (297) | 14 |
| GS linker 2 | 4471-4530 | (60) | 15 |
| HLA-E | 4531-5541 | (1011) | 16 |
| bGH poly(A) signal | 5568-5792 | (225) | 18 |
| RHA-CIITA | 5799-6598 | (800) | 43 |
| EF-1 alpha promoter | 6618-7795 | (1173) | 92 |
| PD-L1 CDS | 7811-8683 | (873) | 9 |
| SV40 poly(A) sequence | 8701-8822 | (122) | 17 |

| | | |
|---|---|---|
| anti-CD30 CAR 5 coding | 2485-3975 | 81 |
| sequence | | |
| Anti-CD30-CAR5-P2A- | 2485-5541 | 99 |
| HLA-E coding sequence | | |
| Anti-CD30-CAR5-P2A- | 2485-5792 | 82 |
| HLA-E (transgene) | | |
| CAGGS- Anti-CD30-CAR5- | 774-5783 | 83 |
| P2A-HLA-E (promoter- | | |
| transgene) | | |
| Total plasmid | 12,224 | 84 |

TABLE 19
Elements of anti-CD30 CAR 6-P2A-HLA-E Donor Plasmid

| | | |
|---|---|---|
| | Location | SEQ |
| Element | (size in bp) | ID NO: |

| | | | |
|---|---|---|---|
| LHA-CIITA | 11,205-766 | (800) | 42 |
| CMV enhancer | 795-1174 | (380) | 4 |
| chicken β-actin promoter | 1177-1452 | (276) | 5 |
| chimeric intron | 1453-2461 | (1009) | 6 |
| CD8a signal peptide | 2500-2568 | (63) | 69 |
| 5F11_vL_vH | 2569-3270 | (700) | 86 |
| CD8TM | 3271-3528 | (264) | 71 |
| 41BB co-stim domain | 3529-3654 | (126) | 80 |
| CD3Z domain | 3655-3990 | (336) | 73 |
| P2A | 4000-4056 | (57) | 10 |
| B2M signal sequence | 4057-4116 | (60) | 11 |
| HLA-G peptide | 4117-4143 | (27) | 12 |
| GS linker | 4144-4188 | (45) | 13 |
| B2M | 4189-4485 | (297) | 14 |
| GS linker2 | 4486-4545 | (60) | 15 |
| HLA-E | 4546-5556 | (1011) | 16 |
| bGH poly(A) signal | 5583-5807 | (225) | 18 |
| RHA-CIITA | 5814-6613 | (800) | 43 |
| EF-1 alpha promoter | 6633-7810 | (1178) | 92 |
| PD-L1 CDS | 7826-8698 | (873) | 9 |
| SV40 poly(A) signal | 8716-8837 | (122) | 17 |

| | | |
|---|---|---|
| anti-CD30 CAR 6 coding | 2500-3990 | 87 |
| sequence | | |
| Anti-CD30-CAR6-P2A- | 2500-5556 | 100 |
| HLA-E coding sequence | | |
| Anti-CD30-CAR6-P2A- | 2500-5807 | 88 |
| HLA-E (transgene) | | |
| CAGGS- Anti-CD30-CAR6- | 795-5807 | 89 |
| P2A-HLA-E (promoter- | | |
| transgene) | | |
| Total plasmid | 11,238 bp | 90 |

The CIITA-T6 gRNA (Table 14) was used to facilitate insertion of the anti-CD30 CAR transgenes at the targeted CIITA gene locus. The target sequence of CIITA-T6 is not present in the donor plasmid and thus the donor plasmid itself would not be targeted by this gRNA. CIITA-T6 induced CRISPR cutting in the human genome at exon 2 of CIITA would lead to a frameshift and loss of CIITA protein. Each CD30 CAR donor plasmid was introduced along with a RNP complex made up of the CIITA targeting gRNA and Cas9 protein. Per 1 million of human embryonic stem cells, 2 μg of plasmid DNA was delivered along with the RNP via electroporation. Electroporation was carried out using the Neon Electroporator with the RNP mixture of Cas9 protein and guide RNA at a molar ratio of 1:5 with absolute values of 125 pmol Cas9 and 625 pmol gRNA per 2 million cells. To form the RNP complex, gRNA and Cas9 were combined in one vessel with R-buffer (Neon Transfection Kit) to a total volume of 25-50 μL and incubated for 15 min at room temperature (RT). Cells were dissociated using ACCUTASE®, then resuspended in STEMFLEX™ media, counted using an NC-200 (ChemoMetec) and centrifuged. A total of 2×10⁶ cells were resuspended with the RNP complex and R-buffer was added to a total volume of 115 μL. This mixture was then electroporated with 3 pulses for 30 ms at 1000 V. Following electroporation, the cells were pipetted out into a well of a 6 well plate filled with STEMFLEX™ media with REVITACELL™ Supplement (100×) and BIOLAMININ 521 CTG at 1:10 dilution. Cells were cultured in a normoxia incubator (37° C., 8% CO₂).

At 2 days post electroporation, the cells were enriched for transfection via fluorescence activated cell sorting (FACS) using an antibody against HLA-E (see Table 3). Plasmid integration analysis revealed that 1/46 cell clones was free of integrated plasmid. However, if PD-L1 positive cells were removed prior to the cell sorting, 24/82 cell clones were plasmid free. Thus, FACS was performed using PD-L1 negative cells. Seven to ten days post electroporation, the cells were again enriched for HLA-E trimer knock in cells using FACS. These enriched cells represent bulk KI population of anti-CD30 CAR-P2A-HLA-E trimer positive cells. PCR for the genotyping of the edited clones was performed and the resulting amplified DNA was assessed for cutting efficiency by TIDE analysis.

### Example 12: Generation of Human Pluripotent Stem Cells with SERPINB9-P2A-IL15/IL15Rα Fusion Knock-In and B2M Knock-Out, Anti-CD30 CAR-P2A-HLA-E Trimer Knock-In and CIITA Knock-Out, CISH Knock-Out, and Fas Knock-Out

iPSC cells were generated to have SERPINB9-P2A-IL15/IR15α KI and B2M KO, anti-CD30 CAR-P2A-HLA-E KI and CIITA KO, as well as CISH KO and FAS KO, generally as described in Examples 9 and 11, with modifications.

First, SERPINB9-P2A-IL15/IR15α was knocked into the cells using the SERPINB9-P2A-IL15/IR15α plasmid (SEQ ID NO: 68) and the B2M-T2 gRNA (SEQ ID NO: 1). The iPSCs were passaged the day before electroporation and seeded as 10 million cells per T75 flask. On day of electroporation, the cells were split again and electroporated using the Neon Electroporator with the RNP mixture of Cas9 protein (Biomay) and guide RNA (IDT) at a molar ratio of 5:1 (gRNA:Cas9) with absolute values of 625 pmol gRNA and 125 pmol Cas9 per 2 million cells. To form the RNP complex, gRNA and Cas9 were combined in one vessel with R-buffer (Neon Transfection Kit) to a total volume of 25-50 μL and incubated for 15 min at room temperature (RT). This mixture was then combined with the cells to a total volume of ˜115 μL using R-buffer. This mixture was then electroporated with 3 pulses for 30 ms at 1000 V. Following electroporation, the cells were pipetted out into a 6 well plate filled with STEMFLEX™ media with REVITACELL™ Supplement (100×) and BIOLAMININ 521 CTG at 1:10 dilution. Cells were cultured in a normoxia incubator (37° C., 8% CO₂).

On day 2 post electroporation, the PD-L1 negative cells were FACS-sorted (FACS #1) for IL15 positive cells to enrich for transfected cells. At 7 to 10 days post electroporation, the cells were FACS-sorted (FACS #2) again for IL15+ cells to enrich for knock in positive cells (e.g., L5V018B cells). The cells were allowed to expand, and then FAS was knocked out using the FAS Ex1 T9 gRNA (SEQ ID NO: 62). The knockout edits were performed using an RNP of 5:1 (gRNA:Cas9) with absolute values of 625 pmol gRNA and 125 pmol Cas9 per 1 million cells. This mixture was then electroporated with 1 pulse for 20 ms at 1500 V followed by 1 pulse for 100 ms at 500 V. The cells were electroporated with RNP targeting FAS twice 3 days apart to ensure near 100% knockout. Following knockout of FAS, the cells were treated with RNP targeting CISH (CISH Ex1 T18 gRNA (SEQ ID NO:50)) and were also electroporated twice 3 days apart to ensure near 100% knockout of CISH. After this targeting, the bulk population represents an enriched population of SERPINB9-P2A-IL15/IR15α KI cells with knockouts of B2M, FAS, and CISH.

This population was expanded and the cells were electroporated with a plasmid encoding anti-CD30 CAR-P2A-HLA-E trimer (e.g., SEQ ID NOS: 77, 84, or 90) and RNP targeting CIITA. This electroporation for KI was done the same way as the electroporation for KI of SERPINB9-P2A-IL15/IR15α above. At 2 days post electroporation, the cells were enriched for transfection by performing FACS (FACS #3) for HLA-E. At 7 to 10 days post electroporation, the cells were FACS (FACS #4) sorted again for HLA-E to enrich for HLA-E knock in positive cells. After FACS #4, the cells were bulk sorted to remove residual PD-L1 positive cells. This population represents an enriched bulk of SERPINB9-P2A-IL15/IR15α KI and anti-CD30 CAR-P2A-HLA-E KI double positive cells with a knockout of B2M, FAS, CISH, and CIITA (e.g., termed L5V024B (anti-CD30 CAR4), L5V025B (anti-CD30 CAR5), or L5V026B (anti-CD30 CAR6) cells). The cells were differentiated essentially as described in Example 3 and characterized. Some of the cells from the bulk population cells were single cell sorted for IL15 and HLA-E double positive cells and plated on 96 well plates for the generation of single cell clones.

### Example 13: Characterization of iNK Cells Derived from SERPINB9 KI, IL1S/IL15Rα KI, Anti-CD30 CAR KI, HLA-E KI, B2M KO, CIITA KO, CISH KO, FAS KO Cells

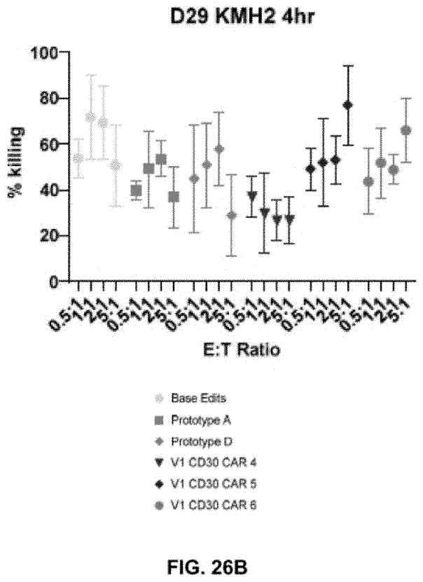

FIG. 25 presents expression patterns of CD45 and CD56 during iNK differentiation of the cells with base edits (e.g., SERPINB9-P2A-IL15/IR15α KI, B2M KO), prototype edits (e.g., SERPINB9-P2A-IL15/IR15α KI, B2M KO, CISH KO, FAS KO), and the CAR inserts (e.g., SERPINB9-P2A-IL15/IR15α KI, anti-CD30 CAR-P2A-HLA-E KI, B2M KO, FAS KO, CISH KO, and CIITA KO). By day 36, more than 99% of all the cell lines were CD45+/CD56+, indicating efficient iNK differentiation.

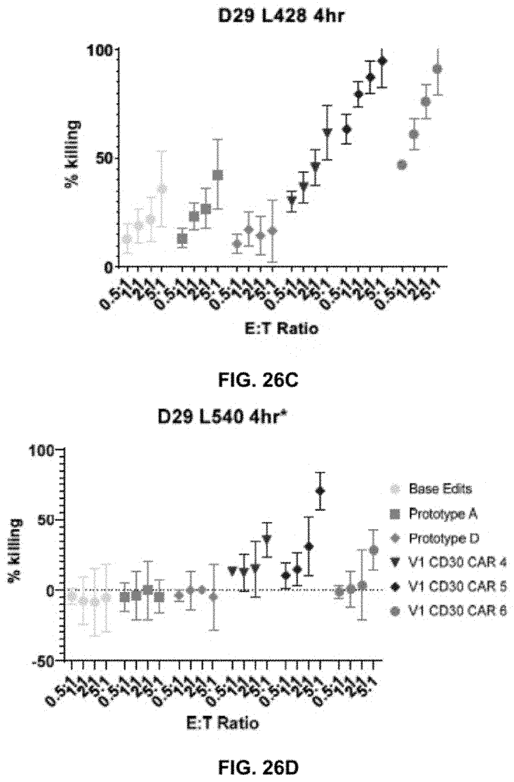

Co-incubation of day 29 iNK cells with various CD30+ cancer cells revealed that the cells with the anti-CD30 CARS were more effective killers than the cells with base edits or prototype edits (see FIGS. 26A-26D). Some of the anti-CD30 CAR cells had more than 90% killing after 4 hrs at the highest effector-target ratio (5:1). In general, CAR5 outperformed CAR4 and CAR6 in the CD30 cancer cell cytotoxicity assay.

### Example 14: In Vivo Testing of iNK Cells Derived from SERPINB9 KI, IL1S/IL15Rα KI, Anti-CD30 CAR KI, HLA-E KI, B2M KO, CIITA KO, CISH KO, FAS KO Cells

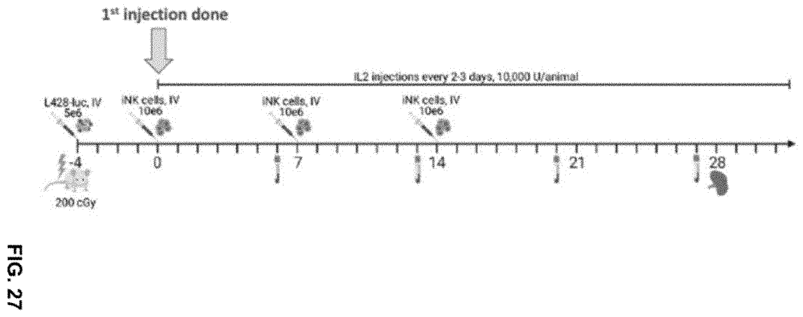

Mice were intravenously injected with 5×10⁶ L428 cancer cells labeled with luciferase. Four days later (day 0), 10×10⁶ iNK cells comprising CAR5 (2:1 E:T ratio) were intravenously injected into the mice. Two more intravenous injections of 10 million iNK cells at days 7 and 14 of iNK cells will be given, and the organs will be harvested at day 28 for cancer cell localization. FIG. 27 presents a schematic of the protocol.

What is claimed is:

1. An in vitro method for generating an engineered cell, the method comprising delivering to a cell:
(a) an RNA-guided nuclease and a gRNA targeting a target site in a B2M gene locus; and
(b) a vector comprising a nucleic acid, the nucleic acid comprising: (i) nucleotide sequence encoding a SERPINB9 protein; (ii) a nucleotide sequence having sequence homology with a genomic region located left of the target site in the B2M gene locus; and (iii) a nucleotide sequence having sequence homology with a genomic region located right of the target site in the B2M gene locus, wherein (i) is flanked by (ii) and (iii);
wherein the B2M gene locus is cleaved at the target site and the nucleotide sequences encoding the SERPINB9 protein are inserted into the B2M gene locus, thereby disrupting the B2M gene.

2. The in vitro method of claim 1, wherein the gRNA comprises a spacer sequence corresponding to a sequence consisting of SEQ ID NO: 1.

3. The in vitro method of claim 1, wherein the RNA-guided nuclease and the gRNA targeting a target site in a B2M gene locus are delivered as a ribonucleoprotein (RNP) complex.

4. The in vitro method of claim 1, wherein the nucleotide sequence of (b)(i) further comprises a nucleotide sequence encoding a IL15/IL15Rα fusion protein.

5. The in vitro method of claim 4, wherein the nucleotide sequence of (b)(i) comprises the nucleotide sequence encoding the SERPINB9 protein linked to a nucleotide sequence encoding a P2A peptide sequence linked to the nucleotide sequence encoding the IL15/IL15Rα fusion protein to form a SERPINB9-P2A-IL15/IL15Rα construct.

6. The in vitro method of claim 5, wherein the SERPINB9-P2A-IL15/IL15Rα construct consists essentially of SEQ ID NO: 37.

7. The in vitro method of claim 5, wherein the SERPINB9-P2A-IL15/IL15Rα construct is operably linked to an exogenous promoter.

8. The in vitro method of claim 7, wherein the exogenous promoter is a CAG, CMV, EF1α, PGK, or UBC promoter.

9. The in vitro method of claim 7, wherein the exogenous promoter is CAG and CAG-SERPINB9-P2A-IL15/IL15Rα consists essentially of SEQ ID NO: 38.

10. The in vitro method of claim 1, wherein the nucleotide sequence of (b)(i) further comprises a nucleotide sequence encoding an HLA-E trimer.

11. The in vitro method of claim 10, wherein the nucleotide sequence of (b)(i) comprises the nucleotide sequence encoding the SERPINB9 linked to a nucleotide sequence encoding a P2A peptide sequence linked to the nucleotide sequence encoding the HLA-E trimer to form a SERPINB9-P2A-HLA-E construct.

12. The in vitro method of claim 11, wherein the SERPINB9-P2A-HLA-E construct consists essentially of SEQ ID NO: 21.

13. The in vitro method of claim 11, wherein the SERPINB9-P2A-HLA-E construct is operably linked to an exogenous promoter.

14. The in vitro method of claim 13, wherein the exogenous promoter is a CAG, CMV, EF1α, PGK, or UBC promoter.

15. The in vitro method of claim 14, wherein the exogenous promoter is CAG and CAG-SERPINB9-P2A-HLA-E consists essentially of SEQ ID NO: 22

16. The in vitro method of claim 1, wherein the nucleotide sequence of (b)(ii) consists essentially of SEQ ID NO: 3, and the nucleotide sequence of (b)(iii) consists essentially of SEQ ID NO: 19.

17. The in vitro method of claim 1, wherein the first vector consists essentially of SEQ ID NO: 39.

18. The in vitro method of claim 1, wherein the first vector consists essentially of SEQ ID NO: 23.

19. The in vitro method of claim 1, further comprising delivering to the cell an RNA-guided nuclease and a gRNA targeting a target site in a CISH gene locus.

20. The in vitro method of claim 19, wherein the RNA-guided nuclease and gRNA are delivered as a ribonucleoprotein (RNP) complex.

21. The in vitro method of claim 19, wherein the gRNA targeting a target site in a CISH gene locus comprises a spacer sequence corresponding to a sequence consisting of any one of SEQ ID NOS: 49-60.

22. The in vitro method of claim 1, further comprising delivering to the cell an RNA-guided nuclease and a gRNA targeting a target site in a FAS gene locus.

23. The in vitro method of claim 22, wherein the RNA-guided nuclease and gRNA are delivered as a ribonucleoprotein (RNP) complex.

24. The in vitro method of claim 22, wherein the gRNA targeting a target site in a FAS gene locus comprises a spacer sequence corresponding to a sequence consisting of any one of SEQ ID NOS: 61-67.

25. The in vitro method of claim 1, wherein the cell is a pluripotent stem cell or an adult stem cell.

26. The in vitro method of claim 25, wherein the cell is an induced pluripotent stem cell, or an embryonic stem cell.

27. The in vitro method of claim 1, wherein the cell is a terminally differentiated somatic cell or a lineage restricted progenitor cell.

28. The in vitro method of claim 27, wherein the lineage restricted progenitor cell is hematopoietic progenitor cells, mesodermal cells, definitive hemogenic endothelium, definitive hematopoietic stem or progenitor cells, CD34+ cells, multipotent progenitors (MPP), common lymphoid progenitor cells, T cell progenitors, NK cell progenitors, pancreatic endoderm progenitors, pancreatic endocrine progenitors, mesenchymal progenitor cells, muscle progenitor cells, blast cells, or neural progenitor cells, and the fully differentiated somatic cell is selected from a hematopoietic cell, a pancreatic beta cell, an epithelial cell, an endodermal cell, a macrophages, a hepatocyte, an adipocyte, a kidney cell, a blood cell, a cardiomyocyte, or an immune system cell.

29. The in vitro method of claim 1, wherein the cell is a mammalian cell.
